(19) United States
(12) Patent Application Publication
(10) Pub. No.: US 2024/0140919 A1
(43) Pub. Date: May 2, 2024

(54) MODULATORS OF EUKARYOTIC INITIATION FACTOR 2

(71) Applicant: Denali Therapeutics Inc., South San Francisco, CA (US)

(72) Inventors: Robert A. Craig II, II, South San Francisco, CA (US); Javier de Vicente Fidalgo, Foster City, CA (US); Anthony A. Estrada, Danville, CA (US); Jianwen A. Feng, San Mateo, CA (US); Brian M. Fox, Redwood City, CA (US); Katrina W. Lexa, San Francisco, CA (US); Maksim Osipov, Redwood City, CA (US); Zachary K. Sweeney, Redwood City, CA (US); Arun Tottumkara, San Francisco, CA (US)

(21) Appl. No.: 18/526,983

(22) Filed: Dec. 1, 2023

Related U.S. Application Data
(63) Continuation of application No. 18/301,190, filed on Apr. 14, 2023, now abandoned.
Application No. 18/301,190 is a continuation of application No. 17/822,366, filed on Aug. 25, 2022, now abandoned.
Application No. 17/822,366 is a continuation of application No. 17/574,473, filed on Jan. 12, 2022, now abandoned.
Application No. 17/574,473 is a continuation of application No. 16/982,471, filed on Sep. 18, 2020, now abandoned.
(60) Provisional application No. 62/647,345, filed on Mar. 23, 2018.
(60) Provisional application No. 62/802,128, filed on Feb. 6, 2019.

(51) Int. Cl. C07D 271/07; C07C 235/22; C07C 255/46; C07D 205/04; C07D 205/12; C07D 209/52; C07D 211/58; C07D 221/20; C07D 233/32; C07D 401/04; C07D 413/04

(52) U.S. Cl. CPC C07D 271/07; C07C 235/22; C07C 255/46; C07D 205/04; C07D 205/12; C07D 209/52; C07D 211/58; C07D 221/20; C07D 233/32; C07D 401/04; C07D 413/04

(57) ABSTRACT

The present disclosure relates generally to eukaryotic initiation factor 2B modulators of formula A, or a pharmaceutically acceptable salt, stereoisomer, or mixture of stereoisomers thereof and methods of making and using thereof.

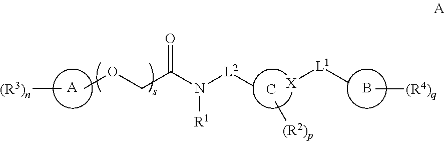

### CROSS REFERENCE TO RELATED APPLICATIONS

This application claims the benefit under 35 U.S.C. § 119(e) to U.S. Provisional Application Nos. 62/647,345, filed Mar. 23, 2018, and 62/802,128, filed Feb. 6, 2019, each of which is incorporated by reference in its entirety.

### FIELD

The present disclosure relates generally to small molecule modulators of eukaryotic initiation factor 2B and their use as therapeutic agents, for example, in treating diseases mediated thereby such as Alzheimer's, Parkinson's, ALS, frontotemporal dementia, and cancer.

### BACKGROUND

Neurodegenerative diseases, such as Parkinson's disease (PD), amyotrophic lateral sclerosis (ALS), Alzheimer's disease (AD), and Frontotemporal dementia (FTD) have a negative effect on the lives of millions of people.

The multi-subunit protein complexes eukaryotic initiation factor 2B and eukaryotic initiation factor 2 are required for protein synthesis initiation and regulation in eukaryotic cells. Eukaryotic initiation factor 2B is composed of five subunits (α, β, γ, δ and ε), and eukaryotic initiation factor 2 is composed of three subunits (α, δ and γ). Eukaryotic initiation factor 2B functions as a guanine nucleotide exchange factor (GEF) that catalyzes the exchange of guanosine-5′-diphosphate (GDP) with guanosine-5′-triphosphate (GTP) on eukaryotic initiation factor 2, thereby allowing the GTP bound eukaryotic initiation factor 2 to bind to the initiating methionine transfer RNA and initiate protein synthesis.

Eukaryotic initiation factor 2B is active when complexed as a ten subunit dimer. Eukaryotic initiation factor 2 is active when bound to GTP and inactive when bound to GDP. Moreover, when the a subunit of eukaryotic initiation factor 2 is phosphorylated on serine 51, it inhibits and regulates the guanine nucleotide exchange activity of eukaryotic initiation factor 2B. In its phosphorylated form, eukaryotic initiation factor 2 remains in an inactive GDP bound state and translation initiation is blocked.

The interaction between eukaryotic initiation factor 2B and eukaryotic initiation factor 2 plays an important role in the integrated stress response (ISR) pathway. Activation of this pathway leads in part to ATF4 (Activating Transcription Factor 4) expression and stress granule formation. Aberrant TSR activation is found in multiple neurodegenerative diseases, with a strong functional link to pathology characterized by the RNA-binding/stress-granule protein TAR DNA binding protein (TARDBP), also known as TDP43. Activation of eIF2B inhibits the ISR and ISR dependent stress granule formation and is found to be neuroprotective in multiple disease models.

Impairment of eukaryotic initiation factor 2B activity is correlated to activation of the ISR pathway that is implicated in a variety neurodegenerative diseases including Parkinson's disease, amyotrophic lateral sclerosis (ALS), Alzheimer's disease and frontotemporal dementia. Mutations in TDP43 and other RNA-binding proteins/stress-granule proteins alter stress-granule dynamics and cause ALS. Inhibition of the ISR pathway can block and promote the dissolution of stress-granules. In addition, mutations in the human eukaryotic initiation factor 2B subunits have been identified as causing leukoencephalopathy with vanishing white matter (VWM) and childhood ataxia with central nervous system hypomyelination (CACH). In VWM/CACH patients, white matter lesions severely deteriorate and neurological disorders are exacerbated after stresses, and their eukaryotic initiation factor 2B guanine nucleotide exchange activities are generally lower than normal.

### DESCRIPTION

Provided herein are compounds, or a pharmaceutically acceptable salt, isotopically enriched analog, stereoisomer, mixture of stereoisomers or prodrug thereof, that are useful in treating and/or preventing diseases mediated, at least in part, by eukaryotic initiation factor 2B, such as neurodegenerative diseases (e.g., neurodegeneration in prion disease) and cancer.

In some embodiments, provided are compounds that modulate the activity of eukaryotic initiation factor 2B. In some embodiments, the compounds modulate the regulation of eukaryotic initiation factor 2B. In some aspects the compounds modulate the inhibition of eukaryotic initiation factor 2B by phosphorylated eukaryotic initiation factor 2. In some embodiments, the compounds interfere with the interaction between eukaryotic initiation factor 2B and phosphorylated eukaryotic initiation factor 2. In some embodiments, the phosphorylated eukaryotic initiation factor 2 is phosphorylated on its alpha subunit (eukaryotic initiation factor 2 α phosphate).

In some embodiments, provided are compounds that act as activators of eukaryotic initiation factor 2B by increasing its GDP/GTP nucleotide exchange activity. In some embodiments, the compounds promote eukaryotic initiation factor 2B dimer formation. In other embodiments, the compounds enhances the guanine nucleotide exchange factor (GEF) activity of eukaryotic initiation factor 2B. In other embodiments, the compounds increases the guanine nucleotide exchange factor (GEF) activity of eukaryotic initiation factor 2B on its eukaryotic initiation factor 2/GDP substrate.

In some embodiments, provided are compounds that desensitizes cells to the deleterious effects of eukaryotic initiation factor 2B inhibition. In some embodiments the deleterious effects include ATF4 expression and stress granule formation.

In another embodiment, provided is a pharmaceutical composition comprising a compound as described herein, or a pharmaceutically acceptable salt, isotopically enriched analog, stereoisomer, mixture of stereoisomers or prodrug thereof, and a pharmaceutically acceptable carrier.

In another embodiment, provided is a method for treating a disease or condition mediated, at least in part, by eukaryotic initiation factor 2B, the method comprising administering an effective amount of the pharmaceutical composition comprising a compound as described herein, or a pharmaceutically acceptable salt, isotopically enriched analog, stereoisomer, mixture of stereoisomers or prodrug thereof.

In another embodiment, provided is a method for treating a disease or condition mediated, at least in part, by regulation of eukaryotic initiation factor 2B, the method comprising administering an effective amount of the pharmaceutical composition comprising a compound as described herein, or a pharmaceutically acceptable salt, isotopically enriched analog, stereoisomer, mixture of stereoisomers or prodrug thereof, and a pharmaceutically acceptable carrier, to a subject in need thereof.

In another embodiment, provided is a method for promoting or stabilizing eukaryotic initiation factor 2B dimer formation, the method comprising administering an effective amount of the pharmaceutical composition comprising a compound as described herein, or a pharmaceutically acceptable salt, isotopically enriched analog, stereoisomer, mixture of stereoisomers or prodrug thereof, and a pharmaceutically acceptable carrier, to a subject in need thereof.

In another embodiment, provided is a method for promoting eukaryotic initiation factor 2B activity, the method comprising administering an effective amount of the pharmaceutical composition comprising a compound as described herein, or a pharmaceutically acceptable salt, isotopically enriched analog, stereoisomer, mixture of stereoisomers or prodrug thereof, and a pharmaceutically acceptable carrier, to a subject in need thereof.

In another embodiment, provided is a method for desensitizing cells to eukaryotic initiation factor 2 phosphorylation, the method comprising administering an effective amount of the pharmaceutical composition comprising a compound as described herein, or a pharmaceutically acceptable salt, isotopically enriched analog, stereoisomer, mixture of stereoisomers or prodrug thereof, and a pharmaceutically acceptable carrier, to a subject in need thereof.

In another embodiment, provided is a method for inhibiting the integrated stress response pathway, the method comprising administering an effective amount of the pharmaceutical composition comprising a compound as described herein, or a pharmaceutically acceptable salt, isotopically enriched analog, stereoisomer, mixture of stereoisomers or prodrug thereof, and a pharmaceutically acceptable carrier, to a subject in need thereof.

In another embodiment, provided is a method for inhibiting stress granule formation, the method comprising administering an effective amount of the pharmaceutical composition comprising a compound as described herein, or a pharmaceutically acceptable salt, isotopically enriched analog, stereoisomer, mixture of stereoisomers or prodrug thereof, and a pharmaceutically acceptable carrier, to a subject in need thereof.

In another embodiment, provided is a method for inhibiting ATF4 expression, the method comprising administering an effective amount of the pharmaceutical composition comprising a compound as described herein, or a pharmaceutically acceptable salt, isotopically enriched analog, stereoisomer, mixture of stereoisomers or prodrug thereof, and a pharmaceutically acceptable carrier, to a subject in need thereof.

In another embodiment, provided is a method for inhibiting ATF4 translation, the method comprising administering an effective amount of the pharmaceutical composition comprising a compound as described herein, or a pharmaceutically acceptable salt, isotopically enriched analog, stereoisomer, mixture of stereoisomers, or prodrug thereof, and a pharmaceutically acceptable carrier, to a subject in need thereof.

The disclosure also provides compositions, including pharmaceutical compositions, kits that include the compounds, and methods of using (or administering) and making the compounds. The disclosure further provides compounds or compositions thereof for use in a method of treating a disease, disorder, or condition that is mediated, at least in part, by eukaryotic initiation factor 2B. Moreover, the disclosure provides uses of the compounds or compositions thereof in the manufacture of a medicament for the treatment of a disease, disorder, or condition that is mediated, at least in part, by eukaryotic initiation factor 2B.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### 1. DEFINITIONS

As used in the present specification, the following words, phrases and symbols are generally intended to have the meanings as set forth below, except to the extent that the context in which they are used indicates otherwise.

A dash (“—”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl) or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, a divalent heteroaryl group, etc., may also be referred to as an “alkylene” group or an “alkylenyl” (for example, methylenyl, ethylenyl, and propylenyl) group, an “arylene” group or an “arylenyl” group (for example, phenylenyl or napthylenyl), or a “heteroarylene” group (for example quinolindiyl), respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkenyl” refers to an alkyl group containing at least one carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl) or 2 to 4 carbon atoms (i.e., C₂₋₄ alkenyl). Examples of alkenyl groups include, e.g., ethenyl, propenyl, butadienyl (including 1,2-butadienyl and 1,3-butadienyl).

“Alkynyl” refers to an alkyl group containing at least one carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl) or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy and 1,2-di methylbutoxy.

“Alkoxyalkyl” refers to the group “alkyl-O-alkyl”.

“Alkylthio” refers to the group “alkyl-S—”. “Alkylsulfinyl” refers to the group “alkyl-S(O)—”. “Alkylsulfonyl” refers to the group “alkyl-S(O)₂—”. “Alkylsulfonylalkyl” refers to -alkyl-S(O)₂-alkyl.

“Acyl” refers to a group —C(O)R^{y}, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of acyl include, e.g., formyl, acetyl, cyclohexylcarbonyl, cyclohexylmethyl-carbonyl and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein, or R^{y} and R^{z} are taken together to form a cycloalkyl or heterocyclyl; each of which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Arylalkyl” or “Aralkyl” refers to the group “aryl-alkyl-”.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl or heteroaryl; each of which may be optionally substituted, as defined herein.

“Cyanoalkyl” refers to refers to an alkyl group as defined above, wherein one or more (e.g., 1 or 2) hydrogen atoms are replaced by a cyano (—CN) group.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Cycloalkoxy” refers to “—O-cycloalkyl.”

“Cycloalkylalkyl” refers to the group “cycloalkyl-alkyl-”.

“Cycloalkylalkoxy” refers to “—O-alkyl-cycloalkyl.”

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Halogen” or “halo” refers to atoms occupying group VIIA of the periodic table, such as fluoro, chloro, bromo or iodo.

“Haloalkyl” refers to an unbranched or branched alkyl group as defined above, wherein one or more (e.g., 1 to 6 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

“Haloalkoxy” refers to an alkoxy group as defined above, wherein one or more (e.g., 1 to 6 or 1 to 3) hydrogen atoms are replaced by a halogen.

“Hydroxyalkyl” refers to an alkyl group as defined above, wherein one or more (e.g., 1 to 6 or 1 to 3) hydrogen atoms are replaced by a hydroxy group.

“Heteroalkyl” refers to an alkyl group in which one or more of the carbon atoms (and any associated hydrogen atoms) are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.) and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkyl includes 1 to 10 carbon atoms, 1 to 8 carbon atoms, or 1 to 4 carbon atoms; and 1 to 3 heteroatoms, 1 to 2 heteroatoms, or 1 heteroatom.

“Heteroalkylene” refers to a divalent alkyl group (i.e., alkylene) in which one or more of the carbon atoms (and any associated hydrogen atoms) are each independently replaced with the same or different heteroatomic group. The term “heteroalkylene” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of heteroalkylene groups include, e.g., —CH₂OCH₂—, —CH(CH₃)OCH₂—, —CH₂CH₂OCH₂—, —CH₂CH₂OCH₂CH₂OCH₂—, —CH₂SCH₂—, —CH(CH₃)SCH₂—, —CH₂CH₂SCH₂—, —CH₂CH₂SCH₂CH₂SCH₂—, —CH₂S(O)₂CH₂—, —CH(CH₃)S(O)₂CH₂—, —CH₂CH₂S(O)₂CH₂—, —CH₂CH₂S(O)₂CH₂CH₂OCH₂—, —CH₂NR^{y}CH₂—, —CH(CH₃)NR^{y}CH₂—, —CH₂CH₂NR^{y}CH₂—, —CH₂CH₂NR^{y}CH₂CH₂NR^{y}CH₂—, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkyl includes 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more (e.g., 1 to 3) oxo (═O) or N-oxide (—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), tetrahydropyranyl, thiomorpholinyl, thiamorpholinyl, 1-oxo-thiomorpholinyl and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

“Heterocyclylalkyl” refers to the group “heterocyclyl-alkyl-”.

“Oxime” refers to the group —CR^{y}(═NOH) wherein R^{y} is hydrogen, alkyl, alkenyl, alkynyl, cycloalkyl, heterocyclyl, aryl, heteroalkyl or heteroaryl; each of which may be optionally substituted, as defined herein.

“Sulfonyl” refers to the group —S(O)₂R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfonyl are methylsulfonyl, ethylsulfonyl, phenylsulfonyl and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfinyl are methylsulfinyl, ethylsulfinyl, phenylsulfinyl and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5 or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5 or 1 to 3) hydrogen atom is replaced by a bond to a non-hydrogen atom such as, but not limited to alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanadino, halo, haloalkyl, haloalkoxy, hydroxyalkyl, heteroalkyl, heteroaryl, heteroarylalkyl, heterocyclyl, heterocyclylalkyl, —NHNH₂, ═NNH₂, imino, imido, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide or —Si(R^{y})₃, wherein each R^{y} is independently hydrogen, alkyl, alkenyl, alkynyl, heteroalkyl, cycloalkyl, aryl, heteroaryl or heterocyclyl.

In certain embodiments, “substituted” includes any of the above alkyl, alkenyl, alkynyl, cycloalkyl, heterocyclyl, aryl or heteroaryl groups in which one or more (e.g., 1 to 5 or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(═O)R^{h}, —NR^{g}C(═O)NR^{g}R^{h}, —NR^{g}C(═O)OR^{h}, —NR^{g}S(═O)₁₋₂R^{h}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, —NR^{g}S(═O)₁₋₂NR^{g}R^{h}, ═NSO₂R^{g}, ═NOR^{g}, —S(═O)₁₋₂NR^{g}R^{h}, —SF₅, —SCF₃ or —OCF₃. In certain embodiments, “substituted” also means any of the above groups in which one or more (e.g., 1 to 5 or 1 to 3) hydrogen atoms are replaced with —C(═O)R^{g}, —C(═O)OR^{g}, —C(═O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5 or 1 to 3) hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} and Rⁱ are taken together with the atoms to which they are attached to form a heterocyclyl ring optionally substituted with oxo, halo or alkyl optionally substituted with oxo, halo, amino, hydroxyl, or alkoxy.

In certain embodiments, as used herein, the phrase “one or more” refers to one to five. In certain embodiments, as used herein, the phrase “one or more” refers to one to three.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as 3H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also or a pharmaceutically acceptable salt, isotopically enriched analog, deuterated analog, stereoisomer, mixture of stereoisomers, and prodrugs of the compounds described herein.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds of the invention, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present invention contemplates various stereoisomers or mixtures thereof and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein and the like. Preparation, selection and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

### 2. COMPOUNDS

Provided herein are compounds that are modulators of eukaryotic initiation factor 2B. In certain embodiments, provided is a compound of Formula A:

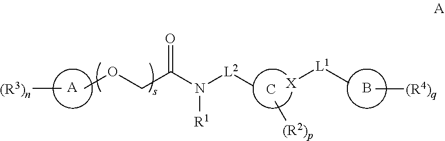

or a pharmaceutically acceptable salt, isotopically enriched analog, stereoisomer, mixture of stereoisomers or prodrug thereof, wherein:

A and B are independently C₃₋₁₀ cycloalkyl, heterocyclyl, aryl, or heteroaryl, provided that at least one of A or B is C₃₋₁₀ cycloalkyl;

X is C or N;

C is cycloalkyl when X is C or heterocyclyl when X is N;

L¹ is —NR⁹C(O)CH₂O—, —C(O)NR⁹CH₂O—, a heteroalkylene optionally substituted with one to six R⁵, or L¹ is a heterocyclyl or heteroaryl, each of which is optionally substituted with one to six R¹³;

provided that when C is a bicyclo[1.1.1]pentane or bicyclo[2.1.1]hexane, then L¹ is —NR⁹C(O)CH₂O— or —C(O)NR⁹CH₂O— and L² is a bond;

L² is a bond or a C₁₋₂ alkylene optionally substituted with one to four R⁵;

n is 0, 1, 2, 3, 4, 5, or 6;

p is 0, 1, 2, 3, 4, 5, 6, 7, or 8;

q is 0, 1, 2, 3, 4, 5, or 6;

s is 0 or 1;

R¹ is hydrogen, C₁₋₁₂ alkyl, C₂₋₁₂ alkenyl, C₂₋₁₂ alkynyl, C₃₋₁₀ cycloalkyl or heterocyclyl, each of which, other than hydrogen, is optionally substituted with one to six R¹¹;

each R² is independently halo, cyano, —NR⁶R⁷, hydroxyl, oxo, —C(O)OR⁶, —OC(O)NR⁶R⁷, —C(O)NR⁶R⁷, —NR⁶C(O)R⁷, C₁₋₁₂ alkoxy, C₁₋₁₂ haloalkoxy, C₁₋₂ alkyl, C₁₋₁₂ haloalkyl, heteroaryl, heterocyclyl, and cycloalkyl, wherein each of heterocyclyl, heteroaryl, and cycloalkyl are independently optionally substituted with one to six cyano, halo, C₁₋₁₂ alkyl, or C₁₋₁₂ haloalkyl, or two R² on non-adjacent ring atoms together form a bond, C₁₋₃ alkylene optionally substituted with one to six R⁵, or C₁₋₂ heteroalkylene optionally substituted with one to four R⁵, provided that when C is cyclobutyl then R² is not oxo;

each R⁵ is independently halo, C₁₋₆ alkyl or C₁₋₆ haloalkyl;

R³ and R⁴ are independently R¹¹;

each R¹¹ is independently halo, cyano, nitro, oxo, —OR⁶, —SR⁶, —SF₅, —NR⁶R⁷, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R⁶, —C(O)OR⁶, —OC(O)OR⁶, —OC(O)R⁶, —C(O)NR⁶R⁷, —OC(O)NR⁶R⁷, —NR⁶C(O)NR⁷R⁸, —S(O)₁₋₂R⁶, —S(O)₁₋₂NR⁶, —NR⁶S(O)₁₋₂R⁷, —NR⁶S(O)₁₋₂NR⁷R⁸, —NR⁶C(O)R⁷ or —NR⁶C(O)OR⁷, wherein each C₁₋₁₂ alkyl, C₂₋₁₂ alkenyl, C₂₋₁₂ alkynyl, C₃₋₁₀ cycloalkyl, heterocyclyl, aryl and heteroaryl of R¹¹ is independently optionally substituted with one to six R¹²;

each of R⁶, R⁷, and R⁸ is independently hydrogen, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R²⁰, —C(O)OR²⁰, —C(O)NR²⁰R²¹, —S(O)₁₋₂R²⁰ or —S(O)₁₋₂NR²⁰, wherein each C₁₋₁₂ alkyl, C₂₋₁₂ alkenyl, C₂₋₁₂ alkynyl, C₃₋₁₀ cycloalkyl, heterocyclyl, aryl and heteroaryl of R⁶, R⁷, and R⁸ is independently optionally substituted with one to six R¹²; or

two of R⁶, R⁷, and R⁸ are taken together with the atoms to which they are attached to form heterocyclyl independently optionally substituted by one to six halo, or C₁₋₂ alkyl independently optionally substituted by one to six oxo, halo, hydroxyl or amino;

R⁹ is independently hydrogen or C₁₋₁₂ alkyl optionally substituted with one to six halo;

each R¹² is independently halo, cyano, nitro, oxo, —OR³⁰, —SR³⁰, —SF₅, —NR³⁰R³¹, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R³⁰, —C(O)OR³⁰, —OC(O)OR³⁰, —OC(O)R³⁰, —C(O)NR³⁰R³¹, —OC(O)NR³⁰R³¹, —NR³⁰C(O)NR³⁰R³¹, —S(O)₁₋₂R³⁰, —S(O)₁₋₂NR³⁰, —NR³⁰S(O)₁₋₂R³¹, —NR³⁰S(O)₁₋₂NR³⁰R³¹, —NR³⁰C(O)R³¹ or —NR³⁰C(═O)OR³¹, wherein each C₁₋₁₂ alkyl, C₂₋₁₂ alkenyl, C₂₋₁₂ alkynyl, C₃₋₁₀ cycloalkyl, heterocyclyl, aryl and heteroaryl of R¹² is independently optionally substituted with one to six halo or C₁₋₁₂ alkyl independently optionally substituted by one to six oxo, halo, hydroxyl or amino;

each R¹³ is independently halo, cyano, nitro, oxo, —OR³⁰, —SR³⁰, —SF₅, —NR³⁰R³¹, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R³⁰, —C(O)OR³⁰, —OC(O)OR³⁰, —OC(O)R³⁰, —C(O)NR³⁰R³¹, —OC(O)NR³⁰R³¹, —NR³⁰C(O)NR³⁰R³¹, —S(O)₁₋₂R³⁰, —S(O)₁₋₂NR³⁰, —NR³⁰S(O)₂R³¹, —NR³⁰S(O)₁₋₂NR³⁰R³¹, —NR³⁰C(O)R³¹ or —NR³⁰C(═O)OR³¹, wherein each C₁₋₁₂ alkyl, C₂₋₁₂ alkenyl, C₂₋₁₂ alkynyl, C₃₋₁₀ cycloalkyl, heterocyclyl, aryl and heteroaryl of R¹³ is independently optionally substituted with one to six halo or C₁₋₁₂ alkyl independently optionally substituted by one to six oxo, halo, hydroxyl or amino;

each R²⁰ and R²¹ is independently hydrogen or C₁₋₁₂ alkyl independently optionally substituted with one to six oxo, halo, hydroxyl or amino; or

R²⁰ and R²¹ are taken together with the atoms to which they are attached to form heterocyclyl independently optionally substituted by one to six halo or C₁₋₁₂ alkyl independently optionally substituted by one to six oxo, halo, hydroxyl or amino; and

each R³⁰ and R³¹ is independently hydrogen or C₁₋₁₂ alkyl independently optionally substituted with one to six oxo, halo, hydroxyl or amino;

or R³⁰ and R³¹ are taken together with the atoms to which they are attached to form heterocyclyl independently optionally substituted by one or to six halo or C₁₋₁₂ alkyl independently optionally substituted by one to six oxo, halo, hydroxyl or amino.

In certain embodiments, when X is C and L¹ is 5-membered heterocyclyl attached to X at a nitrogen atom, then R² is not oxo.

In certain embodiments, provided is a compound of formula I:

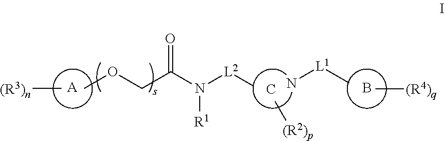

or a pharmaceutically acceptable salt, isotopically enriched analog, stereoisomer, mixture of stereoisomers or prodrug thereof, wherein

L¹ is a heteroalkylene optionally substituted with one to six R⁵ or L¹ is a heterocyclyl or heteroaryl, each of which is optionally substituted with one to six R¹³.

In certain embodiments, the compound is not 2-(4-chloro-2-methylphenoxy)-N-[1-(5-cyclopropyl-1H-pyrazol-3-yl)-4-piperidinyl]-acetamide.

In certain embodiments, the compound is not N-[1-(5-cyclopropyl-1H-pyrazol-3-yl)-4-piperidinyl]-2-phenoxy-acetamide.

In certain embodiments, when s is 0, two of R² on non-adjacent ring atoms together form a bond, C₁₋₃ alkylene optionally substituted with one to six R⁵, or C₁₋₂ heteroalkylene optionally substituted with one to four R⁵.

In certain embodiments, provided is a compound of formula IA:

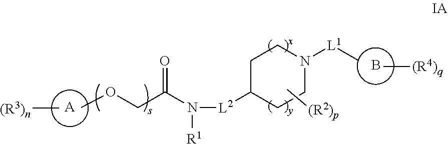

or a pharmaceutically acceptable salt, isotopically enriched analog, stereoisomer, mixture of stereoisomers or prodrug thereof, wherein x and y are independently 0, 1, or 2.

In certain embodiments, provided is a compound of Formula IA-1:

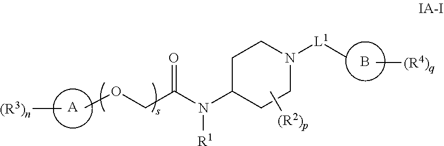

or a pharmaceutically acceptable salt, isotopically enriched analog, stereoisomer, mixture of stereoisomers or prodrug thereof.

In certain embodiments, provided is a compound of Formula IA-2:

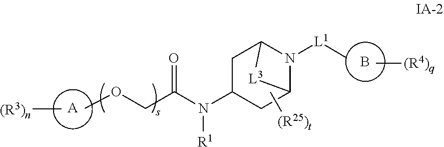

or a pharmaceutically acceptable salt, isotopically enriched analog, stereoisomer, mixture of stereoisomers or prodrug thereof, wherein

L³ is a bond or C₁₋₃ alkylene optionally substituted with one to six R⁵, or C₁₋₂ heteroalkylene optionally substituted with one to four R⁵;

R²⁵ is independently halo, C₁₋₆ alkyl or C₁₋₆ haloalkyl; and

t is 0, 1, 2, 3, 4, 5, 6, 7, or 8.

In certain embodiments, provided is a compound of Formula IA-3:

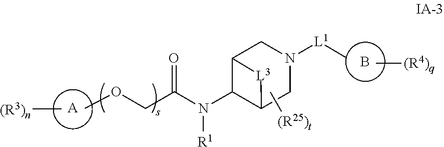

or a pharmaceutically acceptable salt, isotopically enriched analog, stereoisomer, mixture of stereoisomers or prodrug thereof, wherein

L³ is a bond or C₁₋₃ alkylene optionally substituted with one to six R⁵, or C₁₋₂ heteroalkylene optionally substituted with one to four R⁵;

R²⁵ is independently halo, C₁₋₆ alkyl or C₁₋₆ haloalkyl; and

t is 0, 1, 2, 3, 4, 5, 6, 7, or 8.

In certain embodiments, provided is a compound of Formula IA-4:

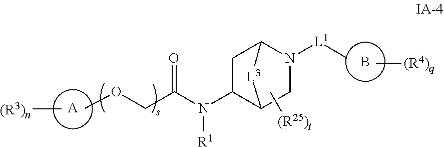

or a pharmaceutically acceptable salt, isotopically enriched analog, stereoisomer, mixture of stereoisomers or prodrug thereof, wherein

L³ is a bond or C₁₋₃ alkylene optionally substituted with one to six R⁵, or C₁₋₂ heteroalkylene optionally substituted with one to four R⁵;

R²⁵ is independently halo, C₁₋₆ alkyl or C₁₋₆ haloalkyl; and

t is 0, 1, 2, 3, 4, 5, 6, 7, or 8.

In certain embodiments, provided is a compound of Formula IA-5:

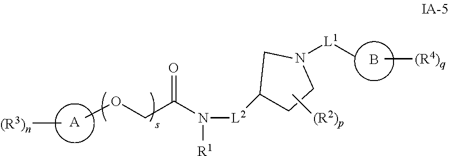

or a pharmaceutically acceptable salt, isotopically enriched analog, stereoisomer, mixture of stereoisomers or prodrug thereof.

In certain embodiments, provided is a compound of Formula IA-6:

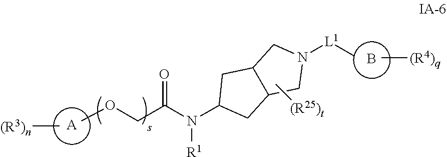

or a pharmaceutically acceptable salt, isotopically enriched analog, stereoisomer, mixture of stereoisomers or prodrug thereof.

In certain embodiments, provided is a compound of Formula IA-7:

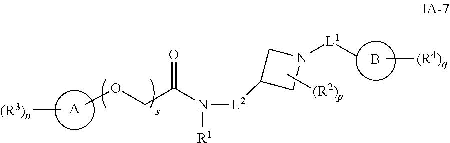

or a pharmaceutically acceptable salt, isotopically enriched analog, stereoisomer, mixture of stereoisomers or prodrug thereof.

In certain embodiments, L³ is —CH₂—, —CH₂CH₂—, —CH₂CH₂CH₂—, or —CH₂OCH₂—, each of which is optionally substituted with one to six R⁵.

In certain embodiments, provided is a compound is represented by Formula II:

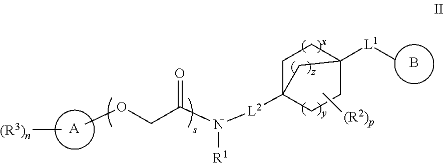

or a pharmaceutically acceptable salt, isotopically enriched analog, stereoisomer, mixture of stereoisomers or prodrug thereof, wherein x, y, and z are independently 0, 1, or 2, and B is optionally substituted with (R⁴)_{q}.

In certain embodiments, provided is a compound is represented by Formula IT:

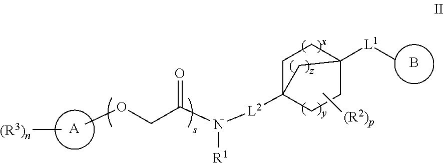

or a pharmaceutically acceptable salt, isotopically enriched analog, stereoisomer, mixture of stereoisomers or prodrug thereof, wherein x, y, and z are independently 0, 1, or 2.

The compound of claim 7, wherein z is 0.

In certain embodiments, provided is a compound is represented by Formula IIA:

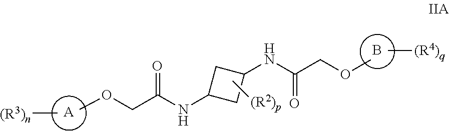

or a pharmaceutically acceptable salt, isotopically enriched analog, stereoisomer, mixture of stereoisomers or prodrug thereof.

In certain embodiments, provided is a compound is represented by Formula IIB:

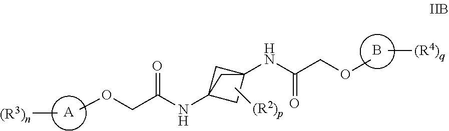

or a pharmaceutically acceptable salt, isotopically enriched analog, stereoisomer, mixture of stereoisomers or prodrug thereof.

In certain embodiments, provided is a compound is represented by Formula IIC:

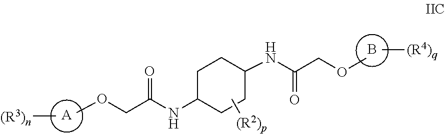

or a pharmaceutically acceptable salt, isotopically enriched analog, stereoisomer, mixture of stereoisomers or prodrug thereof.

In certain embodiments, provided is a compound is represented by Formula IID:

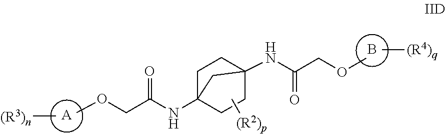

or a pharmaceutically acceptable salt, isotopically enriched analog, stereoisomer, mixture of stereoisomers or prodrug thereof.

In certain embodiments, provided is a compound is represented by Formula IIE:

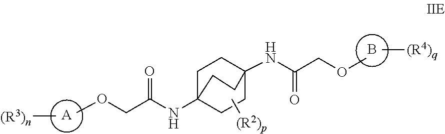

or a pharmaceutically acceptable salt, isotopically enriched analog, stereoisomer, mixture of stereoisomers or prodrug thereof.

In certain embodiments, provided is a compound is represented by Formula IIIA:

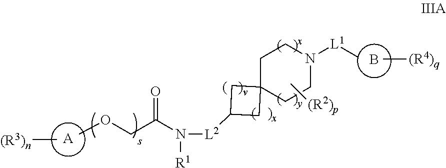

or a pharmaceutically acceptable salt, isotopically enriched analog, stereoisomer, mixture of stereoisomers or prodrug thereof, wherein x and y are independently 0, 1, or 2, and v and w are independently 1 or 2.

In certain embodiments, provided is a compound is represented by Formula IIIB:

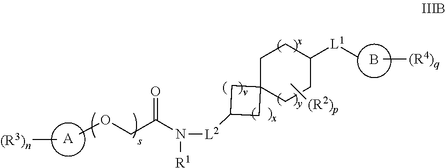

or a pharmaceutically acceptable salt, isotopically enriched analog, stereoisomer, mixture of stereoisomers or prodrug thereof, wherein x and y are independently 0, 1, or 2, and v and w are independently 1 or 2.

In certain embodiments, v, w, x, and y are each 1.

In certain embodiments, v and w are each 1, and x and y are each 0.

In certain embodiments, L¹ is an optionally substituted heteroaryl ring.

In certain embodiments, L¹ is a six membered C₂₋₄ heteroaryl ring optionally substituted with one to six R¹³.

In certain embodiments, L¹ is a pyrimidyl optionally substituted with one to six R¹³.

In certain embodiments, L¹ is a five membered C₂₋₄ heteroaryl ring optionally substituted with one to six R¹³.

In certain embodiments, L¹ is a five membered C₂₋₄ heteroaryl ring having 1 to 3 nitrogen ring atoms and optionally substituted with one to six R¹³.

In certain embodiments, L¹ is a pyrazolyl, triazolyl, oxazolyl, imidazolyl, oxadiazolyl, or isoxazole, each optionally substituted with one to six R¹³.

In certain embodiments, L¹ is a triazolyl, oxazolyl, imidazolyl, oxadiazolyl, or isoxazolyl, each optionally substituted with one to six R¹³.

In certain embodiments, L¹ is a heterocyclyl ring optionally substituted with one to six R¹³.

In certain embodiments, L¹ is a five membered C₂₋₄ heterocyclyl optionally substituted with one to six R¹³.

In certain embodiments, L¹ is a five membered C₂₋₄ heterocyclyl ring having 1 to 3 nitrogen ring atoms and optionally substituted with one to six R¹³.

In certain embodiments, L¹ is a imidazolidinonyl, dihydroisoxazolyl or oxazolidinyl, each optionally substituted with one to six R¹³. In certain embodiments, L¹ is a dihydroisoxazolyl or oxazolidinyl, each optionally substituted with one to six R¹³.

In certain embodiments, L¹ is substituted with one to five R³ where each R¹³ is independently selected from halo, cyano, oxo, C₁₋₆ alkyl, C₁₋₆ alkoxy, C₁₋₆ haloalkyl, or C₁₋₆ haloalkoxy.

In certain embodiments, L¹ is —C(O)CH₂O—, —OCH₂CH₂O—, —CH₂CH₂O—, or —CH₂CF₂CH₂O—.

In certain embodiments, L¹ is —C(O)CH₂O—, —OCH₂CH₂O—, or —CH₂CH₂O—.

In certain embodiments, L² is a bond.

In certain embodiments, L² is CH₂.

In certain embodiments, R¹ is H.

In certain embodiments, p is 0.

In certain embodiments, p or t is 1 or 2.

In certain embodiments, R² or R²⁵ is halo.

In certain embodiments, R² or R²⁵ is C₁₋₆ alkoxy.

In certain embodiments, R² or R²⁵ is methoxy.

In certain embodiments, p is 0.

In certain embodiments, q is 1.

In certain embodiments, q is 2.

In certain embodiments, n is 1.

In certain embodiments, n is 2.

In certain embodiments, R³ and R⁴ are independently hydroxyl, halo(C₁₋₆ alkoxy), halo, heteroaryl, heterocyclyl, cycloalkyl, cycloalkoxy, phenyl, C₁₋₆ alkoxycarbonyl, cyano, halo(C₁₋₆ alkyl), halo(C₁₋₆ alkoxy)cycloalkoxy, halo(C₁₋₆ alkoxy)alkyl, halo(heterocyclyl) or halophenoxy.

In certain embodiments, R³ or R⁴ is halo(C₁₋₆ alkoxy).

In certain embodiments, R³ or R⁴ is trifluoromethoxy.

In certain embodiments, A is C₃₋₁₀ cycloalkyl, aryl, or heteroaryl, each of which is optionally substituted with (R³)ₙ.

The compound of any one of the preceding claims, wherein A is cyclobutyl, triazolyl or phenyl, each of which is optionally substituted with (R³)ₙ.

The compound of any one of the preceding claims, wherein A is cyclobutyl, triazolyl, phenyl, benzothiazolyl, quinolinyl, or chromanyl each of which is optionally substituted with (R³)ₙ.

The compound of any one of the preceding claims, wherein A is phenyl optionally substituted with (R³)ₙ.

In certain embodiments, A is phenyl optionally substituted with one to six R³ independently selected from halo, cyano, C₁₋₁₂ alkyl optionally substituted with one to six halo, or C₁₋₁₂ alkoxy optionally substituted with one to six halo.

In certain embodiments, A is phenyl substituted with chloro, fluoro or a combination thereof.

In certain embodiments, the -A(R³)ₙ is 4-chlorophenyl, 4-fluorophenyl, 4-chloro-3-fluorophenyl, 4-chloro-2-fluorophenyl, 2,4-difluorophenyl, 3,4-difluorophenyl, 4-methylphenyl, 2-((trifluoromethoxy)methyl)cyclopropyl or 3-(trifluoromethoxy)cyclobutyl.

In certain embodiments, -A(R³)ₙ is 4-chlorophenyl, 4-fluorophenyl, 4-chloro-3-fluorophenyl, 4-chloro-2-fluorophenyl, 2,4-difluorophenyl, 3,4-difluorophenyl, 4-methylphenyl, 2-((trifluoromethoxy)methyl)cyclopropyl, 3-(trifluoromethoxy)cyclobutyl, 5-chlorobenzo[d]thiazolyl, 6-(trifluoromethyl)quinolinyl, 6-chloroquinolinyl, 6-flouroquinolinyl, 6,7-difluoroquinolinyl, or 6-(trifluoromethyl)chromanyl.

In certain embodiments, B is cyclopropyl, cyclobutyl, cyclopentyl, phenyl, azetidinyl, pyrrolidinyl, or tetrahydrofuranyl, each optionally substituted with (R⁴)_{q}.

In certain embodiments, B is cyclobutyl optionally substituted with (R⁴)_{q}.

The compound of any one of the preceding claim wherein B is phenyl optionally substituted with (R⁴)_{q}.

In certain embodiments, at least one R⁴ is (4-chloro-3-fluoro-phenoxy)methyl, 1,1,1-trifluoroethyl, 3-(trifluoromethoxy)cyclobutoxymethyl, 3-(trifluoromethoxy)propyl, 3,3-difluoro-1-methyl-propyl, benzyl, cyclobutoxymethyl, cyclobutylmethyl, cyclobutylmethyl, cyclopropylethyl, cyclopropylmethyl, methyl, trifluoroethyl, trifluoromethoxy, (trifluoromethoxy)cyclobutoxy, trifluoromethoxyethyl, trifluoromethoxymethyl, (3,3-difluoroazetidin-1-yl)methyl, (3,3-difluoropyrrolidin-1-yl)methyl, or 2,2-difluoro-1,1-dimethyl-ethyl. In certain embodiments, q is 1, and R⁴ is (4-chloro-3-fluoro-phenoxy)methyl, 1,1,1-trifluoroethyl, 3-(trifluoromethoxy)cyclobutoxymethyl, 3-(trifluoromethoxy)propyl, 3,3-difluoro-1-methyl-propyl, benzyl, cyclobutoxymethyl, cyclobutylmethyl, cyclobutylmethyl, cyclopropylethyl, cyclopropylmethyl, methyl, trifluoroethyl, trifluoromethoxy, (trifluoromethoxy)cyclobutoxy, trifluoromethoxyethyl, trifluoromethoxymethyl, (3,3-difluoroazetidin-1-yl)methyl, (3,3-difluoropyrrolidin-1-yl)methyl, or 2,2-difluoro-1,1-dimethyl-ethyl.

In certain embodiments, the -L¹-B—(R⁴)_{q} or —B(R⁴)_{q} moiety is (4-chloro-3-fluoro-phenoxy)methyl, 1-fluorocyclopropyl, 1,1,1-trifluoroethyl, 2-methylcyclopropyl, 2,2-difluorocyclopropyl, 3-(difluoromethoxy)cyclobutyl, 3-(trifluoromethoxy)cyclobutoxymethyl, 3-(trifluoromethoxy)cyclobutyl, 3-(trifluoromethoxy)propyl, 3-(trifluoromethyl)cyclobutyl, 3-cyanocyclobutyl, 3,3-difluoro-1-methyl-propyl, 4-chloro-3-fluoro-phenyl, 4-chlorophenyl, benzyl, cyanocyclobutyl, cyclobutoxymethyl, cyclobutyl, cyclobutylmethyl, cyclobutylmethyl, cyclopentyl, cyclopropyl, cyclopropylethyl, cyclopropylmethyl, hydroxycyclobutyl, methyl, N-tert-butoxy(carbonyl)azetidin-3-yl, N-tert-butoxy(carbonyl)pyrrolidin-3-yl, tetrahydrofuranyl, trifluoroethyl, trifluoromethoxy, (trifluoromethoxy)cyclobutoxy, trifluoromethoxyethyl, trifluoromethoxymethyl, 3-(1,1-difluoroethyl)cyclobutyl, 3-(1,1,1-trifluoroethyl)azetidinyl, 3-(triazol-2-yl)cyclobutyl, 3-(trifluoromethylthio)cyclobutyl, 3-(cyclopropyl)cyclobutyl, (3,3-difluoroazetidin-1-yl)methyl, (3,3-difluoropyrrolidin-1-yl)methyl, 1-(2,2,2-trifluoro-1-methyl-ethyl)azetidin-3-yl, 1-(2,2,2-trifluoroethyl)azetidin-3-yl, 1-(2,2,2-trifluoroethyl)pyrazol-3-yl, 1-(2,2,2-trifluoroethyl)pyrazol-4-yl, 1-(2,2-difluoroethyl)azetidin-3-yl, 1-tert-butoxycarbonyl-2-methylazetidin-3-yl, 2-(4-chloro-3-fluoro-phenyl, 2-(difluoromethyl)cyclopropyl, 2-(trifluoromethoxymethyl)cyclopropyl, 2,2-difluoro-1,1-dimethyl-ethyl, 2-methyl-1-(2,2,2-trifluoroethyl)azetidin-3-yl, 3-(trifluoromethoxymethyl)cyclobutyl, 3-(trifluoromethyl)azetidin-1-yl, 3-fluoro-1-(2,2,2-trifluoroethyl)azetidin-3-yl, 4-(2,2,2-trifluoroethyl)morpholin-2-yl, 4-tert-butoxycarbonyl-morpholin-2-yl, 5-(trifluoromethoxymethyl)tetrahydrofuran-2-yl, 2-((trifluoromethoxy)methyl)cyclopropyl, 5-fluoro-3-pyridyl, 1-(2,2,2-trifluoroethyl)pyrrolidin-3-yl, 3-ethoxycyclobutanyl, or 3-isopropoxycyclobutanyl.

In certain embodiments, the -L¹-B—(R⁴)_{q} or —B(R⁴)_{q} moiety is (4-chloro-3-fluoro-phenoxy)methyl, 1-fluorocyclopropyl, 1,1,1-trifluoroethyl, 2-methylcyclopropyl, 2,2-difluorocyclopropyl, 3-(difluoromethoxy)cyclobutyl, 3-(trifluoromethoxy)cyclobutoxymethyl, 3-(trifluoromethoxy)cyclobutyl, 3-(trifluoromethoxy)propyl, 3-(trifluoromethyl)cyclobutyl, 3-cyanocyclobutyl, 3,3-difluoro-1-methyl-propyl, 4-chloro-3-fluoro-phenyl, 4-chlorophenyl, benzyl, cyanocyclobutyl, cyclobutoxymethyl, cyclobutyl, cyclobutylmethyl, cyclobutylmethyl, cyclopentyl, cyclopropyl, cyclopropylethyl, cyclopropylmethyl, hydroxycyclobutyl, methyl, N-tert-butoxy(carbonyl)azetidin-3-yl, N-tert-butoxy(carbonyl)pyrrolidin-3-yl, tetrahydrofuranyl, trifluoroethyl, trifluoromethoxy, (trifluoromethoxy)cyclobutoxy, trifluoromethoxyethyl, trifluoromethoxymethyl, 3-(1,1-difluoroethyl)cyclobutyl, 3-(1,1,1-trifluoroethyl)azetidinyl, 3-(triazol-2-yl)cyclobutyl, 3-(trifluoromethylthio)cyclobutyl, 3-(cyclopropyl)cyclobutyl, (3,3-difluoroazetidin-1-yl)methyl, (3,3-difluoropyrrolidin-1-yl)methyl, 1-(2,2,2-trifluoro-1-methyl-ethyl)azetidin-3-yl, 1-(2,2,2-trifluoroethyl)azetidin-3-yl, 1-(2,2,2-trifluoroethyl)pyrazol-3-yl, 1-(2,2,2-trifluoroethyl)pyrazol-4-yl, 1-(2,2-difluoroethyl)azetidin-3-yl, 1-tert-butoxycarbonyl-2-methylazetidin-3-yl, 2-(4-chloro-3-fluoro-phenyl, 2-(difluoromethyl)cyclopropyl, 2-(trifluoromethoxymethyl)cyclopropyl, 2,2-difluoro-1,1-dimethyl-ethyl, 2-methyl-1-(2,2,2-trifluoroethyl)azetidin-3-yl, 3-(trifluoromethoxymethyl)cyclobutyl, 3-(trifluoromethyl)azetidin-1-yl, 3-fluoro-1-(2,2,2-trifluoroethyl)azetidin-3-yl, 4-(2,2,2-trifluoroethyl)morpholin-2-yl, 4-tert-butoxycarbonyl-morpholin-2-yl, 5-(trifluoromethoxymethyl)tetrahydrofuran-2-yl, 2-((trifluoromethoxy)methyl)cyclopropyl or 5-fluoro-3-pyridyl.

In certain embodiments, the —B(R⁴)_{q} moiety is 1-fluorocyclopropyl, 2-methylcyclopropyl, 2,2-difluorocyclopropyl, 3-(difluoromethoxy)cyclobutyl, 3-(trifluoromethoxy)cyclobutyl, 3-(trifluoromethyl)cyclobutyl, 3-cyanocyclobutyl, 4-chloro-3-fluoro-phenyl, 4-chlorophenyl, cyanocyclobutyl, cyclobutyl, cyclopentyl, cyclopropyl, hydroxycyclobutyl, N-tert-butoxy(carbonyl)azetidin-3-yl, N-tert-butoxy(carbonyl)pyrrolidin-3-yl, tetrahydrofuranyl, 3-(1,1-difluoroethyl)cyclobutyl, 3-(1,1,1-trifluoroethyl)azetidinyl, 3-(triazol-2-yl)cyclobutyl, 3-(trifluoromethylthio)cyclobutyl, 3-(cyclopropyl)cyclobutyl, 1-(2,2,2-trifluoro-1-methyl-ethyl)azetidin-3-yl, 1-(2,2,2-trifluoroethyl)azetidin-3-yl, 1-(2,2,2-trifluoroethyl)pyrazol-3-yl, 1-(2,2,2-trifluoroethyl)pyrazol-4-yl, 1-(2,2-difluoroethyl)azetidin-3-yl, 1-tert-butoxycarbonyl-2-methylazetidin-3-yl, 2-(4-chloro-3-fluoro-phenyl, 2-(difluoromethyl)cyclopropyl, 2-(trifluoromethoxymethyl)cyclopropyl, 2-methyl-1-(2,2,2-trifluoroethyl)azetidin-3-yl, 3-(trifluoromethoxymethyl)cyclobutyl, 3-(trifluoromethyl)azetidin-1-yl, 3-fluoro-1-(2,2,2-trifluoroethyl)azetidin-3-yl, 4-(2,2,2-trifluoroethyl)morpholin-2-yl, 4-tert-butoxycarbonyl-morpholin-2-yl, 5-(trifluoromethoxymethyl)tetrahydrofuran-2-yl, 2-((trifluoromethoxy)methyl)cyclopropyl, 5-fluoro-3-pyridyl, 1-(2,2,2-trifluoroethyl)pyrrolidin-3-yl, 3-ethoxycyclobutanyl, 3-(2,2,2-trifluoroethyl)cyclobutyl, or 3-isopropoxycyclobutanyl.

In certain embodiments, the —B(R⁴)_{q} moiety is 1-fluorocyclopropyl, 2-methylcyclopropyl, 2,2-difluorocyclopropyl, 3-(difluoromethoxy)cyclobutyl, 3-(trifluoromethoxy)cyclobutyl, 3-(trifluoromethyl)cyclobutyl, 3-cyanocyclobutyl, 4-chloro-3-fluoro-phenyl, 4-chlorophenyl, cyanocyclobutyl, cyclobutyl, cyclopentyl, cyclopropyl, hydroxycyclobutyl, N-tert-butoxy(carbonyl)azetidin-3-yl, N-tert-butoxy(carbonyl)pyrrolidin-3-yl, tetrahydrofuranyl, 3-(1,1-difluoroethyl)cyclobutyl, 3-(1,1,1-trifluoroethyl)azetidinyl, 3-(triazol-2-yl)cyclobutyl, 3-(trifluoromethylthio)cyclobutyl, 3-(cyclopropyl)cyclobutyl, 1-(2,2,2-trifluoro-1-methyl-ethyl)azetidin-3-yl, 1-(2,2,2-trifluoroethyl)azetidin-3-yl, 1-(2,2,2-trifluoroethyl)pyrazol-3-yl, 1-(2,2,2-trifluoroethyl)pyrazol-4-yl, 1-(2,2-difluoroethyl)azetidin-3-yl, 1-tert-butoxycarbonyl-2-methylazetidin-3-yl, 2-(4-chloro-3-fluoro-phenyl, 2-(difluoromethyl)cyclopropyl, 2-(trifluoromethoxymethyl)cyclopropyl, 2-methyl-1-(2,2,2-trifluoroethyl)azetidin-3-yl, 3-(trifluoromethoxymethyl)cyclobutyl, 3-(trifluoromethyl)azetidin-1-yl, 3-fluoro-1-(2,2,2-trifluoroethyl)azetidin-3-yl, 4-(2,2,2-trifluoroethyl)morpholin-2-yl, 4-tert-butoxycarbonyl-morpholin-2-yl, 5-(trifluoromethoxymethyl)tetrahydrofuran-2-yl, 2-((trifluoromethoxy)methyl)cyclopropyl, 5-fluoro-3-pyridyl, 1-(2,2,2-trifluoroethyl)pyrrolidin-3-yl, 3-ethoxycyclobutanyl, or 3-isopropoxycyclobutanyl.

In certain embodiments, the —B(R⁴)_{q} moiety is 1-fluorocyclopropyl, 2-methylcyclopropyl, 2,2-difluorocyclopropyl, 3-(difluoromethoxy)cyclobutyl, 3-(trifluoromethoxy)cyclobutyl, 3-(trifluoromethoxy)cyclopropyl, 3-(trifluoromethyl)cyclobutyl, 3-cyanocyclobutyl, 4-chloro-3-fluoro-phenyl, 4-chlorophenyl, phenyl, 3-cyanocyclobutyl, cyclobutyl, cyclopentyl, cyclopropyl, cyanocyclopropyl, hydroxycyclobutyl, N-tert-butoxy(carbonyl)azetidin-3-yl, N-(2,2,2-trifluoroethyl)azetidin-3-yl, N-tert-butoxy(carbonyl)pyrrolidin-3-yl, tetrahydrofuranyl, 3-(difluoromethoxy)cyclobutyl, 3-(1,1-difluoroethyl)cyclobutyl, 3-(1,1,1-trifluoroethyl)azetidinyl, 3-(triazol-2-yl)cyclobutyl, 3-(trifluoromethylthio)cyclobutyl, 3-(2,2,2-trifluoroethyl)cyclobutyl, or 3-(cyclopropyl)cyclobutyl.

In certain embodiments, the —B(R⁴)_{q} moiety is 1-fluorocyclopropyl, 2-methylcyclopropyl, 2,2-difluorocyclopropyl, 3-(difluoromethoxy)cyclobutyl, 3-(trifluoromethoxy)cyclopropyl, 3-(trifluoromethyl)cyclobutyl, 3-cyanocyclobutyl, 4-chloro-3-fluoro-phenyl, 4-chlorophenyl, phenyl, 3-cyanocyclobutyl, cyclobutyl, cyclopentyl, cyclopropyl, cyanocyclopropyl, hydroxycyclobutyl, N-tert-butoxy(carbonyl)azetidin-3-yl, N-(2,2,2-trifluoroethyl)azetidin-3-yl, N-tert-butoxy(carbonyl)pyrrolidin-3-yl, tetrahydrofuranyl, 3-(difluoromethoxy)cyclobutyl, 3-(trifluoromethoxy)cyclobutyl, 3-(1,1-difluoroethyl)cyclobutyl, 3-(1,1,1-trifluoroethyl)azetidinyl, 3-(triazol-2-yl)cyclobutyl, 3-(trifluoromethylthio)cyclobutyl, or 3-(cyclopropyl)cyclobutyl.

In certain embodiments, the —B(R⁴)_{q} moiety is cyclopropyl, 3,3-difluorocyclobutyl, 3-(trifluoromethyl)cyclobutyl, spiro[3.3]heptan-2-yl, 3,3-dimethylcyclobutyl, 3-cyanocyclobutyl, 3-cyano-1-methylcyclobutyl, 3-fluorocyclobutyl, 3-cyano-3-methylcyclobutyl, 3-(triazol-2-yl)cyclobutyl, 3-(difluoromethoxy)cyclobutyl, 3-methoxycyclobutyl, 3-methylcyclobutyl, 3-(difluoromethyl)cyclobutyl.

In certain embodiments, the —B(R⁴)_{q} moiety is 4-chloro-3-fluoro-phenyl, N-(2,2,2-trifluoroethyl)azetidin-3-yl, 3-(difluoromethoxy)cyclobutyl, or 3-(trifluoromethoxy)cyclobutyl.

In certain embodiments, the -L¹-B—(R⁴)_{q} moiety is (4-chloro-3-fluoro-phenoxy)methyl]-1,3,4-oxadiazol-2-yl, 1-(3-cyanocyclobutyl)triazol-4-yl, 1-(3-hydroxycyclobutyl)triazol-4-yl, 1-(4-chlorophenyl)triazol-4-yl, 1-benzyltriazol-4-yl, 1-cyclobutyltriazol-4-yl, 1H-1,2,3-triazol-4-yl, 2-(3-cyanocyclobutyl)triazol-4-yl, 2-(trifluoromethoxy)ethyl]-1,3,4-oxadiazol-2-yl, 2-cyclobutyltriazol-4-yl, 3-[(trifluoromethoxy)cyclobutoxy]-imidazol-1-yl, 3-cyanocyclobutyl)triazol-4-yl, 3-cyclobutylisoxazol-5-yl, 4-(cyclobutylmethyl)imidazol-1-yl, 4-[3-(trifluoromethoxy)cyclobutyl]imidazol-1-yl, 4-cyclobutylimidazol-1-yl, 4-cyclobutyloxazol-2-yl, 5-((4-chloro-3-fluorophenoxy)methyl)-4H-1,2,4-triazol-3-yl, 5-(1-fluorocyclopropyl)-1,3,4-oxadiazol-2-yl, 5-(2-cyclopropylethyl)-1,3,4-oxadiazol-2-yl, 5-(2,2-difluorocyclopropyl)-1,3,4-oxadiazol-2-yl, 5-(2,2,2-trifluoroethyl)-1,3,4-oxadiazol-2-yl, 5-(3-cyanocyclobutyl)-1,3,4-oxadiazol-2-yl, 5-(3,3-difluoro-1-methyl-propyl)-1,3,4-oxadiazol-²-yl, 5-(4-chloro-3-fluoro-phenyl)-1,3,4-oxadiazol-2-yl, 5-(cyclobutoxymethyl)-1,3,4-oxadiazol-2-yl, 5-(cyclobutylmethyl)-1,3,4-oxadiazol-2-yl, 5-(cyclopropylmethyl)-1,3,4-oxadiazol-2-yl, 5-(trifluoromethoxymethyl)-1,3,4-oxadiazol-2-yl, 5-[(4-chloro-3-fluoro-phenoxy)methyl]-1,3,4-oxadiazol-2-yl, 5-[[3-(trifluoromethoxy)cyclobutoxy]methyl]-1,3,4-oxadiazol-2-yl, 5-[2-methylcyclopropyl]-1,3,4-oxadiazol-2-yl, 5-[3-(trifluoromethoxy)cyclobutyl]-1,3,4-oxadiazol-2-yl, 5-[3-(trifluoromethoxy)propyl]-1,3,4-oxadiazol-2-yl, 5-[3-(trifluoromethyl)cyclobutyl]-1,3,4-oxadiazol-2-yl, 5-[N-(1,1,1-trifluoroethyl)azetidin-3-yl]-1,3,4-oxadiazol-2-yl, 5-[N-(1,1,1-trifluoroethyl)pyrrolidin-3-yl]-1,3,4-oxadiazol-2-yl, 5-[N-tert-butoxy(carbonyl)azetidin-3-yl]-1,3,4-oxadiazol-2-yl, 5-[N-tert-butoxy(carbonyl)pyrrolidin-3-yl]-1,3,4-oxadiazol-2-yl, 5-cyclobutyl-1,3,4-oxadiazol-2-yl, 5-cyclobutyl-4,5-dihydroisoxazol-3-yl, 5-cyclobutylisoxazol-3-yl, 5-cyclobutyloxazol-2-yl, 5-cyclopentyl-4,5-dihydroisoxazol-3-yl, oxazolidin-2-one-5-yl, (3-(trifluoromethoxy)cyclobutoxy)eth-2-yl, 1-(3-(trifluoromethoxy)cyclobutyl)-1H-pyrazol-4-yl, 2,2-difluoro-3-(3-(trifluoromethoxy)cyclobutoxy)propyl, 2-(3-(trifluoromethoxy)cyclobutyl)pyrimidin-4-yl, 3-(2,2,2-trifluoroethyl)cyclobutoxy)methyl, 2-oxo-3-[3-(trifluoromethoxy)cyclobutyl]imidazolidin-1-yl, or 2-(3-(trifluoromethoxy)cyclobutoxy)acetyl.

In certain embodiments, the -L¹-B—(R⁴)_{q} moiety is (4-chloro-3-fluoro-phenoxy)methyl]-1,3,4-oxadiazol-2-yl, 1-(3-cyanocyclobutyl)triazol-4-yl, 1-(3-hydroxycyclobutyl)triazol-4-yl, 1-(4-chlorophenyl)triazol-4-yl, 1-benzyltriazol-4-yl, 1-cyclobutyltriazol-4-yl, 1H-1,2,3-triazol-4-yl, 2-(3-cyanocyclobutyl)triazol-4-yl, 2-(trifluoromethoxy)ethyl]-1,3,4-oxadiazol-2-yl, 2-cyclobutyltriazol-4-yl, 3-[(trifluoromethoxy)cyclobutoxy]-imidazol-1-yl, 3-cyanocyclobutyl)triazol-4-yl, 3-cyclobutylisoxazol-5-yl, 4-(cyclobutylmethyl)imidazol-1-yl, 4-[3-(trifluoromethoxy)cyclobutyl]imidazol-1-yl, 4-cyclobutylimidazol-1-yl, 4-cyclobutyloxazol-2-yl, 5-((4-chloro-3-fluorophenoxy)methyl)-4H-1,2,4-triazol-3-yl, 5-(1-fluorocyclopropyl)-1,3,4-oxadiazol-2-yl, 5-(2-cyclopropylethyl)-1,3,4-oxadiazol-2-yl, 5-(2,2-difluorocyclopropyl)-1,3,4-oxadiazol-2-yl, 5-(2,2,2-trifluoroethyl)-1,3,4-oxadiazol-2-yl, 5-(3-cyanocyclobutyl)-1,3,4-oxadiazol-2-yl, 5-(3,3-difluoro-1-methyl-propyl)-1,3,4-oxadiazol-2-yl, 5-(4-chloro-3-fluoro-phenyl)-1,3,4-oxadiazol-2-yl, 5-(cyclobutoxymethyl)-1,3,4-oxadiazol-2-yl, 5-(cyclobutylmethyl)-1,3,4-oxadiazol-2-yl, 5-(cyclopropylmethyl)-1,3,4-oxadiazol-2-yl, 5-(trifluoromethoxymethyl)-1,3,4-oxadiazol-2-yl, 5-[(4-chloro-3-fluoro-phenoxy)methyl]-1,3,4-oxadiazol-2-yl, 5-[[3-(trifluoromethoxy)cyclobutoxy]methyl]-1,3,4-oxadiazol-2-yl, 5-[2-methylcyclopropyl]-1,3,4-oxadiazol-2-yl, 5-[3-(trifluoromethoxy)cyclobutyl]-1,3,4-oxadiazol-2-yl, 5-[3-(trifluoromethoxy)propyl]-1,3,4-oxadiazol-2-yl, 5-[3-(trifluoromethyl)cyclobutyl]-1,3,4-oxadiazol-2-yl, 5-[N-(1,1,1-trifluoroethyl)azetidin-3-yl]-1,3,4-oxadiazol-2-yl, 5-[N-(1,1,1-trifluoroethyl)pyrrolidin-3-yl]-1,3,4-oxadiazol-2-yl, 5-[N-tert-butoxy(carbonyl)azetidin-3-yl]-1,3,4-oxadiazol-2-yl, 5-[N-tert-butoxy(carbonyl)pyrrolidin-3-yl]-1,3,4-oxadiazol-2-yl, 5-cyclobutyl-1,3,4-oxadiazol-2-yl, 5-cyclobutyl-4,5-dihydroisoxazol-3-yl, 5-cyclobutylisoxazol-3-yl, 5-cyclobutyloxazol-2-yl, 5-cyclopentyl-4,5-dihydroisoxazol-3-yl, oxazolidin-2-one-5-yl, (3-(trifluoromethoxy)cyclobutoxy)eth-2-yl, 1-(3-(trifluoromethoxy)cyclobutyl)-1H-pyrazol-4-yl, 2,2-difluoro-3-(3-(trifluoromethoxy)cyclobutoxy)propyl, 2-(3-(trifluoromethoxy)cyclobutyl)pyrimidin-4-yl, or 2-(3-(trifluoromethoxy)cyclobutoxy)acetyl.

In certain embodiments, -L¹-B—(R⁴)_{q} is (4-chloro-3-fluoro-phenoxy)methyl]-1,3,4-oxadiazol-2-yl, 1-(3-cyanocyclobutyl)triazol-4-yl, 1-(3-hydroxycyclobutyl)triazol-4-yl, 1-(4-chlorophenyl)triazol-4-yl, 1-benzyltriazol-4-yl, 1-cyclobutyltriazol-4-yl, 1H-1,2,3-triazol-4-yl, 2-(3-cyanocyclobutyl)triazol-4-yl, 2-(trifluoromethoxy)ethyl]-1,3,4-oxadiazol-2-yl, 2-cyclobutyltriazol-4-yl, 3-[(trifluoromethoxy)cyclobutoxy]-imidazol-1-yl, 3-cyanocyclobutyl)triazol-4-yl, 3-cyclobutylisoxazol-5-yl, 4-(cyclobutylmethyl)imidazol-1-yl, 4-[3-(trifluoromethoxy)cyclobutyl]imidazol-1-yl, 4-cyclobutylimidazol-1-yl, 4-cyclobutyloxazol-2-yl, 5-((4-chloro-3-fluorophenoxy)methyl)-4H-1,2,4-triazol-3-yl, 5-(1-fluorocyclopropyl)-1,3,4-oxadiazol-2-yl, 5-(2-cyclopropylethyl)-1,3,4-oxadiazol-2-yl, 5-(2,2-difluorocyclopropyl)-1,3,4-oxadiazol-2-yl, 5-(2,2,2-trifluoroethyl)-1,3,4-oxadiazol-2-yl, 5-(3-cyanocyclobutyl)-1,3,4-oxadiazol-2-yl, 5-(3,3-difluoro-1-methyl-propyl)-1,3,4-oxadiazol-2-yl, 5-(4-chloro-3-fluoro-phenyl)-1,3,4-oxadiazol-2-yl, 5-(cyclobutoxymethyl)-1,3,4-oxadiazol-2-yl, 5-(cyclobutylmethyl)-1,3,4-oxadiazol-²-yl, 5-(cyclopropyl methyl)-1,3,4-oxadiazol-2-yl, 5-(trifluoromethoxymethyl)-1,3,4-oxadiazol-2-yl, 5-[(4-chloro-3-fluoro-phenoxy)methyl]-1,3,4-oxadiazol-2-yl, 5-[[3-(trifluoromethoxy)cyclobutoxy]methyl]-1,3,4-oxadiazol-2-yl, 5-[2-methylcyclopropyl]-1,3,4-oxadiazol-2-yl, 5-[3-(trifluoromethoxy)cyclobutyl]-1,3,4-oxadiazol-2-yl, 5-[3-(trifluoromethoxy)propyl]-1,3,4-oxadiazol-2-yl, 5-[3-(trifluoromethyl)cyclobutyl]-1,3,4-oxadiazol-2-yl, 5-[N-(1,1,1-trifluoroethyl)azetidin-3-yl]-1,3,4-oxadiazol-2-yl, 5-[N-(1,1,1-trifluoroethyl)pyrrolidin-3-yl]-1,3,4-oxadiazol-2-yl, 5-[N-tert-butoxy(carbonyl)azetidin-3-yl]-1,3,4-oxadiazol-2-yl, 5-[N-tert-butoxy(carbonyl)pyrrolidin-3-yl]-1,3,4-oxadiazol-2-yl, 5-cyclobutyl-1,3,4-oxadiazol-²-yl, 5-cyclobutyl-4,5-dihydroisoxazol-3-yl, 5-cyclobutylisoxazol-3-yl, 5-cyclobutyloxazol-2-yl, 5-cyclopentyl-4,5-dihydroisoxazol-3-yl, oxazolidin-2-one-5-yl, or (3-(trifluoromethoxy)cyclobutoxy)eth-2-yl.

In certain embodiments, provided is a compound selected from Table 1:

TABLE 1

| Ex. | |
|---|---|
| No. | Compound |
| 1 | 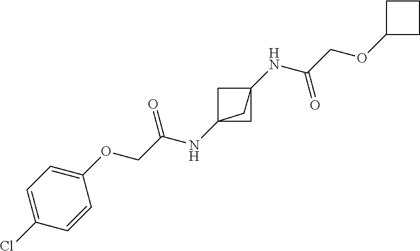 [SMILES: O=C(COc1ccc(Cl)cc1)NC12CC(NC(=O)COC3CCC3)(C1)C2] |
| 2 | 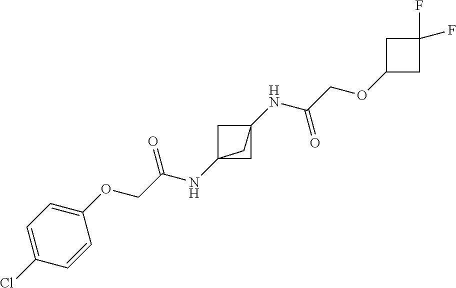 [SMILES: O=C(COc1ccc(Cl)cc1)NC12CC(NC(=O)COC3CC(F)(F)C3)(C1)C2] |
| 3 | 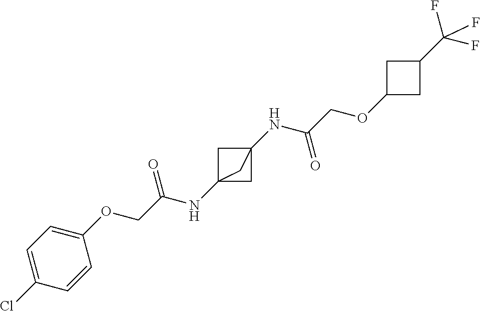 [SMILES: O=C(COc1ccc(Cl)cc1)NC12CC(NC(=O)COC3CC(C(F)(F)F)C3)(C1)C2] |
| | First eluting isomer |
| 3 | 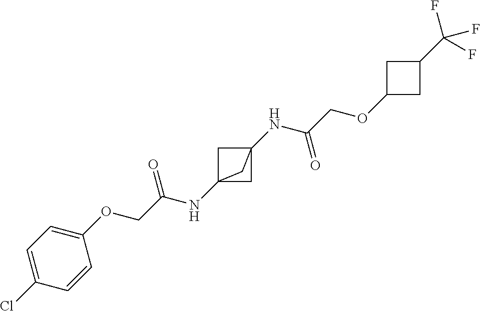 [SMILES: O=C(COc1ccc(Cl)cc1)NC12CC(NC(=O)COC3CC(C(F)(F)F)C3)(C1)C2] |
| | Second eluting isomer |
| 4 | 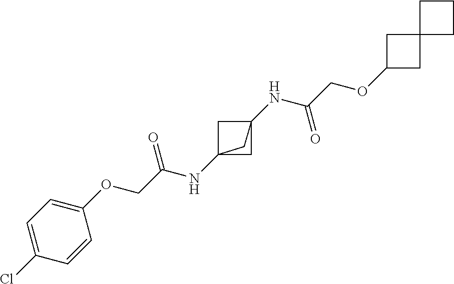 [SMILES: O=C(COc1ccc(Cl)cc1)NC12CC(NC(=O)COC3CC4(CCC4)C3)(C1)C2] |
| 5 | 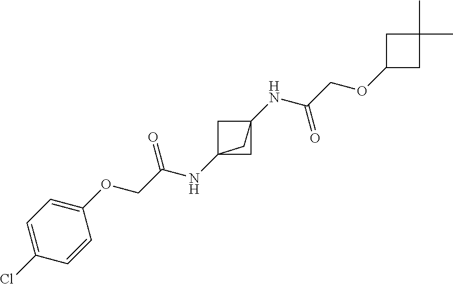 [SMILES: CC1(C)CC(OCC(=O)NC23CC(NC(=O)COc4ccc(Cl)cc4)(C2)C3)C1] |
| 6 | 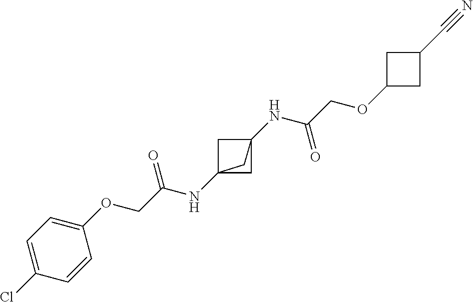 [SMILES: N#CC1CC(OCC(=O)NC23CC(NC(=O)COc4ccc(Cl)cc4)(C2)C3)C1] |
| 7 | 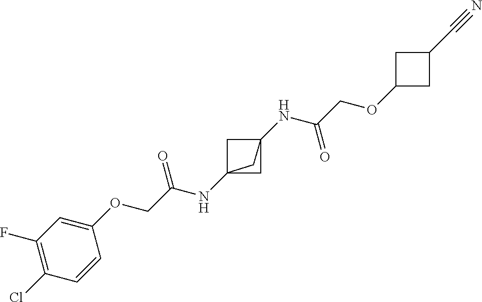 [SMILES: N#CC1CC(OCC(=O)NC23CC(NC(=O)COc4ccc(Cl)c(F)c4)(C2)C3)C1] |
| 8 | 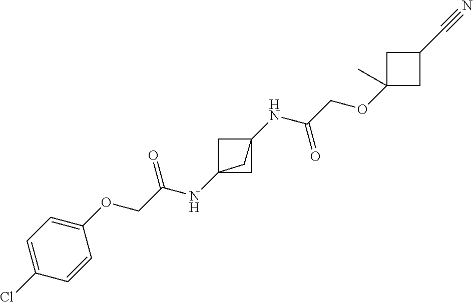 [SMILES: CC1(OCC(=O)NC23CC(NC(=O)COc4ccc(Cl)cc4)(C2)C3)CC(C#N)C1] |
| 9 | 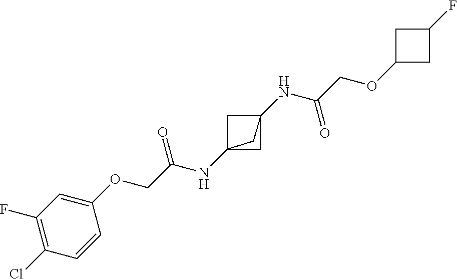 [SMILES: O=C(COc1ccc(Cl)c(F)c1)NC12CC(NC(=O)COC3CC(F)C3)(C1)C2] |
| 10 | 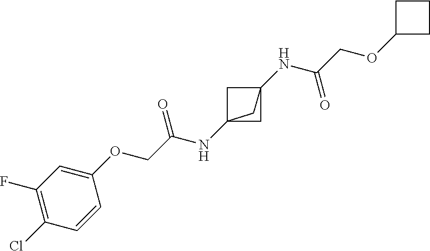 [SMILES: O=C(COc1ccc(Cl)c(F)c1)NC12CC(NC(=O)COC3CCC3)(C1)C2] |
| 11 | 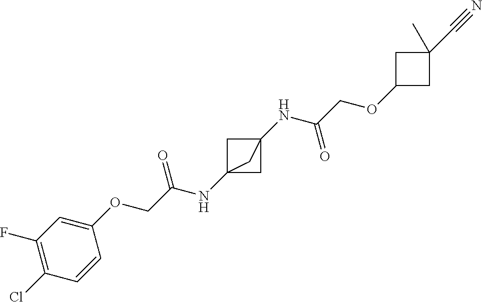 [SMILES: CC1(C#N)CC(OCC(=O)NC23CC(NC(=O)COc4ccc(Cl)c(F)c4)(C2)C3)C1] |
| 12 | 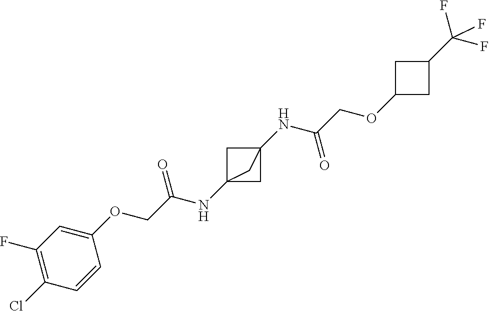 [SMILES: O=C(COc1ccc(Cl)c(F)c1)NC12CC(NC(=O)COC3CC(C(F)(F)F)C3)(C1)C2] |
| | (First eluting isomer) |
| 12 | 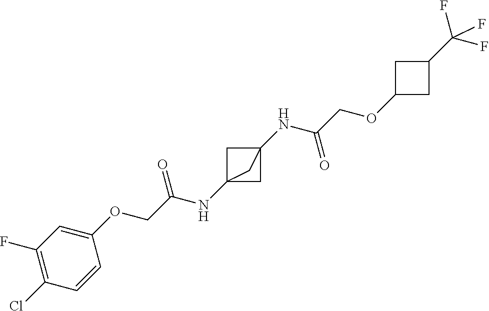 [SMILES: O=C(COc1ccc(Cl)c(F)c1)NC12CC(NC(=O)COC3CC(C(F)(F)F)C3)(C1)C2] |
| | (Second eluting isomer) |
| 13 | 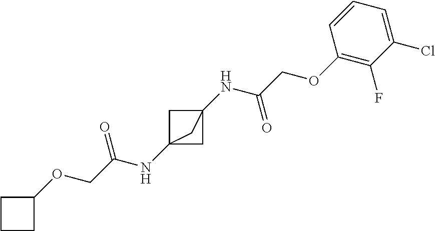 [SMILES: O=C(COc1cccc(Cl)c1F)NC12CC(NC(=O)COC3CCC3)(C1)C2] |
| 14 | 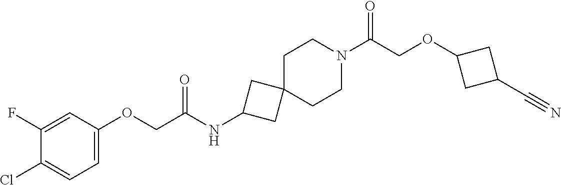 [SMILES: N#CC1CC(OCC(=O)N2CCC3(CC2)CC(NC(=O)COc2ccc(Cl)c(F)c2)C3)C1] |
| 15 | 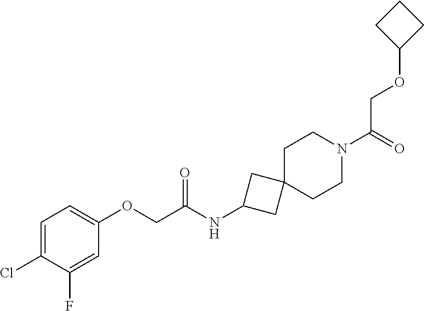 [SMILES: O=C(COc1ccc(Cl)c(F)c1)NC1CC2(CCN(C(=O)COC3CCC3)CC2)C1] |
| 16 | 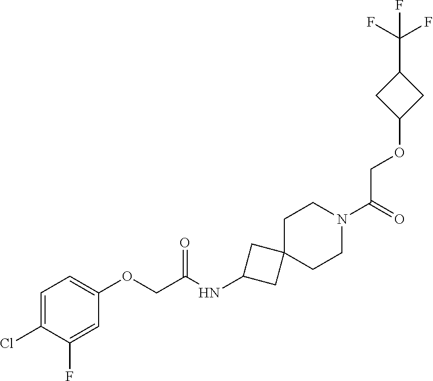 [SMILES: O=C(COc1ccc(Cl)c(F)c1)NC1CC2(CCN(C(=O)COC3CC(C(F)(F)F)C3)CC2)C1] |
| 17 | 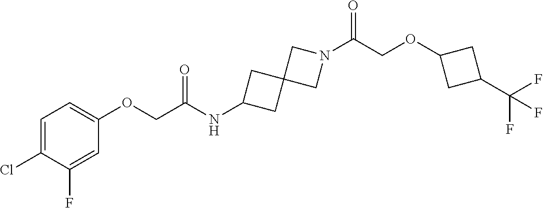 [SMILES: O=C(COc1ccc(Cl)c(F)c1)NC1CC2(C1)CN(C(=O)COC1CC(C(F)(F)F)C1)C2] |
| 18 | 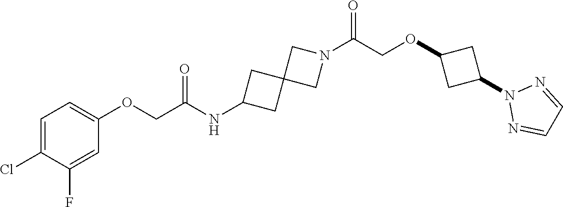 |
| 19 | 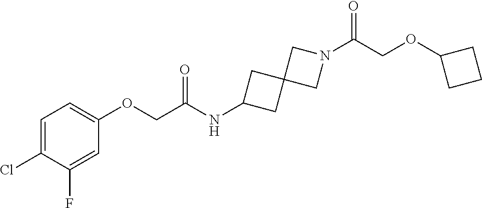 [SMILES: O=C(COc1ccc(Cl)c(F)c1)NC1CC2(C1)CN(C(=O)COC1CCC1)C2] |
| 20 | 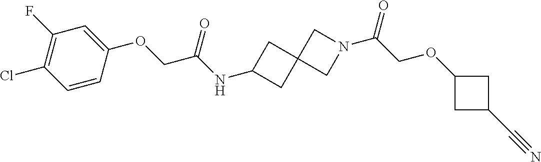 [SMILES: N#CC1CC(OCC(=O)N2CC3(CC(NC(=O)COc4ccc(Cl)c(F)c4)C3)C2)C1] |
| 21 | 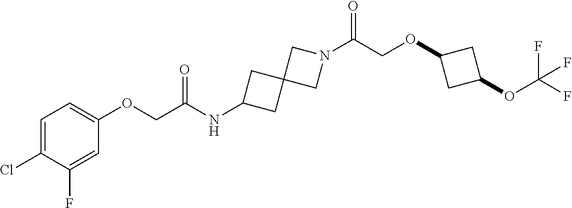 |
| 22 | 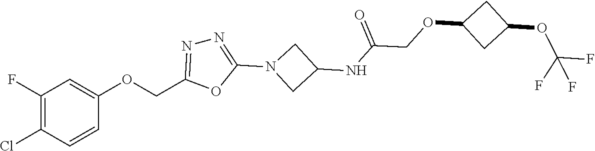 |
| 23 | 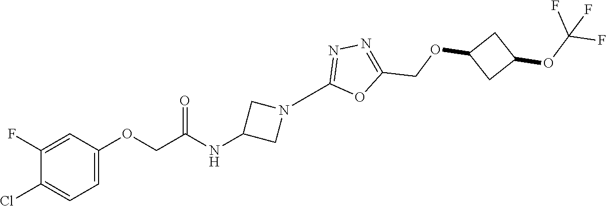 |
| 24 | 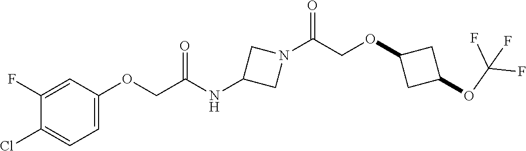 |
| 25 | 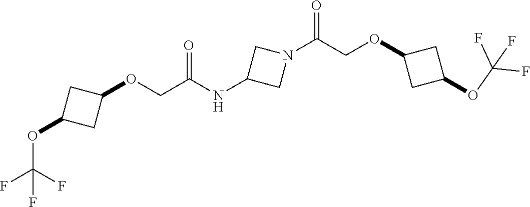 |
| 26 | 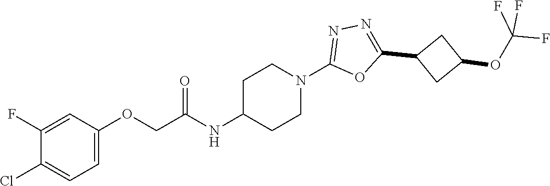 |
| 27 | 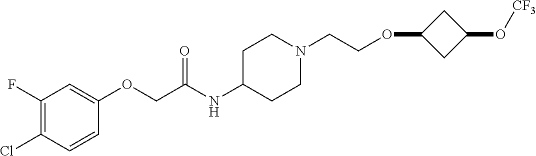 |
| 28 | 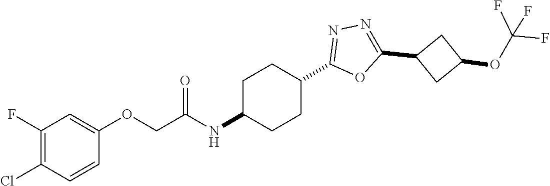 |
| 29 | 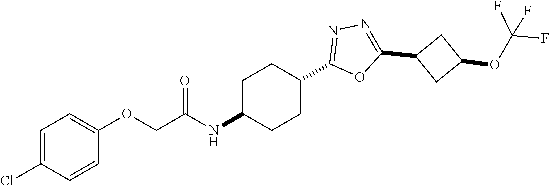 |
| 30 | 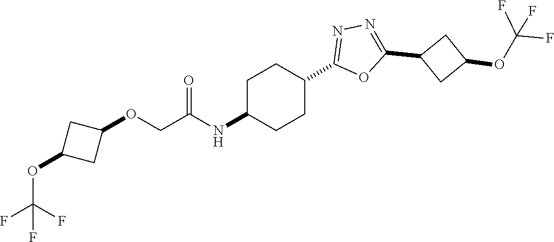 |
| 31 | 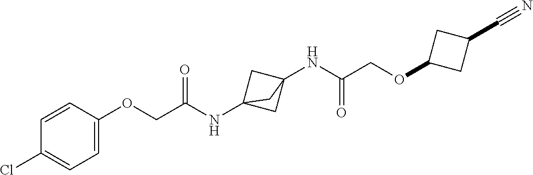 |
| 32 | 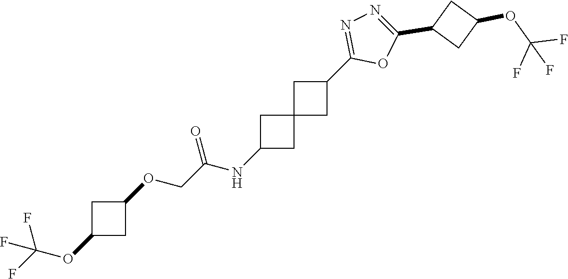 |
| 33 | 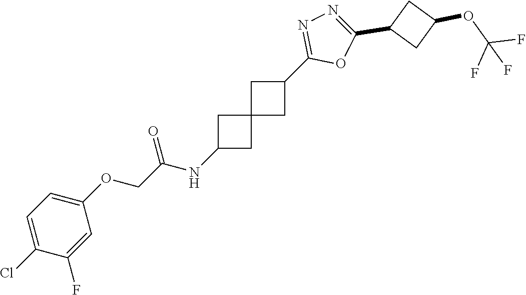 |
| 34 | 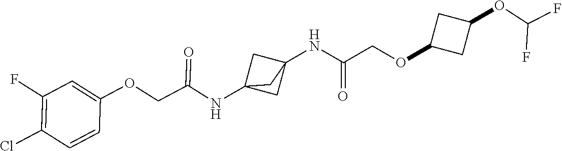 |
| 35 | 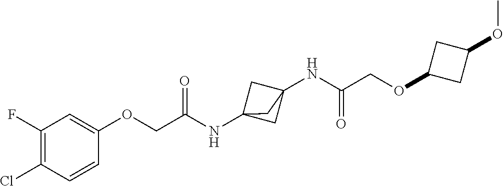 |
| 36 | 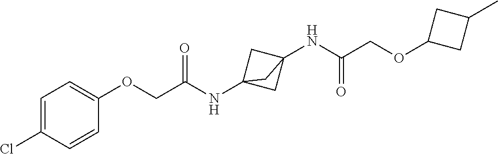 [SMILES: CC1CC(OCC(=O)NC23CC(NC(=O)COc4ccc(Cl)cc4)(C2)C3)C1] |
| 37 | 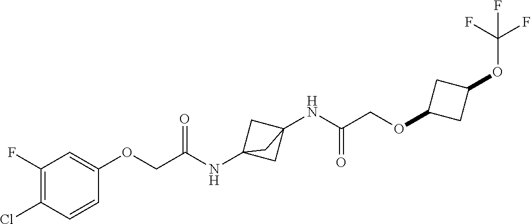 |
| 38 | 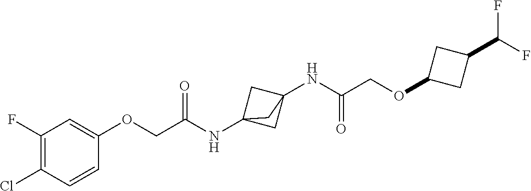 |
| 39 | 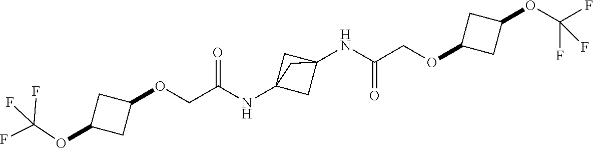 |
| 40 | 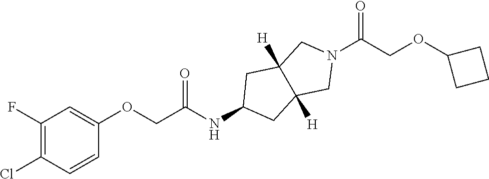 [SMILES: O=C(COc1ccc(Cl)c(F)c1)N[C@H]1C[C@@H]2CN(C(=O)COC3CCC3)C[C@@H]2C1] |
| 41 | 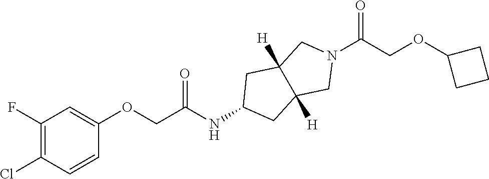 [SMILES: O=C(COc1ccc(Cl)c(F)c1)N[C@@H]1C[C@@H]2CN(C(=O)COC3CCC3)C[C@@H]2C1] |
| 42 | 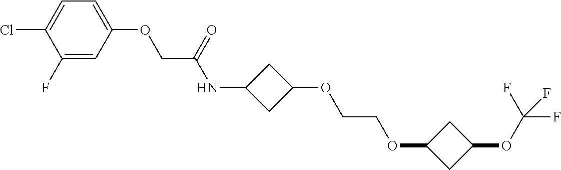 |
| | Isomer 1 |
| 42 | 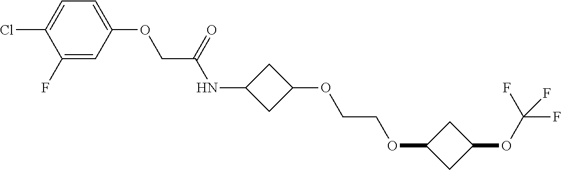 |
| | Isomer 2 |
| 43 | 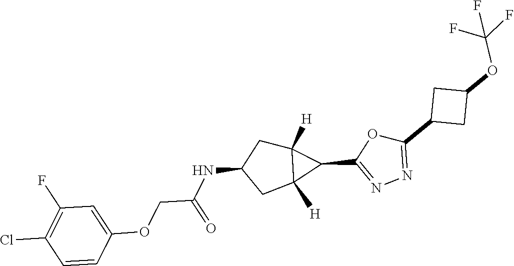 |
| 44 | 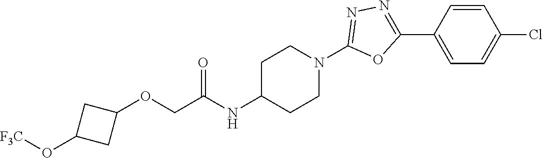 [SMILES: O=C(COC1CC(OC(F)(F)F)C1)NC1CCN(c2nnc(-c3ccc(Cl)cc3)o2)CC1] |
| 45 | 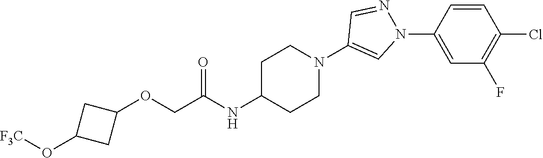 [SMILES: O=C(COC1CC(OC(F)(F)F)C1)NC1CCN(c2cnn(-c3ccc(Cl)c(F)c3)c2)CC1] |
| 46 | 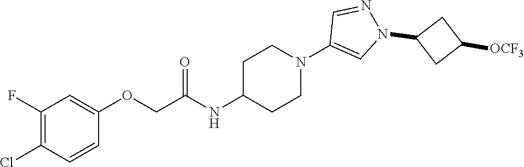 |
| 47 | 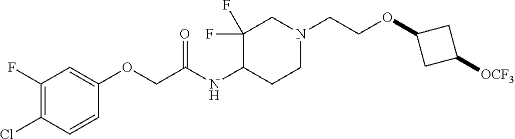 |
| 48 | 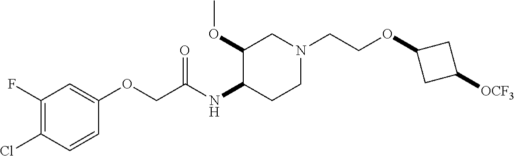 |
| 49 | 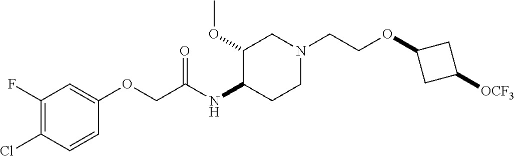 |
| 50 | 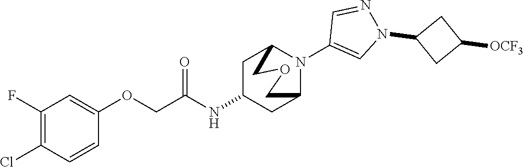 |
| 51 | 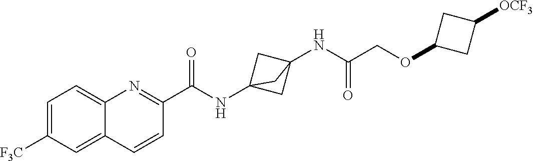 |
| 52 | 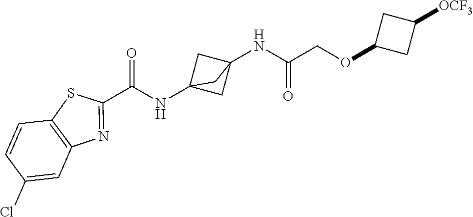 |
| 53 | 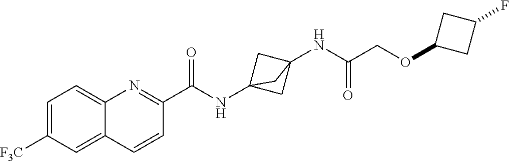 |
| 54 | 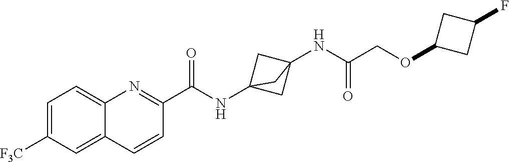 |
| 55 | 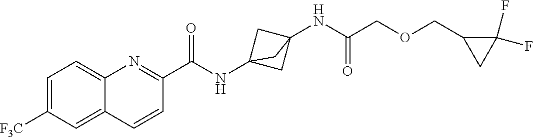 [SMILES: O=C(COCC1CC1(F)F)NC12CC(NC(=O)c3ccc4cc(C(F)(F)F)ccc4n3)(C1)C2] |
| 56 | 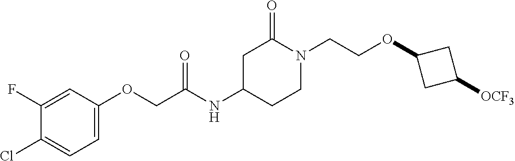 |
| 57 | 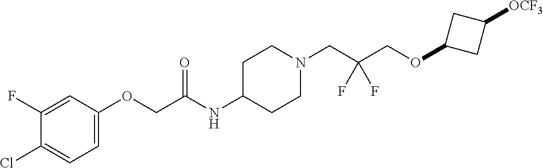 |
| 58 | 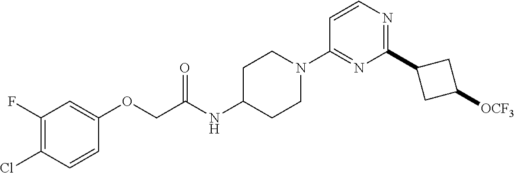 |
| 58 | 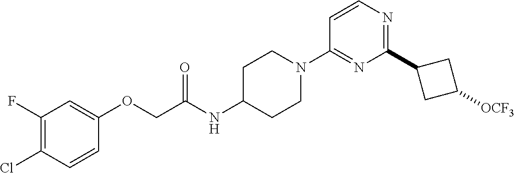 |
| 59 | 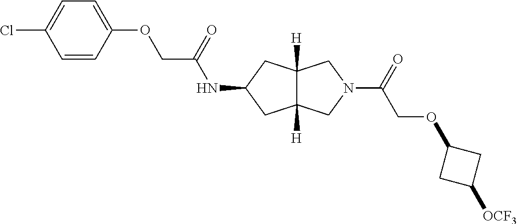 |
| 60 | 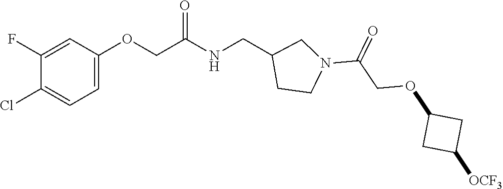 |
| 61 | 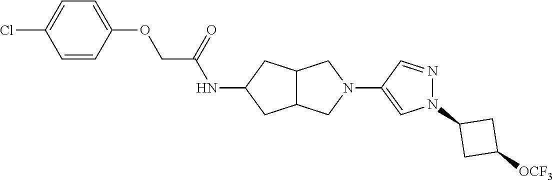 [SMILES: O=C(COc1ccc(Cl)cc1)NC1CC2CN(c3cnn([C@H]4C[C@@H](OC(F)(F)F)C4)c3)CC2C1] |
| 62 | 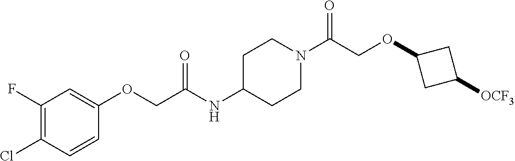 |
| 63 | 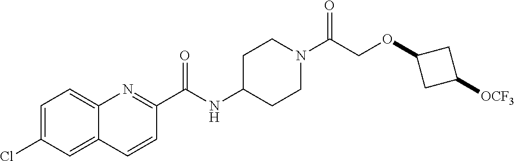 |
| 64 | 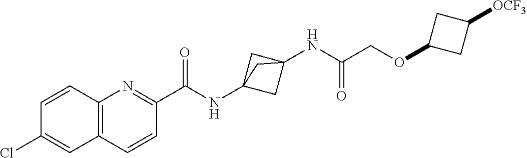 |
| 65 | 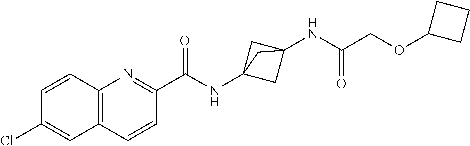 [SMILES: O=C(COC1CCC1)NC12CC(NC(=O)c3ccc4cc(Cl)ccc4n3)(C1)C2] |
| 66 | 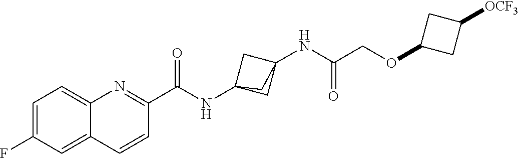 |
| 67 | 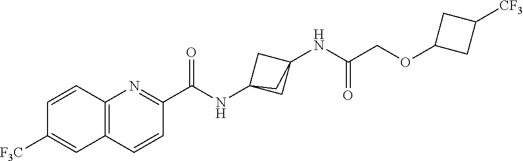 [SMILES: O=C(COC1CC(C(F)(F)F)C1)NC12CC(NC(=O)c3ccc4cc(C(F)(F)F)ccc4n3)(C1)C2] |
| 68 | 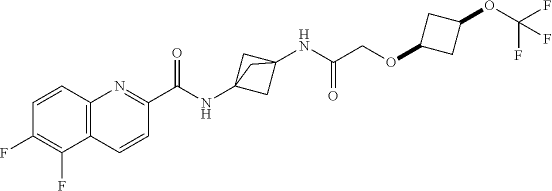 |
| 69 | 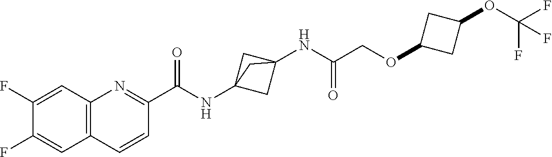 |
| 70 | 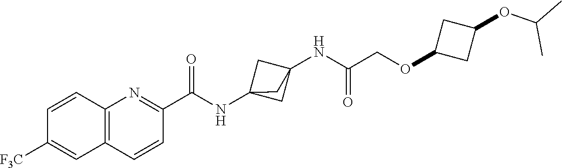 |
| 71 | 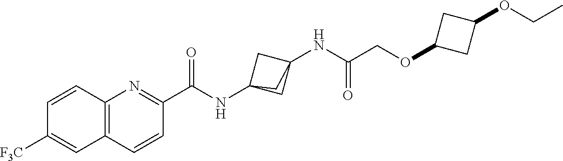 |
| 72 | 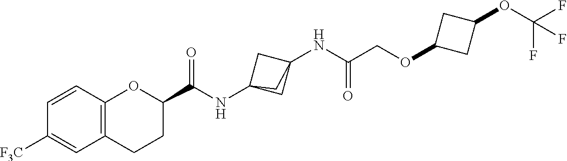 |
| 72 | 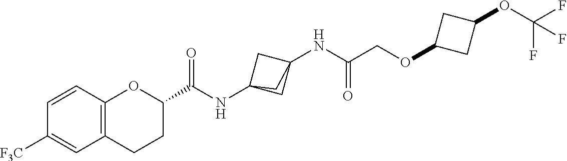 |
| 73 | 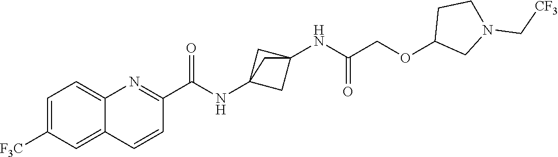 [SMILES: O=C(COC1CCN(CC(F)(F)F)C1)NC12CC(NC(=O)c3ccc4cc(C(F)(F)F)ccc4n3)(C1)C2] |
| 74 | 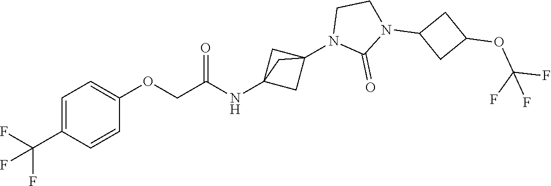 [SMILES: O=C(COc1ccc(C(F)(F)F)cc1)NC12CC(N3CCN(C4CC(OC(F)(F)F)C4)C3=O)(C1)C2] |
| 75 | 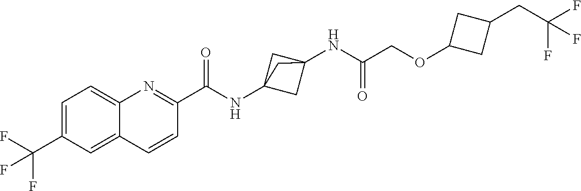 [SMILES: O=C(COC1CC(CC(F)(F)F)C1)NC12CC(NC(=O)c3ccc4cc(C(F)(F)F)ccc4n3)(C1)C2] |

or a pharmaceutically acceptable salt, isotopically enriched analog, prodrug, stereoisomer, or a mixture of stereoisomers thereof.

In certain embodiments, provided is a compound selected from Table 2:

TABLE 2
Structure

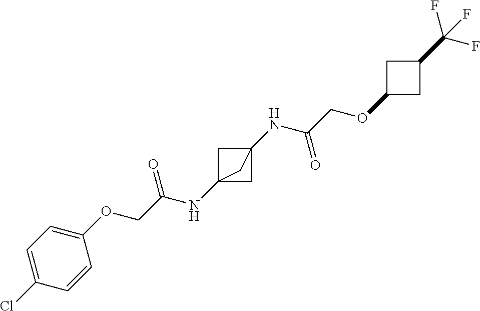

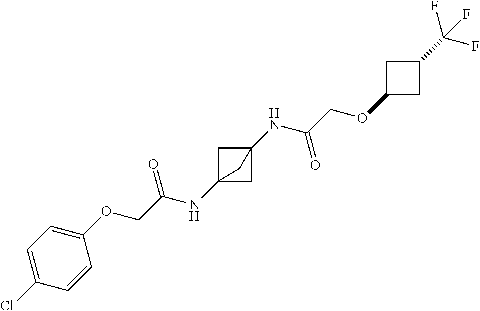

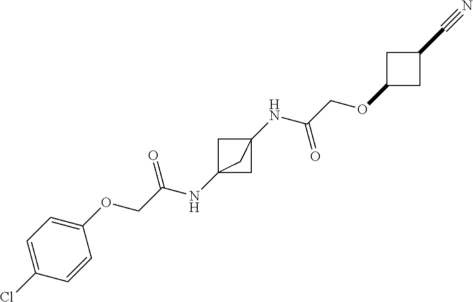

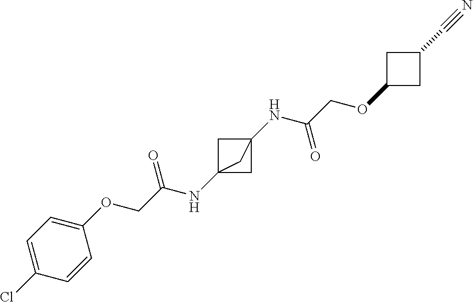

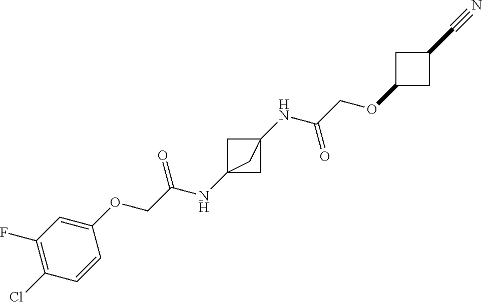

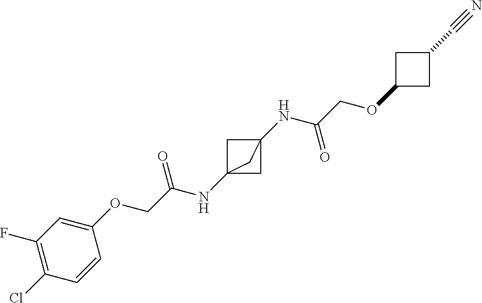

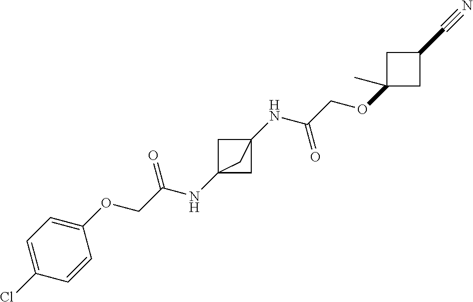

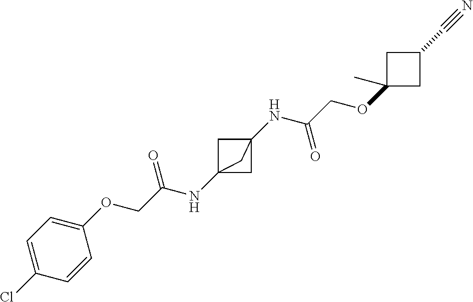

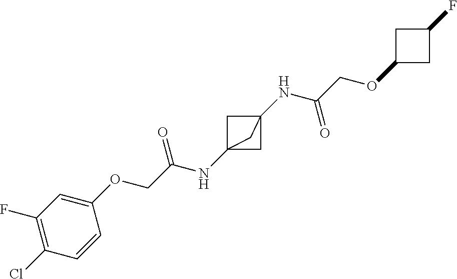

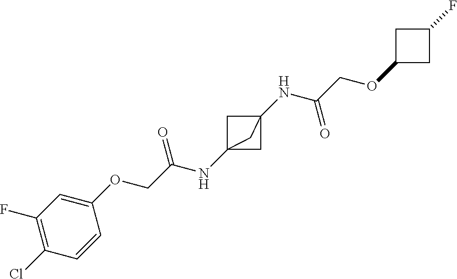

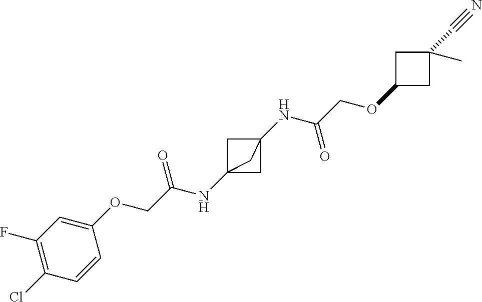

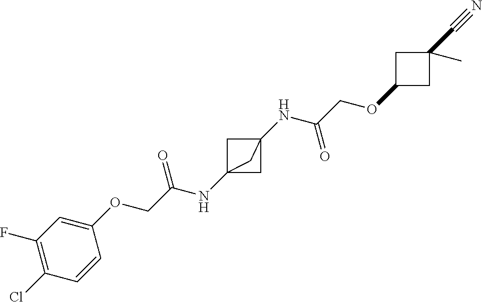

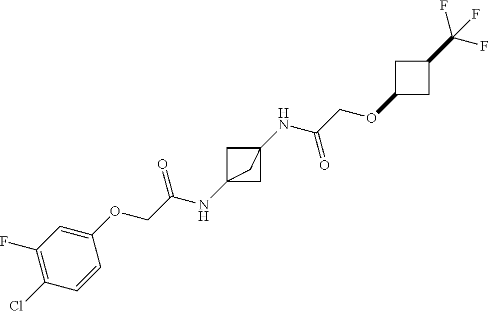

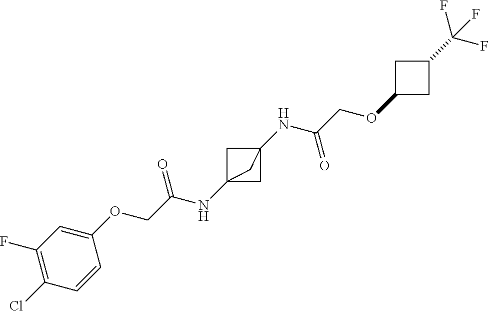

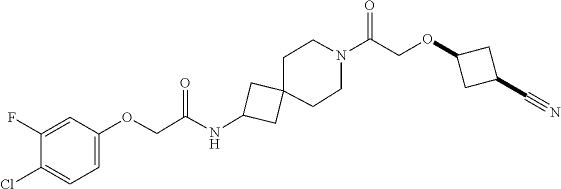

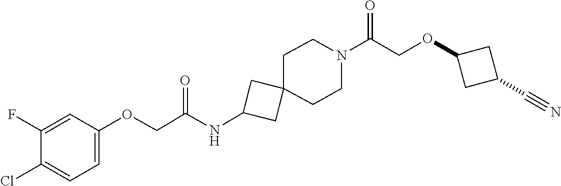

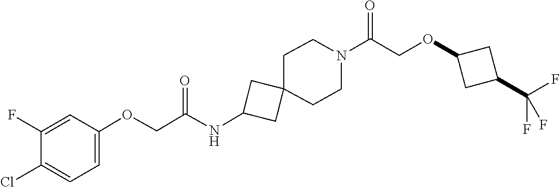

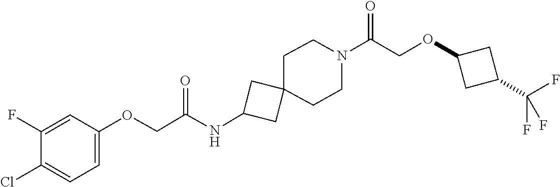

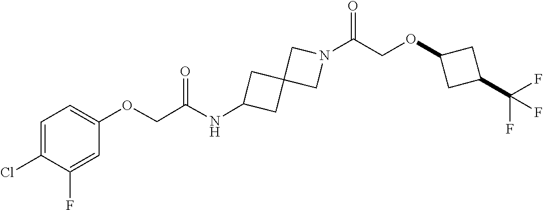

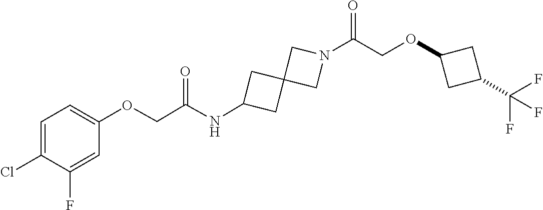

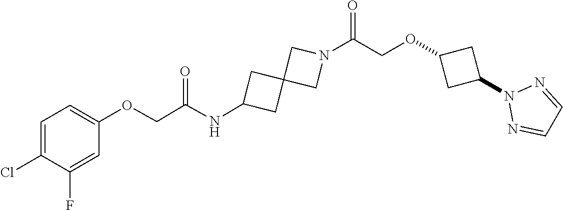

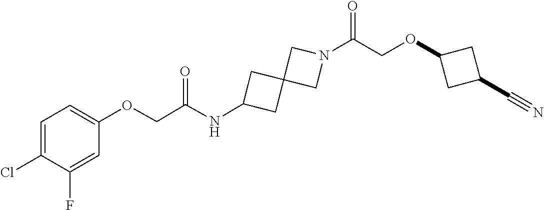

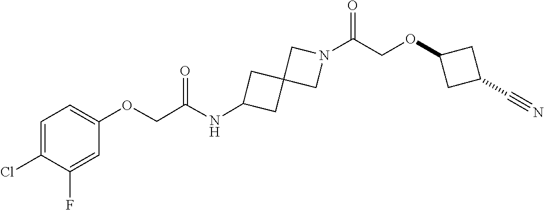

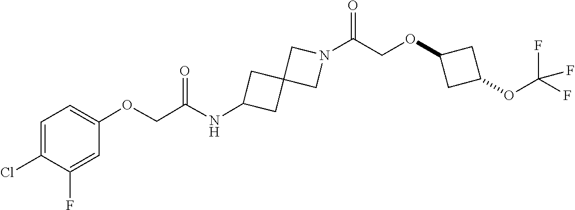

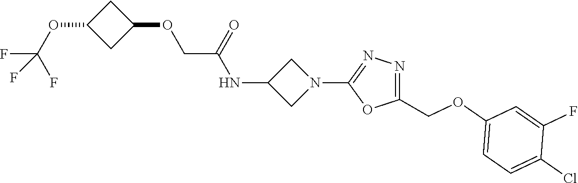 [SMILES: O=C(CO[C@H]1C[C@H](OC(F)(F)F)C1)NC1CN(c2nnc(COc3ccc(Cl)c(F)c3)o2)C1]

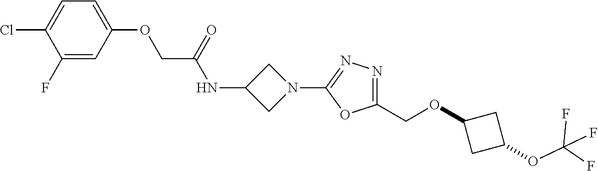

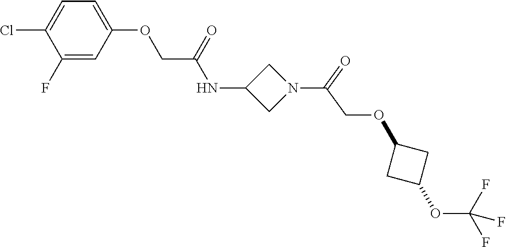

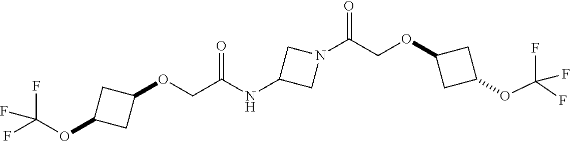

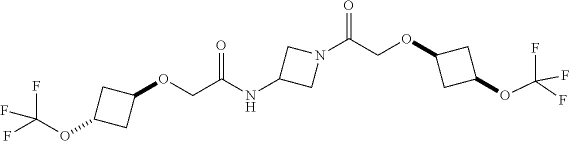

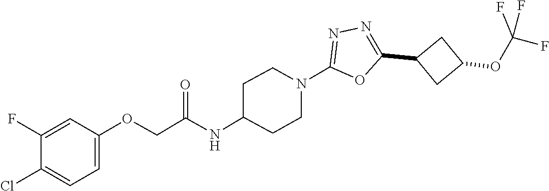

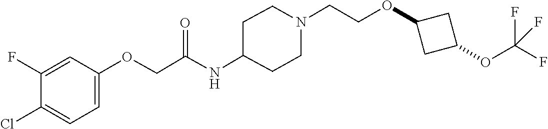

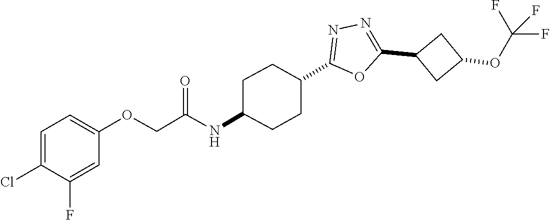

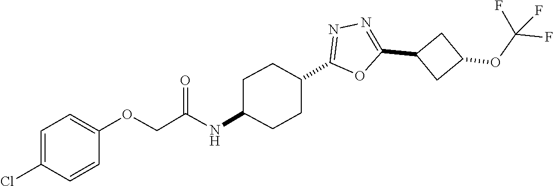

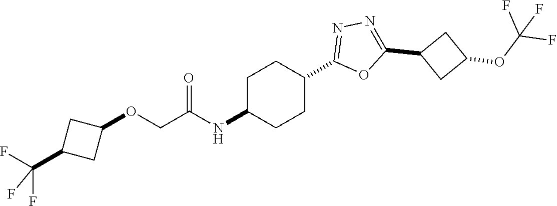

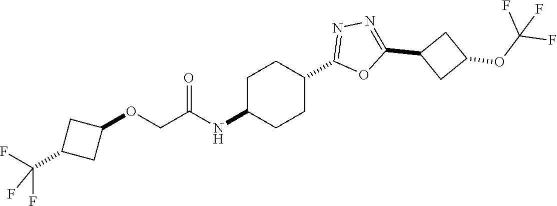

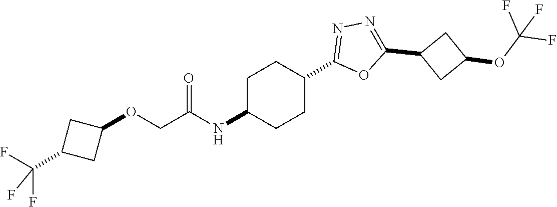

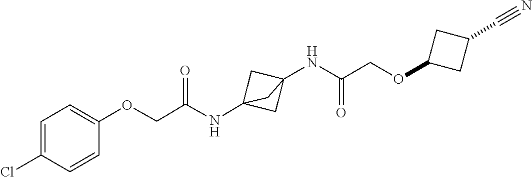

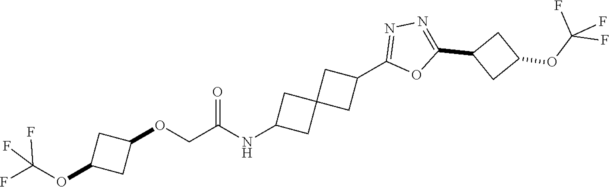

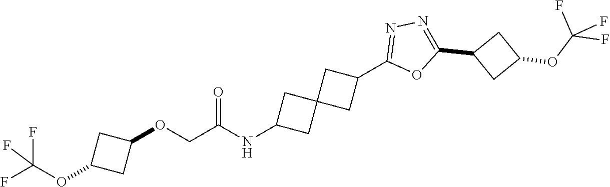

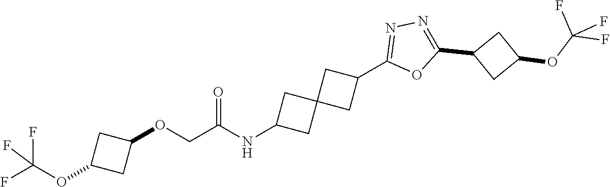

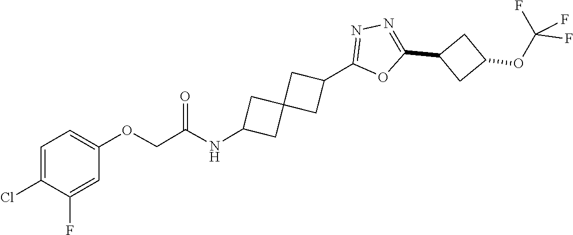

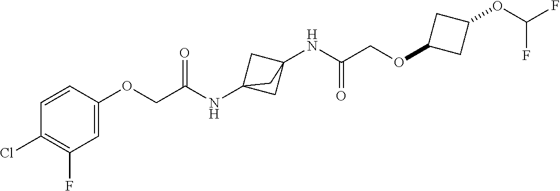

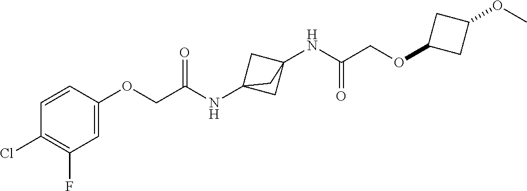

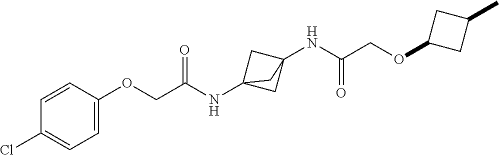

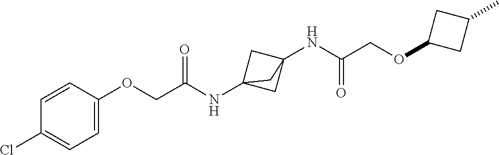

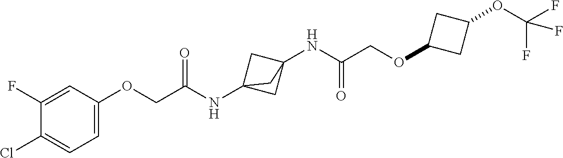

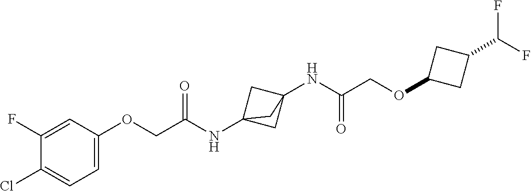

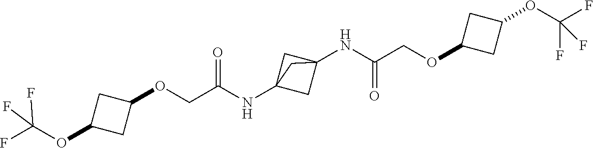

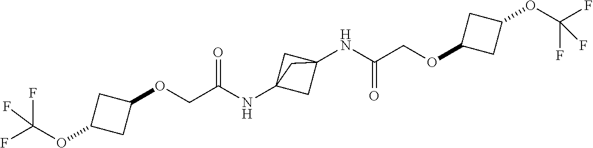

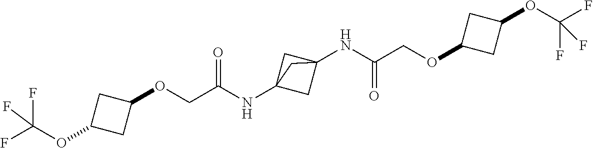

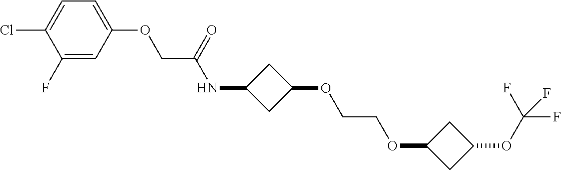

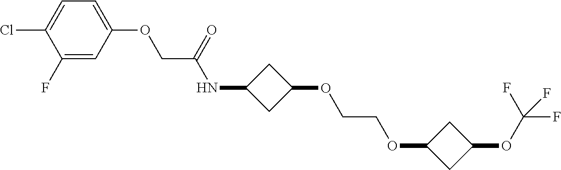

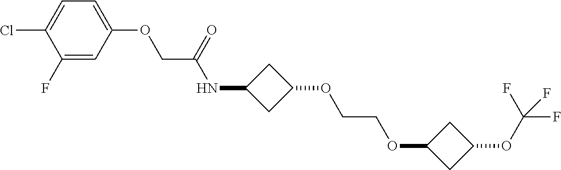

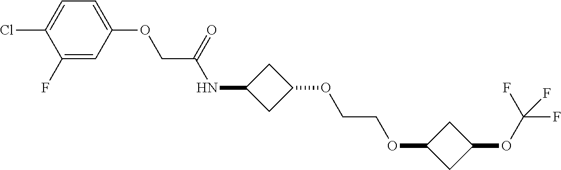

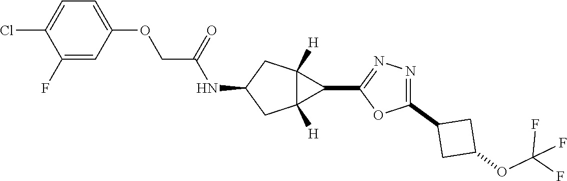

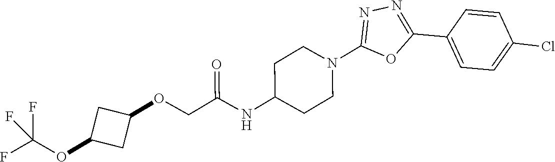

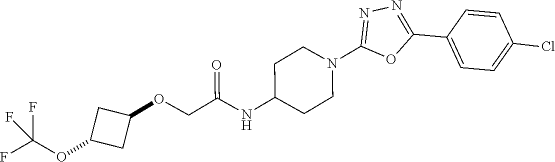

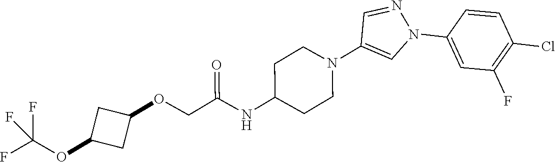

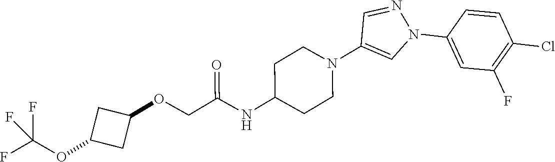

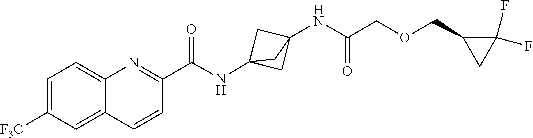 [SMILES: O=C(COC[C@@H]1CC1(F)F)NC12CC(NC(=O)c3ccc4cc(C(F)(F)F)ccc4n3)(C1)C2]

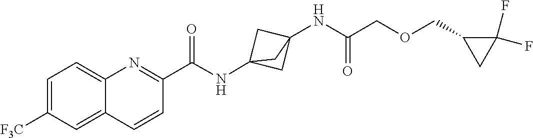 [SMILES: O=C(COC[C@H]1CC1(F)F)NC12CC(NC(=O)c3ccc4cc(C(F)(F)F)ccc4n3)(C1)C2]

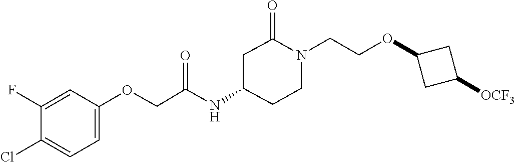

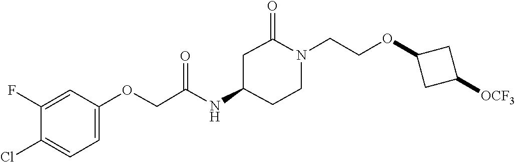

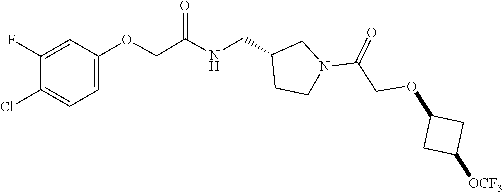

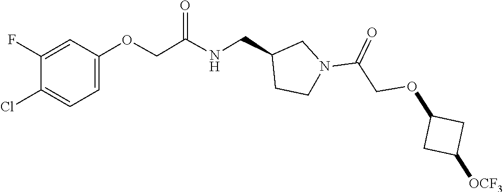

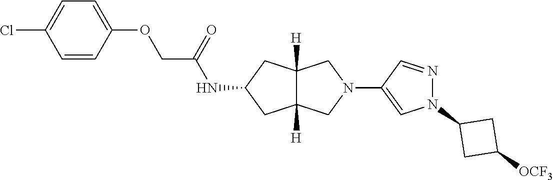 [SMILES: O=C(COc1ccc(Cl)cc1)N[C@@H]1C[C@@H]2CN(c3cnn([C@H]4C[C@@H](OC(F)(F)F)C4)c3)C[C@@H]2C1]

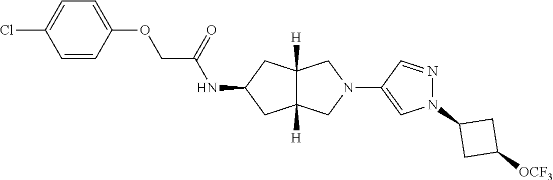 [SMILES: O=C(COc1ccc(Cl)cc1)N[C@H]1C[C@@H]2CN(c3cnn([C@H]4C[C@@H](OC(F)(F)F)C4)c3)C[C@@H]2C1]

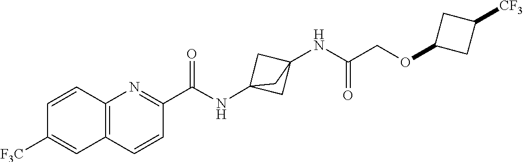

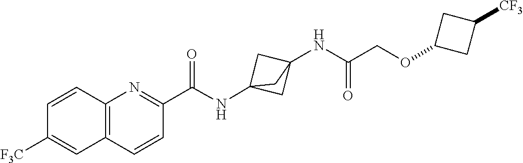

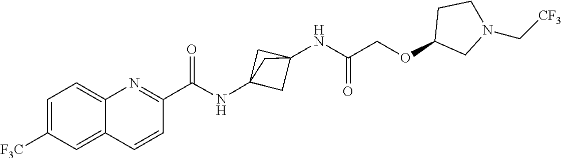 [SMILES: O=C(CO[C@H]1CCN(CC(F)(F)F)C1)NC12CC(NC(=O)c3ccc4cc(C(F)(F)F)ccc4n3)(C1)C2]

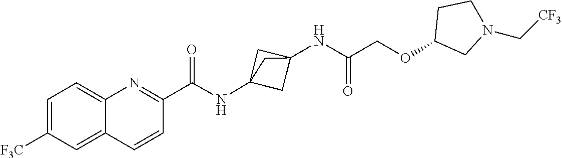 [SMILES: O=C(CO[C@@H]1CCN(CC(F)(F)F)C1)NC12CC(NC(=O)c3ccc4cc(C(F)(F)F)ccc4n3)(C1)C2]

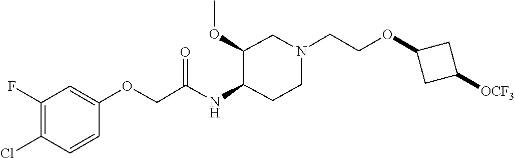

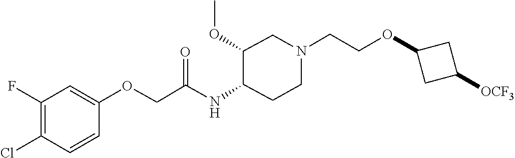

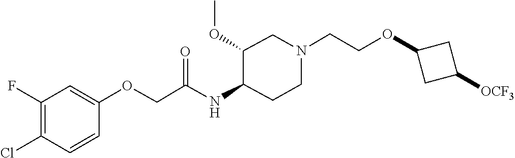

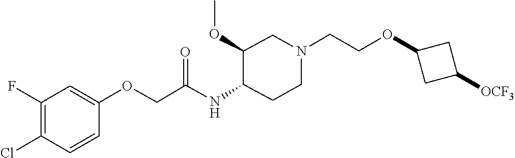

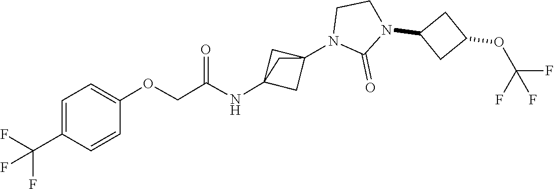

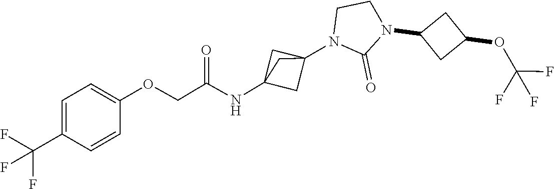

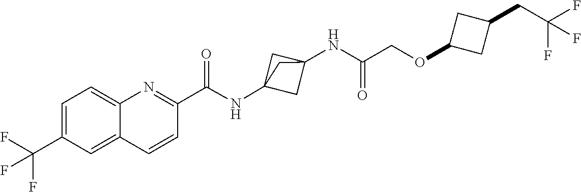

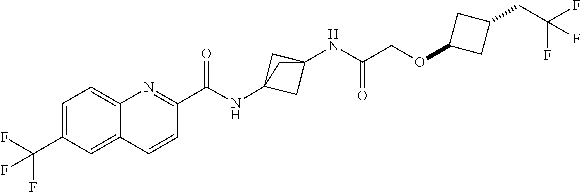

or a pharmaceutically acceptable salt, isotopically enriched analog, prodrug, stereoisomer, or a mixture of stereoisomers thereof.

### 3. METHODS

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In certain embodiments, the subject is a human.

The term “therapeutically effective amount” or “effective amount” of a compound described herein or a pharmaceutically acceptable salt, isotopically enriched analog, stereoisomer, mixture of stereoisomers or prodrug thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of as described herein. The therapeutically effective amount may vary depending on the subject, and disease or condition being treated, the weight and age of the subject, the severity of the disease or condition, and the manner of administering, which can readily be determined by one of ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

In certain embodiments, the compounds disclosed herein can be used to treat cellular proliferative disorders, including both cancerous and non-cancerous cellular proliferative disorders. Treatment of cellular proliferative disorders may comprise, but is not limited to, inhibiting cellular proliferation, including rapid proliferation. It is contemplated that the compounds described herein can be used to treat any type of cancer, including, but not limited to, carcinomas, sarcomas, lymphomas, leukemias and germ cell tumors. Exemplary cancers include, but are not limited to, adrenocortical carcinoma, anal cancer, appendix cancer, basal cell carcinoma, cholangiocarcinoma, bladder cancer, bone cancer, osteosarcoma or malignant Fibrous histiocytoma, brain cancer (e.g., brain stem glioma, astrocytoma (e.g., cerebellar, cerebral, etc.), atypical teratoid/rhabdoid tumor, central nervous system embryonal tumors, malignant glioma, craniopharyngioma, ependymoblastoma, ependymoma, medulloblastoma, medulloepithelioma, pineal parenchymal tumors of intermediate differentiation, supratentorial primitive neuroectodermal tumors and/or pineoblastoma, visual pathway and/or hypothalamic glioma, brain and spinal cord tumors, etc.), breast cancer, bronchial tumors, carcinoid tumor (e.g., gastrointestinal, etc.), carcinoma of unknown primary, cervical cancer, chordoma, chronic myeloproliferative disorders, colon cancer, colorectal cancer, embryonal tumors, cancers of the central nervous system, endometrial cancer, ependymoma, esophageal cancer, Ewing family of tumors, eye cancer (e.g., intraocular melanoma, retinoblastoma, etc.), gallbladder cancer, gastric cancer, gastrointestinal tumor (e.g., carcinoid tumor, stromal tumor (gist), stromal cell tumor, etc.), germ cell tumor (e.g., extracranial, extragonadal, ovarian, etc.), gestational trophoblastic tumor, head and neck cancer, hepatocellular cancer, hypopharyngeal cancer, hypothalamic and visual pathway glioma, intraocular melanoma, islet cell tumors. Kaposi sarcoma, kidney cancer, large cell tumors, laryngeal cancer (e.g., acute lymphoblastic, acute myeloid, etc.), leukemia (e.g., myeloid, acute myeloid, acute lymphoblastic, chronic lymphocytic, chronic myelogenous, multiple myelogenous, hairy cell, etc.), lip and/or oral cavity cancer, liver cancer, lung cancer (e.g., non-small cell, small cell, etc.), lymphoma (e.g., AIDS-related, Burkitt, cutaneous Tcell, Hodgkin, non-Hodgkin, primary central nervous system, cutaneous T-cell, Waldenström macroglobulinemia, etc.), malignant fibrous histiocytoma of bone and/or osteosarcoma, medulloblastoma, medulloepithelioma, merkel cell carcinoma, mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases (e.g., myeloproliferative disorders, chronic, etc.), nasal cavity and/or paranasal sinus cancer, nasopharyngeal cancer, neuroblastoma, oral cancer; oral cavity cancer, oropharyngeal cancer; osteosarcoma and/or malignant fibrous histiocytoma of bone; ovarian cancer (e.g., ovarian epithelial cancer, ovarian germ cell tumor, ovarian low malignant potential tumor, etc.), pancreatic cancer (e.g., islet cell tumors, etc.), papilloinatosis, paranasal sinus and/or nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasma/multiple myeloma, pleuropulmonary blastoma, prostate cancer, rectal cancer, renal cell cancer, transitional cell cancer, respiratory tract carcinoma involving the nut gene on chromosome 15, retinoblastoma, rhabdomyosarcoma, salivary gland cancer, sarcoma (e.g., Ewing family of tumors, Kaposi, soft tissue, uterine, etc.), Sézary syndrome, skin cancer (e.g., non-melanoma, melanoma, merkel cell, etc.), small intestine cancer, squamous cell carcinoma, squamous neck cancer with occult primary, metastatic, stomach cancer, supratentorial primitive neuroectodermal tumors, testicular cancer, throat cancer, thymoma and/or thymic carcinoma, thyroid cancer, transitional cell cancer of the renal, pelvis and/or ureter (e.g., trophoblastic tumor, unknown primary site carcinoma, urethral cancer, uterine cancer, endometrial, uterine sarcoma, etc.), vaginal cancer, visual pathway and/or hypothalamic glioma, vulvar cancer, Wilms tumor, and the like. Examples of noncancerous cellular proliferative disorders include, but are not limited to, fibroadenoma, adenoma, intraductal papilloma, nipple adenoma, adenosis, fibrocystic disease or changes of breast, plasma cell proliferative disorder (PCPD), restenosis, atherosclerosis, rheumatoid arthritis, myofibromatosis, fibrous hamartoma, granular lymphocyte proliferative disorders, benign hyperplasia of prostate, heavy chain diseases (HCDs), lymphoproliferative disorders, psoriasis, idiopathic pulmonary fibrosis, scleroderma, cirrhosis of the liver, IgA nephropathy, mesangial proliferative glomerulonephritis, membranoproliferative glomerulonephritis, hemangiomas, vascular and non-vascular intraocular proliferative disorders, and the like.

In certain embodiments, the compounds disclosed herein can be used to treat lung injury and/or lung inflammation.

In certain embodiments, the compounds disclosed herein can be used to treat cancer, pre-cancerous syndromes and diseases/injuries associated with activated unfolded protein response pathways, such as vanishing white matter (VWM) disease, Alzheimer's disease, neuropathic pain, spinal cord injury, traumatic brain injury, ischemic stroke, stroke, Parkinson's disease, diabetes, metabolic syndrome, metabolic disorders, Huntington's disease, Creutzfeldt-Jakob Disease, fatal familial insomnia, Gerstmann-Straussler-Scheinker syndrome, and related prion diseases, amyotrophic lateral sclerosis, progressive supranuclear palsy, myocardial infarction, cardiovascular disease, inflammation, organ fibrosis, chronic and acute diseases of the liver, fatty liver disease, liver steatosis, liver fibrosis, chronic and acute diseases of the lung, lung fibrosis, chronic and acute diseases of the kidney, kidney fibrosis, chronic traumatic encephalopathy (CTE), neurodegeneration, dementias, frontotemporal dementias, tauopathies, Pick's disease, Niemann-Pick's disease, amyloidosis, cognitive impairment, atherosclerosis, ocular diseases, arrhythmias, in organ transplantation and in the transportation of organs for transplantation.

In embodiments, the compounds disclosed herein can be used to treat or lessen the severity of cancer, Alzheimer's disease, stroke, Type 1 diabetes, Parkinson disease, Huntington's disease, amyotrophic lateral sclerosis, myocardial infarction, cardiovascular disease, atherosclerosis, arrhythmias, or age-related macular degeneration.

In certain embodiments, the compounds disclosed herein can be used to treat neuropathic pain.

In certain embodiments, the compounds disclosed herein can be used to treat or lessen the severity of ocular diseases/angiogenesis. In certain embodiments, the ocular disease includes vascular leakage (e.g., edema or neovascularization for any occlusive or inflammatory retinal vascular disease, such as rubeosis irides, neovascular glaucoma, pterygium, vascularized glaucoma filtering blebs, conjunctival papilloma), choroidal neovascularization (e.g., neovascular age-related macular degeneration (AMD), myopia, prior uveitis, trauma, or idiopathic), macular edema (e.g., post surgical macular edema, macular edema secondary to uveitis including retinal and/or choroidal inflammation, macular edema secondary to diabetes, and macular edema secondary to retinovascular occlusive disease (i.e. branch and central retinal vein occlusion)), retinal neovascularization due to diabetes (e.g., retinal vein occlusion, uveitis, ocular ischemic syndrome from carotid artery disease, ophthalmic or retinal artery occlusion, sickle cell retinopathy, other ischemic or occlusive neovascular retinopathies, retinopathy of prematurity, or Eale's Disease), and genetic disorders (e.g., VonHippel-Lindau syndrome). In certain embodiments, the neovascular age-related macular degeneration is wet age-related macular degeneration. In certain embodiments, the neovascular age-related macular degeneration is dry age-related macular degeneration and the patient is characterized as being at increased risk of developing wet age-related macular degeneration.

In certain embodiments, the compounds disclosed herein can be used to treat viral infections (e.g., to prevent the initiation of viral protein synthesis). Exemplary viruses which can be treated using the compounds disclosed herein include, but are not limited to, picornaviridae (e.g., polioviruses), reoviridae (e.g., rotaviruses), togaviridae (e.g., encephalitis viruses, yellow fever virus, rubella virus, etc.), orthomyxoviridae (e.g., influenza viruses), paramyxoviridae (e.g., respiratory syncytial virus, measles virus, mumps virus, parainfluenza virus, etc.), rhabdoviridae (e.g., rabies virus), coronaviridae, bunyaviridae, flaviviridae, filoviridae, arenaviridae, bunyaviridae and retroviridae (e.g., human T-cell lymphotropic viruses (HTLV), human immunodeficiency viruses (HIV), etc.), papovaviridae (e.g. papilloma viruses), adenoviridae (e.g., adenovirus), herpesviridae (e.g., herpes simplex viruses) and poxyiridae (e.g., variola viruses). In certain embodiments, the viral infection is caused by hepatitis B virus, hepatitis C virus and/or HIV.

In certain embodiments, the compounds disclosed herein can be used to treat disorders associated with viral infections. Such disorders include, but are not limited to neurological symptoms (e.g., encephalitis, meningoencephalitis, paralysis, myelopathy, neuropathy, aseptic meningitis, hemiparesis, dementia, dysphagia, lack of muscular coordination, impaired vision, coma, etc.), wasting symptoms (e.g., inflammatory cell infiltration, perivascular cuffing of blood vessels, demyelination, necrosis, reactive gliosis, etc.), gastroenteritis symptoms (e diarrhea, vomiting, cramps, etc.), hepatitis symptoms (nausea, vomiting, right upper quadrant pain, raised liver enzyme levels (e.g., AST, ALT, etc.), jaundice, etc.), hemorrhagic fever symptoms (e.g., headache, fever, chills body pains, diarrhea, vomiting, dizziness, confusion, abnormal behavior, pharyngitis, conjunctivitis, red face, red neck, hemorrhage, organ failure, etc.), oncogenic symptoms (e.g., sarcomas, leukemias and the like, as well as “rare” malignancies, e.g., Kaposi's sarcoma, oral hairy leukoplasia, lymphomas, etc.), immunodeficiency symptoms (e.g., opportunistic infections, wasting, rare malignancies, neurological disease, fever, diarrhea, skin rashes, etc.), lesions (e.g., warts (e.g., common wart, flat wart, deep hyperkeratotic palmoplantar wart, superficial mosaic type palmoplantar wart, etc.)), epidermodysplasia, mucosal lesions, ulcers and systemic symptoms (e.g., fever, chills, headache, muscle pain, bone pain, joint pain, pharyngitis, tonsillitis, sinusitis, otitis, bronchitis, pneumonia, bronchopneumonia, nausea, vomiting, increased salivation, rash, macules, lymphadenopathy, arthritis, ulcers, photosensitivity, weight loss, irritability, restlessness, anxiety, coma, death, etc.).

In certain embodiments, the compounds disclosed herein can be used to treat disorders characterized by unwanted synthesis and/or abnormal accumulation of one or more mutant and/or wild-type proteins. It is contemplated that the compounds disclosed herein that can inhibit translation initiation and thus can reduce the load on the protein-folding machinery and, accordingly, may reduce the severity of the disorder. Disorders associated with unwanted synthesis and/or abnormal accumulation of one or more mutant and/or wild-type proteins include, but are not limited to, Tay-Sachs disease, cystic fibrosis, phenylketonuria, Fabry disease, Alzheimer's disease, Huntington's disease, Parkinson's disease, frontotemporal dementia, congophilic angiopathy, prion related disorders (i.e., transmissible spongiform encephalopathies such as Creutzfeldt-Jacob disease, kuru, fatal familial insomnia, scrapie, bovine spongiform encephalopathy, etc.), and the like.

It is contemplated that the compounds and compositions disclosed herein are capable of inhibiting neuronal cell death, such as in prion disease. Generally, the method includes administering a therapeutically effective amount of a compound or composition as described herein, to a patient in need thereof.

In some embodiments, the disorder is a neurodegenerative disease. The term “neurodegenerative disease” refers to a disease or condition in which the function of a subject's nervous system becomes impaired. Examples of neurodegenerative diseases include, e.g., Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizacus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, vanishing white matter (VWM) disease, insulin resistance or Tabes dorsalis.

Other embodiments include use of the presently disclosed compounds in therapy. Some embodiments include their use in the treatment of a neurodegenerative disease.

In other embodiments, provided are the presently disclosed compounds for use in the treatment of vanishing white matter (VWM) disease, Alzheimer's disease, Parkinson's disease, dementia, or ALS.

In other embodiments, provided is the use of the presently disclosed compounds for the manufacture of a medicament for treating a neurodegenerative disease.

In other embodiments, provided is the use of the presently disclosed compounds for the manufacture of a medicament for treating vanishing white matter (VWM) disease, Alzheimer's disease, Parkinson's disease, dementia, or ALS.

In other embodiments, provided is the use of the presently disclosed compounds, or a pharmaceutically acceptable salt thereof, for the manufacture of a medicament for treating cancer.

In other embodiments, provided is the presently disclosed compounds, or pharmaceutically acceptable salt thereof, for use in therapy.

In other embodiments, provided is the presently disclosed compounds, or pharmaceutically acceptable salt thereof, for use in treating a neurodegenerative disease.

In other embodiments, provided is the presently disclosed compounds, or pharmaceutically acceptable salt thereof, for use in treating cancer.

### 4. KITS

Provided herein are also kits that include a compound of the disclosure, or a pharmaceutically acceptable salt, isotopically enriched analog, stereoisomer, mixture of stereoisomers or prodrug thereof, and suitable packaging. In certain embodiments, a kit further includes instructions for use. In one aspect, a kit includes a compound of the disclosure, or a pharmaceutically acceptable salt, isotopically enriched analog, stereoisomer, mixture of stereoisomers or prodrug thereof, and a label and/or instructions for use of the compounds in the treatment of the indications, including the diseases or conditions, described herein.

Provided herein are also articles of manufacture that include a compound described herein or a pharmaceutically acceptable salt, isotopically enriched analog, stereoisomer, mixture of stereoisomers or prodrug thereof in a suitable container. The container may be a vial, jar, ampoule, preloaded syringe or intravenous bag.

### 5. PHARMACEUTICAL COMPOSITIONS AND MODES OF ADMINISTRATION

Compounds provided herein are usually administered in the form of pharmaceutical compositions. Thus, provided herein are also pharmaceutical compositions that contain one or more of the compounds described herein a pharmaceutically acceptable salt, stereoisomer, mixture of stereoisomers or prodrug thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, isotopically enriched analog, stereoisomer, mixture of stereoisomers or prodrug thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include, e.g., lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup and methyl cellulose. The formulations can additionally include lubricating agents such as talc, magnesium stearate and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, isotopically enriched analog, stereoisomer, mixture of stereoisomers or prodrug thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, isotopically enriched analog, stereoisomer, mixture of stereoisomers or prodrug thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids or mixtures of polymeric acids with such materials as shellac, cetyl alcohol and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### 6. DOSING

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. In some embodiments, a dosage of from about 0.0001 to about 100 mg per kg of body weight per day, from about 0.001 to about 50 mg of compound per kg of body weight, or from about 0.01 to about 10 mg of compound per kg of body weight may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

### 7. SYNTHESIS OF THE COMPOUNDS

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents and starting materials may be purchased commercially, e.g., from Sigma Aldrich or other chemical suppliers.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein. For example, protecting groups for alcohols, such as hydroxy, include silyl ethers (including trimethylsilyl (TMS), tert-butyldimethylsilyl (TBDMS), tri-iso-propylsilyloxymethyl (TOM), and triisopropylsilyl (TIPS) ethers), which can be removed by acid or fluoride ion, such as NaF, TBAF (tetra-n-butylammonium fluoride), HF-Py, or HF-NEt₃. Other protecting groups for alcohols include acetyl, removed by acid or base, benzoyl, removed by acid or base, benzyl, removed by hydrogenation, methoxyethoxymethyl ether, removed by acid, dimethoxytrityl, removed by acid, methoxymethyl ether, removed by acid, tetrahydropyranyl or tetrahydrofuranyl, removed by acid, and trityl, removed by acid. Examples of protecting groups for amines include carbobenzyloxy, removed by hydrogenolysis p-methoxybenzyl carbonyl, removed by hydrogenolysis, tert-butyloxycarbonyl, removed by concentrated strong acid (such as HCl or CF₃COOH), or by heating to greater than about 80° C., 9-fluorenylmethyloxycarbonyl, removed by base, such as piperidine, acetyl, removed by treatment with a base, benzoyl, removed by treatment with a base, benzyl, removed by hydrogenolysis, carbamate group, removed by acid and mild heating, p-methoxybenzyl, removed by hydrogenolysis, 3,4-dimethoxybenzyl, removed by hydrogenolysis, p-methoxyphenyl, removed by ammonium cerium(IV) nitrate, tosyl, removed by concentrated acid (such as HBr or H₂SO₄) and strong reducing agents (sodium in liquid ammonia or sodium naphthalenide), troc (trichloroethyl chloroformate), removed by Zn insertion in the presence of acetic acid, and sulfonamides (Nosyl & Nps), removed by samarium iodide or tributyltin hydride.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

General Synthesis

In certain embodiments, provided is a method of preparing a compound of Formula A, comprising: i) coupling a compound of Formula 1:

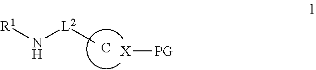

where PG is a protecting group, with a compound of Formula 2:

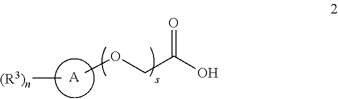

and deprotecting the resulting compound to provide the compound of Formula 3:

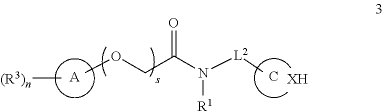

and ii) coupling the compound of Formula 3 with a compound of Formula 4 wherein LG is a leaving group (e.g., halo):

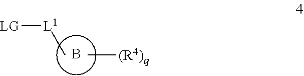

under conditions to provide the compound of Formula A, wherein R¹, R², R³, R⁴, L¹, L², ring A, ring B, ring C, X, n, s, and q are as defined herein.

The following reaction shown in Scheme I illustrates general methods which can be employed for the synthesis of compounds disclosed herein. In Scheme T, wherein R¹, R², R³, R⁴, L¹, L², ring A, ring B, ring C, X, n, s, and q are as defined herein, and PG is an amine protecting group (e.g., tert-butoxycarbonyl).

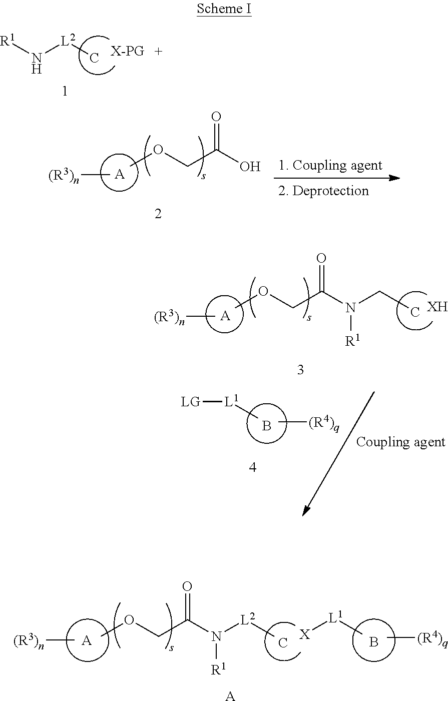

Referring to General Reaction Scheme 1 by coupling suitably protected amine 1 with the desired acid 2 in the presence of a coupling agent. Following any necessary deprotection step, a second coupling step can be performed to provide compounds of Formula A by reacting compound 3 with suitably substituted compound 4. The coupling reactions typically employs a coupling agent, such as a carbodiimide (e.g., N,N′-dicyclohexylcarbodiimide (DCC), N,N′-dicyclopentylcarbodiimide, N,N′-diisopropylcarbodiimide (DIC), 1-ethyl-3-(3-dimethylaminopropyl)carbodiimide (EDC), N-t-butyl-N-methylcarbodiimide (BMC), N-t-butyl-N-ethylcarbodiimide (BEC), 1,3-bis(2,2-dimethyl-1,3-dioxolan-4-ylmethyl)carbodiimide (BDDC), etc.), anhydrides (e.g., symmetric, mixed, or cyclic anhydrides), an activated ester (e.g., phenyl activated ester derivatives, p-hydroxamic activated ester, hexafluoroacetone (HFA), etc.), acylazoles (acylimidazoles using CDI, acylbenzotriazoles, etc.), acyl azides, acid halides, phosphonium salts (HOBt, PyBOP, HOAt, etc.), aminium/uronium salts (e.g., tetramethyl aminium salts, bispyrrolidino aminium salts, bispiperidino aminium salts, imidazolium uronium salts, pyrimidinium uronium salts, uronium salts derived from N,N,N′-trimethyl-N′-phenylurea, morpholino-based aminium/uronium coupling reagents, antimoniate uronium salts, etc.), an organophosphorus reagent (e.g., phosphinic and phosphoric acid derivatives, such as propylphosphonic anhydride), organosulfur reagents (e.g., sulfonic acid derivatives), a triazine coupling reagent (e.g., 2-chloro-4,6-dimethoxy-1,3,5-triazine, 4-(4,6-dimethoxy-1,3,5-triazin-2-yl)-4 methylmorpholinium chloride, 4-(4,6-dimethoxy-1,3,5-triazin-2-yl)-4 methylmorpholinium tetrafluoroborate, etc.), pyridinium coupling reagents (e.g., Mukaiyama's reagent, pyridinium tetrafluoroborate coupling reagents, etc.), and the like (see, e.g., El-Faham, et al. Chem. Rev., 2011, 111(11): 6557-6602; Han, et al. Tetrahedron, 2004, 60:2447-2467).

In some embodiments, the compounds of Formula A are asymmetric. Such compounds can be synthesized according to Scheme 1

Appropriate starting materials and reagents, including compounds 1, 2 and 4 (i.e., diamines, esters and acids) can be purchased or prepared by methods known to one of skill in the art.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

General Experimental Methods

All solvents used were commercially available and were used without further purification. Reactions were typically run using anhydrous solvents under an inert atmosphere of nitrogen.

NMR Spectroscopy: ¹H Nuclear magnetic resonance (NMR) spectroscopy was carried out using a Bruker Avance III equipped with a BBFO 300 MHz probe operating at 300 MHz or one of the following instruments: a Bruker Avance 400 instrument equipped with probe DUAL 400 MHz S1, a Bruker Avance 400 instrument equipped with probe 6 S1 400 MHz 5 mm ¹H-¹³C ID, a Bruker Avance III 400 instrument with nanobay equipped with probe Broadband BBFO 5 mm direct, a Bruker Mercury Plus 400 NMR spectrometer equipped with a Bruker 400 BBO probe with all operating at 400 MHz. All deuterated solvents contained typically 0.03% to 0.05% v/v tetramethylsilane, which was used as the reference signal (set at δ 0.00 for both ¹H and ¹³C). In certain cases, ¹H Nuclear magnetic resonance (NMR) spectroscopy was carried out using a Bruker Advance 400 instrument operating at 400 MHz using the stated solvent at around room temperature unless otherwise stated. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; dt, doublet of triplets; br, broad.

Thin Layer Chromatography: Where thin layer chromatography (TLC) has been used it refers to silica gel TLC using silica gel F254 (Merck) plates, Rf is the distance travelled by the compound divided by the distance travelled by the solvent on a TLC plate. Column chromatography was performed using an automatic flash chromatography system over silica gel cartridges or in the case of reverse phase chromatography over C18 cartridges. Alternatively, thin layer chromatography (TLC) was performed on Alugram® (Silica gel 60 F254) from Mancherey-Nagel and UV was typically used to visualize the spots. Additional visualization methods were also employed in some cases. In these cases the TLC plate was developed with iodine (generated by adding approximately 1 g of I₂ to 10 g silica gel and thoroughly mixing), ninhydrin (available commercially from Aldrich), or Magic Stain (generated by thoroughly mixing 25 g (NH₄)₆Mo₇O₂₄·4H₂O, 5 g (NH₄)₂Ce(IV)(NO₃)₆ in 450 mL water and 50 mL concentrated H₂SO₄) to visualize the compound.

Liquid Chromatography-Mass Spectrometry and HPLC Analysis: HPLC analysis was performed on Shimadzu 20AB HPLC system with a photodiode array detector and Luna-C18(2) 2.0×50 mm, 5 μm column at a flow rate of 1.2 mL/min with a gradient solvent Mobile phase A (MPA, H₂O+0.037% (v/v) TFA): Mobile phase B (MPB, ACN+0.018% (v/v) TFA) (0.01 min, 10% MPB; 4 min, 80% MPB; 4.9 min, 80% MPB; 4.92 min, 10% MPB; 5.5 min, 10% MPB). LCMS was detected under 220 and 254 nm or used evaporative light scattering (ELSD) detection as well as positive electrospray ionization (MS). Semi-preparative HPLC was performed by either acidic or neutral conditions. Acidic: Luna C18 100×30 mm, 5 μm; MPA: HCl/H₂O=0.04%, or formic acid/H₂O=0.2% (v/v); MPB: ACN. Neutral: Waters Xbridge 150×25, 5 m; MPA: 10 mM NH₄HCO₃ in H₂O; MPB: ACN. Gradient for both conditions: 10% of MPB to 80% of MPB within 12 min at a flow rate of 20 mL/min, then 100% MPB over 2 min, 10% MPB over 2 min, UV detector. SFC analysis was performed on Thar analytical SFC system with a UV/Vis detector and series of chiral columns including AD-3, AS-H, OJ-3, OD-3, AY-3 and IC-3, 4.6×100 mm, 3 μm column at a flow rate of 4 mL/min with a gradient solvent Mobile phase A (MPA, CO₂): Mobile phase B (MPB, MeOH+0.05% (v/v) IPAm) (0.01 min, 10% MPB; 3 min, 40% MPB; 3.5 min, 40% MPB; 3.56-5 min, 10% MPB). SFC preparative was performed on Thar 80 preparative SFC system with a UV/Vis detector and series of chiral preparative columns including AD-H, AS-H, OJ-H, OD-H, AY-H and IC-H, 30×250 mm, Sum column at a flow rate of 65 mL/min with a gradient solvent Mobile phase A (MPA, CO₂): Mobile phase B (MPB, MeOH+0.1% (v/v) NH₃H₂O) (0.01 min, 10% MPB; 5 min, 40% MPB; 6 min, 40% MPB; 6.1-10 min, 10% MPB). LC-MS data were also collected using an UPLC-MS Acquity™ system equipped with PDA detector and coupled to a Waters single quadrupole mass spectrometer operating in alternated positive and negative electrospray ionization mode. The column used was a Cortecs UPLC C18, 1.6 μm, 2.1×50 mm. A linear gradient was applied, starting at 95% A (A: 0.1% formic acid in water) and ending at 95% B (B: 0.1% formic acid in MeCN) over 2.0 min with a total run time of 2.5 min. The column temperature was at 40° C. with the flow rate of 0.8 mL/min.

General procedure A, T3P coupling: To a flask containing amine (1 eq), and carboxylic acid (1.5 eq) in DMF or EtOAc (0.1 M-0.2 M) were added either N-methylimidazole, diisopropylethylamine, or triethylamine (3.0-5.0 eq) followed by T3P solution (1.5-3.0 eq., 50% in EtOAc). The resulting reaction mixture was stirred at rt for 4 h, at which point 1M NaOH solution was added followed by EtOAc. The layers were separated, and the aqueous layer was extracted with EtOAc (3×). The combined organic layers were dried over anhydrous MgSO₄, filtered and concentrated under reduced pressure. The crude reaction mixture was purified employing silica flash chromatography or reverse-phase HPLC to provide the desired product.

General Procedure B, Etherification: To a flask containing alkyl bromide (1 equiv.) and alcohol (3 equiv.) in THF (˜0.1M) was added KO^{t}Bu (3 equiv.) at rt. The resulting reaction mixture was stirred at rt for 2 h at which point NH₄Cl solution (saturated, 10 mL) was added followed by EtOAc (10 mL). The layers were separated, and the aqueous layer was extracted with EtOAc (3×10 mL). The combined organic layers were dried over anhydrous MgSO₄, filtered and concentrated under reduced pressure and purified employing either silica gel flash chromatography (0 to 100% EtOAc/hexanes) or reverse-phase preparatory HPLC (Phenomenex Luna 10 μ C18, 100A, 150×30 mm, 5 to 95% MeCN (0.1% formic acid) in H₂O, 25 min, 60 mL/min) to afford the desired product.

General Procedure C, Etherification: To a flask containing alcohol (3 equiv.) in THF (0.1-0.5 M) at 0° C. was added sodium hydride (3 equiv. 60% oil immersion). The resulting slurry was stirred 10 min and the alkyl bromide (1 equiv.) was added. The reaction mixture was allowed to reach rt and was stirred 2 h at which point NH₄Cl solution (saturated, 10 mL) was added followed by EtOAc (10 mL). The layers were separated, and the aqueous layer was extracted with EtOAc (3×10 mL). The combined organic layers were dried over anhydrous MgSO₄, filtered and concentrated under reduced pressure and purified employing either silica gel flash chromatography (0 [symbol] 100% EtOAc/hexanes) or reverse-phase preparatory HPLC (Phenomenex Luna 10 μ C18, 100A, 150×30 mm, 5 [symbol] 95% MeCN (0.1% formic acid) in H₂O, 25 min, 60 mL/min) to afford the desired product.

General Procedure D, Etherification: To a flask containing phenol (1.2 equiv.) and alkyl bromide (1.0 equiv.) in DMF (0.1-0.5M) was added K₂CO₃ (2.0 equiv.). The resulting mixture was stirred at 60° C. for 4 h. The reaction mixture was diluted with sat. NH₄Cl solution (10 mL) followed by EtOAc (10 mL). The layers were separated, and the aqueous layer was extracted with EtOAc (3×10 mL). The combined organic layers were dried over anhydrous MgSO₄, filtered and concentrated under reduced pressure. The crude reaction mixture was purified employing either silica gel flash chromatography (0 to 100% EtOAc/hexanes) or reverse-phase preparatory HPLC (Phenomenex Luna 10 μ C18, 100A, 150×30 mm, 5 to 95% MeCN (0.1% formic acid) in H₂O, 25 min, 60 mL/min) to afford the desired product.

General procedure E, Hydrazide Formation: To a suspension of the methyl ester (1 eq) in EtOH (0.25-0.1M) was added hydrazine hydrate (3-5 eq) and the reaction mixture was heated at 90° C. overnight. The reaction mixture was cooled to rt often causing the product to crystallize out of solution. This solid was collected by removal of the supernatant. If the product did not crystallize, the solution was concentrated, and the crude product was sufficiently pure to use in subsequent steps.

### Intermediate 1

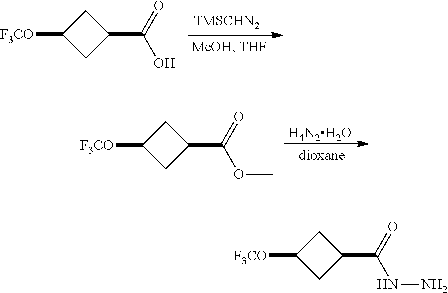

methyl 3-cis-(trifluoromethoxy)cyclobutanecarboxylate: To a solution of 3-cis-(trifluoromethoxy)cyclobutanecarboxylic acid (6.0 g, 32.6 mmol) in THF (48 mL) and MeOH (12 mL) was added dropwise a solution TMSCHN₂ (40.7 mL) at 0° C. under N₂. The reaction mixture was stirred at 25° C. for 3 h. The reaction mixture was quenched with AcOH (3 mL), diluted with H₂O (30 mL), and extracted with EtOAc (3×30 mL). The combined organics were washed with sat. NaHCO₃ (2×30 mL), brine (10 mL), dried over Na₂SO₄, filtered, and concentrated under reduced pressure. The residue was purified by silica gel column chromatography.

3-cis-(trifluoromethoxy)cyclobutanecarbohydrazide: To a solution of methyl 3-cis-(trifluoromethoxy)cyclobutanecarboxylate (4.3 g, 21.7 mmol) in dioxane (50 mL) was added NH₂NH₂·H₂O (21.7 g, 434.0 mmol) in one portion at 25° C. under N₂. The reaction mixture was stirred at 80° C. for 12 h. The reaction mixture cooled and then quenched by the addition of H₂O (30 mL) and extracted with EtOAc (3×30 mL). The combined organics were washed with brine (10 mL), dried over Na₂SO₄, filtered, and concentrated under reduced pressure. ¹H-NMR (400 MHz, CDCl₃): δ 6.75 (br s, 1H), 4.52-4.64 (m, 1H), 3.92 (br s, 2H), 2.54-2.61 (m, 4H) 2.42-2.53 (m, 1H).

### Intermediate 2

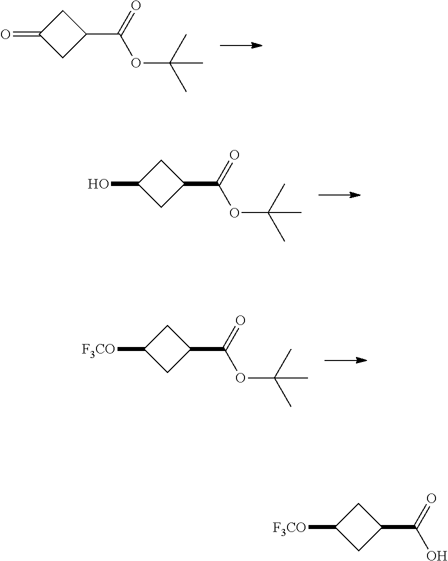 [SMILES: CC(C)(C)OC(=O)C1CC(=O)C1 ; CC(C)(C)OC(=O)[C@H]1C[C@@H](O)C1 ; CC(C)(C)OC(=O)[C@H]1C[C@@H](OC(F)(F)F)C1 ; O=C(O)[C@H]1C[C@@H](OC(F)(F)F)C1]

tert-butyl 3-cis-hydroxycyclobutanecarboxylate: A mixture of tert-butyl 3-oxocyclobutanecarboxylate (70.0 g, 411 mmol) in MeOH (700 mL) was added NaBH₄ (15.6 g, 411 mmol) at −30° C. under N₂ over 2 h. The reaction mixture was stirred at −30° C. for 0.5 h. The reaction mixture was quenched by the addition of ice and aq. sat. NH₄Cl (700 mL) slowly at 0° C. over 30 min. The reaction mixture was concentrated under reduced pressure to leave the aqueous phase that was extracted with EtOAc (3×300 mL). The combined organics were washed with brine (300 mL), dried over anhydrous Na₂SO₄, filtered, and concentrated. Mixture of diastereomers in favor of the cis-product. ¹H-NMR (400 MHz, CDCl₃): δ 4.23-4.04 (m, 1H), 2.79 (br s, 1H), 2.60-2.43 (m, 3H), 2.14-2.05 (m, 2H), 1.43 (s, 9H).

tert-butyl 3-cis-(trifluoromethoxy)cyclobutanecarboxylate: To a reaction flask equipped with a stir bar and covered with tin foil paper in a water bath were added AgOTf (134.3 g, 523 mmol), Selectfluor (92.6 g, 261 mmol), KF (40.5 g, 697 mmol) and tert-butyl 3-cis-hydroxycyclobutanecarboxylate (30.0 g, 174 mmol) under N₂. EtOAc (1000 mL), 2-fluoropyridine (50.7 g, 523 mmol) and TMSCF₃ (74.3 g, 523 mmol) were then added dropwise successively to reaction flask in a water bath while keeping the inner temperature below 30° C. The reaction mixture was stirred at 25° C. for 12 h. The reaction mixture was filtered through a plug of silica and the filtrate was concentrated under reduced pressure. The residue was washed with MTBE (800 mL) and filtered. The filtrate was washed with 1 N aq. CuSO₄ (3×300 mL) and the organics were dried over Na₂SO₄, filtered, and concentrated under reduced pressure. The residue was purified by silica gel column chromatography. ¹H-NMR (400 MHz, CDCl₃): δ 4.60-4.48 (m, 1H), 2.69-2.53 (m, 3H), 2.52-2.37 (m, 2H), 1.46 (s, 9H).

3-cis-(trifluoromethoxy)cyclobutanecarboxylic acid: To a solution of tert-butyl 3-cis-(trifluoromethoxy)cyclobutanecarboxylate (24.0 g, 100.0 mmol) in DCM (250 mL) was added TFA (77.0 g, 675 mmol). The reaction mixture was stirred at 40° C. for 2 h. The reaction mixture was concentrated under reduced pressure. The residue was dissolved in DCM (50 mL), washed with H₂O (3×30 mL), dried over Na₂SO₄, filtered, and concentrated under reduced pressure. ¹H-NMR (400 MHz, DMSO-d6): δ 1239 (s, 1H), 4.74 (quin, J=7.44 Hz, 1H), 2.76-2.64 (m, 1H), 2.63-2.53 (m, 2H), 2.33-2.21 (m, 2H).

### Intermediate 3

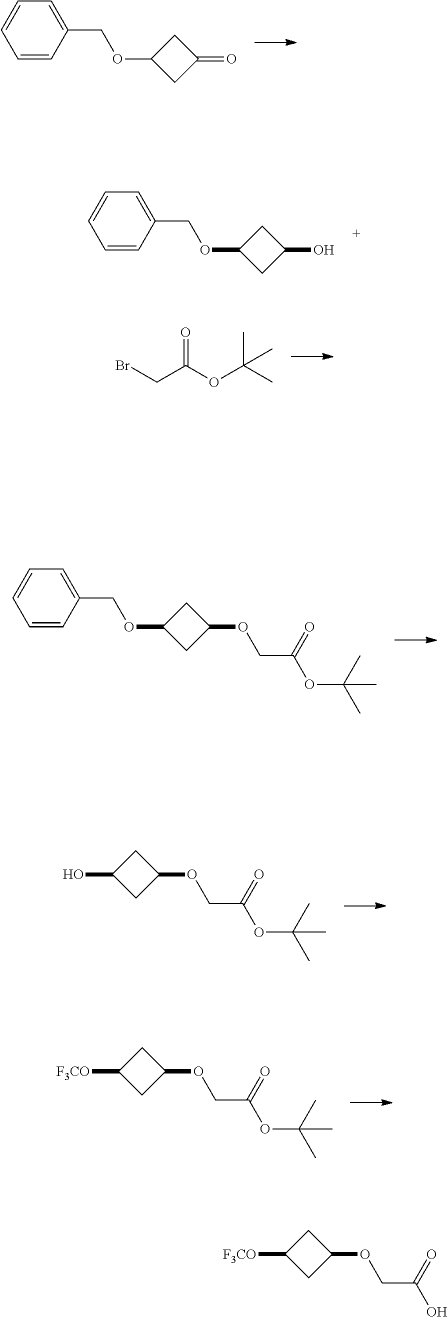

3-cis-(benzyloxy)cyclobutanol: A mixture of 3-benzyloxycyclobutanone (100.0 g, 567 mmol) in MeOH (1000 mL) was added NaBH₄ (21.5 g, 567 mmol) at −30° C. under N₂ over 2 h. The reaction mixture was stirred at −30° C. for 0.5 h. The reaction mixture was quenched by the addition of ice and aq. sat. NH₄Cl (600 mL) slowly at 0° C. over 0.5 h. The reaction mixture was concentrated under reduced pressure to leave the aqueous phase that was extracted with EtOAc (3×200 mL). The combined organics were washed with brine (200 mL), dried over anhydrous Na₂SO₄, filtered, and concentrated under reduced pressure. Mixture of diastereomers in favor of the cis-product. ¹H-NMR (400 MHz, CDCl₃): δ 7.32-7.18 (m, 5H), 4.35 (s, 2H), 3.83 (quin, J=7.17 Hz, 1H), 3.56 (quin, J=6.95 Hz, 1H), 2.69-2.60 (m, 2H), 1.91-1.82 (m, 2H).

tert-butyl 2-(3-cis-(benzyloxy)cyclobutoxy)acetate: To a mixture of 3-cis-(benzyloxy)cyclobutanol (19.7 g, 110 mmol), tert-butyl 2-bromoacetate (32.3 g, 165 mmol), tetrabutylammonium hydrogen sulfate (1.9 g, 5.5 mmol), and water (10 mL) in toluene (400 mL) was added NaOH (66.3 g, 1.6 mol) in water (120 mL). The reaction mixture was stirred at 25° C. for 4 h. The reaction mixture was quenched by addition of ice-water (120 mL) and extracted with MTBE (3×50 mL). The combined organics were washed with brine (100 mL), dried over anhydrous Na₂SO₄, filtered, and concentrated under reduced pressure. ¹H-NMR (400 MHz, CDCl₃): δ 7.39-7.27 (m, 5H), 4.42 (s, 2H), 3.89 (s, 2H), 3.75-3.62 (m, 2H), 2.65 (dtd, J=9.26, 6.28, 6.28, 3.31 Hz, 2H), 2.09-2.00 (m, 2H), 1.48 (s, 9H).

tert-butyl 2-(3-cis-hydroxycyclobutoxy)acetate: To a solution of tert-butyl 2-(3-cis-(benzyloxy)cyclobutoxy)acetate (27.0 g, 92.4 mmol) in MeOH (350 mL) was added Pd/C (3.0 g, 10% Pd on carbon) under N₂. The reaction mixture was degassed under vacuum, purged with H₂ three times, and stirred under H₂ (50 psi) at 50° C. for 12 h. The reaction mixture was filtered and concentrated under reduced pressure. ¹H-NMR (400 MHz, CDCl₃): δ 3.94-3.89 (m, 1H), 3.88 (s, 2H), 3.67 (quin, J=6.89 Hz, 1H), 2.78-2.69 (m, 2H), 2.01-1.92 (m, 2H), 1.80 (br d, J=6.39 Hz, 1H), 1.47 (s, 9H).

tert-butyl 2-(3-cis-(trifluoromethoxy)cyclobutoxy)acetate: To a reaction flask equipped with a stir bar and covered with tin foil paper in a water bath were added AgOTf (57.2 g, 222 mmol), Selectfluor (39.4 g, 111 mmol), KF (17.2 g, 297 mmol), and tert-butyl 2-(3-cis-hydroxycyclobutoxy)acetate (15.0 g, 74.2 mmol) under N₂. And then EtOAc (600 mL), 2-fluoropyridine (21.6 g, 222 mmol) and TMSCF₃ (31.6 g, 222 mmol) were added dropwise successively water bath while keeping the inner temperature below 30° C. The reaction mixture was stirred at 25° C. for 12 h. The reaction mixture was filtered through a plug of silica and the filtrate was concentrated under reduced pressure. The residue was washed with MTBE (800 mL) and filtered. The residue was purified by silica gel column chromatography. ¹H-NMR (400 MHz, CDCl₃): δ 4.35-4.22 (m, 1H), 3.90 (s, 2H), 3.81-3.69 (m, 1H), 2.86-2.72 (m, 2H), 2.40-2.23 (m, 2H), 1.49 (s, 9H).

2-(3-cis-(trifluoromethoxy)cyclobutoxy)acetic acid: To a solution of tert-butyl 2-(3-cis-(trifluoromethoxy)cyclobutoxy)acetate (11.5 g, 42.6 mmol) in DCM (100 mL) was added TFA (30.8 g, 270 mmol) under N₂. The reaction mixture was stirred at 40° C. for 2 h. The reaction mixture was concentrated under reduced pressure. The residue was dissolved in DCM (30 mL), washed with H₂O (3×30 mL), dried over Na₂SO₄, filtered, and concentrated under reduced pressure. ¹H-NMR (400 MHz, CDCl₃): δ 4.26-4.19 (m, 1H), 4.00 (s, 2H), 3.73-3.70 (m, 1H), 2.77-2.74 (m, 2H), 2.27-2.24 (m, 2H).

### Intermediate 4

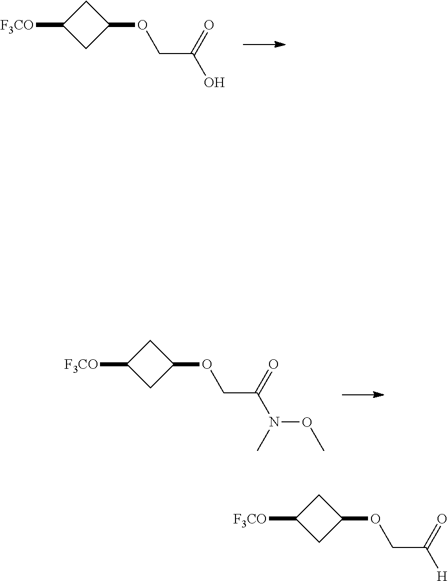

N-methoxy-N-methyl-2-[3-cis-(trifluoromethoxy)cyclobutoxy]acetamide: To a solution of 2-[3-cis-(trifluoromethoxy)cyclobutoxy]acetic acid (1.6 g, 7.7 mmol) in EtOAc (50 mL) was added N,N-diisopropylethylamine (4.47 mL, 25.7 mmol) followed by T3P (2.4 g, 7.7 mmol, 50% in EtOAc). The resulting reaction mixture was stirred 10 min and N,O-dimethylhydroxylamine hydrochloride (500 mg, 5.13 mmol) was added. The reaction mixture was stirred at 23° C. for 4 h. The reaction mixture was quenched by the addition of sat. NH₄Cl and extracted with EtOAc (3×25 mL) The combined organic layers are dried over MgSO₄, filtered, and concentrated under reduced pressure. The crude reaction mixture was sufficiently pure and was used directly. ¹H-NMR (400 MHz; CDCl₃): δ 4.29 (t, J=7.3 Hz, 1H), 4.21 (s, 2H), 3.83 (td, J=7.0, 1.1 Hz, 1H), 3.71 (s, 3H), 3.21 (s, 3H), 2.83 (dtd, J=9.8, 6.6, 3.2 Hz, 2H), 2.38-2.32 (m, 2H).

2-[3-cis-(trifluoromethoxy)cyclobutoxy]acetaldehyde: To a cooled solution of N-methoxy-N-methyl-2-[3-cis-(trifluoromethoxy)cyclobutoxy]acetamide (100 mg, 0.39 mmol) in THF (3.9 mL) at −78° C. was added diisobutylalumninum hydride (0.78 mL, 0.78 mmol, 1 M in hexane). The reaction mixture was stirred at −78° C. for 2 h. The reaction mixture was quenched by the addition of anhydrous EtOAc (1.0 mL) and sat. aq. NH₄Cl (3 mL) and then removed from the cooling bath and allowed to stir for 15 min. The reaction mixture was then dilute with water (20 mL) and extracted with Et₂O (3×15 mL). The combined organics were dried over Na₂SO₄, filtered and concentrated under reduced pressure. The crude aldehyde was used immediately and without further purification.

### Intermediate 5

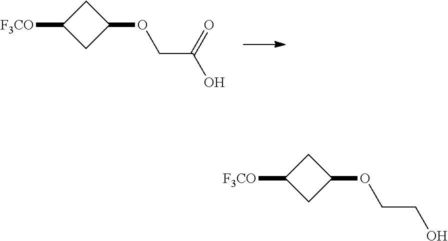

2-[3-cis-(trifluoromethoxy)cyclobutoxy]ethanol: To solution of 2-[3-cis-(trifluoromethoxy)cyclobutoxy]acetic acid (300 mg, 1.4 mmol) in THF (3.5 mL) at 0° C. degrees was added BH₃ (14.0 mL, 14.0 mmol, 1 M in THF). The reaction was stirred at 0° C. degrees for 30 min and then at 23° C. overnight. The reaction mixture was quenched by the addition of 1 N aq. NaOH (12 mL). After stirring for an additional 30 min, the reaction mixture was diluted with EtOAc (15 mL) and water (10 mL). The organics were then separated and the aqueous extracted with EtOAc (3×10 mL). The combined organics were then dried over MgSO₄, filtered, and concentrated under reduced pressure. The crude product was then used in the next transformation without further purification. ¹H-NMR (400 MHz, CDCl₃): δ 4.33 (quintet, J=7.2 Hz, 1H), 3.76 (t, J=4.6 Hz, 2H), 3.70 (td, J=13.1, 6.5 Hz, 1H), 3.50-3.44 (m, 2H), 2.82 (dtd, J=12.9, 6.4, 3.1 Hz, 2H), 2.29-2.22 (m, 2H).

### Intermediate 6

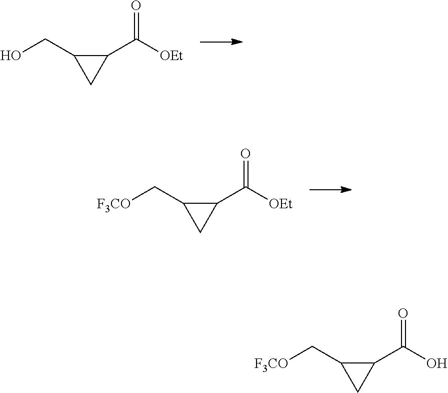 [SMILES: CCOC(=O)C1CC1CO ; CCOC(=O)C1CC1COC(F)(F)F ; O=C(O)C1CC1COC(F)(F)F]

Ethyl 2-((trifluoromethoxy)methyl)cyclopropanecarboxylate: To a solution of AgOTf (11.23 g, 43.70 mmol) in EtOAc (80 mL) at rt was added 1-(chloromethyl)-4-fluoro-1,4-diazoniabicyclo[2.2.2]octane;ditetrafluoroborate (7.74 g, 21.85 mmol), KF (3.39 g, 58.27 mmol) and ethyl 2-(hydroxymethyl)cyclopropanecarboxylate (2.1 g, 14.57 mmol). 2-fluoropyridine (4.24 g, 43.70 mmol) and trimethyl(trifluoromethyl)silane (6.21 g, 43.70 mmol) were added to this mixture and the resulting suspension was stirred at rt for 12 h. The reaction mixture was filtered through a pad of silica and was concentrated under reduced pressure. The residue was purified by silica gel column chromatography (PE:EtOAc=100:1 to 5:1) to provide ethyl 2-(trifluoromethoxymethyl)cyclopropanecarboxylate (cis:trans=1:3).

2-((trifluoromethoxy)methyl)cyclopropanecarboxylic acid: To a solution of ethyl 2-(trifluoromethoxymethyl)cyclopropanecarboxylate (0.86 g, 4.05 mmol) in THF (10 mL) and H₂O (10 mL) at 0° C. was added LiOH·H₂O (510 mg, 12.16 mmol) and the mixture was warmed to rt and stirred for 2 h. The reaction mixture was concentrated under reduced pressure and the residue was diluted with H₂O (20 mL) and MTBE (10 mL). The layers were separated and the aqueous layer was extracted with MTBE (3×10 mL). The aqueous phase was adjusted to pH=1-2 by addition of 2N HCl and was further extracted with DCM:MeOH (6×10 mL, v:v=10:1). The combined organic layers were dried over anhydrous Na₂SO₄, filtered and concentrated under reduced pressure to provide 2-(trifluoromethoxymethyl)cyclopropanecarboxylic acid (cis:trans=1:5). The crude product was used for the next step without further purification. ¹H-NMR (400 MHz, CDCl₃): δ 4.31-3.78 (m, 2H), 1.83-1.96 (m, 1H), 1.68 (dt, J=8.54, 4.44 Hz, 1H), 1.34-1.42 (m, 1H), 0.98-1.08 (m, 1H).

### Intermediate 7

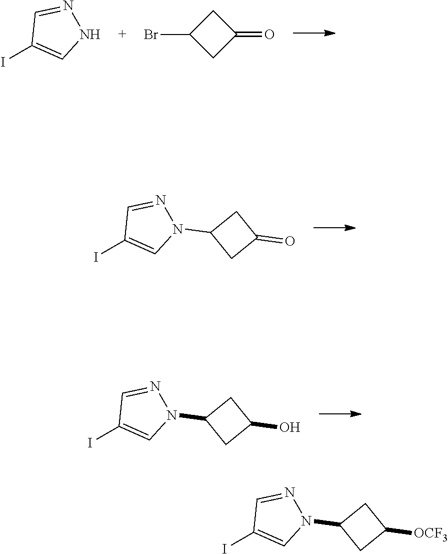

3-(4-iodo-1H-pyrazol-1-yl)cyclobutanone: To a solution of 4-iodo-1H-pyrazole (1.00 g, 5.16 mmol) in DMF (40 mL) was added Cs₂CO₃ (1.68 g, 5.16 mmol), 3-bromocyclobutanone (768 mg, 5.16 mmol) at −10° C., and the mixture was stirred at −10° C. for 1 h. The reaction mixture was diluted with H₂O (90 mL) and was extracted with EtOAc (3×30 mL). The combined organic layers were washed with brine (30 mL), dried over anhydrous Na₂SO₄, filtered and concentrated under reduced pressure. The crude residue was purified by flash silica gel column chromatography to give the desired product. LCMS: m/z=263.0 [M+H]⁺. ¹H-NMR (400 MHz, CDCl₃): δ 7.58 (d, J=5.51 Hz, 2H), 5.02 (tt, J=8.02, 5.98 Hz, 1H), 3.83-3.71 (m, 2H), 3.62-3.52 (m, 2H).

3-cis-(4-iodo-1H-pyrazol-1-yl)cyclobutanol: To a solution of 3-(4-iodo-1H-pyrazol-1-yl)cyclobutanone (0.95 g, 3.63 mmol) in MeOH (10 mL) was added NaBH₄ (137 mg, 3.63 mmol) at −30° C., and the mixture was stirred at −30° C. for 1 h. The reaction mixture was diluted with sat. NH₄Cl (60 mL) at 0° C. and then concentrated under reduced pressure to remove MeOH. The remaining aqueous phase was extracted with EtOAc (3×20 mL), the combined organic layers were washed with brine (30 mL), dried over Na₂SO₄, filtered, and concentrated under reduced pressure. The resulting residue was used directly. LCMS: m/z=265.0 [M+H]⁺. ¹H-NMR (400 MHz, CDCl₃): δ 7.57 (s, 1H), 7.49 (s, 1H), 4.42-4.32 (m, 1H), 4.25-4.14 (m, 1H), 3.05-2.91 (m, 2H), 2.55-2.45 (m, 2H).

4-iodo-1-(3-cis-(trifluoromethoxy)cyclobutyl)-1H-pyrazole: To a mixture of silver trifluoromethanesulfonate (2.92 g, 11.36 mmol), 1-(chloromethyl)-4-fluoro-1,4-diazoniabicyclo[2.2.2]octane ditetrafluoroborate (2.01 g, 5.68 mmol), KF (880 mg, 15.15 mmol) in EtOAc (20 mL) was added 3-cis-(4-iodo-1H-pyrazol-1-yl)cyclobutanol (1.00 g, 3.79 mmol), 2-fluoropyridine (1.10 g, 11.36 mmol) and trimethyl(trifluoromethyl)silane (1.62 g, 11.36 mmol), and the mixture was stirred at 30° C. for 40 h. The reaction mixture was then filtered and was concentrated under reduced pressure. The residue was purified by silica gel column chromatography to give the desired product. LCMS: m/z=333.0 [M+H]. ¹H-NMR (400 MHz, CDCl₃): δ 7.57 (s, 1H), 7.50 (s, 1H), 4.55 (quin, J=7.31 Hz, 1H), 4.50-4.37 (m, 1H), 3.09-2.95 (m, 2H), 2.92-2.82 (m, 2H).

### Example 1

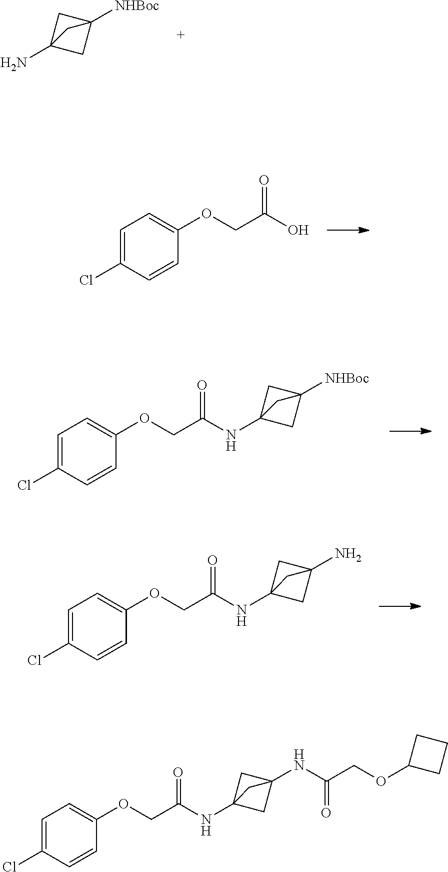 [SMILES: CC(C)(C)OC(=O)NC12CC(N)(C1)C2 ; "+" ; O=C(O)COc1ccc(Cl)cc1 ; CC(C)(C)OC(=O)NC12CC(NC(=O)COc3ccc(Cl)cc3)(C1)C2 ; NC12CC(NC(=O)COc3ccc(Cl)cc3)(C1)C2 ; O=C(COc1ccc(Cl)cc1)NC12CC(NC(=O)COC3CCC3)(C1)C2]

tert-butyl (3-(2-(4-chlorophenoxy)acetamido)bicyclo[1.1.1]pentan-1-yl)carbamate: Prepared employing General Procedure A employing tert-butyl (3-aminobicyclo[1.1.1]pentan-1-yl)carbamate (400 mg, 2.01 mmol), 2-(4-chlorophenoxy)acetic acid (452 mg, 2.42 mmol), N-methylimidazole (497 mg, 6.05 mmol), and T3P solution (769 mg, 50% in EtOAc) in DMF (8.0 mL). Purified employing silica flash chromatography (0 to 10% MeOH/CH₂Cl₂) to provide the desired product. ¹H-NMR (400 MHz; CDCl₃): δ 7.32-7.29 (m, 2H), 6.92-6.90 (m, 1H), 6.89-6.86 (m, 2H), 4.42 (s, 2H), 2.40 (s, 6H), 1.47 (s, 9H).

tert-butyl (3-(2-(4-chlorophenoxy)acetamido)bicyclo[1.1.1]pentan-1-yl)carbamate from the previous step was dissolved in CH₂Cl₂ (5 mL) and TFA (5 mL) and stirred for 4 h, upon which the solution was concentrated. The resulting residue was partitioned between CH₂Cl₂ and 1 M aq. Na₂CO₃ solution (10 mL each). The layers were separated, and the aqueous layer was extracted with CH₂Cl₂ (3×10 mL). The combined organic layers were dried over anhydrous Na₂SO₄, filtered and concentrated under reduced pressure. The crude reaction mixture was used directly.

2-(4-chlorophenoxy)-N-(3-(2-cyclobutoxyacetamido)bicyclo[1.1.1]pentan-1-yl)acetamide: Prepared employing General Procedure B employing N-[3-[(2-bromoacetyl)amino]-1-bicyclo[1.1.1]pentanyl]-2-(4-chlorophenoxy)acetamide (58 mg, 0.15 mmol), cyclobutanol (32 mg, 0.45 mmol), and KO^{t}Bu (50 mg, 0.45 mmol) in THF (1 mL) for 1 h. Purified employing silica gel flash chromatography (30→100% EtOAc/hexanes) to deliver the desired compound. LC/MS: =379.20, 380.97 [M+H]⁺. ¹H-NMR (400 MHz; CDCl₃): δ 7.16-7.12 (m, 2H), 6.83-6.75 (m, 2H), 6.74-6.70 (m, 2H), 4.26 (s, 2H), 3.86-3.79 (m, 1H), 3.64 (s, 2H), 2.35 (s, 6H), 2.12-2.04 (m, 2H), 1.87-1.76 (m, 2H), 1.64-1.55 (m, 2H), 1.44-1.33 (m, 1H).

### Example 2

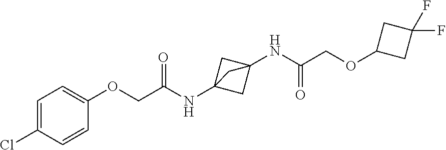 [SMILES: O=C(COc1ccc(Cl)cc1)NC12CC(NC(=O)COC3CC(F)(F)C3)(C1)C2]

2-(4-chlorophenoxy)-N-(3-(2-(3,3-difluorocyclobutoxy)acetamido)bicyclo[1.1.1]pentan-1-yl)acetamide: Prepared employing General Procedure B employing N-[3-[(2-bromoacetyl)amino]-1-bicyclo[1.1.1]pentanyl]-2-(4-chlorophenoxy)acetamide (58 mg, 0.15 mmol), 3,3-difluorocyclobutanol (49 mg, 0.45 mmol), and KO^{t}Bu (50 mg, 0.45 mmol) in THF (1.0 mL) for 1 h. Purified employing silica gel flash chromatography (30-+100% EtOAc/hexanes) to deliver the desired compound. m/z=415.13, 417.02 [M+H]⁺. ¹H-NMR (400 MHz; CDCl₃): δ 7.32-7.29 (m, 2H), 6.94 (s, 1H), 6.90-6.86 (m, 2H), 6.83 (s, 1H), 4.42 (s, 2H), 4.09-4.00 (m, 1H), 3.86 (s, 2H), 297-2.85 (m, 2H), 2.67-2.55 (m, 2H), 2.52 (s, 6H).

### Example 3

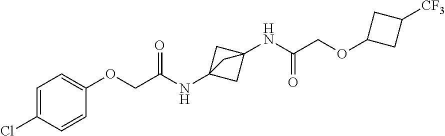 [SMILES: O=C(COc1ccc(Cl)cc1)NC12CC(NC(=O)COC3CC(C(F)(F)F)C3)(C1)C2]

2-(4-chlorophenoxy)-N-(3-(2-(3-(trifluoromethyl)cyclobutoxy)acetamido)bicyclo[1.1.1]pentan-1-yl)acetamide: Prepared employing General Procedure B employing N-[3-[(2-bromoacetyl)amino]-1-bicyclo[1.1.1]pentanyl]-2-(4-chlorophenoxy)acetamide (58 mg, 0.15 mmol), 3-(trifluoromethyl)cyclobutanol (32 mg, 0.45 mmol), and KO^{t}Bu (34 mg, 0.45 mmol) in THE (1 mL) for 1 h. Purified employing silica gel flash chromatography (30-+100% EtOAc/hexanes) to deliver the first eluting isomer and the second eluting isomer.

First eluting isomer: LC/MS: 447.15, 449.00 [M+H]⁺. ¹H-NMR (400 MHz; CDCl₃): δ 7.31-7.27 (m, 2H), 6.97 (s, 1H), 6.88 (dt, J=7.0, 2.6 Hz, 3H), 4.41 (s, 2H), 4.23-4.16 (m, 1H), 3.81 (s, 2H), 2.97-2.86 (m, 1H), 2.51-2.45 (m, 9H), 2.34-227 (m, 2H).

Second eluting isomer: m/z=447.15, 448.96 [M+H]⁺. ¹H-NMR (400 MHz; CDCl₃): δ 7.31-7.27 (m, 2H), 6.95 (s, 1H), 6.90-6.86 (m, 3H), 4.42 (s, 2H), 4.01-3.95 (m, 1H), 3.82 (s, 2H), 2.54-2.49 (m, 8H), 2.19-2.12 (m, 2H).

### Example 4

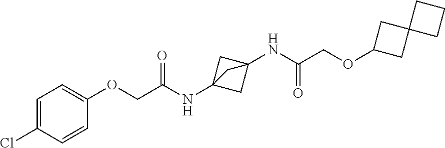 [SMILES: O=C(COc1ccc(Cl)cc1)NC12CC(NC(=O)COC3CC4(CCC4)C3)(C1)C2]

2-(4-chlorophenoxy)-N-(3-(2-(spiro[3.3]heptan-2-yloxy)acetamido)bicyclo[1.1.1]pentan-1-yl)acetamide: Prepared employing General Procedure B employing N-[3-[(2-bromoacetyl)amino]-1-bicyclo[1.1.1]pentanyl]-2-(4-chlorophenoxy)acetamide (20 mg, 0.05 mmol), spiro[3.3]heptan-2-ol (17 mg, 0.15 mmol), and KO^{t}Bu (17 mg, 0.15 mmol) in THF (0.5 mL) for 1 h. Purified employing reverse phase prep HPLC to provide the desired product. LC/MS: 419.25, 421.02 [M+H]⁺. ¹H-NMR (400 MHz; CDCl₃): δ 7.31-7.27 (m, 2H), 6.95-6.91 (m, 2H), 6.90-6.86 (m, 2H), 4.42 (s, 2H), 3.91-3.84 (m, 1H), 3.77 (s, 2H), 2.50 (s, 6H), 2.37-2.31 (m, 2H), 2.01-1.96 (m, 4H), 1.94-1.82 (m, 4H).

### Example 5

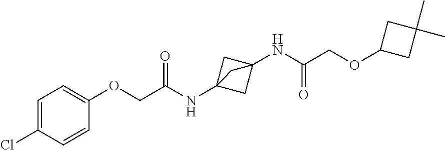 [SMILES: CC1(C)CC(OCC(=O)NC23CC(NC(=O)COc4ccc(Cl)cc4)(C2)C3)C1]

2-(4-chlorophenoxy)-N-(3-(2-(3,3-dimethylcyclobutoxy)acetamido)bicyclo[1.1.1]pentan-1-yl)acetamide: Prepared employing General Procedure B employing N-[3-[(2-bromoacetyl)amino]-1-bicyclo[1.1.1]pentanyl]-2-(4-chlorophenoxy)acetamide (20 mg, 0.05 mmol), 3,3-dimethylcyclobutanol (16 mg, 0.15 mmol), and KO^{t}Bu (17 mg, 0.15 mmol) in THF (0.5 mL) for 1 h. Purified employing reverse phase prep HPLC to provide the desired product. LC/MS: 407.23, 409.00 [M+H]⁺. ¹H-NMR (400 MHz; CDCl₃): δ 7.31-7.27 (m, 2H), 6.94 (d, J=5.4 Hz, 2H), 6.90-6.86 (m, 2H), 4.42 (s, 2H), 4.01 (quintet, J=7.1 Hz, 1H), 3.78 (s, 2H), 2.51 (s, 6H), 2.19-2.06 (m, 2H), 1.77 (ddd, J=9.6, 7.1, 2.7 Hz, 2H), 1.16 (s, 3H), 1.12 (s, 3H).

### Example 6

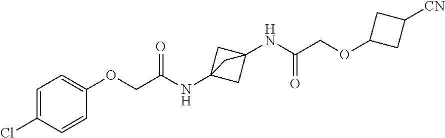 [SMILES: N#CC1CC(OCC(=O)NC23CC(NC(=O)COc4ccc(Cl)cc4)(C2)C3)C1]

2-(4-chlorophenoxy)-N-(3-(2-(3-cyanocyclobutoxy)acetamido)bicyclo[1.1.1]pentan-1-yl)acetamide: Prepared employing General Procedure B employing N-[3-[(2-bromoacetyl)amino]-1-bicyclo[1.1.1]pentanyl]-2-(4-chlorophenoxy)acetamide (20 mg, 0.05 mmol), 3-hydroxycyclobutanecarbonitrile (15 mg, 0.15 mmol), and KO^{t}Bu (17 mg, 0.15 mmol) in THF (0.5 mL) for 1 h. Purified employing reverse phase prep HPLC to provide the desired product. LC/MS: 404.21, 406.01 [M+H]⁺. ¹H-NMR (400 MHz; CDCl₃): δ 7.32-7.29 (m, 2H), 6.95 (s, 1H), 6.90-6.86 (m, 3H), 4.42 (s, 2H), 4.04-3.96 (m, 1H), 3.83 (d, J=1.9 Hz, 2H), 2.80-2.67 (m, 3H), 2.52 (m, 6H), 2.45-2.40 (m, 2H).

### Example 7

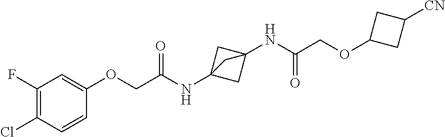 [SMILES: N#CC1CC(OCC(=O)NC23CC(NC(=O)COc4ccc(Cl)c(F)c4)(C2)C3)C1]

2-(4-chloro-3-fluorophenoxy)-N-(3-(2-(3-cyanocyclobutoxy)acetamido)bicyclo[1.1.1]pentan-1-yl)acetamide: Prepared employing General Procedure C employing N-[3-[(2-bromoacetyl)amino]-1-bicyclo[1.1.1]pentanyl]-2-(4-chloro-3-fluoro-phenoxy)acetamide (41 mg, 0.1 mmol), 3-hydroxycyclobutanecarbonitrile (29 mg, 0.3 mmol), and sodium hydride (12.0 mg, 0.3 mmol) in THF (1 mL) for 1 h. Purified employing reverse phase prep HPLC to provide the desired product. LC/MS: 422.19, 423.96 [M+H]⁺. ¹H-NMR (400 MHz; CDCl₃): δ 7.34 (t, J=8.6 Hz, 1H), 6.91 (s, 1H), 6.87 (s, 1H), 6.78 (dd, J=10.3, 2.8 Hz, 1H), 6.70 (ddd, J=8.9, 2.9, 1.3 Hz, 1H), 4.42 (s, 2H), 4.04-3.97 (m, 1H), 3.83 (s, 2H), 2.80-2.67 (m, 3H), 2.53-2.51 (m, 6H), 2.45-2.39 (m, 2H).

### Example 8

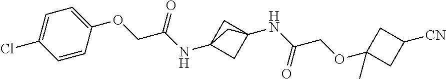 [SMILES: CC1(OCC(=O)NC23CC(NC(=O)COc4ccc(Cl)cc4)(C2)C3)CC(C#N)C1]

2-(4-chlorophenoxy)-N-(3-(2-(3-cyano-1-methylcyclobutoxy)acetamido)bicyclo[1.1.1]pentan-1-yl)acetamide: Prepared employing General Procedure C employing N-[3-[(2-bromoacetyl)amino]-1-bicyclo[1.1.1]pentanyl]-2-(4-chlorophenoxy)acetamide (25 mg, 0.06 mmol), 3-hydroxy-3-methyl-cyclobutanecarbonitrile (22 mg, 0.19 mmol), and sodium hydride (4.6 mg, 0.19 mmol), and tetrabutylammonium iodide (2.4 mg, 0.006 mmol) in THF (0.5 ml) for 16 h. Purified employing reverse phase prep HPLC to provide the desired product. LC/MS: 418.20, 420.01 [M+H]⁺. ¹H-NMR (400 MHz; CDCl₃): δ 7.32-7.29 (m, 2H), 6.96 (s, 1H), 6.94 (s, 1H), 6.90-6.86 (m, 2H), 4.43 (s, 2H), 3.80 (s, 2H), 2.82 (qd, J=8.8, 8.3 Hz, 1H), 2.56-2.49 (m, 8H), 2.46-2.40 (m, 2H).

### Example 9

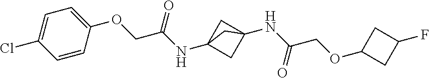 [SMILES: O=C(COc1ccc(Cl)cc1)NC12CC(NC(=O)COC3CC(F)C3)(C1)C2]

2-(4-chlorophenoxy)-N-(3-(2-(3-fluorocyclobutoxy)acetamido)bicyclo[1.1.1]pentan-1-yl)acetamide: Prepared employing General Procedure C employing N-[3-[(2-bromoacetyl)amino]-1-bicyclo[1.1.1]pentanyl]-2-(4-chloro-3-fluoro-phenoxy)acetamide (25 mg, 0.06 mmol), 3-fluorocyclobutanol (17 mg, 0.19 mmol), and sodium hydride (4.4 mg, 0.19 mmol), in THF (0.5 mL) for 16 h. Purified employing reverse phase prep HPLC to provide the desired product. LC/MS: 415.17, 416.98 [M+H]⁺. ¹H-NMR (400 MHz; CDCl₃): δ 7.34 (t, J=8.6 Hz, 1H), 6.90 (s, 1H), 6.85 (s, 1H), 6.78 (dd, J=10.3, 2.8 Hz, 1H), 6.70 (ddd, J=8.9, 2.9, 1.3 Hz, 1H), 5.24 (dtt, J=56.1, 6.5, 3.7 Hz, 1H), 4.42 (s, 2H), 4.34-4.27 (m, 1H), 3.82 (s, 2H), 2.58-2.35 (m, 10H).

### Example 10

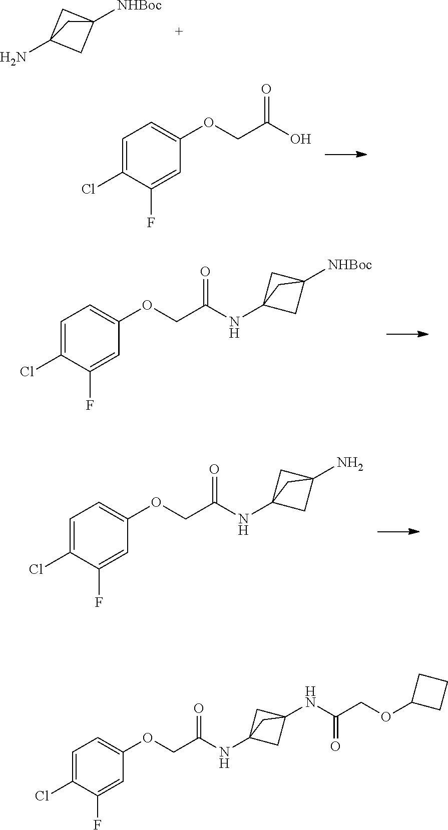 [SMILES: CC(C)(C)OC(=O)NC12CC(N)(C1)C2 ; O=C(O)COc1ccc(Cl)c(F)c1 ; CC(C)(C)OC(=O)NC12CC(NC(=O)COc3ccc(Cl)c(F)c3)(C1)C2 ; NC12CC(NC(=O)COc3ccc(Cl)c(F)c3)(C1)C2 ; O=C(COc1ccc(Cl)c(F)c1)NC12CC(NC(=O)COC3CCC3)(C1)C2]

tert-butyl (3-(2-(4-chloro-3-fluorophenoxy)acetamido)bicyclo[1.1.1]pentan-1-yl)carbamate: Prepared employing General Procedure A employing tert-butyl (3-aminobicyclo[1.1.1]pentan-1-yl)carbamate (400 mg, 2.01 mmol), 2-(4-chlorophenoxy)acetic acid (452 mg, 2.42 mmol), N-methylimidazole (497 mg, 6.05 mmol), and T3P solution (769 mg, 50% in EtOAc) in DMF (8.0 mL). Purified employing silica flash chromatography (0 to 10% MeOH/CH₂Cl₂) to provide the desired product. ¹H-NMR (400 MHz; CDCl₃): δ 7.32-7.29 (m, 2H), 6.92-6.90 (m, 1H), 6.89-6.86 (m, 2H), 4.42 (s, 2H), 2.40 (s, 6H), 1.47 (s, 9H).

N-(3-aminobicyclo[1.1.1]pentan-1-yl)-2-(4-chloro-3-fluorophenoxy)acetamide: tert-butyl (3-(2-(4-chloro-3-fluorophenoxy)acetamido)bicyclo[1.1.1]pentan-1-yl)carbamate from the previous step was dissolved in CH₂Cl₂ (5 mL) and TFA (5 mL) and stirred for 4 h, upon which the solution was concentrated. The resulting residue was partitioned between CH₂Cl₂ and 1 M Na₂CO₃ solution (10 mL each). The layers were separated, and the aqueous layer was extracted with CH₂Cl₂ (3×10 mL). The combined organic layers were dried over anhydrous Na₂SO₄, filtered and concentrated under reduced pressure. The crude reaction mixture was used directly.

2-(4-chloro-3-fluorophenoxy)-N-(3-(2-cyclobutoxyacetamido)bicyclo[1.1.1]pentan-1-yl)acetamide: Prepared employing General Procedure C employing N-[3-[(2-bromoacetyl)amino]-1-bicyclo[1.1.1]pentanyl]-2-(4-chloro-3-fluoro-phenoxy)acetamide (25 mg, 0.06 mmol), cyclobutanol (13 mg, 0.19 mmol), and sodium hydride (4.4 mg, 0.19 mmol), in THF (0.5 mL) for 1 h. Purified employing silica gel flash chromatography (0->80% EtOAc/hexanes) to deliver the desired compound. LC/MS: 397.19, 398.95 [M+H]⁺. 1H-NMR (400 MHz; CDCl₃): δ 7.34 (t, J=8.6 Hz, 1H), 6.94 (s, 1H), 6.90 (s, 1H), 6.78 (dd, J=10.3, 2.8 Hz, 1H), 6.70 (ddd, J=8.9, 2.9, 1.3 Hz, 1H), 4.42 (s, 2H), 4.02-3.95 (m, 1H), 3.80 (s, 2H), 2.51 (s, 6H), 2.28-2.19 (m, 2H), 2.02-1.92 (m, 2H), 1.80-1.71 (m, 1H), 1.60-1.48 (m, 1H).

### Example 11

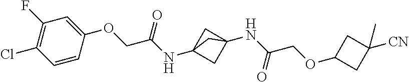 [SMILES: CC1(C#N)CC(OCC(=O)NC23CC(NC(=O)COc4ccc(Cl)c(F)c4)(C2)C3)C1]

2-(4-chloro-3-fluorophenoxy)-N-(3-(2-(3-cyano-3-methylcyclobutoxy)acetamido)bicyclo[1.1.1]pentan-1-yl)acetamide: Prepared employing General Procedure C employing N-[3-[(2-bromoacetyl)amino]-1-bicyclo[1.1.1]pentanyl]-2-(4-chloro-3-fluoro-phenoxy)acetamide (25 mg, 0.06 mmol), 3-hydroxy-1-methyl-cyclobutanecarbonitrile (21 mg, 0.19 mmol), and sodium hydride (4.4 mg, 0.19 mmol), in THF (0.5 mL) for 16 h. Purified employing reverse phase prep HPLC to provide the desired product. LC/MS: 436.19, 437.99 [M+H]⁺. ¹H-NMR (400 MHz; CDCl₃): δ 7.36-7.31 (m, 1H), 6.94-6.76 (m, 3H), 6.71-6.68 (m, 1H), 4.43-4.40 (m, 2H), 4.28-4.08 (m, 1H), 3.85-3.79 (m, 2H), 2.93-2.84 (m, 1H), 2.63-2.56 (m, 1H), 2.54-2.54 (m, 6H), 2.49-2.42 (m, 1H), 2.13-2.06 (m, 1H), 1.60-1.52 (m, 3H).

### Example 12

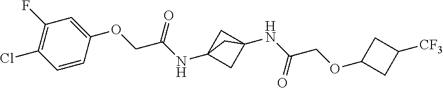 [SMILES: O=C(COc1ccc(Cl)c(F)c1)NC12CC(NC(=O)COC3CC(C(F)(F)F)C3)(C1)C2]

2-(4-chloro-3-fluorophenoxy)-N-(3-(2-(3-(trifluoromethyl)cyclobutoxy)acetamido)bicyclo[1.1.1]pentan-1-yl)acetamide: Prepared employing General Procedure C employing N-[3-[(2-bromoacetyl)amino]-1-bicyclo[1.1.1]pentanyl]-2-(4-chloro-3-fluoro-phenoxy)acetamide (30 mg, 0.07 mmol), 3-(trifluoromethyl)cyclobutanol (31 mg, 0.22 mmol), and sodium hydride (5.3 mg, 0.22 mmol), in THF (0.6 mL) for 1 h. Purified employing silica gel flash chromatography (0480% EtOAc/hexanes) to deliver the first eluting isomer and the second eluting isomer.

First eluting isomer: LC/MS: 465.14, 467.32 [M+H]⁺. ¹H-NMR (400 MHz; CDCl₃): δ 7.35 (t, J=8.6 Hz, 1H), 6.88 (s, 1H), 6.86 (s, 1H), 6.78 (dd, J=10.3, 2.8 Hz, 1H), 6.70 (ddd, J=8.9, 2.9, 1.3 Hz, 1H), 4.42 (s, 2H), 4.24-4.17 (m, 1H), 3.82 (s, 2H), 2.98-2.87 (m, 1H), 2.53-2.46 (m, 8H), 2.35-2.28 (m, 2H).

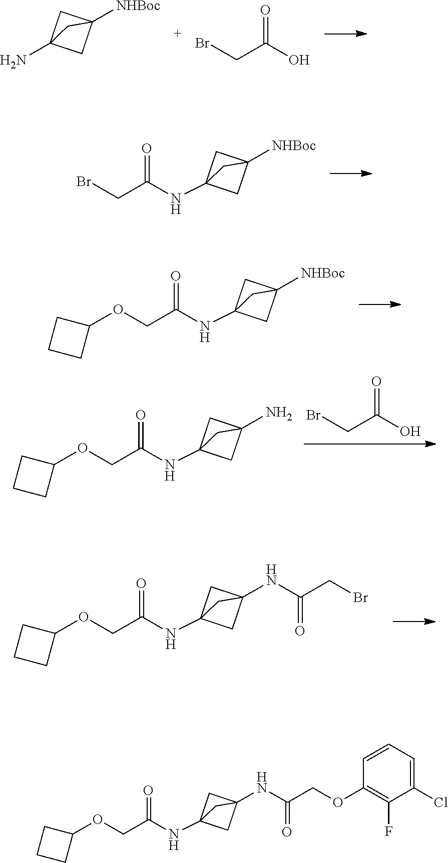

tert-butyl (3-(2-bromoacetamido)bicyclo[1.1.1]pentan-1-yl)carbamate: Prepared employing General Procedure A employing tert-butyl N-(1-amino-3-bicyclo[1.1.1]pentanyl)carbamate (500 mg, 2.52 mmol), bromoacetic acid (421 mg, 3.03 mmol), N,N-diisopropylethylamine (978 mg, 7.57 mmol), and T3P solution (962 mg, 50% in EtOAc) in EtOAc (5.0 mL). The crude reaction mixture was used directly.

N-(3-aminobicyclo[1.1.1]pentan-1-yl)-2-cyclobutoxyacetamide: Prepared employing General Procedure C employing tert-butyl N-[1-[(2-bromoacetyl)amino]-3-bicyclo[1.1.1]pentanyl]carbamate (680 mg, 2.13 mmol), cyclobutanol (461 mg, 6.39 mmol), and sodium hydride (256 mg, 6.39 mmol), in THF (10 mL) for 1 h. The crude reaction mixture was dissolved in DCM (5 mL) at 0° C. and TFA (5 mL) was added. The solution was stirred for 4 h and was concentrated. The crude reaction mixture was used directly.

N-[3-[(2-bromoacetyl)amino]-1-bicyclo[1.1.1]pentanyl]-2-(cyclobutoxy)acetamide: Prepared employing General Procedure A employing N-(1-amino-3-bicyclo[1.1.1]pentanyl)-2-(cyclobutoxy)acetamide (500 mg, 2.52 mmol), bromoacetic acid (421 mg, 3.03 mmol), N,N-diisopropylethylamine (978 mg, 7.57 mmol), and T3P solution (962 mg, 50% in EtOAc) in EtOAc (5.0 mL). The crude reaction mixture was used directly.

2-(3-chloro-2-fluoro-phenoxy)-N-[3-[[2-(cyclobutoxy)acetyl]amino]-1-bicyclo[1.1.1]pentanyl]acetamide: Prepared employing General Procedure D employing N-[3-[(2-bromoacetyl)amino]-1-bicyclo[1.1.1]pentanyl]-2-(cyclobutoxy)acetamide (20 mg, 0.06 mmol), 3-chloro-2-fluoro-phenol (10.6 mg, 0.07 mmol), and K₂CO₃ (16.6 mg, 0.12 mmol), in DMF (0.5 mL) at 60° C. for 4 h. Purified employing reverse-phase HPLC. LC/MS: 397.15, 399.00 [M+H]⁺.

### Example 14

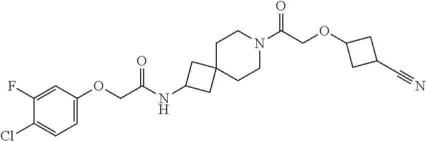 [SMILES: N#CC1CC(OCC(=O)N2CCC3(CC2)CC(NC(=O)COc2ccc(Cl)c(F)c2)C3)C1]

tert-butyl N-[7-[2-(4-chloro-3-fluoro-phenoxy)acetyl]-7-azaspiro[3.5]nonan-2-yl]carbamate: Prepared employing General Procedure A employing tert-butyl 2-amino-7-azaspiro[3.5]nonane-7-carboxylate (240 mg, 1.0 mmol), 2-(4-chloro-3-fluoro-phenoxy)acetic acid (246 mg, 1.2 mmol), N-methylimidazole (493 mg, 3.0 mmol), and T3P solution (382 mg, 50% in EtOAc) in DMF (1.0 mL). Purified by precipitation from the reaction mixture employing 1M NaOH solution. The crude reaction mixture was dissolved in DCM (5 mL) at 0° C. and TFA (5 mL) was added. The solution was stirred for 4 h and was concentrated to provide 2-(4-chloro-3-fluorophenoxy)-N-(7-azaspiro[3.5]nonan-2-yl)acetamide.

N-[7-(2-bromoacetyl)-7-azaspiro[3.5]nonan-2-yl]-2-(4-chloro-3-fluoro-phenoxy)acetamide: Prepared employing General Procedure A employing N-(7-azaspiro[3.5]nonan-2-yl)-2-(4-chloro-3-fluoro-phenoxy)acetamide (238 mg, 0.73 mmol), bromoacetic acid (152 mg, 1.09 mmol), N,N-diisopropylethylamine (0.38 mL, 2.18 mmol), and T3P solution (347 mg, 50% in EtOAc) in EtOAc (4.0 mL). The crude reaction mixture was used directly.

2-(4-chloro-3-fluoro-phenoxy)-N-[7-[2-(3-cyanocyclobutoxy)acetyl]-7-azaspiro[3.5]nonan-2-yl]acetamide: Prepared employing General Procedure C employing N-[7-(2-bromoacetyl)-7-azaspiro[3.5]nonan-2-yl]-2-(4-chloro-3-fluoro-phenoxy)acetamide (25 mg, 0.06 mmol), 3-hydroxycyclobutanecarbonitrile (16 mg, 0.17 mmol), and sodium hydride (4.0 mg, 0.17 mmol), in THF (0.5 mL) for 1 h. Purified employing reverse-phase prep HPLC. LC/MS: 464.30, 466.06 [M+H]⁺. ¹H-NMR (400 MHz; CDCl₃): δ 7.13 (t, J=8.6 Hz, 1H), 6.56 (dd, J=10.3, 2.8 Hz, 1H), 6.48 (ddd, J=8.9, 2.8, 1.2 Hz, 1H), 6.39-6.32 (m, 1H), 4.32-4.22 (m, 3H), 3.86-3.78 (m, 3H), 3.35-3.09 (m, 4H), 2.55-2.48 (m, 2H), 2.45-2.37 (m, 1H), 2.22-2.15 (m, 4H), 1.57-1.35 (m, 9H).

### Example 15

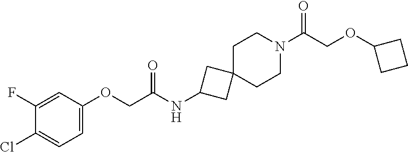 [SMILES: O=C(COc1ccc(Cl)c(F)c1)NC1CC2(CCN(C(=O)COC3CCC3)CC2)C1]

2-(4-chloro-3-fluoro-phenoxy)-N-[7-[2-(cyclobutoxy)acetyl]-7-azaspiro[3.5]nonan-2-yl]acetamide: Prepared employing General Procedure C employing N-[7-(2-bromoacetyl)-7-azaspiro[3.5]nonan-2-yl]-2-(4-chloro-3-fluoro-phenoxy)acetamide (30 mg, 0.07 mmol), cyclobutanol (15 mg, 0.20 mmol), and sodium hydride (4.8 mg, 0.20 mmol), in THF (0.5 mL) for 1 h. Purified employing reverse-phase prep HPLC. ¹H-NMR (400 MHz; CDCl₃): δ 7.35 (t, J=8.6 Hz, 1H), 6.79 (dd, J=10.3, 2.8 Hz, 1H), 6.70 (ddd, J=8.9, 2.9, 1.3 Hz, 1H), 6.61-6.57 (m, 1H), 4.52-4.48 (m, 1H), 4.45 (s, 2H), 4.05-3.99 (m, 3H), 3.56-3.36 (m, 4H), 2.44-2.38 (m, 2H), 2.24-2.19 (m, 2H), 1.99-1.93 (m, 2H), 1.78-1.49 (m, 8H).

### Example 16

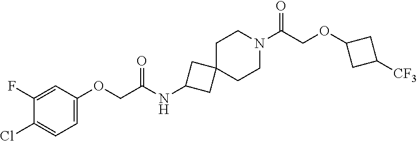 [SMILES: O=C(COc1ccc(Cl)c(F)c1)NC1CC2(CCN(C(=O)COC3CC(C(F)(F)F)C3)CC2)C1]

2-(4-chloro-3-fluoro-phenoxy)-N-[7-[2-[3-(trifluoromethyl)cyclobutoxy]acetyl]-7-azaspiro[3.5]nonan-2-yl]acetamide: Prepared employing General Procedure C employing N-[7-(2-bromoacetyl)-7-azaspiro[3.5]nonan-2-yl]-2-(4-chloro-3-fluoro-phenoxy)acetamide (30 mg, 0.07 mmol), 3-(trifluoromethyl)cyclobutanol (28.1 mg, 0.20 mmol), and sodium hydride (4.8 mg, 0.20 mmol), in THF (0.5 mL) for 1 h. Purified employing reverse-phase prep HPLC. LC/MS: 507.24, 509.04 [M+H]⁺. ¹H-NMR (400 MHz; CDCl₃): δ 7.35 (t, J=8.6 Hz, 1H), 7.35 (t, J=8.6 Hz, 1H), 6.79 (dd, J=10.3, 2.9 Hz, 1H), 6.79 (dd, J=10.3, 2.9 Hz, 1H), 6.70 (ddd, J=8.9, 2.9, 1.3 Hz, 1H), 6.70 (ddd, J=8.9, 2.9, 1.3 Hz, 1H), 6.61-6.56 (m, 1H), 6.61-6.56 (m, 1H), 4.55-4.44 (m, 3H), 4.55-4.44 (m, 3H), 4.24-3.99 (m, 3H), 4.24-3.99 (m, 3H), 3.58-3.30 (m, 4H), 3.58-3.30 (m, 4H), 2.51-2.31 (m, 6H), 2.51-2.31 (m, 6H), 2.19-2.12 (m, 1H), 2.19-2.12 (m, 1H), 1.79-1.56 (m, 7H), 1.79-1.56 (m, 7H).

### Example 17

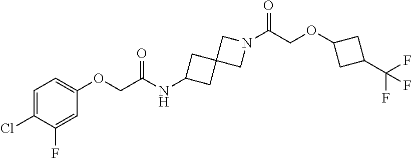 [SMILES: O=C(COc1ccc(Cl)c(F)c1)NC1CC2(C1)CN(C(=O)COC1CC(C(F)(F)F)C1)C2]

2-(4-chloro-3-fluoro-phenoxy)-N-[2-[2-[3-(trifluoromethyl)cyclobutoxy]acetyl]-2-azaspiro[3.3]heptan-6-yl]acetamide: Prepared employing General Procedure C employing N-[2-(2-bromoacetyl)-2-azaspiro[3.3]heptan-6-yl]-2-(4-chloro-3-fluoro-phenoxy)acetamide (30 mg, 0.07 mmol), 3-(trifluoromethyl)cyclobutanol (30 mg, 0.21 mmol), and sodium hydride (5.1 mg, 0.21 mmol), in THF (0.5 mL) for 1 h. Purified employing reverse-phase prep HPLC. ¹H-NMR (400 MHz; CDCl₃): δ 7.35 (t, J=8.6 Hz, 1H), 6.77 (dd, J=10.3, 2.8 Hz, 1H), 6.70-6.67 (m, 1H), 6.62-6.55 (m, 1H), 4.44-4.32 (m, 4H), 4.22-4.12 (m, 2H), 4.01 (s, 1H), 3.96-3.91 (m, 3H), 2.72-2.66 (m, 2H), 2.56-2.43 (m, 3H), 2.34-2.09 (m, 4H). LC/MS: 479.21, 480.98 [M+H]⁺.

### Example 18

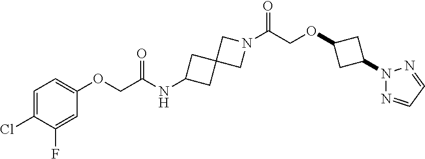

2-(4-chloro-3-fluoro-phenoxy)-N-[2-[2-[3-(triazol-2-yl)cyclobutoxy]acetyl]-2-azaspiro[3.3]heptan-6-yl]acetamide: Prepared employing General Procedure C employing N-[2-(2-bromoacetyl)-2-azaspiro[3.3]heptan-6-yl]-2-(4-chloro-3-fluoro-phenoxy)acetamide (30 mg, 0.07 mmol), 3-(triazol-2-yl)cyclobutanol (15 mg, 0.21 mmol), and sodium hydride (5.1 mg, 0.21 mmol), in THF (0.5 mL) for 1 h. Purified employing reverse-phase prep HPLC. ¹H-NMR (400 MHz; CDCl₃): δ 7.64-7.63 (m, 2H), 7.35 (t, J=8.6 Hz, 1H), 6.77 (dd, J=10.2, 2.8 Hz, 1H), 6.70-6.67 (m, 1H), 6.63-6.57 (m, 1H), 4.83-4.75 (m, 1H), 4.46-4.36 (m, 4H), 4.29 (s, 1H), 4.13 (d, J=0.3 Hz, 1H), 4.01-3.95 (m, 4H), 2.99-2.93 (m, 2H), 2.76-2.67 (m, 4H), 2.24-2.18 (m, 2H). LC/MS: 478.29, 480.05 [M+H]⁺.

### Example 19

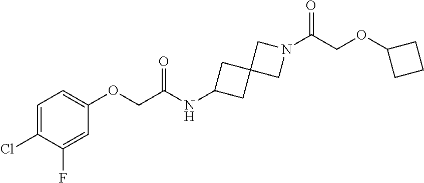 [SMILES: O=C(COc1ccc(Cl)c(F)c1)NC1CC2(C1)CN(C(=O)COC1CCC1)C2]

2-(4-chloro-3-fluoro-phenoxy)-N-[2-[2-(cyclobutoxy)acetyl]-2-azaspiro[3.3]heptan-6-yl]acetamide: Prepared employing General Procedure C employing N-[2-(2-bromoacetyl)-2-azaspiro[3.3]heptan-6-yl]-2-(4-chloro-3-fluoro-phenoxy)acetamide (30 mg, 0.07 mmol), cyclobutanol (15 mg, 0.21 mmol), and sodium hydride (5.1 mg, 0.21 mmol), in THF (0.5 mL) for 1 h. Purified employing reverse-phase prep HPLC. ¹H-NMR (400 MHz; CDCl₃): δ 7.35 (t, J=8.6 Hz, 1H), 6.79-6.75 (m, 1H), 6.69 (ddd, J=8.9, 2.9, 1.3 Hz, 1H), 6.58 (s, 1H), 4.44-4.34 (m, 3H), 4.24-4.23 (m, 1H), 4.12-4.11 (m, 1H), 4.01-3.92 (m, 4H), 2.71-2.66 (m, 2H), 2.27-2.17 (m, 4H), 2.00-1.90 (m, 2H), 1.79-1.71 (m, 2H), 1.59-1.47 (m, 1H). LC/MS: 411.18, 413.03 [M+H]⁺.

### Example 20

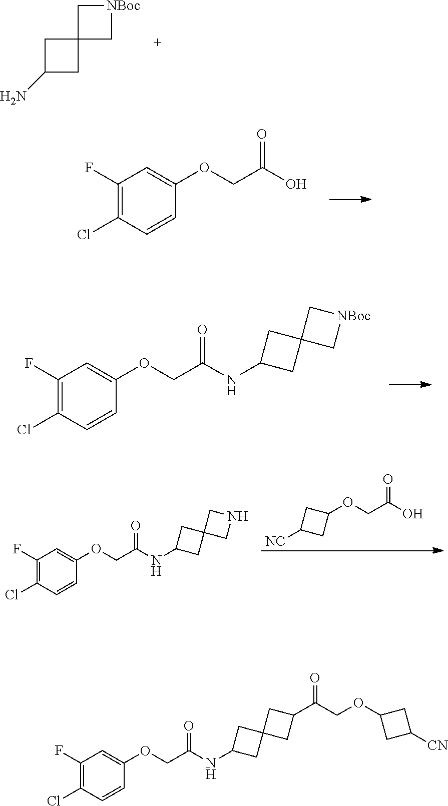 [SMILES: CC(C)(C)OC(=O)N1CC2(CC(N)C2)C1 ; O=C(O)COc1ccc(Cl)c(F)c1 ; CC(C)(C)OC(=O)N1CC2(CC(NC(=O)COc3ccc(Cl)c(F)c3)C2)C1 ; O=C(COc1ccc(Cl)c(F)c1)NC1CC2(CNC2)C1 ; N#CC1CC(OCC(=O)O)C1 ; N#CC1CC(OCC(=O)C2CC3(CC(NC(=O)COc4ccc(Cl)c(F)c4)C3)C2)C1]

tert-butyl 6-[[2-(4-chloro-3-fluoro-phenoxy)acetyl]amino]-2-azaspiro[3.3]heptane-2-carboxylate: To a mixture of HATU (99 mg, 0.26 mmol) and 2-(4-chloro-3-fluoro-phenoxy)acetic acid (48 mg, 0.24 mmol) in DMF (5 mL) was added tert-butyl 6-amino-2-azaspiro[3.3]heptane-2-carboxylate (50 mg, 0.24 mmol) and Na₂CO₃ (50 mg, 0.47 mmol), and the mixture was stirred at 20° C. for 16 h. The mixture was poured into ice-water (15 mL) and extracted with EtOAc (3×5 mL). The combined organic phases were washed with brine (3×5 mL), dried over anhydrous Na₂SO₄, filtered and concentrated under reduced pressure. The residue was purified by prep-TLC (SiO₂, PE:EtOAc=3:1) to provide desired compound. LC-MS m/z: =343.1, 345.1 [M+H-56]⁺. ¹H-NMR (400 MHz, CDCl₃): δ 7.34 (t, J=8.6 Hz, 1H), 6.76 (dd, J=2.8, 10.3 Hz, 1H), 6.67 (td, J=1.4, 8.9 Hz, 11H), 6.53 (br d, J=7.4 Hz, 1H), 4.42 (s, 2H), 4.40-4.33 (m, 1H), 3.98 (s, 2H), 3.87 (s, 2H), 2.64 (ddd, J=2.9, 7.5, 10.0 Hz, 2H), 2.13 (dt, J=2.9, 9.3 Hz, 2H), 1.44 (s, 9H)

N-(2-azaspiro[3.3]heptan-6-yl)-2-(4-chloro-3-fluoro-phenoxy)acetamide: A solution of tert-butyl 6-[[2-(4-chloro-3-fluoro-phenoxy)acetyl]amino]-2-azaspiro[3.3]heptane-2-carboxylate (90 mg, 0.23 mmol) in HCl/EtOAc (4 M, 5 mL) was stirred at 20° C. for 1 h. The mixture was concentrated under reduced pressure to provide desired compound (95%, HCl salt). LC-MS m/z: =335.1, 337.1 [M+H]⁺.

2-(4-chloro-3-fluoro-phenoxy)-N-[2-[2-(3-cyanocyclobutoxy)acetyl]-2-azaspiro[3.3]heptan-6-yl]acetamide: To a solution of 2-(3-cyanocyclobutoxy)acetic acid (9.0 mg, 0.06 mmol) and HATU (25 mg, 0.066 mmol) in DMF (3 mL) was added N-(2-azaspiro[3.3]heptan-6-yl)-2-(4-chloro-3-fluoro-phenoxy)acetamide (20 mg, 0.06 mmol, HCl salt) and Na₂CO₃ (19 mg, 0.18 mmol). The mixture was stirred at 20° C. for 2 h, and was concentrated under reduced pressure. The residue was purified by prep-HPLC (TFA) to provide title compound (trans:cis=3:1). ¹H-NMR (400 MHz, CDCl₃): δ 7.34 (t, J=8.6 Hz, 1H), 6.76 (dd, J=2.6, 10.2 Hz, 1H), 6.68 (td, J=1.4, 8.9 Hz, 1H), 6.57 (br t, J=7.4 Hz, 1H), 4.43 (s, 2H), 4.42-4.34 (m, 1H), 4.33-4.27 (m, 1H), 4.23-4.15 (m, 1H), 4.11 (s, 1H), 4.00 (s, 1H), 3.97 (d, J=7.8 Hz, 0.75H), 3.92 (d, J=3.9 Hz, 2H), 3.09 (br dd, J=4.9, 9.0 Hz, 0.25H), 2.79-2.60 (m, 5H), 2.50-2.32 (m, 2H), 2.26-2.15 (m, 2H). LC-MS m/z: =436.3, 438.3 [M+H]⁺.

### Example 21

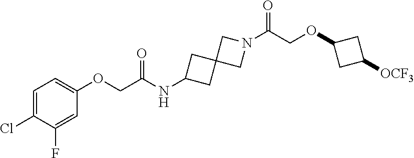

2-(4-chloro-3-fluoro-phenoxy)-N-[2-[2-[3-cis-(trifluoromethoxy)cyclobutoxy]acetyl]-2-azaspiro[3.3]heptan-6-yl]acetamide: Prepared employing General Procedure A employing N-(2-azaspiro[3.3]heptan-6-yl)-2-(4-chloro-3-fluoro-phenoxy)acetamide (17 mg, 0.06 mmol), 2-[3-(trifluoromethoxy)cyclobutoxy]acetic acid (10 mg, 0.05 mmol), N,N-diisopropylethylamine (18 mg, 0.14 mmol), and T3P solution (35 μL, 50% in EtOAc) in EtOAc (500 μL). Purified employing reverse-phase HPLC to provide the desired product. ¹H-NMR (400 MHz; CDCl₃): δ 7.38-7.32 (m, 1H), 6.79-6.74 (m, 1H), 6.71-6.66 (m, 1H), 6.64-6.54 (m, 1H), 4.46-4.27 (m, 5H), 4.24-4.18 (m, 1H), 4.15-4.09 (m, 1H), 4.03-3.97 (m, 1H), 3.96-3.90 (m, 2H), 3.75-3.66 (m, 1H), 2.86-2.76 (m, 2H), 2.73-2.64 (m, 2H), 2.30-2.15 (m, 4H). LC/MS: 495.22, 497.03 [M+H]⁺.

### Example 22

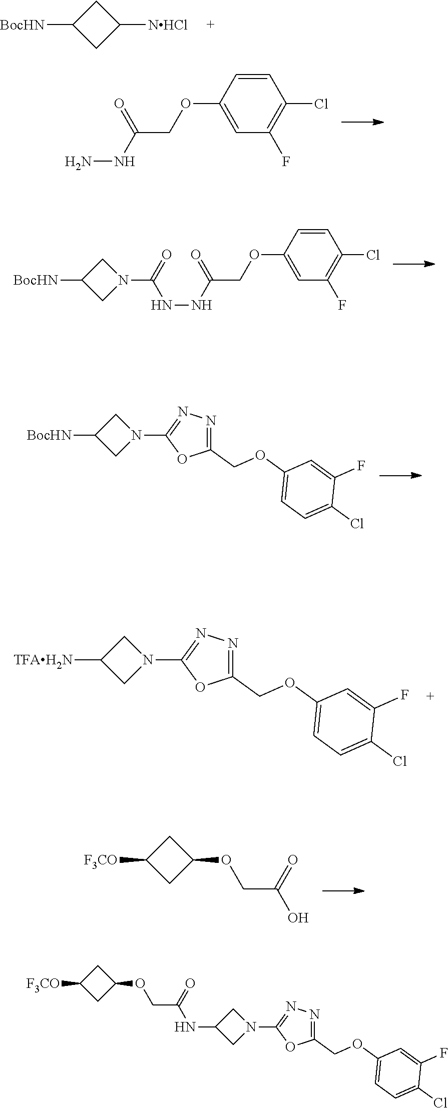

tert-butyl (1-(2-(2-(4-chloro-3-fluorophenoxy)acetyl)hydrazinecarbonyl)azetidin-3-yl)carbamate: To a solution of tert-butyl N-(azetidin-3-yl)carbamate hydrochloride (500 mg, 2.40 mmol) in THF (6 mL) was added CDI (393 mg, 2.42 mmol). The reaction mixture was stirred 16 h at 25° C. To the reaction mixture were then added NEt₃ (0.33 mL, 2.40 mmol) and 2-(4-chloro-3-fluorophenoxy)acetohydrazide (577 mg, 2.64 mmol) as a solution in THF (4 mL). The reaction mixture was then stirred for an additional 16 h at 25° C. The reaction mixture was concentrated under reduced pressure. The crude residue was purified by silica gel column chromatography.

tert-butyl (1-(5-((4-chloro-3-fluorophenoxy)methyl)-1,3,4-oxadiazol-2-yl)azetidin-3-yl)carbamate: To a solution of tert-butyl (1-(2-(2-(4-chloro-3-fluorophenoxy)acetyl)hydrazinecarbonyl)azetidin-3-yl)carbamate (220 mg, 0.53 mmol) in DCM (30 mL) were added pyridine (0.85 mL, 10.6 mmol) and Tf₂O (0.44 mL, 2.64 mmol) at −10° C. The reaction mixture was stirred at 25° C. for 3 h. The reaction mixture was quenched by the addition of sat. NaHCO₃ (10 mL) at −10° C. and exacted with EtOAc (3×10 mL). The combined organic layers were dried over Na₂SO₄, filtered, and concentrated under reduced pressure. The crude residue was purified by preparative TLC. LC-MS: m/z=399.1, 401.1 [M+H]⁺.

1-(5-((4-chloro-3-fluorophenoxy)methyl)-1,3,4-oxadiazol-2-yl)azetidin-3-amine TFA salt: To a solution of tert-butyl (1-(5-((4-chloro-3-fluorophenoxy)methyl)-1,3,4-oxadiazol-2-yl)azetidin-3-yl)carbamate (90 mg, 0.22 mmol) in DCM (5 mL) was added TFA (1.67 mL, 22.6 mmol) at 0° C. The reaction mixture was stirred at 25° C. for 3 h. The reaction mixture was concentrated under reduced pressure. LC-MS: m/z=299.1, 301.1 [M+H]⁺.

N-[1-[5-[(4-chloro-3-fluoro-phenoxy)methyl]-1,3,4-oxadiazol-2-yl]azetidin-3-yl]-2-[3-cis-(trifluoromethoxy)cyclobutoxy]acetamide: Prepared according to General Procedure A employing 1-(5-((4-chloro-3-fluorophenoxy)methyl)-1,3,4-oxadiazol-2-yl)azetidin-3-amine TFA salt (130 mg, 0.44 mmol) and 2-[3-cis-(trifluoromethoxy)cyclobutoxy]acetic acid (93 mg, 0.44 mmol) in EtOAc (4 mL). LC-MS: m/z=495.3, 497.3 [M+H]⁺. ¹H-NMR (400 MHz, CDCl₃): δ 7.31 (t, J=8.60 Hz, 1H), 6.95 (br d, J=7.65 Hz, 1H), 6.84 (dd, J=10.35, 2.82 Hz, 1H), 6.77 (ddd, J=8.85, 2.89, 1.19 Hz, 1H), 5.08 (s, 2H), 5.01-4.89 (m, 1H), 4.52 (t, J=8.41 Hz, 2H), 4.34 (quin, J=7.15 Hz, 1H), 4.11 (dd, J=9.03, 5.65 Hz, 2H), 3.88 (s, 2H), 3.74 (quin, J=6.87 Hz, 1H), 2.84 (did, J=9.93, 6.67, 6.67, 3.20 Hz, 2H), 2.34-2.24 (m, 2H).

### Example 23

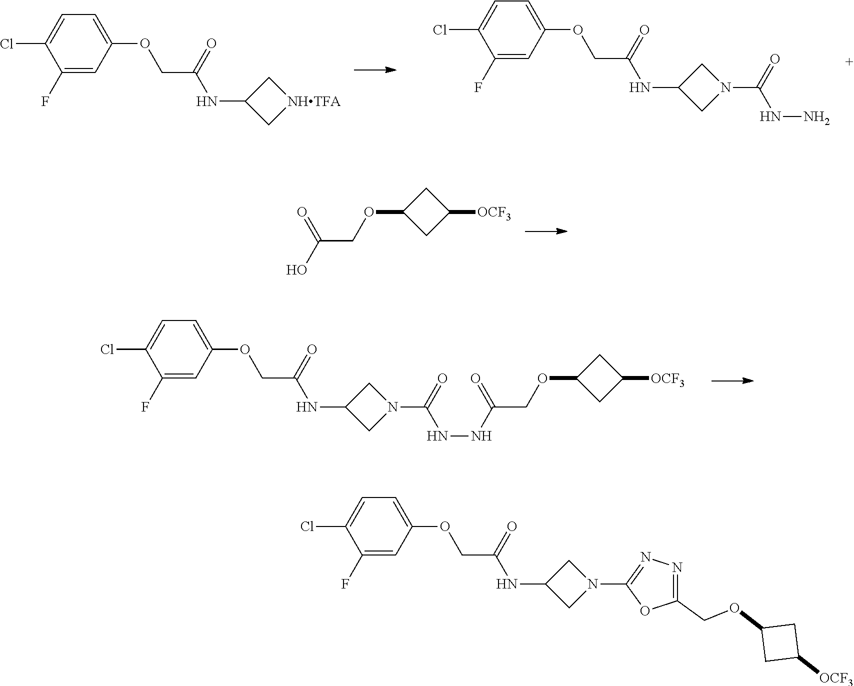

2-(4-chloro-3-fluorophenoxy)-N-(1-(hydrazinecarbonyl)azetidin-3-yl)acetamide: To a mixture of N-(azetidin-3-yl)-2-(4-chloro-3-fluoro-phenoxy)acetamide TFA salt (1.0 g, 2.68 mmol) and CDI (478 mg, 2.95 mmol) in DCM (15 mL) was added NEt₃ (1.09 g, 10.73 mmol). The reaction mixture was stirred at 25° C. for 0.5 h. To the reaction mixture was then added NH₂NH₂—H₂O (137 mg, 2.68 mmol). The reaction mixture was stirred at 40° C. for a further 20 h. The reaction mixture was quenched by the addition of sat. NaHCO₃ (60 mL) and extracted with DCM:i-PrOH (3×20 mL, v:v=3:1). The combined organic layers were washed with brine (30 mL), dried over Na₂SO₄, filtered, and concentrated under reduced pressure. LC-MS: m/z=317.1, 319.1 [M+H]⁺.

2-(4-chloro-3-fluorophenoxy)-N-(1-(2-(2-((3-cis-(trifluoromethoxy)cyclobutoxy)acetyl)hydrazinecarbonyl)azetidin-3-yl)acetamide: Prepared using General Procedure A employing 2-[3-cis-(trifluoromethoxy)cyclobutoxy]acetic acid (135 mg, 0.63 mmol) and 2-(4-chloro-3-fluorophenoxy)-N-(1-(hydrazinecarbonyl)azetidin-3-yl)acetamide (200 mg, 0.63 mmol) in EtOAc (5 mL). LC-MS: m/z=513.2, 515.1 [M+H]⁺.

2-(4-chloro-3-fluoro-phenoxy)-N-[1-[5-[[3-cis-(trifluoromethoxy)cyclobutoxy]methyl]-1,3,4-oxadiazol-2-yl]azetidin-3-yl]acetamide: To a solution of 2-(4-chloro-3-fluorophenoxy)-N-(1-(2-(2-((3-cis-(trifluoromethoxy)cyclobutoxy)acetyl)hydrazinecarbonyl)azetidin-3-yl)acetamide (60 mg, 0.12 mmol) in DCM (6 mL) were added TsCl (44 mg, 0.23 mmol) and NEt₃ (47 mg, 0.46 mmol). The reaction mixture was stirred at 25° C. for 15 h. The reaction mixture was quenched by the addition of sat. NaHCO₃ (30 mL) and extracted with DCM:i-PrOH (3×10 mL, v:v=3:1). The combined organic layers were washed with brine (10 mL), dried over Na₂SO₄, filtered, and concentrated under reduced pressure. The residue was purified by preparative HPLC. LC-MS: m/z=495.3, 497.3 [M+H]⁺. ¹H-NMR (400 MHz, CDCl₃): δ 7.35 (t, J=8.6 Hz, 1H), 7.08 (br d, J=7.5 Hz, 1H), 6.78 (dd, J=2.8, 10.2 Hz, 1H), 6.70 (dd, J=1.6, 8.8 Hz, 1H), 5.06-4.93 (m, 1H), 4.54 (t, J=8.3 Hz, 2H), 4.48 (d, J=8.3 Hz, 4H), 4.29 (quin, J=7.2 Hz, 1H), 4.17-4.08 (m, 2H), 3.75 (quin, J=6.9 Hz, 1H), 2.78 (dtd, J=3.2, 6.6, 9.8 Hz, 2H), 2.29-2.17 (m, 2H).

### Example 24

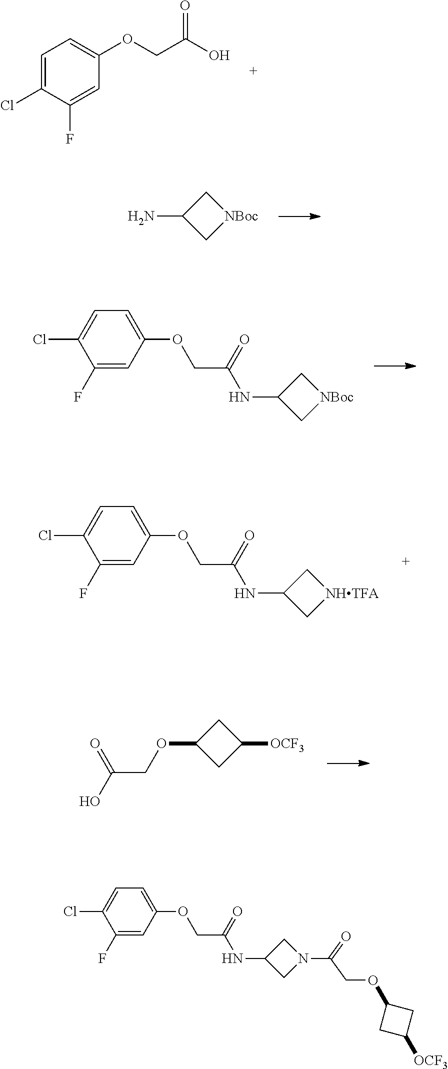

tert-butyl 3-(2-(4-chloro-3-fluorophenoxy)acetamido)azetidine-1-carboxylate: To a solution of 2-(4-chloro-3-fluorophenoxy)acetic acid (261 mg, 1.28 mmol) in DMF (2 mL) were added HATU (485 mg, 1.28 mmol), Na₂CO₃ (246 mg, 2.32 mmol), and tert-butyl 3-aminoazetidine-1-carboxylate (200 mg, 1.16 mmol). The reaction mixture was stirred at 25° C. for 2 h. The reaction mixture was quenched by the addition of H₂O (60 mL) and extracted with EtOAc (3×20 mL). The combined organic layers were washed with H₂O (3×20 mL), brine (20 ml), and then dried over Na₂SO₄, filtered, and concentrated under reduced pressure. The crude residue was purified by silica gel column chromatography. LC-MS: m/z=303.1, 305.1 [M−55]⁺

N-(azetidin-3-yl)-2-(4-chloro-3-fluorophenoxy)acetamide TFA salt: A solution of tert-butyl 3-(2-(4-chloro-3-fluorophenoxy)acetamido)azetidine-1-carboxylate (500 mg, 1.39 mmol) in DCM (10 mL) was added TFA (2.0 mL) at 0° C. The reaction mixture was stirred at 0° C. for 3 h. The reaction mixture was concentrated under reduced pressure. LC-MS: m/z=259.1, 261.1 [M+H]⁺.

2-(4-chloro-3-fluoro-phenoxy)-N-[1-[2-[3-cis-(trifluoromethoxy)cyclobutoxy]acetyl]azetidin-3-yl]acetamide: To a solution of 2-(3-cis-(trifluoromethoxy)cyclobutoxy)acetic acid (37 mg, 0.18 mmol) in DMF (1 ML) was added HATU (67 mg, 0.177 mmol), Na₂CO₃ (51 mg, 0.482 mmol), and N-(azetidin-3-yl)-2-(4-chloro-3-fluorophenoxy)acetamide TFA salt (60 mg, 0.16 mmol). The reaction mixture was stirred at 25° C. for 2 h. The reaction mixture was quenched by the addition of H₂O (15 mL) and extracted with EtOAc (3×5 mL). The combined organic layers were washed with brine (10 mL), dried over Na₂SO₄, filtered, and concentrated under reduced pressure. The crude residue was purified by preparative HPLC. LC-MS: m/z=455.3, 457.3 [M+H]⁺. ¹H-NMR (400 MHz, CDCl₃): δ 7.35 (t, J=8.6 Hz, 1H), 7.05 (br s, 1H), 6.78 (dd, J=2.8, 10.2 Hz, 1H), 6.70 (td, J=1.4, 8.8 Hz, 1H), 4.88-4.77 (m, 1H), 4.65 (br t, J=8.9 Hz, 1H), 4.48 (s, 2H), 4.42 (br t, J=9.5 Hz, 1H), 4.30 (quin, J=7.2 Hz, 1H), 4.18 (br dd, J=5.1, 10.0 Hz, 1H), 4.01-3.96 (m, 1H), 3.95 (s, 2H), 3.69 (quin, J=6.9 Hz, 1H), 2.86-2.74 (m, 2H), 2.28-2.17 (m, 2H).

### Example 25

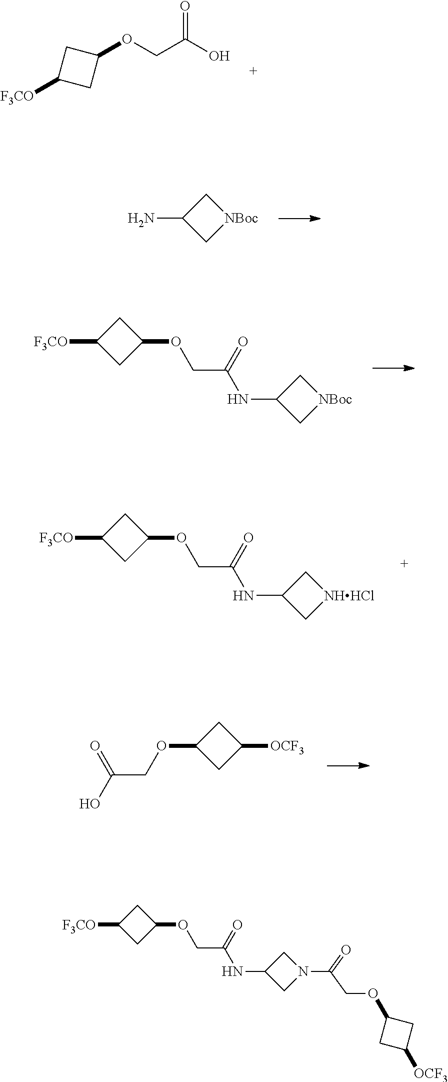

tert-butyl 3-[[2-[3-cis-(trifluoromethoxy)cyclobutoxy]acetyl]amino]azetidine-1-carboxylate: To a mixture of 2-[3-cis-(trifluoromethoxy)cyclobutoxy]acetic acid (200 mg, 0.93 mmol) and tert-butyl 3-aminoazetidine-1-carboxylate (177 mg, 1.03 mmol) in DMF (5 mL) was added HATU (533 mg, 1.40 mmol) and DIPEA (483 mg, 3.74 mmol) at 25° C. under N₂. The reaction mixture was stirred at 25° C. for 5 h. The reaction mixture was quenched by pouring onto ice-water (30 mL) and extracted with EtOAc (3×8 mL). The combined organic layers were washed with brine (4×10 mL), dried over Na₂SO₄, filtered, and concentrated under reduced pressure. The crude residue was purified by silica gel column chromatography.

N-(azetidin-3-yl)-2-[3-cis-(trifluoromethoxy)cyclobutoxy]acetamide hydrochloride: tert-Butyl 3-[[2-[3-cis-(trifluoromethoxy)cyclobutoxy]acetyl]amino]azetidine-1-carboxylate (310 mg, 0.84 mmol) was dissolved in HCl/EtOAc (10 mL). The reaction mixture was stirred at 25° C. for 3 h. The reaction mixture concentrated under reduced pressure.

2-[3-cis(trifluoromethoxy)cyclobutoxy]-N-[1-[2-[3-cis-(trifluoromethoxy)cyclobutoxy]acetyl]azetidin-3-yl]acetamide: To a mixture of N-(azetidin-3-yl)-2-[3-cis-(trifluoromethoxy)cyclobutoxy]acetamide hydrochloride (200 mg, 0.66 mmol) and 2-[3-cis-(trifluoromethoxy)cyclobutoxy]acetic acid (141 mg, 0.66 mmol) in DMF (3 mL) were added HATU (374 mg, 0.98 mmol) and DIEA (339 mg, 2.63 mmol) under N₂. The reaction mixture was stirred at 25° C. for 2 h. The reaction mixture was quenched by pouring onto ice-water (20 mL) and extracted with EtOAc (3×5 mL). The combined organic layers were washed with brine (4×5 mL), dried over Na₂SO₄, filtered, and concentrated under reduced pressure. The crude residue was purified by preparative HPLC. LC-MS: m/z=465.4 [M+H]⁺. ¹H-NMR (400 MHz, CDCl₃): δ 6.88 (br d, J=7.72 Hz, 1H), 4.83-4.72 (m, 1H), 4.67-4.59 (m, 1H), 4.45-4.23 (m, 3H), 4.16 (dd, J=9.92, 5.07 Hz, 1H), 3.98-3.91 (m, 3H), 3.88 (s, 2H), 3.79-3.65 (m, 2H), 2.83 (ttd, J=13.05, 13.05, 6.63, 6.63, 3.20 Hz, 4H), 2.37-2.18 (m, 4H).

### Example 26

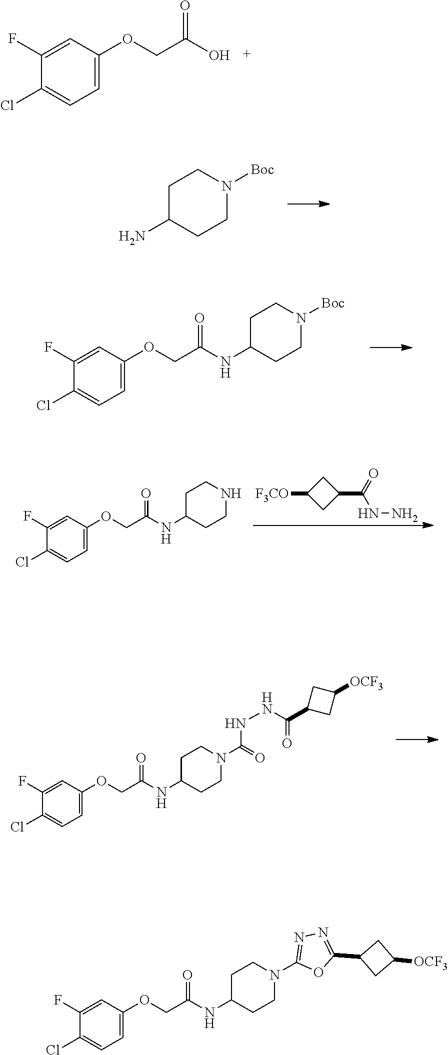

Tert-butyl 4-[[2-(4-chloro-3-fluoro-phenoxy)acetyl]amino]piperidine-1-carboxylate: To a mixture of 2-(4-chloro-3-fluoro-phenoxy)acetic acid (1.0 g, 4.89 mmol) and tert-butyl 4-aminopiperidine-1-carboxylate (979 mg, 4.89 mmol) in DMF (20 mL) at rt were added HATU (2.79 g, 7.33 mmol) and DIPEA (2.53 g, 19.55 mmol). The mixture was stirred at 25° C. for 12 h and was poured into ice-water (100 mL). EtOAc (20 mL) was added, the layers were separated, and the aqueous phase was extracted with EtOAc (3×20 mL). The combined organic phases were washed with brine (5×20 mL), dried with anhydrous Na₂SO₄, filtered and concentrated under reduced pressure. The residue was purified by silica gel column chromatography (PE:MTBE=100:1 to 1:1) to provide tert-butyl 4-[[2-(4-chloro-3-fluoro-phenoxy) acetyl] amino] piperidine-1-carboxylate. ¹H-NMR (400 MHz, CDCl₃): δ 7.33 (t, J=8.60 Hz, 1H), 6.76 (dd, J=10.36, 2.87 Hz, 1H), 6.68 (ddd, J=8.82, 2.87, 1.32 Hz, 1H), 6.33 (br d, J=8.16 Hz, 1H), 4.45 (s, 2H) 3.95-4.13 (m, 3H) 2.88 (br t, J=12.24 Hz, 2H), 1.87-1.98 (m, 1H), 1.87-1.98 (m, 1H), 1.46 (s, 9H).

2-(4-chloro-3-fluoro-phenoxy)-N-(4-piperidyl)acetamide: tert-butyl 4-[[2-(4-chloro-3-fluoro-phenoxy)acetyl]amino]piperidine-1-carboxylate (1.7 g, 4.39 mmol) was stirred at 25° C. for 3 h in HCl/EtOAc (20 mL). The mixture was concentrated under reduced pressure to provide 2-(4-chloro-3-fluoro-phenoxy)-N-(4-piperidyl) acetamide HCl salt, which was used directly. ¹H-NMR (400 MHz, DMSO): δ 8.94 (br s, 2H) 8.39 (d, J=7.50 Hz, 1H) 7.49 (t, J=8.93 Hz, 1H) 7.07 (dd, J=11.47, 2.87 Hz, 1H) 6.75-6.88 (m, 1H) 3.83-3.95 (m, 2H) 3.25 (br d, J=13.01 Hz, 2H) 2.85-3.03 (m, 2H) 1.87 (br dd, J=13.78, 3.42 Hz, 2H) 1.63-1.76 (m, 2H).

2-(4-chloro-3-fluoro-phenoxy)-N-[1-[[[3-cis-(trifluoromethoxy)cyclobutanecarbonyl]amino]carbamoyl]-4-piperidyl]acetamide: To a solution of 3-cis-(trifluoromethoxy)cyclobutanecarbohydrazide (0.1 g, 0.51 mmol) in THF (5 mL) at rt were added CDI (90 mg, 0.56 mmol) and TEA (153 mg, 1.51 mmol). The reaction mixture was stirred at 25° C. for 12 h, 2-(4-chloro-3-fluoro-phenoxy)-N-(4-piperidyl)acetamide HCl salt (163 mg, 0.50 mmol) was added and the reaction was stirred at rt for 12 h. LCMS showed about 50% of the desired product and the reaction was stirred further at 75° C. for 12 h. The reaction mixture was poured into ice-water (30 mL) and EtOAc was added (10 mL). The layers were separated and the aqueous phase was extracted with EtOAc (3×10 mL). The combined organic phases were washed with brine (30 mL), dried with anhydrous Na₂SO₄, filtered and concentrated under reduced pressure to provide 2-(4-chloro-3-fluoro-phenoxy)-N-[1-[[[3-cis-(trifluoromethoxy)cyclobutanecarbonyl]amino]carbamoyl]-4-piperidyl]acetamide, which was used directly.

2-(4-chloro-3-fluoro-phenoxy)-N-[1-[5-[3-cis-(trifluoromethoxy)cyclobutyl]-1,3,4-oxadiazol-2-yl]-4-piperidyl]acetamide: To a mixture of 2-(4-chloro-3-fluoro-phenoxy)-N-[1-[[[3-cis-(trifluoromethoxy)cyclobutanecarbonyl]amino]carbamoyl]-4-piperidyl]acetamide (30 mg, 0.059 mmol) in POCl₃ (2.0 mL) was stirred at 110° C. for 2 h. The mixture was concentrated under reduced pressure and the residue was treated with aq. NaHCO₃ (5 mL). EtOAc (5 mL) was added, the layers were separated, and the aqueous phase was extracted with EtOAc (3×5 mL). The combined organic phases were washed with brine (10 mL), dried with anhydrous Na₂SO₄, filtered and concentrated under reduced pressure. The residue was purified by prep HPLC (TFA) to provide 2-(4-chloro-3-fluoro-phenoxy)-N-[1-[5-[3-cis-(trifluoromethoxy)cyclobutyl]-1,3,4-oxadiazol-2-yl]-4-piperidyl]acetamide. ¹H-NMR (400 MHz, CDCl₃): δ 7.34 (t, J=8.60 Hz, 1H), 6.76 (dd, J=10.23, 2.82 Hz, 1H), 6.64-6.71 (m, 1H), 6.42 (br d, J=8.16 Hz, 1H), 4.67 (quin, J=7.53 Hz, 1H), 4.47 (s, 2H), 4.08-4.18 (m, 1H), 4.02 (br d, J=13.43 Hz, 2H), 3.15-3.28 (m, 3H), 2.76-2.84 (m, 2H), 2.55-2.67 (m, 2H), 2.03-2.15 (m, 2H), 1.59 (qd, J=12.05, 4.52 Hz, 2H). LC-MS m/z: =493.3, 495.3 [M+H]⁺.

### Example 27

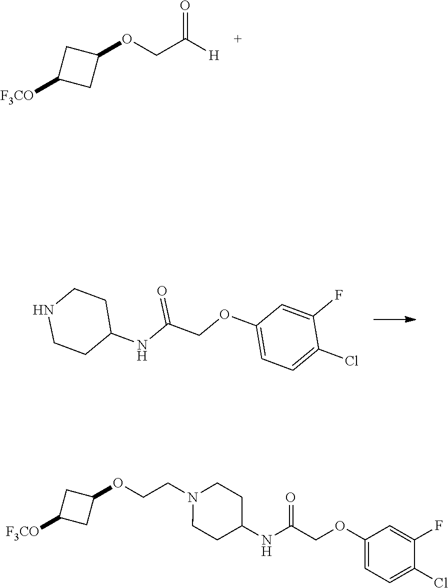 [SMILES: O=CCO[C@H]1C[C@@H](OC(F)(F)F)C1 ; O=C(COc1ccc(Cl)c(F)c1)NC1CCNCC1 ; O=C(COc1ccc(Cl)c(F)c1)NC1CCN(CCO[C@H]2C[C@@H](OC(F)(F)F)C2)CC1]

2-(4-chloro-3-fluoro-phenoxy)-N-[1-[2-[3-cis-(trifluoromethoxy)cyclobutoxy]ethyl]-4-piperidyl]acetamide: 2-(4-chloro-3-fluoro-phenoxy)-N-(4-piperidyl)acetamide (125.01 mg, 0.44 mmol), 2-[3-(trifluoromethoxy)cyclobutoxy]acetaldehyde (172.77 mg, 0.87 mmol), and acetic acid (0.14 mL, 2.4 mmol) were dissolved in DCE (8 mL). Sodium triacetoxyborohydride (138.6 mg, 0.65 mmol) was then added. The reaction mixture was stirred at rt overnight. The reaction mixture was diluted with aq. 1 NaOH (50 mL) and DCM (50 mL). The layers were separated and the aqueous layer was extracted with DCM (3×150 mL) The combined organic layers were dried over anhydrous Na₂SO₄, filtered, and concentrated under reduced pressure. The crude material mixture was purified by preparative HPLC. The acetonitrile was evaporated and the solution was eluted with ethyl acetate and sat. aq. sodium bicarbonate. The layers were separated and the combined organic layers were dried over anhydrous Na₂SO₄, filtered, and concentrated under reduced pressure. ¹H-NMR (400 MHz; CDCl₃): δ 7.35 (t, J=8.6 Hz, 1H), 6.79 (dd, J=10.3, 2.8 Hz, 1H), 6.71 (ddd, J=8.9, 2.8, 1.3 Hz, 1H), 6.54-6.46 (m, 1H), 4.52-4.45 (m, 2H), 4.35-4.31 (m, 1H), 4.06-4.00 (m, 1H), 3.78-3.67 (m, 4H), 3.50-3.48 (m, 1H), 2.84-2.77 (m, 5H), 2.56-2.45 (m, 2H), 2.27-2.22 (m, 3H), 2.06-2.02 (m, 2H). LC-MS m/z=470.60 [M+H]⁺.

### Example 28

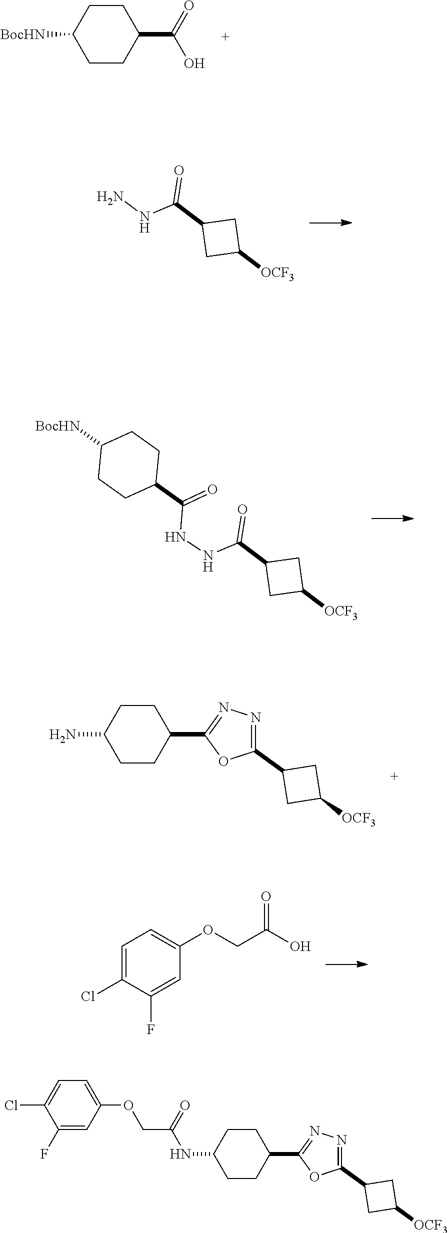

tert-butyl N-[4-[[[3-cis-(trifluoromethoxy)cyclobutanecarbonyl]amino]carbamoyl]cyclohexyl]carbamate: Prepared employing General Procedure A employing 4-(tert-butoxycarbonylamino)cyclohexanecarboxylic acid (221 mg, 0.91 mmol), 3-(trifluoromethoxy)cyclobutanecarbohydrazide (150 mg, 0.76 mmol), Et₃N (0.53 mL, 3.79 mmol), and T3P (2.7 mL, 2.27 mmol, 50% in EtOAc) in EtOAc (3.8 mL). The aqueous was extracted with 1:1 CHCl₃:i-PrOH. The product was carried on without further purification.

4-[5-[3-cis-(trifluoromethoxy)cyclobutyl]-1,3,4-oxadiazol-2-yl]cyclohexanamine: To a solution of tert-butyl N-[4-[[[3-cis-(trifluoromethoxy)cyclobutanecarbonyl]amino]carbamoyl]cyclohexyl]carbamate (321 mg, 0.76 mmol) in 1,4-dioxane (3.8 mL) was added POCl₃ (0.35 mL, 3.79 mmol). The reaction mixture was stirred at 100° C. for 1 h. The reaction mixture was cooled to 0° C., quenched by the addition of sat. NaHCO₃ (15 mL), and extracted with 1:1 CHCl₃:i-PrOH (5×20 mL). The combined organics were then dried over MgSO₄, filtered, and concentrated under reduced pressure. The product was carried on without further purification. LC-MS, m/z=306.5 [M+H]⁺.

2-(4-chloro-3-fluoro-phenoxy)-N-[4-[5-[3-cis-(trifluoromethoxy)cyclobutyl]-1,3,4-oxadiazol-2-yl]cyclohexyl]acetamide: Prepared using General Procedure A employing 4-[5-[3-cis-(trifluoromethoxy)cyclobutyl]-1,3,4-oxadiazol-2-yl]cyclohexanamine (50 mg, 0.16 mmol), 2-(4-chloro-3-fluoro-phenoxy)acetic acid (50 mg, 0.25 mmol), Et₃N (0.11 mL, 0.82 mmol), and T3P (0.29 mL, 0.49 mmol, 50% in EtOAc) in EtOAc (1.6 mL). The crude reaction mixture was purified employing reverse-phase HPLC. LC-MS, m/z=492.5 [M+H]⁺. ¹H-NMR (400 MHz; CDCl₃): δ 7.38-7.34 (m, 1H), 6.79 (dd, J=10.3, 2.8 Hz, 1H), 6.71 (ddd, J=8.9, 2.9, 1.2 Hz, 1H), 6.35 (d, J=8.1 Hz, 1H), 4.72 (t, J=7.5 Hz, 1H), 4.47 (s, 2H), 3.97 (dt, J=8.0, 3.9 Hz, 1H), 3.35 (ddd, J=10.2, 7.7, 2.4 Hz, 1H), 2.92-2.85 (m, 3H), 2.72-2.64 (m, 2H), 2.27-2.18 (m, 4H), 1.83-1.74 (m, 2H), 1.43-1.37 (m, 2H).

### Example 29

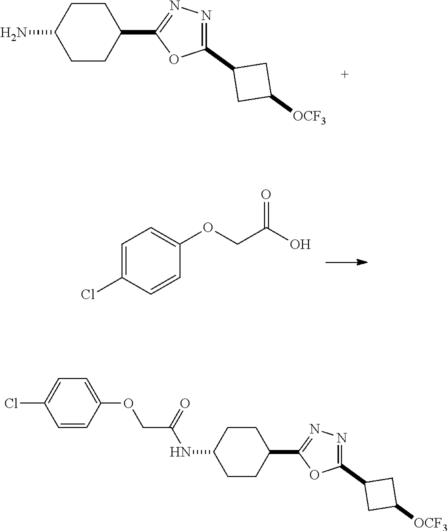

2-(4-chlorophenoxy)-N-[4-[5-[3-cis-(trifluoromethoxy)cyclobutyl]-1,3,4-oxadiazol-2-yl]cyclohexyl]acetamide: Prepared using General Procedure A employing 4-[5-[3-cis-(trifluoromethoxy)cyclobutyl]-1,3,4-oxadiazol-2-yl]cyclohexanamine (50 mg, 0.16 mmol), 2-(4-chlorophenoxy)acetic acid (46 mg, 0.25 mmol) and Et₃N (0.11 mL, 0.82 mmol) and T3P (0.29 mL, 0.49 mmol, 50% in EtOAc) in EtOAc (1.6 mL). The crude reaction mixture was purified employing reverse-phase HPLC. LC-MS, m/z=474.3 [M+H]⁺. ¹H-NMR (400 MHz; CDCl₃): δ 7.32-7.30 (m, 2H), 6.90-6.86 (m, 2H), 6.40 (d, J=8.1 Hz, 1H), 4.72 (t, J=7.5 Hz, 1H), 4.48 (s, 2H), 3.97 (d, J=8.1 Hz, 1H), 3.35 (ddd, J=10.2, 7.7, 2.4 Hz, 1H), 2.92-2.85 (m, 3H), 2.72-2.67 (m, 2H), 2.27-2.17 (m, 4H), 1.80-1.74 (m, 2H), 1.42-1.36 (m, 2H).

### Example 30

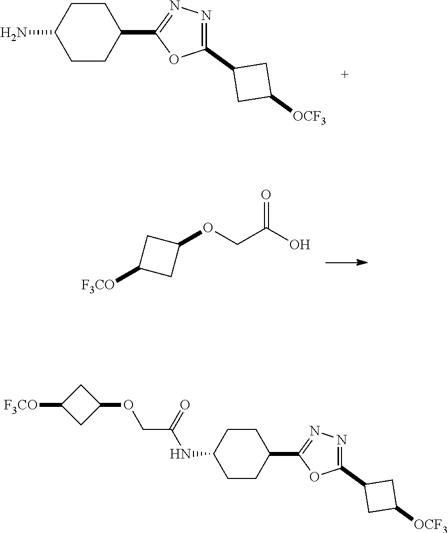

2-[3-cis-(trifluoromethoxy)cyclobutoxy]-N-[4-[5-[3-cis-(trifluoromethoxy)cyclobutyl]-1,3,4-oxadiazol-2-yl]cyclohexyl]acetamide: Prepared using General Procedure A employing 4-[5-[3-cis-(trifluoromethoxy)cyclobutyl]-1,3,4-oxadiazol-2-yl]cyclohexanamine (70 mg, 0.23 mmol), 2-[3-cis-(trifluoromethoxy)cyclobutoxy]acetic acid (74 mg, 0.34 mmol), Et₃N (0.16 mL, 1.15 mmol) and T3P (0.41 mL, 0.69 mmol, 50% in EtOAc) in EtOAc (1.1 mL). The crude reaction mixture was purified employing reverse phase HPLC to provide the desired product. The crude reaction mixture was purified employing reverse-phase HPLC. LC-MS, m/z=502.8 [M+H]⁺. ¹H-NMR (400 MHz; CDCl₃): δ 6.37 (d, J=8.3 Hz, 1H), 4.72 (qd, J=8.8, 7.1 Hz, 1H), 4.34 (quintet, J=7.2 Hz, 1H), 3.88-3.86 (m, 2H), 3.73 (quintet, J=6.9 Hz, 1H), 3.38-3.29 (m, 1H), 2.90-2.77 (m, 6H), 2.71-2.63 (m, 2H), 2.30-2.15 (m, 6H), 1.83-1.71 (m, 2H), 1.42-1.32 (m, 2H).

### Example 31

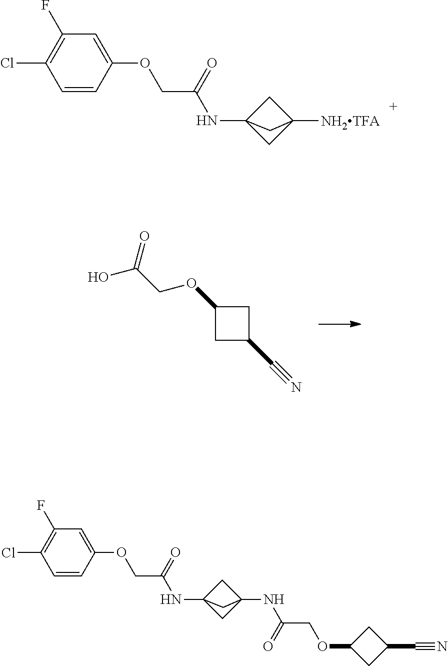

2-(4-chlorophenoxy)-N-[3-[[2-(3-cis-cyanocyclobutoxy)acetyl]amino]-1-bicyclo[1.1.1]pentanyl]acetamide: Prepared using General Procedure A employing 2-(3-cyanocyclobutoxy)acetic acid (127 mg, 0.82 mmol), N-(1-amino-3-bicyclo[1.1.1]pentanyl)-2-(4-chlorophenoxy)acetamide TFA salt (208 mg, 0.55 mmol), Et₃N (0.38 mL, 2.73 mmol), and T3P (1.95 mL, 1.64 mmol, 50% in EtOAc) in EtOAc (5.5 mL). The crude reaction mixture was purified employing reverse-phase HPLC. LC-MS, m/z=404.3 [M+H]⁺. ¹H-NMR (400 MHz; CDCl₃): δ 7.28-7.24 (m, 2H), 7.05 (s, 1H), 6.93 (s, 1H), 6.87-6.83 (m, 2H), 4.39 (s, 2H), 4.00-3.96 (m, 1H), 3.80 (s, 2H), 2.76-2.67 (m, 3H), 2.49 (s, 6H), 2.42-2.35 (m, 2H).

### Example 32

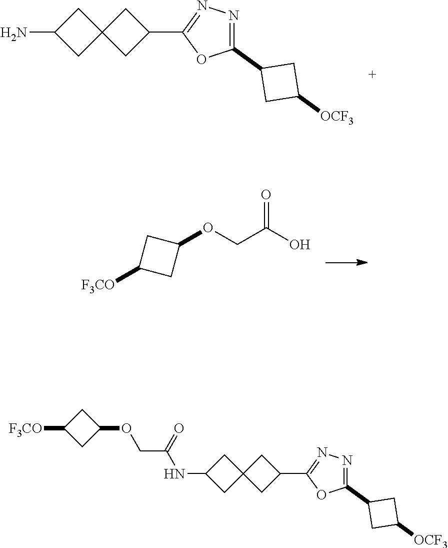

2-[3-cis-(trifluoromethoxy)cyclobutoxy]-N-[6-[5-[3-cis-(trifluoromethoxy)cyclobutyl]-1,3,4-oxadiazol-2-yl]spiro[3.3]heptan-2-yl]acetamide: 3-methyl-3-[2-[5-[3-(trifluoromethoxy)cyclobutyl]-1,3,4-oxadiazol-2-yl]propyl]cyclobutanamine; 2,2,2-trifluoroacetic acid (80 mg, 0.1800 mmol), 2-[3-(trifluoromethoxy)cyclobutoxy]acetic acid (46 mg, 0.21 mmol) were dissolved in EtOAc (1.8 mL) followed by the addition of triethylamine (0.12 mL, 0.89 mmol) and T3P solution (0.32 mL, 0.54 mmol). The reaction was heated to 100° C. in the sealed vial overnight. The resulting heterogeneous reaction mixture was stirred for 20 h. The consumption of starting material was complete by HPLC-MS analysis, and the reaction mixture was treated with aq. sat. NaHCO₃ solution (5 mL) and EtOAc (10 mL) The layers were separated and the aqueous layer was extracted with EtOAc (3×10 mL) The combined organic layers were dried over anhydrous MgSO4, filtered, and concentrated under reduced pressure The crude reaction mixture was purified employing reverse phase HPLC to provide the desired product. LC-MS, m/z=514.3 [M+H]⁺. ¹H-NMR (400 MHz; CDCl₃): δ 6.56 (d, J=8.0 Hz, 1H), 4.71 (quintet, J=7.5 Hz, 1H), 4.36 (tq, J=15.1, 7.6 Hz, 2H), 3.88-3.78 (m, 2H), 3.75-3.66 (m, 1H), 3.62 (quintet, J=8.6 Hz, 1H), 3.33 (tt, J=10.1, 7.8 Hz, 1H), 2.85 (ddtd, J=16.8, 11.7, 5.7, 2.9 Hz, 4H), 2.71-2.60 (m, 3H), 2.55-2.38 (m, 5H), 2.31-2.24 (m, 2H), 2.03 (ddd, J=20.9, 11.4, 9.3 Hz, 2H).

### Example 33

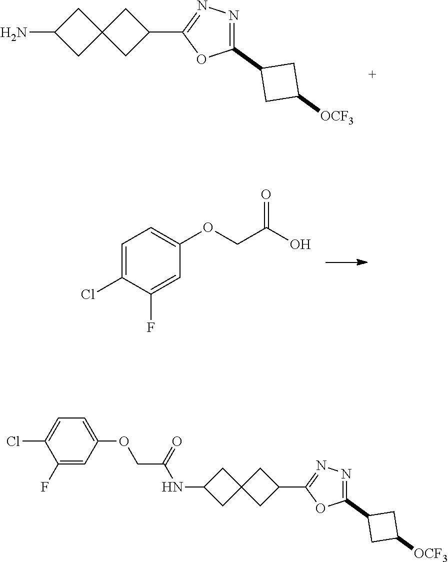

2-(4-chloro-3-fluoro-phenoxy)-N-[6-[5-[3-cis-(trifluoromethoxy)cyclobutyl]-1,3,4-oxadiazol-2-yl]spiro[3.3]heptan-2-yl]acetamide: 3-methyl-3-[2-[5-[3-(trifluoromethoxy)cyclobutyl]-1,3,4-oxadiazol-2-yl]propyl]cyclobutanamine; 2,2,2-trifluoroacetic acid (80 mg, 0.18 mmol), 2-(4-chloro-3-fluoro-phenoxy)acetic acid (44 mg, 0.21 mmol) were dissolved in EtOAc (1.8 mL) followed by the addition of triethylamine (0.12 mL, 0.89 mmol) and T3P solution (0.32 mL, 0.54 mmol). The reaction was heated to 100° C. in the sealed vial overnight. The resulting heterogeneous reaction mixture was stirred for 20 h. The consumption of starting material was complete by HPLC-MS analysis, and the reaction mixture was treated with aq. sat. NaHCO₃ solution (5 mL) and EtOAc (10 mL) The layers were separated and the aqueous layer was extracted with EtOAc (3×10 mL) The combined organic layers were dried over anhydrous MgSO4, filtered, and concentrated under reduced pressure The crude reaction mixture was purified employing reverse phase HPLC to provide the desired product. LC-MS, m/z=504.3 [M+H]⁺. ¹H-NMR (400 MHz; CDCl₃): δ 7.36-7.31 (m, 1H), 6.76 (dt, J=10.2, 3.1 Hz, 1H), 6.68 (ddd, J=8.9, 2.8, 1.2 Hz, 1H), 6.57 (d, J=7.8 Hz, 1H), 4.71 (quintet, J=7.5 Hz, 1H), 4.48-4.39 (m, 3H), 3.62 (quintet, J=8.5 Hz, 1H), 3.33 (tt, J=10.1, 7.8 Hz, 1H), 2.91-2.83 (m, 2H), 2.71-2.63 (m, 3H), 2.57-2.38 (m, 5H), 2.04 (ddd, J=20.4, 11.3, 9.0 Hz, 2H).

### Example 34

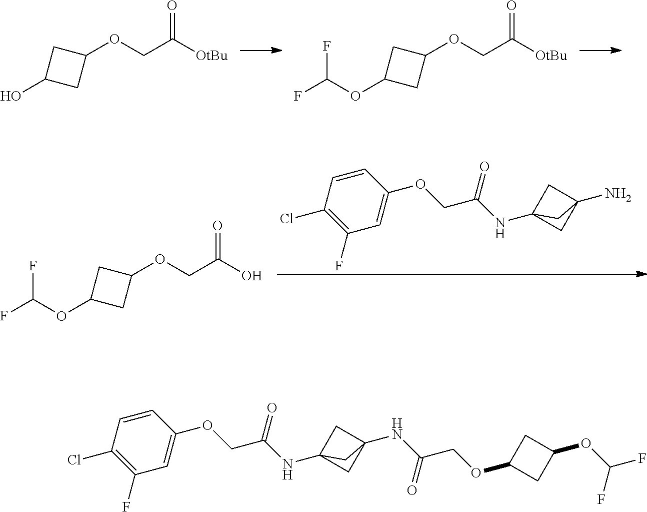

tert-butyl 2-(3-(difluoromethoxy)cyclobutoxy)acetate: To a solution of tert-butyl 2-(3-hydroxycyclobutoxy)acetate (250 mg, 1.24 mmol) in dry CH₃CN (5 mL) was added CuI (47 mg, 0.25 mmol), the mixture was heated to 50° C., and a solution of 2,2-difluoro-2-fluorosulfonyl-acetic acid (0.19 mL, 1.85 mmol) in dry CH₃CN (5 mL) was added dropwise over a period of 5 min. The reaction mixture was heated to 50° C. for 2 h, and was diluted with sat. NaHCO₃ (10 mL). The mixture was adjusted to pH<7 by addition of 6 N HCl, and was then extracted with DCM:i-PrOH (v:v=3:1, 3×10 mL). The combined organic layers were washed with brine (10 mL), dried over anhydrous Na₂SO₄, filtered and concentrated under reduced pressure to provide tert-butyl 2-(3-(difluoromethoxy)cyclobutoxy)acetate, which was directly used for next step. ¹H-NMR (400 MHz, CDCl₃): δ 5.93-6.38 (m, 1H), 4.26 (quin, 1=7.25 Hz, 1H), 4.03-4.05 (m, 2H), 3.74-3.81 (m, 1H), 2.77 (dtd, J=9.69, 6.48, 6.48, 3.33 Hz, 2H), 2.19-2.30 (m, 2H), 1.35 (s, 6H).

2-(3-(difluoromethoxy)cyclobutoxy)acetic acid: To a solution of tert-butyl 2-(3-(difluoromethoxy)cyclobutoxy)acetate (200 mg, 0.79 mmol) in DCM (10 mL) was added TFA (0.18 mL, 2.38 mmol), the mixture was heated to 30° C. for 2 h. The residue was treated with with sat. NaHCO₃ (10 mL) and was extracted with DCM:i-PrOH (v:v=3:1, 3×10 mL). The combined organic phases were washed with brine (10 mL), dried over anhydrous Na₂SO₄, filtered and concentrated under reduced pressure to provide 2-(3-(difluoromethoxy)cyclobutoxy)acetic acid. ¹H-NMR (400 MHz, CDCl₃): δ 5.95-6.38 (m, 1H), 4.23-4.31 (m, 1H), 4.07 (s, 2H), 3.77-3.83 (m, 1H), 2.78 (dtd, J=9.76, 6.57, 6.57, 3.20 Hz, 2H), 2.24 (dtd, J=10.02, 7.41, 7.41, 3.01 Hz, 2H).

2-(4-chloro-3-fluoro-phenoxy)-N-[3-[[2-[3-cis-(difluoromethoxy)cyclobutoxy]acetyl]amino]-1-bicyclo[1.1.1]pentanyl]acetamide: To a solution of N-(3-aminobicyclo[1.1.1]pentan-1-yl)-2-(4-chloro-3-fluorophenoxy)acetamide (130 mg, 0.4 mmol, HCl salt) in DMF (5 mL) was added 2-(3-(difluoromethoxy)cyclobutoxy)acetic acid (80 mg, 0.4 mmol), Na₂CO₃ (86 mg, 0.8 mmol) and HATU (170 mg, 0.4 mmol). The mixture was stirred at 20° C. for 2 h, was filtered through a pad of celite to provide residue, which was purified by prep-HPLC (TFA) to deliver 2-(4-chloro-3-fluoro-phenoxy)-N-[3-[[2-[3-cis-(difluoromethoxy)cyclobutoxy]acetyl]amino]-1-bicyclo[1.1.1]pentanyl]acetamide. LC-MS: m/z=463.3 [M+H]⁺. ¹H-NMR (400 MHz, CDCl₃): δ 7.33 (t, J=8.60 Hz, 1H), 6.93 (br d, J=4.41 Hz, 2H), 6.77 (dd, J=10.25, 2.76 Hz, 1H), 6.68 (dt, J=8.82, 1.32 Hz, 1H), 5.97-6.36 (m, 1H), 4.42 (s, 2H), 4.29 (quin, J=7.17 Hz, 1H), 3.83 (s, 2H), 3.64-3.77 (m, 1H), 2.72-2.84 (m, 2H), 2.51 (s, 6H), 2.19 (dtd, J=10.01, 7.35, 7.35, 3.09 Hz, 2H).

### Example 35

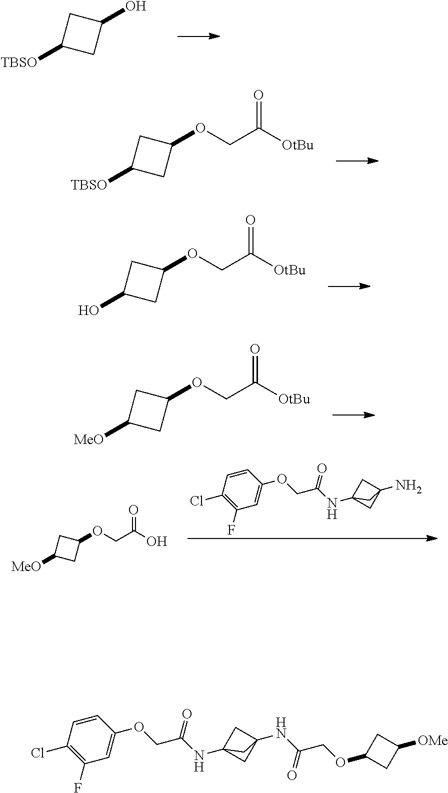

Tert-butyl 2-[3-[tert-butyl(dimethyl)silyl]oxycyclobutoxy]acetate: To a mixture of 3-[tert-butyl(dimethyl)silyl]oxycyclobutanol (3.5 g, 17.3 mmol) in THE (30 mL) at 0° C. was added NaH (830 mg, 20.8 mmol, 60% purity). After 30 min, tert-butyl 2-bromoacetate (3.71 g, 19.0 mmol) was added, and the mixture was stirred for 12 h at rt. The mixture was poured into aq. NH₄Cl (50 mL) and EtOAc (25 mL) was added. The layers were separated and the aqueous phase was extracted with EtOAc (3×15 mL). The combined organic phases were washed with brine (15 mL), dried with anhydrous Na₂SO₄, filtered and concentrated under reduced pressure. The residue was purified by silica gel column chromatography (PE:EtOAc=100:1 to 1:1) to provide tert-butyl 2-[3-[tert-butyl (dimethyl)silyl]oxycyclobutoxy]acetate.

Tert-butyl 2-(3-hydroxycyclobutoxy) acetate: To a mixture of tert-butyl 2-[3-[tert-butyl (dimethyl)silyl]oxycyclobutoxy]acetate (4.0 g, 12.6 mmol) in THE (50 mL) at rt was added TBAF (1 M in THF, 19 mL) and the mixture was stirred for 4 h. The reaction mixture was poured into ice-water (100 mL) and EtOAc (50 mL). The layers were separated and the aqueous phase was extracted with EtOAc (3×50 mL). The combined organic phases were washed with brine (50 mL), dried with anhydrous Na₂SO₄, filtered and concentrated under reduced pressure. The residue was purified by silica gel column chromatography (PE:EtOAc=100:1 to 1:1) to provide tert-butyl 2-(3-hydroxycyclobutoxy) acetate. ¹H-NMR (400 MHz, CDCl₃): δ 3.83-3.95 (m, 2H), 3.68 (quin, J=6.89 Hz, 1H), 2.74 (dtd, J=9.56, 6.46, 6.46, 3.09 Hz, 2H), 1.97 (dtd, J=9.78, 7.35, 7.35, 2.87 Hz, 2H), 1.48 (s, 9H).

Tert-butyl 2-(3-methoxycyclobutoxy) acetate: To a mixture of tert-butyl 2-(3-hydroxycyclobutoxy) acetate (100 mg, 0.49 mmol) and NaHCO₃ (831 mg, 9.89 mmol) in DCM at 0° C. (20 mL) was added trimethyloxonium tetrafluoroborate (731 mg, 4.94 mmol), and the reaction mixture was stirred at 20° C. for 12 h. The mixture was poured into ice-water (20 mL), the layers were separated and the aqueous phase was extracted with DCM (3×30 mL). The combined organic phases were washed with brine (30 mL), dried with anhydrous Na₂SO₄, filtered and concentrated under reduced pressure to provide tert-butyl 2-(3-methoxycyclobutoxy)acetate. ¹H-NMR (400 MHz, CDCl₃): δ 3.86-3.94 (m, 2 H), 3.61-3.78 (m, 1H), 3.50 (quin, J=7.00 Hz, 1H), 3.20-3.28 (m, 3H), 2.66 (dtd, J=9.40, 6.44, 6.44, 3.09 Hz, 2H), 1.91-2.01 (m, 2H) 1.48 (s, 9H).

2-(3-methoxycyclobutoxy) acetic acid: To a mixture of tert-butyl 2-(3-methoxycyclobutoxy)acetate (80 mg, 0.37 mmol) in DCM at rt (5 mL) was added TFA (169 mg, 1.48 mmol), and the mixture was stirred at 35° C. for 6 h. The mixture was concentrated and the residue was diluted with aq. NaHCO₃ (10 mL). The aqueous phase was extracted with MTBE (5 mL) and the aqueous layer was adjusted the pH to 3 by addition of HCl (2 N). The aqueous phase was extracted with DCM:MeOH (v:v=3:1, 3×5 mL), dried with anhydrous Na₂SO₄, filtered and concentrated under reduced pressure to provide 2-(3-methoxycyclobutoxy) acetic acid. ¹H-NMR (400 MHz, CDCl₃): δ 6.39 (br s, 2H), 4.05 (s, 2H), 3.72-3.83 (m, 1H), 3.49-3.60 (m, 1H), 3.26 (s, 3H), 2.69 (dtd, J=9.52, 6.44, 6.44, 3.07 Hz, 2H), 1.91-2.04 (m, 2H).

2-(4-chloro-3-fluoro-phenoxy)-N-[3-[[2-(3-cis-methoxycyclobutoxy)acetyl]amino]-1-bicyclo[1.1.1]pentanyl]acetamide: To a mixture of 2-(3-methoxycyclobutoxy)acetic acid (35 mg, 0.22 mmol) and N-(1-amino-3-bicyclo[1.1.1]pentanyl)-2-(4-chloro-3-fluoro-phenoxy)acetamide HCl salt (62 mg, 0.19 mmol) in DMF (5 mL) at rt was added DIEA (113 mg, 0.87 mmol) and HATU (125 mg, 0.33 mmol) and the mixture was stirred at 20° C. for 2 h. The reaction mixture was poured into ice-water (15 mL) and EtOAc (10 mL). The layers were separated and the aqueous phase was extracted with EtOAc (3×5 mL). The combined organic phases were washed with brine (4×5 mL), dried with anhydrous Na₂SO₄, filtered and concentrated under reduced pressure. The residue was purified by prep-HPLC (TFA) to provide 2-(4-chloro-3-fluoro-phenoxy)-N-[3-[[2-(3-methoxycyclobutoxy)acetyl]amino]-1-bicyclo[1.1.1]pentanyl]acetamide. ¹H-NMR (400 MHz, CDCl₃): δ 7.34 (t, J=8.60 Hz, 1H), 6.99 (s, 1H), 6.89 (s, 1H), 6.77 (dd, J=10.14, 2.87 Hz, 1H), 6.69 (br d, J=8.82 Hz, 1H), 4.42 (s, 2H), 3.84 (s, 2H), 3.70 (quin, J=6.95 Hz, 1H), 3.56 (quin, J=6.78 Hz, 1H), 3.26 (s, 3H), 2.64-2.74 (m, 2H), 2.51 (s, 6H), 1.93 (dtd, J=9.78, 7.24, 7.24, 2.87 Hz, 2H). LC-MS m/z: =427.4, 429.4 [M+H]⁺.

### Example 36

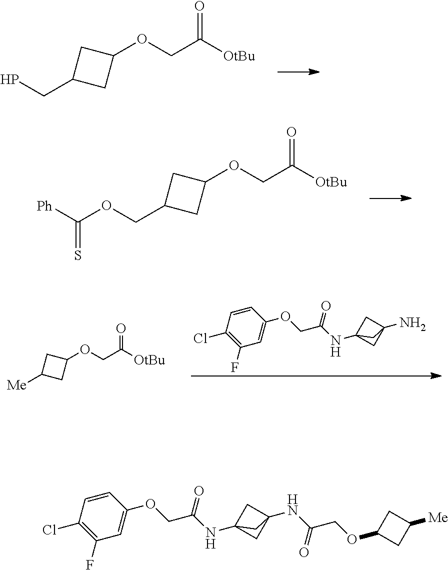

tert-butyl 2-(3-(((phenoxycarbonothioyl)oxy)methyl)cyclobutoxy)acetate: To a solution of tert-butyl 2-(3-(hydroxymethyl)cyclobutoxy)acetate (500 mg, 2.31 mmol) in DCM (10 mL) was added pyridine (0.63 mL, 7.85 mmol) and DMAP (28 mg, 0.23 mmol). O-phenyl carbonochloridothioate (439 mg, 2.54 mmol) was added, and the resulting mixture was stirred at 20° C. for 3 h. Aqueous 10% KF (20 mL) was added to the reaction at 0° C., the layers were separated, and the aqueous layer was extracted with EtOAc (2×20 mL). The combined organic layers were washed with brine (20 mL), dried over anhydrous Na₂SO₄, filtered and concentrated under reduced pressure to provide the residue, which was purified by silica gel column chromatography (PE:MTBE=100:1 to 1:100) to provide tert-butyl 2-(3-(((phenoxycarbonothioyl)oxy)methyl)cyclobutoxy)acetate. ¹H-NMR (400 MHz, CDCl₃): 7.37-7.44 (m, 2H) 7.24-7.29 (m, 1H) 7.07-7.12 (m, 2H) 4.46-4.53 (m, 2H) 3.94-4.04 (m, 1H) 3.88 (s, 2H) 2.41-2.50 (m, 2H) 2.25-2.36 (m, 1H) 1.84-1.93 (m, 2H) 1.47 (s, 9H).

tert-butyl 2-(3-methylcyclobutoxy)acetate: To a solution of tert-butyl 2-(3-(((phenoxycarbonothioyl)oxy)methyl)cyclobutoxy)acetate (300 mg, 0.85 mmol) in PhMe (30 mL) was added Bu₃SnH (496 mg, 1.70 mmol) and AIBN (154 mg, 0.94 mmol). The mixture was stirred under illumination at 120° C. for 4 h. Sat. NH₄Cl 10 mL was added to the reaction mixture, the layers were separated and the aqueous layer was extracted with EtOAc (2×10 mL). The combined organic layers were washed with brine (10 mL), dried over anhydrous Na₂SO₄ and concentrated under reduced pressure to provide a residue, which was purified by silica gel column chromatography (PE:MTBE=100:1 to 1:100) to provide tert-butyl 2-(3-methylcyclobutoxy)acetate.

2-(3-methylcyclobutoxy)acetic acid: To a solution of tert-butyl 2-(3-methylcyclobutoxy)acetate (120 mg, 0.60 mmol) in DCM (20 mL) was added TFA (273 mg, 2.40 mmol), and the mixture was heated to 30° C. and stirred for 12 h. The reaction was concentrated to provide 2-(3-methylcyclobutoxy)acetic acid, which was used directly for the next step.

2-(4-chlorophenoxy)-N-[3-[[2-(3-cis-methylcyclobutoxy)acetyl]amino]-1-bicyclo[1.1.1]pentanyl]acetamide: To a solution of 2-(3-methylcyclobutoxy)acetic acid (62 mg, 0.43 mmol) in DMF (2 mL) was added N-(3-aminobicyclo[1.1.1]pentan-1-yl)-2-(4-chlorophenoxy)acetamide (130 mg, 0.43 mmol, HCl), Na₂CO₃ (91 rug, 0.86 mmol) and HATU (180 mg, 0.47 mmol) and the mixture was stirred at 20° C. for 2 h. The reaction mixture was filtered through a pad of celite to provide residue, which was purified by prep-HPLC (TFA) and then by SFC to provide the desired product. LC-MS: m/z=393.4, 395.4 [M+H]⁺. ¹H-NMR (400 MHz, CDCl₃): δ 7.24-7.29 (m, 2H), 6.91 (br d, J=7.78 Hz, 2H), 6.82-6.87 (m, 2H), 4.39 (s, 2H), 3.77-3.83 (m, 1H), 3.76 (s, 2H), 2.48 (s, 6H), 2.34-2.44 (m, 2H), 1.80-1.93 (m, 1H), 1.44-1.56 (m, 2H), 1.08 (d, J=6.65 Hz, 3H).

### Example 37

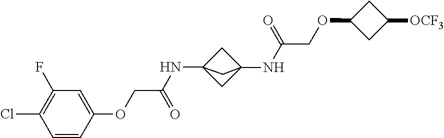 [SMILES: O=C(COc1ccc(Cl)c(F)c1)NC12CC(NC(=O)CO[C@H]3C[C@@H](OC(F)(F)F)C3)(C1)C2]

2-(4-chloro-3-fluoro-phenoxy)-N-[3-[[2-[3-cis-(trifluoromethoxy)cyclobutoxy]acetyl]amino]-1-bicyclo[1.1.1]pentanyl]acetamide: To a mixture of 2-[3-cis-(trifluoromethoxy)cyclobutoxy]acetic acid (50 mg, 0.23 mmol) and N-(1-amino-3-bicyclo[1.1.1]pentanyl)-2-(4-chloro-3-fluoro-phenoxy)acetamide HCl salt (90 mg, 0.28 mmol) in DMF (5 mL) at rt was added HATU (107 mg, 0.28 mmol) and DIEA (151 mg, 1.17 mmol). The reaction mixture was stirred for 2 h, was poured into ice-water (10 mL). EtOAc (10 mL) was added, the layers were separated, and the aqueous phase was extracted with EtOAc (3×10 mL). The combined organic phases were washed with brine (4×10 mL), dried with anhydrous Na₂SO₄, filtered and concentrated under reduced pressure. The residue was purified by prep-HPLC (TFA) to provide 2-(4-chloro-3-fluoro-phenoxy)-N-[3-[[2-[3-cis-(trifluoromethoxy)cyclobutoxy]acetyl]amino]-1-bicyclo[1.1.1]pentanyl]acetamide. ¹H-NMR (400 MHz, CDCl₃): δ 7.34 (t, J=8.60 Hz, 1H), 6.89 (br s, 2H), 6.77 (dd, J=10.16, 2.76 Hz, 1H), 6.69 (br d, J=8.78 Hz, 1H), 4.42 (s, 2H), 4.33 (quin, J=7.15 Hz, 1H), 3.84 (s, 2H), 3.72 (quin, J=6.78 Hz, 1H), 2.78-2.88 (m, 2H), 2.52 (s, 6H), 2.22-2.34 (m, 2H). LC-MS m/z: =481.4, 483.4 [M+H]⁺.

### Example 38

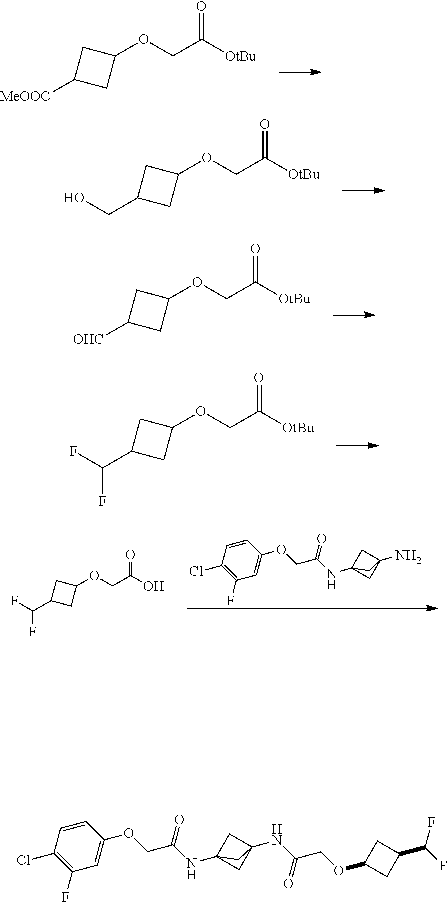

tert-butyl 2-(3-(hydroxymethyl)cyclobutoxy)acetate: To a solution of methyl tert-butyl 2-(3-(hydroxymethyl)cyclobutoxy)acetate (3.0 g, 12.3 mmol) in THE (30 mL) was added LiAlH(OtBu)₃ (30.7 mL, 1 M in THF) at 20° C., and the mixture was stirred at 84° C. for 5 h. The mixture was diluted with sat. NH₄Cl (30 mL) stirred for 5 min, and EtOAc (30 mL) was added. The layers were separated and the aqueous layer was extracted with EtOAc (3×30 mL). The combined organic phases were washed with brine (30 mL), dried over Na₂SO₄ and concentrated under reduced pressure to provide the residue, which was purified by silica gel column chromatography (PE:MTBE=100:1 to 1:100) to provide tert-butyl 2-(3-(hydroxymethyl)cyclobutoxy)acetate. ¹H-NMR (400 MHz, CDCl₃): 3.95 (quin, J=7.28 Hz, 1H), 3.86 (s, 2H), 3.59 (d, J=6.15 Hz, 2H), 2.30-2.40 (m, 2H), 1.97-2.06 (m, 1H), 1.69-1.80 (m, 2H), 1.46 (s, 9H).

tert-butyl 2-(3-formylcyclobutoxy)acetate: To a solution of tert-butyl 2-(3-(hydroxymethyl)cyclobutoxy)acetate (500 mg, 2.31 mmol) in DCM (20 ml) was added Dess-Martin (0.86 mL, 2.77 mmol) in one portion, and the mixture was stirred at 20° C. for 3 h. The mixture was diluted with 10% sodium thiosulphate solution (20 mL), and the layers were separated. The organic layer was washed with 10% sodium thiosulphate solution (20 mL), with 0.5 M NaOH (2×20 mL) and brine (20 mL). The organic phase were dried over Na₂SO₄ and concentrated under reduced pressure to provide the residue, which was purified by silica gel column chromatography (PE:MTBE=100:1 to 1:100) to provide tert-butyl 2-(3-formylcyclobutoxy)acetate. ¹H-NMR (400 MHz, CDCl₃): 9.66 (d, J=2.65 Hz, 1H), 4.10 (quin, J=7.33 Hz, 1H), 3.90 (s, 2H), 2.63-2.74 (m, 1H), 2.43-2.52 (m, 2H), 2.20-2.30 (m, 2H), 1.48 (s, 9H).

tert-butyl 2-(3-(difluoromethyl)cyclobutoxy)acetate: To a solution of tert-butyl 2-(3-formylcyclobutoxy)acetate (460 mg, 2.15 mmol) in dry DCM (40 mL) was added DAST solution (0.85 mL, 6.44 mmol) at −30° C. and the mixture was stirred at 20° C. for 16 h. After cooling to 0° C., sat. NH₄Cl₃ (20 mL) was added to the reaction mixture and the layers were separated. The organic layer was washed with brine (20 mL), dried over Na₂SO₄ and concentrated under reduced pressure to provide the residue, which was purified by silica gel column chromatography (PE:MTBE=100:1 to 1:100) to deliver tert-butyl 2-(3-(difluoromethyl)cyclobutoxy)acetate. ¹H-NMR (400 MHz, CDCl₃): δ 5.57-5.90 (m, 1H), 3.96-4.06 (m, 1H), 3.89 (s, 2H), 2.33-2.42 (m, 2H), 2.19-2.32 (m, 1H), 1.99-2.09 (m, 2H), 1.47-1.50 (m, 9H).

2-(3-(difluoromethyl)cyclobutoxy)acetic acid: To a solution of tert-butyl 2-(3-(difluoromethyl)cyclobutoxy)acetate (150 mg, 0.60 mmol) in DCM (10 mL) was added TFA (190 μL, 2.54 mmol), the mixture was heated to 30° C. and stirred for 6 h. The reaction mixture was concentrated under reduced pressure to provide 2-(3-(difluoromethyl)cyclobutoxy)acetic acid, which was used directly for the next step. ¹H-NMR (400 MHz, CDCl₃): δ 5.55-5.93 (m, 1H), 4.09-4.12 (m, 1H), 4.02 (s, 2H), 2.33-2.42 (m, 2H), 2.19-2.31 (m, 1H), 1.95-2.10 (m, 2H).

2-(4-chloro-3-fluoro-phenoxy)-N-[3-[[2-[3-cis-(difluoromethyl)cyclobutoxy]acetyl]amino]-1-bicyclo[1.1.1]pentanyl]acetamide: To a solution of N-(3-aminobicyclo[1.1.1]pentan-1-yl)-2-(4-chloro-3-fluorophenoxy)acetamide (130 mg, 0.4 mmol, HCl salt) in DMF (5 mL) was added 2-(3-(difluoromethyl)cyclobutoxy)acetic acid (80 mg, 0.4 mmol), Na₂CO₃ (86 mg, 0.8 mmol) and HATU (170 mg, 0.4 mmol), and the mixture was stirred at 20° C. for 2 h. The reaction mixture was filtered through a pad of celite to provide residue, which was purified by prep-HPLC (TFA) to provide 2-(4-chloro-3-fluorophenoxy)-N-(3-(2-(cis-3-(difluoromethyl)cyclobutoxy)acetamido)bicyclo[1.1.1]pentan-1-yl)acetamide. ¹H-NMR (400 MHz, CDCl₃): δ 7.33 (t, J=8.60 Hz, 1H), 6.91 (br d, J=4.77 Hz, 2H), 6.77 (dd, J=10.23, 2.70 Hz, 1H), 6.69 (br d, J=8.91 Hz, 1H), 5.60-5.93 (m, 1H), 4.41 (s, 2H), 3.97 (quin, J=6.78 Hz, 1H), 3.82 (s, 2H), 2.51 (s, 6H), 2.35-2.44 (m, 2H), 2.32 (br dd, J=7.65, 3.14 Hz, 1H), 1.95-2.09 (m, 2H). LC-MS: m/z=447.4, 449.2 [M+H]⁺.

### Example 39

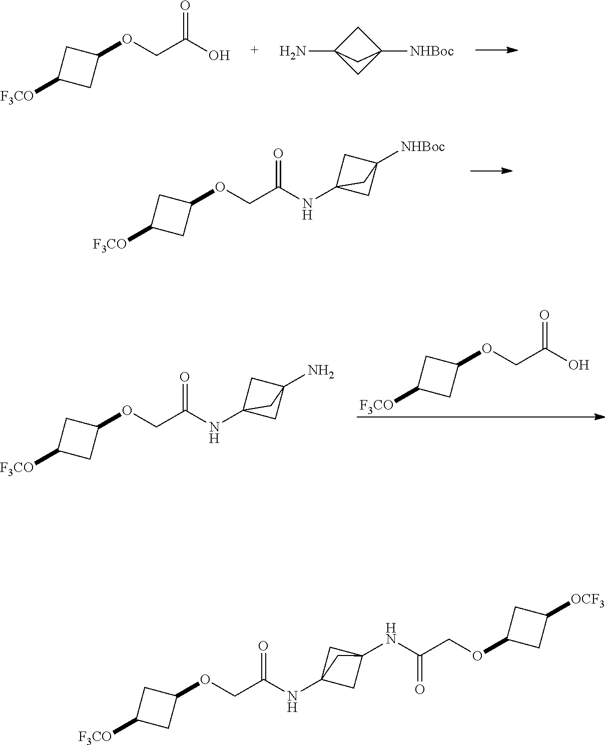

N-[1-[[2-[3-(trifluoromethoxy)cyclobutoxy]acetyl]amino]-3-bicyclo[1.1.1]pentanyl]carbamate: To a mixture of 2-[3-cis-(trifluoromethoxy)cyclobutoxy]acetic acid (20.0 mg, 0.09 mmol) and tert-butyl N-(1-amino-3-bicyclo[1.1.1]pentanyl)carbamate (18.5 mg, 0.09 mmol) in DMF (2.0 mL) was added HATU (53.3 mg, 0.140 mmol) and DIEA (48.3 mg, 0.37 mmol) and the mixture was stirred at rt for 12 h. The reaction mixture was poured into ice-water (10 mL), and EtOAc (10 mL). The layers were separated and the aqueous phase was extracted with EtOAc (3×5 mL). The combined organic phases were washed with brine (4×10 mL), dried with anhydrous Na₂SO₄, filtered and concentrated under reduced pressure to provide tert-butyl N-[1-[[2-[3-cis-(trifluoromethoxy)cyclobutoxy]acetyl]amino]-3-bicyclo[1.1.1]pentanyl]carbamate, which was used directly.

N-(1-amino-3-bicyclo [1.1.1] pentanyl)-2-[3-cis-(trifluoromethoxy) cyclobutoxy] acetamide HCl salt: To a mixture of tert-butyl N-[1-[[2-[3-cis-(trifluoromethoxy) cyclobutoxy] acetyl] amino]-3-bicyclo [1.1.1] pentanyl] carbamate (40 mg, 0.10 mmol) in HCl/EtOAc (5 mL) was stirred at 20° C. for 3 h. The mixture was concentrated to afford N-(1-amino-3-bicyclo [1.1.1] pentanyl)-2-[3-cis-(trifluoromethoxy) cyclobutoxy] acetamide HCl salt.

2-[3-cis-(trifluoromethoxy)cyclobutoxy]-N-[3-[[2-[3-cis-(trifluoromethoxy)cyclobutoxy]acetyl]amino]-1-bicyclo[1.1.1]pentanyl]acetamide: To a mixture of N-(1-amino-3-bicyclo[1.1.1]pentanyl)-2-[3-cis-(trifluoromethoxy)cyclobutoxy]acetamide HCl salt (30 mg, 0.090 mmol) and 2-[3-cis-(trifluoromethoxy)cyclobutoxy]acetic acid (19.4 mg, 0.090 mmol) in DMF (5.0 mL) were added HATU (51.7 mg, 0.136 mmol) and DIEA (58.6 mg, 0.453 mmol) and the mixture was stirred at 20° C. for 12 h. The mixture was poured into ice-water (15 mL) and EtOAc (10 mL). The layers were separated and the aqueous phase was extracted with EtOAc (3×5 mL). The combined organic phases were washed with brine (5 mL), dried with anhydrous Na₂SO₄, filtered and concentrated under reduced pressure. The residue was purified by prep HPLC (neutral) to provide 2-[3-cis-(trifluoromethoxy) cyclobutoxy]-N-[3-[[2-[3-cis-(trifluoromethoxy) cyclobutoxy] acetyl] amino]-1-bicyclo [1.1.1] pentanyl] acetamide. ¹H-NMR (400 MHz, CDCl₃): δ 6.83 (s, 2H), 4.23-4.43 (m, 2H), 3.81 (s, 4H), 3.71 (t, J=6.90 Hz, 2H), 2.82 (dtd, J=9.93, 6.64, 6.64, 3.14 Hz, 4H), 2.48 (s, 6H), 2.21-2.38 (m, 4H). LC-MS m/z: =491.4 [M+H]⁺.

### Example 40

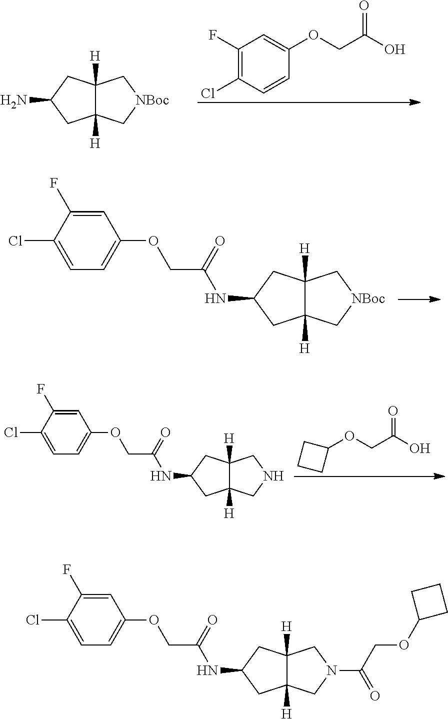

(3aR,5s,6aS)-tert-butyl 5-(2-(4-chloro-3-fluorophenoxy)acetamido)hexahydrocyclopenta[c]pyrrole-2(1H)-carboxylate: To a solution of tert-butyl (3aS,6aR)-5-amino-3,3a,4,5,6,6a-hexahydro-1H-cyclopenta[c]pyrrole-2-carboxylate, HCl salt (50 mg, 0.190 mmol) in DMF (1 mL) was added 2-(4-chloro-3-fluoro-phenoxy)acetic acid (42 mg, 0.209 mmol), HATU (79 mg, 0.209 mmol) and Na₂CO₃ (60 mg, 0.571 mmol), and the mixture was stirred at 25° C. for 3 h. The reaction mixture was diluted with H₂O (30 mL) and EtOAc (10 mL). The layers were separated and the aqueous layer was extracted with EtOAc (3×10 mL). The combined organic layers were washed with H₂O (10 mL) and brine (10 ml), dried over Na₂SO₄, filtered and concentrated under reduced pressure to provide (3aR,5s,6aS)-tert-butyl 5-(2-(4-chloro-3-fluorophenoxy)acetamido)hexahydrocyclopenta[c]pyrrole-2(1H)-carboxylate. ¹H-NMR (400 MHz, CDCl₃): δ 8.02 (s, 1H), 7.33 (t, J=8.7 Hz, 1H), 6.76 (dd, J=2.9, 10.4 Hz, 1H), 6.67 (ddd, J=1.1, 2.9, 8.8 Hz, 1H), 4.53-4.46 (m, 1H), 4.43 (s, 2H), 3.57 (br s, 2H), 3.15 (br d, J=13.2 Hz, 2H), 2.80-2.73 (m, 2H), 2.00-1.93 (m, 2H), 1.75 (td, J=7.2, 13.9 Hz, 2H), 1.46 (s, 9H).

2-(4-chloro-3-fluorophenoxy)-N-((3aR,5s,6aS)-octahydrocyclopenta[c]pyrrol-5-yl)acetamide: A mixture of (3aR,5s,6aS)-tert-butyl 5-(2-(4-chloro-3-fluorophenoxy)acetamido)hexahydrocyclopenta[c]pyrrole-2(1H)-carboxylate (100 mg, 0.24 mmol) in HCl/EtOAc (4 M, 5 mL) was stirred at 25° C. for 2 h. The mixture was concentrated under reduced pressure to provide (3aR,5s,6aS)-tert-butyl 2-(4-chloro-3-fluorophenoxy)-N-((3aR,5s,6aS)-octahydrocyclopenta[c]pyrrol-5-yl)acetamide, HCl salt. ¹H-NMR (400 MHz, MeOD): δ 7.39 (t, J=8.7 Hz, 1H), 6.94 (dd, J=2.8, 11.0 Hz, 1H), 6.83 (td, J=1.3, 9.0 Hz, 1H), 4.53 (s, 2H), 4.39 (quin, J=7.6 Hz, 1H), 3.53 (br d, J=3.8 Hz, 2H), 3.03-2.96 (m, 4H), 1.91-1.84 (m, 4H). LC-MS m/z: =313.2, 315.2 [M+H]⁺.

2-(4-chloro-3-fluoro-phenoxy)-N-[(3aS,6aR)-2-[2-(cyclobutoxy)acetyl]-3,3a,4,5,6,6a-hexahydro-1H-cyclopenta[c]pyrrol-5-yl]acetamide: To a mixture of 2-(4-chloro-3-fluorophenoxy)-N-((3aR,5s,6aS)-octahydrocyclopenta[c]pyrrol-5-yl)acetamide, HCl salt (0.07 g, 0.20 mmol) in DMF (2 mL) was added 2-(cyclobutoxy)acetic acid (26 mg, 0.20 mmol), HATU (83 mg, 0.22 mmol) and Na₂CO₃ (63 mg, 0.60 mmol) and the mixture was stirred at 20° C. for 2 h. To the reaction mixture was added H₂O (30 mL) and EtOAc (10 mL) The layers were separated and the aqueous layer was extracted with EtOAc (3×10 mL). The combined organic layers were washed with H₂O (10 mL) and brine (10 ml), dried over anhydrous Na₂SO₄, filtered and concentrated under reduced pressure to provide a residue, which was purified by prep-HPLC (TFA) to provide 2-(4-chloro-3-fluorophenoxy)-N-((3aR,5s,6aS)-2-(2-cyclobutoxyacetyl)octahydrocyclopenta[c]pyrrol-5-yl)acetamide. ¹H-NMR (400 MHz, CDCl₃): δ 7.33 (t, J=8.6 Hz, 1H), 6.75 (dd, J=2.8, 10.3 Hz, 1H), 6.67 (td, J=1.3, 8.8 Hz, 1H), 6.41 (br d, J=7.2 Hz, 1H), 4.52-4.45 (m, 1H), 4.43 (s, 2H), 4.07-3.98 (m, 1H), 3.97 (s, 2H), 3.80-3.66 (m, 2H), 3.42-3.27 (m, 2H), 2.94-2.72 (m, 2H), 2.29-2.16 (m, 2H), 2.04-1.89 (m, 4H), 1.80 (td, J=6.8, 13.4 Hz, 2H), 1.70 (q, J=10.0 Hz, 1H), 1.57-1.40 (m, 1H). LC-MS m/z: =425.4, 427.4 [M+H]⁺.

### Example 41

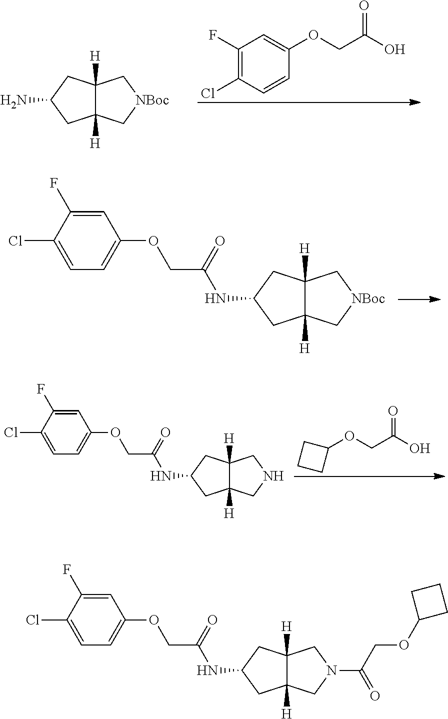

(3aR,5r,6aS)-tert-butyl 5-(2-(4-chloro-3-fluorophenoxy)acetamido)hexahydrocyclopenta[c]pyrrole-2(1H)-carboxylate: To a solution of tert-butyl (3aS,6aR)-5-amino-3,3a,4,5,6,6a-hexahydro-1H-cyclopenta[c]pyrrole-2-carboxylate (30 mg, 0.13 mmol) in DMF (1 mL) was added 2-(4-chloro-3-fluoro-phenoxy)acetic acid (27 mg, 0.13 mmol), Na₂CO₃ (28 mg, 0.27 mmol) and HATU (56 mg, 0.15 mmol) and the mixture was stirred at 20° C. for 2 h. The reaction mixture was diluted with water (5 mL) and extracted EtOAc (10 mL). The layers were separated and the aqueous layer was extracted with EtOAc (3×5 mL). The combined organic phases were washed with brine (3 mL), dried over anhydrous Na₂SO₄, filtered and concentrated under reduced pressure to provide (3aR,5r,6aS)-tert-butyl 5-(2-(4-chloro-3-fluorophenoxy)acetamido)hexahydrocyclopenta[c]pyrrole-2(1H)-carboxylate, which was used in the next step without further purification. ¹H-NMR (400 MHz, MeOD): δ ppm 7.32 (d, J=8.5 Hz, 1H), 6.76 (dd, J=2.8, 10.2 Hz, 1H), 6.68 (br d, J=8.9 Hz, 1H), 4.50 (br d, J=7.0 Hz, 1H), 4.43 (s, 2H), 3.56 (br s, 2H), 3.28-3.16 (m, 2H), 1.96 (br s, 4H), 1.83-1.76 (m, 2H), 1.46 (s, 9H).

2-(4-chloro-3-fluorophenoxy)-N-((3aR,5r,6aS)-octahydrocyclopenta[c]pyrrol-5-yl)acetamide: To a mixture of tert-butyl (3aR,5r,6aS)-tert-butyl 5-(2-(4-chloro-3-fluorophenoxy)acetamido)hexahydrocyclopenta[c]pyrrole-2(1H)-carboxylate (60 mg, 0.15 mmol) in EtOAc (5 mL) was added HCl/EtOAc (5.5 mL, 4 M), and the mixture was stirred at 25° C. for 2 h. The mixture was concentrated under reduced pressure and used directly. LC-MS: m/z=313.1, 315.1 [M+H]⁺.

2-(4-chloro-3-fluoro-phenoxy)-N-[(3aS,6aR)-2-[2-(cyclobutoxy)acetyl]-3,3a,4,5,6,6a-hexahydro-1H-cyclopenta[c]pyrrol-5-yl]acetamide: To a solution of 2-(4-chloro-3-fluorophenoxy)-N-((3aR,5r,6aS)-octahydrocyclopenta[c]pyrrol-5-yl)acetamide (45 mg, 0.14 mmol) in DMF (2 mL) was added 2-(cyclobutoxy)acetic acid (19 mg, 014 mmol), Na₂CO₃ (31 mg, 0.29 mmol) and HATU (60 mg, 0.16 mmol), and the mixture was stirred at 20° C. for 2 h. The reaction mixture was filtered through a pad of celite to provide a residue, which was purified by prep-HPLC (TFA) to provide 2-(4-chloro-3-fluorophenoxy)-N-((3aR,5r,6aS)-2-(2-cyclobutoxyacetyl)octahydrocyclopenta[c]pyrrol-5-yl)acetamide. ¹H-NMR (400 MHz, CDCl₃): δ 7.34 (t, J=8.6 Hz, 1H), 6.76 (dd, J=2.9, 10.1 Hz, 1H), 6.67 (ddd, J=1.1, 2.8, 8.9 Hz, 1H), 6.37 (br d, J=6.8 Hz, 1H), 4.53-4.45 (m, 1H), 4.44 (s, 2H), 4.07-3.99 (m, 1H), 3.97 (s, 2H), 3.79-3.68 (m, 2H), 3.41-3.28 (m, 2H), 2.95-2.76 (m, 2H), 2.29-2.16 (m, 2H), 2.05-1.87 (m, 4H), 1.86-1.76 (m, 2H), 1.74-1.66 (m, 1H), 1.55-1.44 (m, 1H). LC-MS: m/z=425.4 [M+H]⁺.

### Example 42

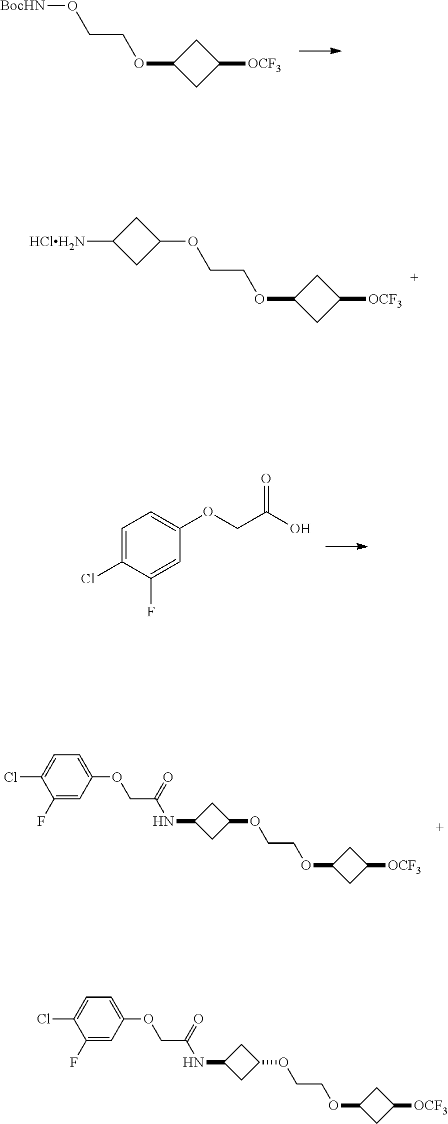

3-(2-(3-cis-(trifluoromethoxy)cyclobutoxy)ethoxy)cyclobutanamine: To a solution of tert-butyl N-[3-[2-[3-cis-(trifluoromethoxy)cyclobutoxy]ethoxy]cyclobutyl]carbamate (300 mg, 0.81 mmol) in EtOAc (5 mL) was added HCl/EtOAc (4 M, 2.03 mL) at 0° C. The reaction mixture was stirred at 25° C. for 2 h. The reaction mixture was concentrated under reduced pressure. 2-(4-chloro-3-fluoro-phenoxy)-N-[3-[2-[3-cis-(trifluoromethoxy)cyclobutoxy]ethoxy]-cis-cyclobutyl]acetamide and 2-(4-chloro-3-fluoro-phenoxy)-N-[3-[2-[3-cis-(trifluoromethoxy)cyclobutoxy]ethoxy]-trans-cyclobutyl]acetamide: To a solution of 2-(4-chloro-3-fluoro-phenoxy) acetic acid (128 mg, 0.63 mmol) in DMF (3.0 mL) was added HATU (239 mg, 0.63 mmol) at 0° C. under N₂. The reaction mixture was stirred for 30 min at 0° C. followed by the addition of 3-(2-(3-cis-(trifluoromethoxy)cyclobutoxy)ethoxy)cyclobutanamine hydrochloride (160 mg, 0.52 mmol) and DIPEA (250 mg, 1.94 mmol) at 0° C. The reaction mixture was stirred at 25° C. for 5.5 h. The reaction mixture was quenched by the addition of H₂O (16 mL) at 0° C. and then extracted with EtOAc (3×9 mL). The combined organic layers were washed with H₂O (3×5 mL) and brine (3×3 mL), dried over Na₂SO₄, filtered, and concentrated under reduced pressure. The residue was purified sequentially by preparative HPLC and SFC.

Isomer 1, peak 1 in SFC: LC-MS m/z: =4563, 458.3 [M+H]⁺. ¹H-NMR (400 MHz, CDCl₃): δ 7.34 (t, J=8.60 Hz, 1H), 6.76 (dd, J=10.29, 2.89 Hz, 1H), 6.68 (ddd, J=8.88, 2.85, 1.19 Hz, 1H), 6.56 (br d, J=7.78 Hz, 1H), 4.48-4.37 (m, 2H), 4.30 (quin, J=7.28 Hz, 1H), 4.20-4.08 (m, 1H), 3.78 (quin, J=6.96 Hz, 1H), 3.67 (quin, J=6.96 Hz, 1H), 3.54-3.45 (m, 1H), 3.54-3.45 (m, 3H), 2.84-2.73 (m, 4H), 2.30-2.20 (m, 2H), 1.98-1.85 (m, 2H).

Isomer 2, peak 2 in SFC: LC-MS: m/z=456.3, 458.3 [M+H]⁺. 1H-NMR (400 MHz, CDCl3): δ 7.34 (t, J=8.60 Hz, 1H), 6.77 (dd, J=10.29, 2.89 Hz, 1H), 6.69 (ddd, J=8.91, 2.82, 1.19 Hz, 1H), 6.58 (br d, J=6.40 Hz, 1H), 4.57-4.47 (m, 1H), 4.43 (s, 2H), 4.30 (quin, J=7.28 Hz, 1H), 4.21-4.11 (m, 1H), 3.68 (quin, J=6.96 Hz, 1H), 3.49 (dtt, J=7.65, 5.16, 5.16, 2.56, 2.56 Hz, 4H), 2.78 (dtd, J=9.74, 6.61, 6.61, 3.26 Hz, 2H), 2.55-2.43 (m, 2H), 2.30-2.18 (m, 4H).

### Example 43

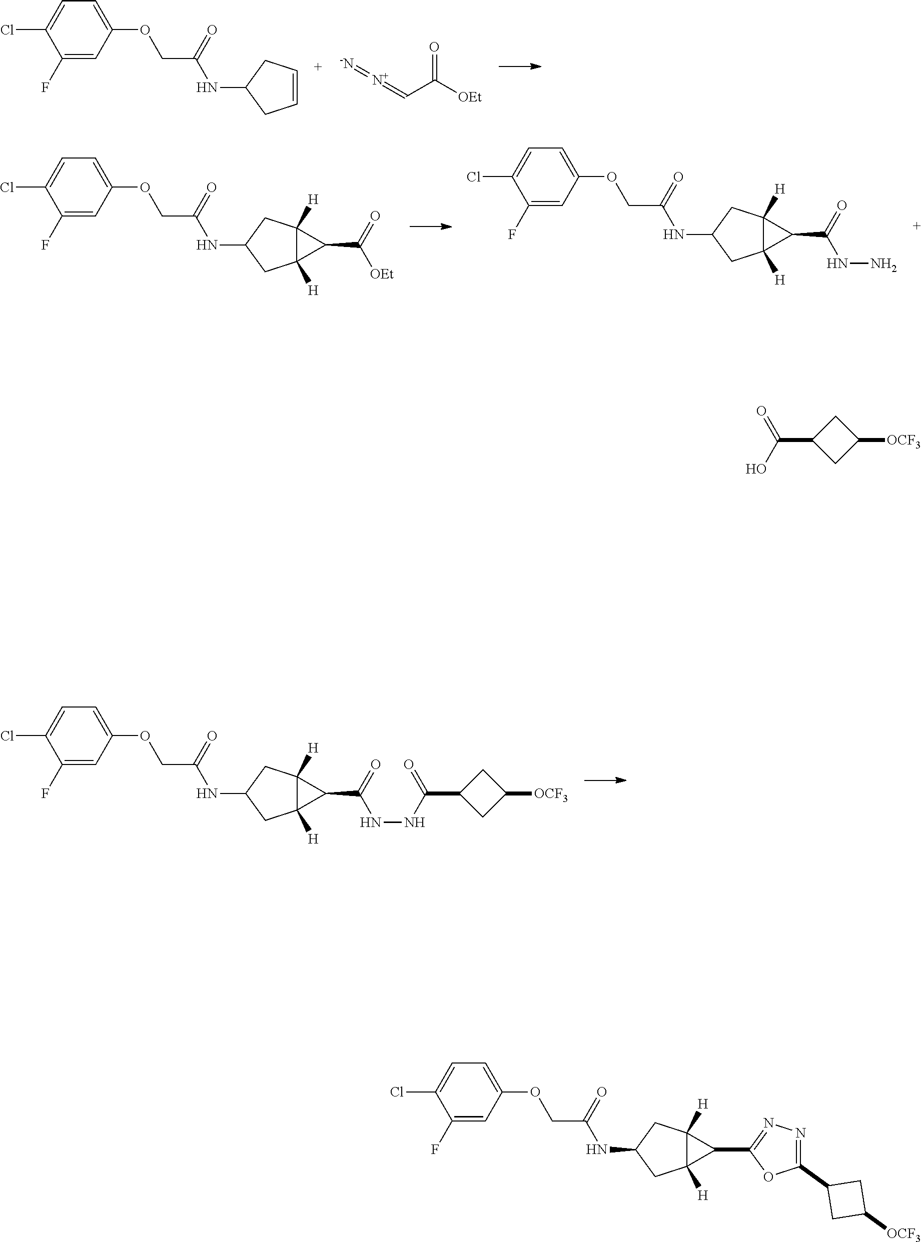

2-(4-chloro-3-fluorophenoxy)-N-(cyclopent-3-en-1-yl)acetamide: To a solution of 2-(4-chloro-3-fluorophenoxy)acetic acid (10.0 g, 48.9 mmol) in DMF (50 mL) was added HATU (18.6 g, 48.9 mmol) at 0° C. After addition, the mixture was stirred at 0° C. for 30 min, and then cyclopent-3-en-1-amine hydrochloride (4.9 g, 40.7 mmol) and DIPEA (19.5 g, 150.7 mmol) was added dropwise at 0° C. The reaction mixture was stirred at 25° C. for 3 h. The reaction mixture was quenched by pouring onto ice-water (200 mL) and stirred for 30 min at 25° C. The product was separated by filtration and the filter cake was dried under reduced pressure. The crude residue was purified by silica gel column chromatography. LC-MS: m/z=270.1, 272.1 [M+H]⁺.

syn-syn-ethyl 3-(2-(4-chloro-3-fluorophenoxy)acetamido)bicyclo[3.1.0]hexane-6-carboxylate & anti-syn-ethyl 3-(2-(4-chloro-3-fluorophenoxy)acetamido)bicyclo[3.1.0]hexane-6-carboxylate: To a solution of 2-(4-chloro-3-fluorophenoxy)-N-cyclopent-3-en-1-yl-acetamide (3.0 g, 11.1 mmol) and Rh2(OAc)4 (1.2 g, 2.8 mmol) in DCM (10 mL) was added a solution of ethyl 2-diazoacetate (5.1 g, 44.5 mmol) in DCM (40 mL) at 25° C. under N2 over 12 h. The reaction mixture was stirred at 25° C. for an additional 1 h. The reaction mixture was concentrated under reduced pressure. The crude residue was purified by silica gel column chromatography. LC-MS: m/z=356.1, 358.1 [M+H]⁺.

syn-syn-2-(4-chloro-3-fluorophenoxy)-N-6-(hydrazinecarbonyl)bicyclo[3.1.0]hexan-3-yl)acetamide & anti-syn-2-(4-chloro-3-fluorophenoxy)-N-6-(hydrazinecarbonyl)bicyclo[3.1.0]hexan-3-yl)acetamide: To a solution of ethyl 3-(2-(4-chloro-3-fluorophenoxy)acetamido)bicyclo[3.1.0]hexane-6-carboxylate (0.7 g, 2.0 mmol, mixture of isomers) in EtOH (10 ML) was added NH2NH2·H₂O (1.0 g, 19.7 mmol, 98% purity) at 25° C. The reaction mixture was stirred at 80° C. for 12 h. The reaction mixture was concentrated under reduced pressure. The crude residue was purified by silica gel column chromatography. LC-MS: m/z=342.3, 344.3 [M+H]+.

syn-syn-2-(4-chloro-3-fluorophenoxy)-N-6-(2-(3-cis-(trifluoromethoxy)cyclobutanecarbonyl)hydrazinecarbonyl)bicyclo[3.1.0]hexan-3-yl)acetamide & anti-syn-2-(4-chloro-3-fluorophenoxy)-N-6-(2-(3-cis-(trifluoromethoxy)cyclobutanecarbonyl)hydrazinecarbonyl)bicyclo[3.1.0]hexan-3-yl)acetamide: Prepared using General Procedure A employing 2-(4-chloro-3-fluorophenoxy)-N-6-(hydrazinecarbonyl)bicyclo[3.1.0]hexan-3-yl)acetamide (100 mg, 0.29 mmol, mixture of isomers) and 3-cis-(trifluoromethoxy)cyclobutanecarboxylic acid (54 mg, 0.29 mmol) and TEA (118 mg, 1.2 mmol) in DMF (4 mL). The crude residue was purified by silica gel column chromatography. LC-MS: m/z=508.1, 510.2 [M+H]+.

2-(4-chloro-3-fluoro-phenoxy)-N-[(1R,5S)-6-[5-[3-(trifluoromethoxy)cyclobutyl]-1,3,4-oxadiazol-2-yl]-3-bicyclo[3.1.0]hexanyl]acetamide: To a solution of 2-(4-chloro-3-fluorophenoxy)-N-6-(2-(3-cis-(trifluoromethoxy)cyclobutanecarbonyl)hydrazinecarbonyl)bicyclo[3.1.0]hexan-3-yl)acetamide (90 mg, 0.18 mmol, mixture of isomers) in CH₃CN (3 mL) was added p-TsCl (68 mg, 0.35 mmol) and DIPEA (115 mg, 0.89 mmol). The reaction mixture was stirred at 80° C. for 12 h. The reaction mixture was concentrated under reduced pressure. The crude residue was suspended in sat. NaHCO₃ (10 mL) and extracted with EtOAc (3×10 mL). The combined organic layers were washed with brine (5 mL), dried over Na₂SO₄, filtered, and concentrated under reduced pressure. The residue was purified by preparative HPLC. LC-MS: m/z=490.4, 492.3 [M+H]⁺. ¹H-NMR (400 MHz, MeOD): δ 7.38 (t, J=8.82 Hz, 1H), 6.94 (dd, J=11.03, 2.87 Hz, 1H), 6.82 (ddd, J=8.93, 2.87, 1.21 Hz, 1H), 4.83-4.79 (m, 1H), 4.52 (s, 2H), 4.37 (br 1, J=8.49 Hz, 1H), 3.44-3.33 (m, 1H), 2.86 (did, J=9.81, 7.33, 733, 2.87 Hz, 2H), 2.62-2.51 (m, 2H), 2.49-2.36 (m, 2H), 2.04 (d, J=3.75 Hz, 2H), 2.00 (d, J=2.21 Hz, 1H), 1.98-1.94 (m, 2H).

### Example 44

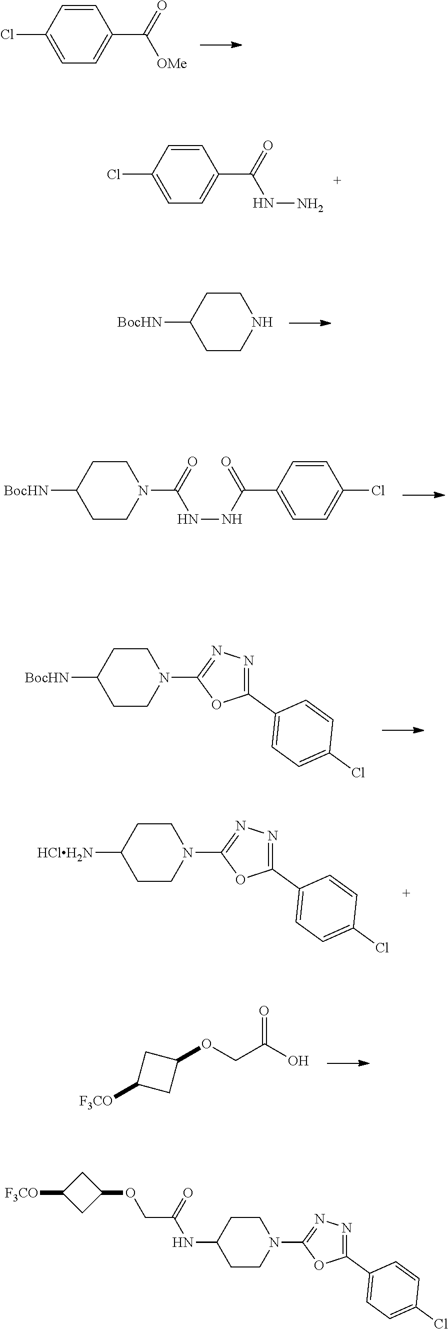

4-chlorobenzohydrazide: Prepared using General Procedure E employing methyl 4-chlorobenzoate (3.0 g, 17.6 mmol) in EtOH (50 mL).

tert-butyl (1-(2-(4-chlorobenzoyl)hydrazinecarbonyl)piperidin-4-yl)carbamate: To a solution of 4-chlorobenzohydrazide (0.50 g, 2.93 mmol) in MeCN (10 mL) was added CDI (522 mg, 3.22 mmol). The mixture was stirred at 15° C. for 5 h. To the mixture were then added tert-butyl N-(4-piperidyl)carbamate (586 mg, 2.93 mmol) and NEt3 (326 mg, 3.22 mmol). The resultant reaction mixture was stirred at 15° C. for 15 h and then 100° C. for 15 h. The reaction mixture was quenched by the addition of H₂O (60 mL) and extracted with EtOAc (3×20 mL). The combined organic layers were washed with brine (30 mL), dried over Na2SO4, filtered, and concentrated under reduced pressure. The crude residue was purified by silica gel column chromatography. LC-MS: m/z=297.1, 299.1 [M+H+.

tert-butyl (1-(5-(4-chlorophenyl)-1,3,4-oxadiazol-2-yl)piperidin-4-yl)carbamate: tert-Butyl N-[1-[[(4-chlorobenzoyl)amino]carbamoyl]-4-piperidyl]carbamate (0.50 g, 1.26 mmol), tosyl chloride (600 mg, 3.15 mmol) and DIPEA (814 mg, 6.30 mmol) were dissolved in MeCN (10 mL). The reaction mixture was stirred at 15° C. for 15 h. The reaction mixture was quenched by the addition of sat. NaHCO₃ (30 mL) and extracted with EtOAc (3×10 mL). The combined organic layers were washed with brine (20 mL), dried over Na₂SO₄, filtered, and concentrated under reduced pressure. The crude residue was purified by preparative TLC. LCMS: m/z=379.2, 381.2 [M+H]⁺.

1-(5-(4-chlorophenyl)-1,3,4-oxadiazol-2-yl)piperidin-4-amine: tert-Butyl (1-(5-(4-chlorophenyl)-1,3,4-oxadiazol-2-yl)piperidin-4-yl)carbamate (0.1 g, 0.263 mmol) was dissolved in HCl/EtOAc (4 M, 5.00 mL). The reaction mixture was stirred at 15° C. for 1 h. The reaction mixture was then concentrated under reduced pressure. LC-MS: m/z=279.1, 281.1 [M+H]+.

N-[1-[5-(4-chlorophenyl)-1,3,4-oxadiazol-2-yl]-4-piperidyl]-2-[3-cis-(trifluoromethoxy)cyclobutoxy]acetamide: To a mixture of 2-[3-cis-(trifluoromethoxy)cyclobutoxy]acetic acid (59 mg, 0.28 mmol) in DMF (5.0 mL) was added HATU (106 mg, 0.28 mmol), DIEA (98 mg, 0.76 mmol) and 1-[5-(4-chlorophenyl)-1,3,4-oxadiazol-2-yl]piperidin-4-amine hydrochloride (80 mg, 0.25 mmol) at 0° C. The reaction mixture was stirred at 15° C. for 15 h. The reaction mixture was quenched by the addition of H₂O (30 mL) and extracted with EtOAc (3×10 mL). The combined organic layers were washed with brine (10 mL), dried over Na₂SO₄, filtered, and concentrated under reduced pressure. The crude residue was purified by preparative HPLC. LC-MS: m/z=475.3, 477.3 [M+H]⁺. ¹H-NMR (400 MHz, CDCl₃): δ 7.89-7.80 (m, 2H) 7.48-7.38 (m, 2H) 6.42 (br d, J=8.03 Hz, 1H) 4.33 (quin, J=7.12 Hz, 1H) 4.14-4.03 (m, 3H) 3.86 (s, 2H) 3.72 (quin, J=6.90 Hz, 1H) 3.32-3.18 (m, 2H) 2.82 (dtd, J=9.91, 6.68, 6.68, 3.20 Hz, 2H) 2.35-2.18 (m, 2H) 2.08 (br dd, J=12.80, 2.89 Hz, 2H) 1.62 (qd, J=12.03, 4.33 Hz, 2H).

### Example 45

The following compound can be made via similar procedures as those described throughout.

| Ex. | Compound |
|---|---|
| 45 | 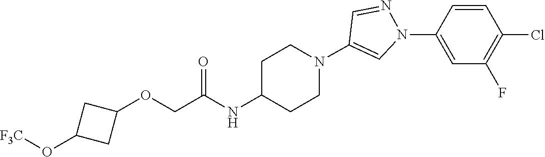 [SMILES: O=C(COC1CC(OC(F)(F)F)C1)NC1CCN(c2cnn(-c3ccc(Cl)c(F)c3)c2)CC1] |

### Example 46

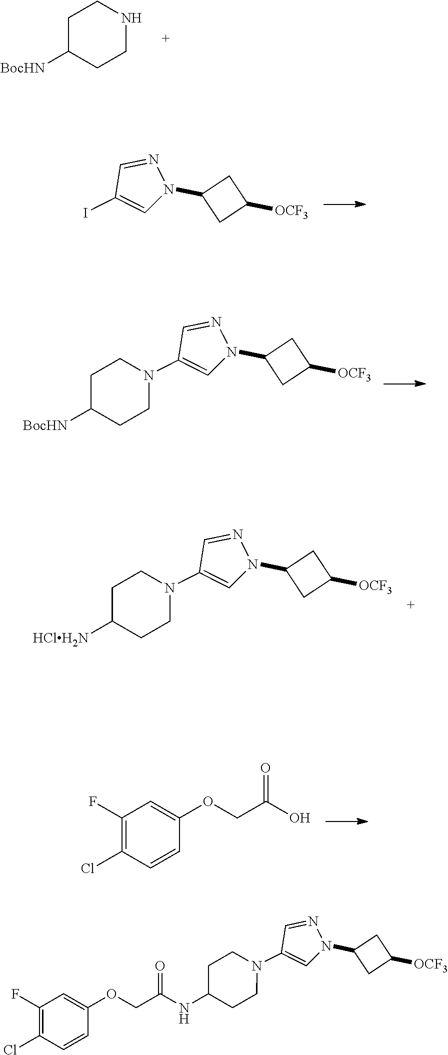

tert-butyl N-[1-[1-[3-cis-(trifluoromethoxy)cyclobutyl]pyrazol-4-yl]-4-piperidyl]carbamate: A mixture of tert-butyl N-(4-piperidyl)carbamate (995 mg, 4.97 mmol), 4-iodo-1-(3-cis-(trifluoromethoxy)cyclobutyl)-1H-pyrazole (330 mg, 0.99 mmol), copper(J) iodide (76 mg, 0.40 mmol), L-proline (92 mg, 0.80 mmol), and K₂CO₃ (687 mg, 4.97 mmol) in DMSO (3.0 mL) was degassed and purged with N₂ for 3 times. The reaction mixture was stirred at 100° C. for 15 h, was partitioned between H₂O (10 mL) and EtOAc (20 mL), and the organic phase was separated, washed with brine (3×10 mL), dried over anhydrous Na₂SO₄, filtered, and concentrated under reduced pressure. The residue was purified by silica gel column chromatography to give the desired product. LCMS: m/z=405.2 [M+H]⁺.

1-(1-(3-cis-(trifluoromethoxy)cyclobutyl)-1H-pyrazol-4-yl)piperidin-4-amine HCl salt: A solution of tert-butyl N-[1-[1-[3-cis-(trifluoromethoxy)cyclobutyl]pyrazol-4-yl]-4-piperidyl]carbamate (75 mg, 0.18 mmol) in HCl (2 mL, 1 M in EtOAc) was stirred at 20° C. for 30 min. The mixture was concentrated under reduced pressure and the residue was used directly.

2-(4-chloro-3-fluorophenoxy)-N-(1-(1-(3-cis-(trifluoromethoxy)cyclobutyl)-1H-pyrazol-4-yl)piperidin-4-yl)acetamide: To a solution of 1-(1-(3-cis-(trifluoromethoxy)cyclobutyl)-1H-pyrazol-4-yl)piperidin-4-amine HCl salt (65 mg, 0.17 mmol) and 2-(4-chloro-3-fluoro-phenoxy)acetic acid (35 mg, 0.17 mmol) in DMF (2.0 mL) were added HATU (79 mg, 0.21 mmol) and N,N-diisopropylethylamine (111 mg, 0.86 mmol), and the mixture was stirred at 20° C. for 1 h. The reaction mixture was diluted with H₂O (1 mL). This solution was directly purified employing reverse-phase HPLC to give the desired product. LCMS: m/z=491.4 [M+H]⁺. ¹H-NMR (400 MHz, CDCl₃): δ 7.34 (t, J=8.8 Hz, 1H), 7.26 (s, 1H), 7.01 (s, 1H), 6.77 (dd, J=2.8, 10.2 Hz, 1H), 6.69 (td, J=1.6, 8.8 Hz, 1H), 6.37 (br d, J=8.4 Hz, 1H), 4.53 (quin, J=7.2 Hz, 1H), 4.46 (s, 2H), 4.39-4.29 (m, 1H), 4.06-3.94 (m, 1H), 3.36-3.23 (m, 2H), 3.02-2.92 (m, 2H), 2.87-2.77 (m, 2H), 2.72 (dt, J=2.4, 11.6 Hz, 2H), 2.03 (br d, J=12.0 Hz, 2H), 1.65 (dq, J=4.0, 11.6 Hz, 2H).

### Example 47

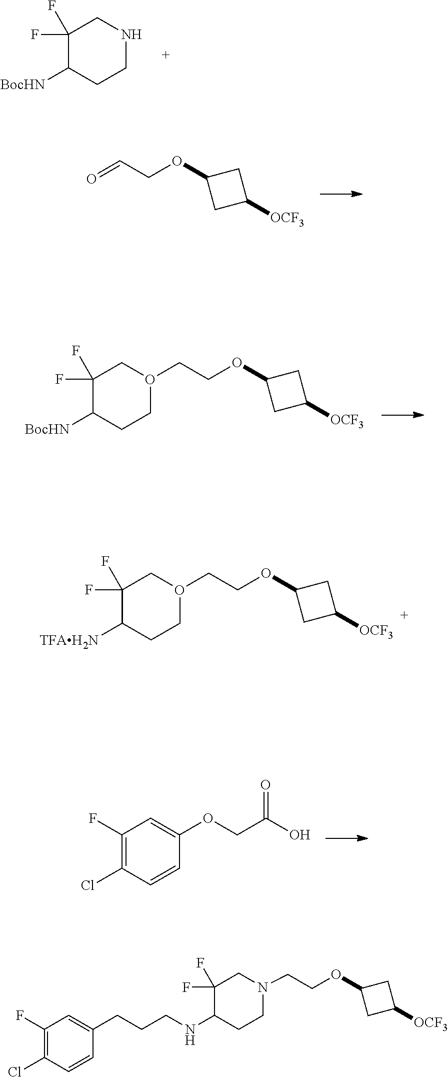

tert-butyl N-[3,3-difluoro-1-[2-[3-cis-(trifluoromethoxy)cyclobutoxy]ethyl]-4-piperidyl]carbamate: To a mixture of tert-butyl N-(3,3-difluoro-4-piperidyl)carbamate (138 mg, 0.58 mmol), 2-[3-cis-(trifluoromethoxy)cyclobutoxy]acetaldehyde (77 mg, 0.39 mmol), and acetic acid (40 μL, 0.78 mmol) in DCM (3.9 mL) was added sodium cyanoborohydride (37 mg, 0.58 mmol), and the reaction mixture was stirred at 23° C. for 16 h. The reaction mixture was diluted with aqueous 1 N NaOH (10 mL) and DCM (20 mL). The layers were separated, and the aqueous layer was extracted with DCM (3×10 mL). The combined organic layers were dried over anhydrous Na₂SO₄, filtered, and concentrated under reduced pressure. The crude material was used directly.

3,3-difluoro-1-[2-[3-cis-(trifluoromethoxy)cyclobutoxy]ethyl]piperidin-4-amine TFA salt: To a mixture of tert-butyl N-[3,3-difluoro-1-[2-[3-cis-(trifluoromethoxy)cyclobutoxy]ethyl]-4-piperidyl]carbamate (162 mg, 0.39 mmol) in DCM (3.9 mL) was added trifluoroacetic acid (0.3 mL, 3.87 mmol). The reaction mixture was stirred at 23° C. for 12 h. The volatiles were removed under reduced pressure and the crude material was used directly.

2-(4-chloro-3-fluoro-phenoxy)-N-[3,3-difluoro-1-[2-[3-cis-(trifluoromethoxy)cyclobutoxy]ethyl]-4-piperidyl]acetamide: Prepared using General Procedure A employing 3,3-difluoro-1-[2-[3-cis-(trifluoromethoxy)cyclobutoxy]ethyl]piperidin-4-amine TFA salt (168 mg, 0.39 mmol), 2-(4-chloro-3-fluoro-phenoxy)acetic acid (119 mg, 0.58 mmol), Et₃N (0.27 mL, 1.94 mmol), and T3P solution (0.69 mL, 1.17 mmol, 50% in EtOAc) in EtOAc (3.9 mL). Purified employing reverse-phase HPLC to give the desired product. LCMS: m/z=505.2 [M+H]⁺. ¹H-NMR (400 MHz, CDCl₃): δ 7.35 (t, J=8.6 Hz, 1H), 6.79 (dd, J=10.2, 2.8 Hz, 1H), 6.73-6.69 (m, 2H), 4.53 (d, J=1.7 Hz, 2H), 4.35-4.28 (m, 2H), 3.67-3.63 (m, 1H), 3.51 (t, J=5.5 Hz, 2H), 3.34-3.28 (m, 1H), 3.02 (d, J=11.6 Hz, 1H), 2.82-2.70 (m, 4H), 2.53 (dd, J=29.6, 12.5 Hz, 1H), 2.39-2.36 (m, 1H), 2.26-2.20 (m, 2H), 2.03-1.97 (m, 1H), 1.80-1.76 (m, 1H).

### Example 48

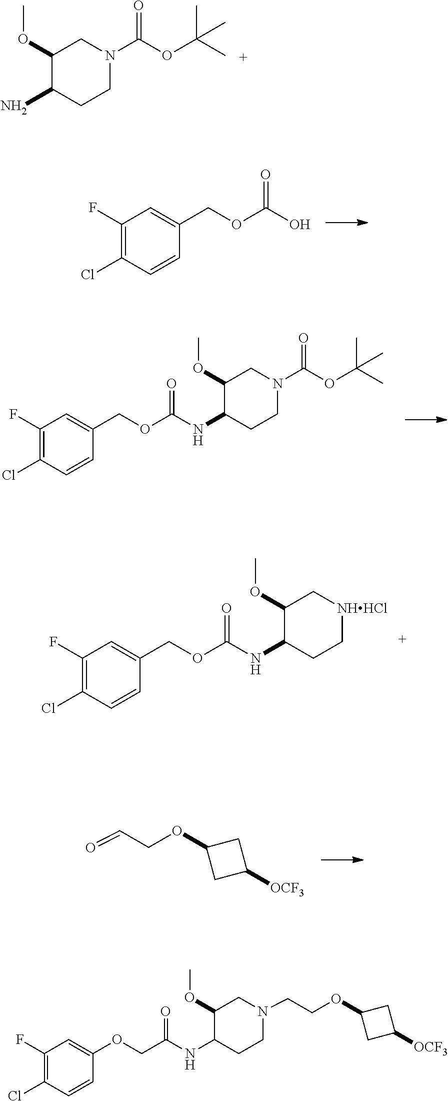

tert-butyl cis-4-[[2-(4-chloro-3-fluoro-phenoxy)acetyl]amino]-3-methoxy-piperidine-1-carboxylate: Prepared using General Procedure A employing tert-butyl cis-4-amino-3-methoxy-piperidine-1-carboxylate (1.00 g, 4.34 mmol), 2-(4-chloro-3-fluoro-phenoxy)acetic acid (1.07 g, 5.21 mmol), N,N-diisopropylethylamine (2.27 mL, 13.0 mmol), and T3P solution (1.66 g, 5.21 mmol, 50% in EtOAc) in EtOAc (8.7 mL). The crude material was used directly.

2-(4-chloro-3-fluoro-phenoxy)-N-[cis-3-methoxy-4-piperidyl]acetamide HCl salt: To tert-butyl cis-4-[[2-(4-chloro-3-fluoro-phenoxy)acetyl]amino]-3-methoxy-piperidine-1-carboxylate was added HCl (2.0 mL, 4 N in 1,4-dioxane). The reaction mixture was stirred at 23° C. for 16 h. The volatiles were removed under reduced pressure and the crude material was used directly.

2-(4-chloro-3-fluoro-phenoxy)-N-[cis-3-methoxy-1-[2-[3-cis-(trifluoromethoxy)cyclobutoxy]ethyl]-4-piperidyl]acetamide: To a mixture of 2-(4-chloro-3-fluoro-phenoxy)-N-[cis-3-methoxy-4-piperidyl]acetamide HCl salt (133 mg, 0.38 mmol), 2-[3-cis-(trifluoromethoxy)cyclobutoxy]acetaldehyde (62. mg, 0.31 mmol), and acetic acid (20 μL, 0.31 mmol) in DCM (3.1 mL) was added sodium cyanoborohydride (30 mg, 0.47 mmol), and the reaction mixture was stirred at 23° C. for 16 h. The reaction was diluted with aqueous 1 N NaOH (10 mL) and DCM (20 mL). The layers were separated, and the aqueous layer was extracted with DCM (3×10 mL). The combined organic layers were dried over anhydrous Na₂SO₄, filtered, and concentrated under reduced pressure. The crude reaction mixture was purified employing reverse-phase HPLC to give the desired product. LCMS: m/z=499.3 [M+H]⁺. ¹H-NMR (400 MHz, CDCl₃): δ 7.37-7.33 (m, 1H), 6.94-6.87 (m, 1H), 6.79-6.75 (m, 1H), 6.71 (ddd, J=8.9, 2.8, 1.2 Hz, 1H), 4.53-4.46 (m, 2H), 4.36-4.29 (m, 1H), 4.15-4.10 (m, 1H), 3.72-3.62 (m, 1H), 3.47-3.35 (m, 3H), 2.85-2.75 (m, 2H), 2.27-2.19 (m, 2H), 1.85-1.74 (m, 1H), 1.74-1.49 (m, 10H).

### Example 49

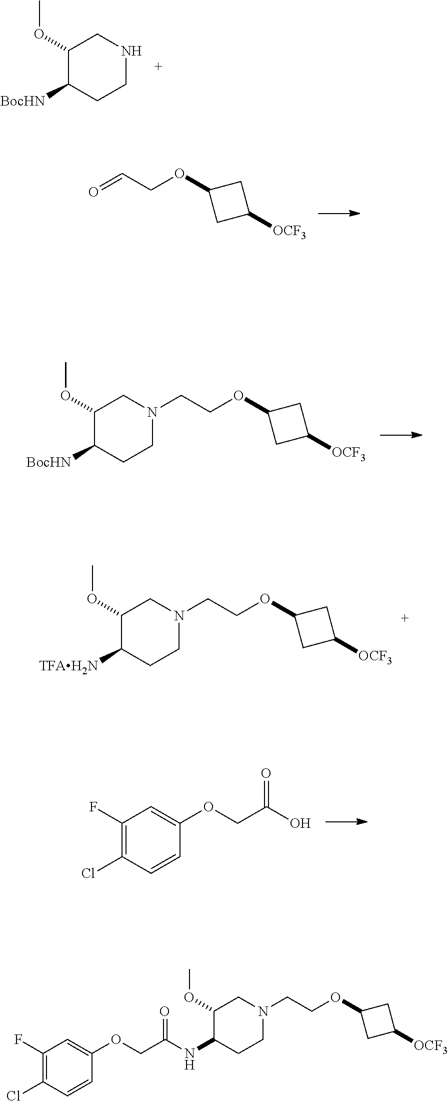

tert-butyl N-[trans-3-methoxy-1-[2-[3-cis-(trifluoromethoxy)cyclobutoxy]ethyl]-4-piperidyl]carbamate: To a mixture of tert-butyl N-[trans-3-methoxy-4-piperidyl]carbamate (167 mg, 0.73 mmol), 2-[3-cis-(trifluoromethoxy)cyclobutoxy]acetaldehyde (96 mg, 0.48 mmol), and acetic acid (30 μL, 0.48 mmol) in DCM (4.8 mL) was added sodium cyanoborohydride (46 mg, 0.73 mmol), and the reaction mixture was stirred at 23° C. for 16 h. The reaction was diluted with aqueous 1 N NaOH (10 mL) and DCM (20 mL). The layers were separated, and the aqueous layer was extracted with DCM (3×10 mL). The combined organic layers were dried over anhydrous Na₂SO₄, filtered, and concentrated under reduced pressure. The crude material was used directly.

trans-3-methoxy-1-[2-[3-cis-(trifluoromethoxy)cyclobutoxy]ethyl]piperidin-4-amine TFA salt: To a solution of tert-butyl N-[trans-3-methoxy-1-[2-[3-cis-(trifluoromethoxy)cyclobutoxy]ethyl]-4-piperidyl]carbamate (200 mg, 0.48 mmol) in DCM (2.4 mL) was added trifluoroacetic acid (0.56 mL, 7.27 mmol). The reaction mixture was stirred at 23° C. for 16 h. The volatiles were removed under reduced pressure and the crude material was used directly.

2-(4-chloro-3-fluoro-phenoxy)-N-[trans-3-methoxy-1-[2-[3-cis-(trifluoromethoxy)cyclobutoxy]ethyl]-4-piperidyl]acetamide: Prepared using General Procedure A employing trans-3-methoxy-1-[2-[3-cis-(trifluoromethoxy)cyclobutoxy]ethyl]piperidin-4-amine TFA salt (206 mg, 0.48 mmol), 2-(4-chloro-3-fluoro-phenoxy)acetic acid (148 mg, 0.72 mmol), Et₃N (0.34 mL, 2.42 mmol), and T3P solution (0.86 mL, 1.45 mmol, 50% in EtOAc) in EtOAc (2.4 mL). Purified employing reverse-phase HPLC to give the desired product. LCMS: m/z=499.3 [M+H]⁺. ¹H-NMR (400 MHz, CDCl₃): δ 7.37-7.33 (m, 1H), 6.79 (dd, J=10.3, 2.8 Hz, 1H), 6.71 (ddd, J=8.9, 2.8, 1.2 Hz, 1H), 6.42 (d, J=7.5 Hz, 1H), 4.49 (s, 2H), 4.32 (quintet, J=7.2 Hz, 1H), 3.89-3.81 (m, 1H), 3.69-3.62 (m, 1H), 3.53 (t, J=5.5 Hz, 2H), 3.37 (s, 3H), 3.35-3.29 (m, 2H), 2.96-2.93 (m, 1H), 2.79 (tdt, J=9.8, 6.6, 3.3 Hz, 2H), 2.75-2.66 (m, 2H), 2.32-2.20 (m, 3H), 2.15 (tdd, J=10.1, 5.6, 4.3 Hz, 2H), 1.66-1.61 (m, 1H).

### Example 50

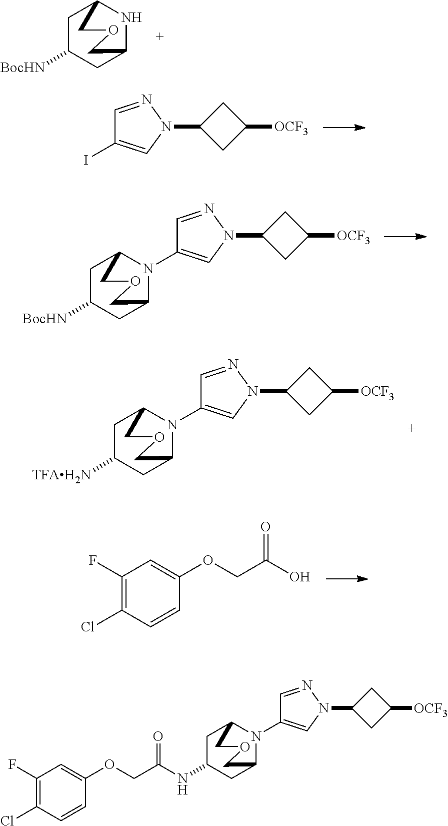

tert-butyl N-[(1R,5S,7r)-9-[1-[3-cis-(trifluoromethoxy)cyclobutyl]pyrazol-4-yl]-3-oxa-9-azabicyclo[3.3.1]nonan-7-yl]carbamate: To a solution of tert-butyl N-[(1R,5S,7r)-3-oxa-9-azabicyclo[3.3.1]nonan-7-yl]carbamate (438 mg, 1.81 mmol) in DMSO (3.0 mL) was added 4-iodo-1-(3-cis-(trifluoromethoxy)cyclobutyl)-1H-pyrazole (200 mg, 0.60 mmol), copper(I) iodide (46 mg, 0.24 mmol), and L-proline (55 mg, 0.48 mmol). The resulting reaction mixture was stirred at 100° C. for 16 h. Additional copper(I) iodide (46 mg, 0.24 mmol), and L-proline (55 mg, 0.48 mmol) were then added and the reaction was left to stir at 80° C. for an additional 72 h. The reaction mixture was quenched by the addition of sat. NaHCO₃ (5 mL) and EtOAc (10 mL). The layers were separated, and the aqueous layer was extracted with EtOAc (3×10 mL). The combined organic layers were dried over anhydrous MgSO₄, filtered, and concentrated under reduced pressure. The crude reaction mixture was purified employing reverse phase HPLC to give the desired product.

(1R,5S,7r)-9-[1-[3-cis-(trifluoromethoxy)cyclobutyl]pyrazol-4-yl]-3-oxa-9-azabicyclo[3.3.1]nonan-7-amine TFA salt: To a vial containing tert-butyl N-[(1R,5S,7r)-9-[1-[3-cis-(trifluoromethoxy)cyclobutyl]pyrazol-4-yl]-3-oxa-9-azabicyclo[3.3.1]nonan-7-yl]carbamate (25 mg, 0.06 mmol) were added DCM (0.56 mL) and trifluoroacetic acid (0.06 mL, 0.84 mmol). After stirring for 12 h at 23° C., the volatiles were removed under reduced pressure. The crude residue was used directly.

2-(4-chloro-3-fluoro-phenoxy)-N-[(1S,5R,7r)-9-[1-[3-cis-(trifluoromethoxy)cyclobutyl]pyrazol-4-yl]-3-oxa-9-azabicyclo[3.3.1]nonan-7-yl]acetamide: Prepared using General Procedure A employing (1R,5S,7r)-9-[1-[3-cis-(trifluoromethoxy)cyclobutyl]pyrazol-4-yl]-3-oxa-9-azabicyclo[3.3.1]nonan-7-amine TFA salt (25 mg, 0.05 mmol), 2-(4-chloro-3-fluoro-phenoxy)acetic acid (13 mg, 0.07 mmol), Et₃N (0.04 mL, 0.27 mmol), and T3P solution (0.10 mL, 0.16 mmol, 50% in EtOAc) in EtOAc (1.1 mL). Purified employing reverse-phase HPLC to give the desired product. LCMS: m/z=533.3 [M+H]⁺. ¹H-NMR (400 MHz, CDCl₃): δ 7.33 (t, J=8.6 Hz, 1H), 7.19 (t, J=1.6 Hz, 1H), 6.97-6.96 (m, 1H), 6.76 (dd, J=10.3, 2.8 Hz, 1H), 6.68 (ddd, J=8.9, 2.9, 1.3 Hz, 1H), 6.07-6.03 (m, 1H), 5.30-5.23 (m, 1H), 4.60-4.52 (m, 1H), 4.43-4.42 (m, 2H), 4.37 (tt, J=9.0, 7.4 Hz, 1H), 4.02-3.93 (m, 4H), 3.38-3.36 (m, 2H), 3.04-2.96 (m, 2H), 2.88-2.80 (m, 2H), 2.06-2.00 (m, 2H), 1.86-1.78 (m, 2H).

### Example 51

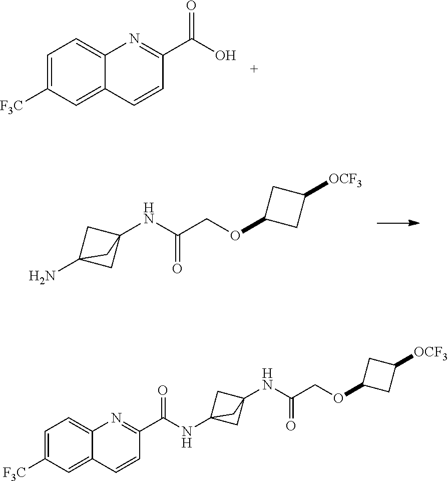

N-[1-[[2-[3-cis-(trifluoromethoxy)cyclobutoxy]acetyl]amino]-3-bicyclo[1.1.1]pentanyl]-6-(trifluoromethyl)quinoline-2-carboxamide: Prepared using General Procedure A employing N-(1-amino-3-bicyclo[1.1.1]pentanyl)-2-[3-cis-(trifluoromethoxy)cyclobutoxy]acetamide (50 mg, 0.17 mmol), 6-(trifluoromethyl)quinoline-2-carboxylic acid (61 mg, 0.25 mmol), Et₃N (0.12 mL, 0.85 mmol), and T3P solution (0.32 mL, 0.51 mmol, 50% in EtOAc) in EtOAc (1.7 mL). Purified employing reverse-phase HPLC to give the desired product. LCMS: m/z=518.4 [M+H]⁺. ¹H-NMR (400 MHz, CDCl₃): δ 8.65 (s, 1H), 8.44 (d, J=8.5 Hz, 1H), 8.40 (d, J=8.5 Hz, 1H), 8.26 (dd, J=8.9, 0.6 Hz, 1H), 8.22 (s, 1H), 7.96 (dd, J=8.9, 2.0 Hz, 1H), 6.93 (s, 1H), 4.35 (t, J=7.2 Hz, 1H), 3.86 (s, 2H), 3.75 (t, J=6.8 Hz, 1H), 2.88-2.82 (m, 2H), 2.65 (s, 6H), 2.33-2.29 (m, 2H).

### Example 52

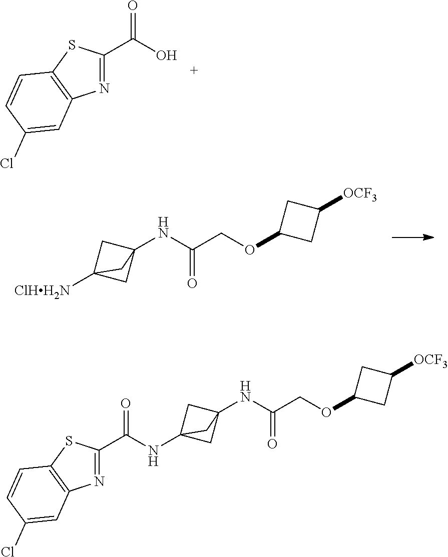

5-chloro-N-[1-[[2-[3-cis-(trifluoromethoxy)cyclobutoxy]acetyl]amino]-3-bicyclo[1.1.1]pentanyl]-1,3-benzothiazole-2-carboxamide: Prepared using General Procedure A employing N-(1-amino-3-bicyclo[1.1.1]pentanyl)-2-[3-cis-(trifluoromethoxy)cyclobutoxy]acetamide HCl salt (80 mg, 0.24 mmol), 5-Chloro-1,3-benzothiazole-2-carboxylic acid (78 mg, 0.36 mmol), Et₃N (0.17 mL, 1.21 mmol), and T3P solution (0.43 mL, 0.73 mmol, 50% in EtOAc) in EtOAc (2.4 mL). The crude reaction mixture was purified employing reverse-phase HPLC to give the desired product. LCMS: m/z=490.3 [M+H]⁺. ¹H-NMR (400 MHz, CDCl₃): δ 8.03-8.03 (m, 1H), 7.90-7.88 (m, 2H), 7.47 (dd, J=8.6, 2.0 Hz, 1H), 6.94 (s, 1H), 4.34 (t, J=7.2 Hz, 1H), 3.84 (d, J=2.5 Hz, 2H), 3.73 (t, J=6.8 Hz, 1H), 2.83 (did, J=9.9, 6.7, 3.2 Hz, 2H), 2.61 (s, 6H), 2.31-2.27 (m, 2H).

### Example 53

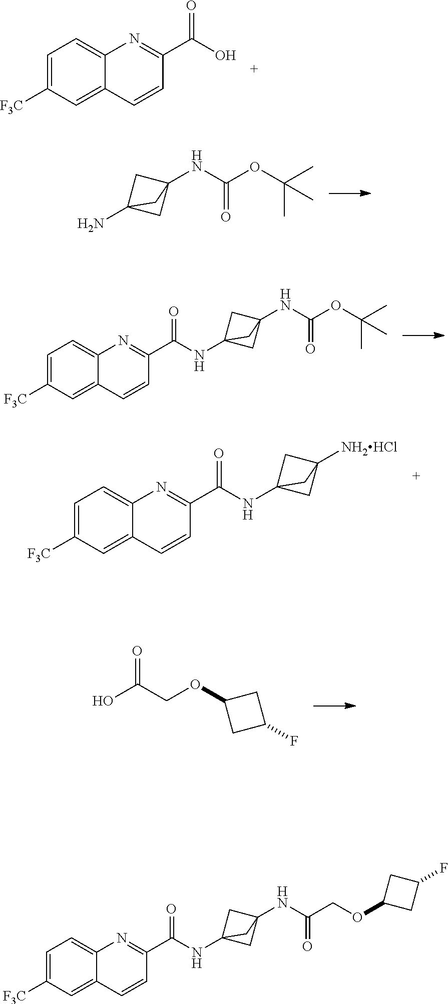

tert-butyl N-[1-[[6-(trifluoromethyl)quinoline-2-carbonyl]amino]-3-bicyclo[1.1.1]pentanyl]carbamate: Prepared using General Procedure A employing 2-methyl-2-propanyl (3-aminobicyclo[1.1.1]pent-1-yl)carbamate (99 mg, 0.50 mmol), 6-(trifluoromethyl)quinoline-2-carboxylic acid (145 mg, 0.60 mmol), Et₃N (0.35 mL, 2.50 mmol), and T3P solution (0.89 mL, 1.50 mmol, 50% in EtOAc) in EtOAc (3.0 mL). The crude reaction mixture was purified employing reverse-phase HPLC to give the desired product.

N-(1-amino-3-bicyclo[1.1.1pentanyl)-6-(trifluoromethyl)quinoline-2-carboxamide HCl salt: To a solution of tert-butyl N-[1-[[6-(trifluoromethyl)quinoline-2-carbonyl]amino]-3-bicyclo[1.1.1]pentanyl]carbamate (211 mg, 0.50 mmol) in 1,4-dioxane (0.50 mL) was added HCl (0.63 mL, 2.5 mmol, 4 N in 1,4-dioxane). The reaction mixture was stirred for 16 h at 23° C. The volatiles were then removed under reduced pressure and the crude residue was used directly.

N-[1-[[2-(3-trans-fluorocyclobutoxy)acetyl]amino]-3-bicyclo[1.1.1]pentanyl]-6-(trifluoromethyl)quinoline-2-carboxamide: General Procedure A employing N-(1-amino-3-bicyclo[1.1.1]pentanyl)-6-(trifluoromethyl)quinoline-2-carboxamide HCl salt (70 mg, 0.20 mmol), 2-(3-trans-fluorocyclobutoxy)acetic acid (43 mg, 0.29 mmol), Et₃N (0.14 mL, 0.98 mmol), and T3P solution (0.35 mL, 0.59 mmol, 50% in EtOAc) in EtOAc (2.0 mL). The crude reaction mixture was purified employing reverse-phase HPLC to give the desired product. LCMS: m/z=452.6 [M+H]⁺. ¹H-NMR (400 MHz, CDCl₃): δ 8.66 (s, 1H), 8.44 (d, J=8.5 Hz, 1H), 8.40 (d, J=8.5 Hz, 1H), 8.26 (dd, J=8.9, 0.7 Hz, 1H), 8.23 (t, J=0.9 Hz, 1H), 7.96 (dd, J=8.9, 2.0 Hz, 1H), 6.91 (s, 1H), 5.26 (dtt, J=56.1, 6.5, 3.8 Hz, 1H), 4.36-4.30 (m, 1H), 3.85 (s, 2H), 2.65 (s, 6H), 2.59-2.37 (m, 4H).

### Example 54

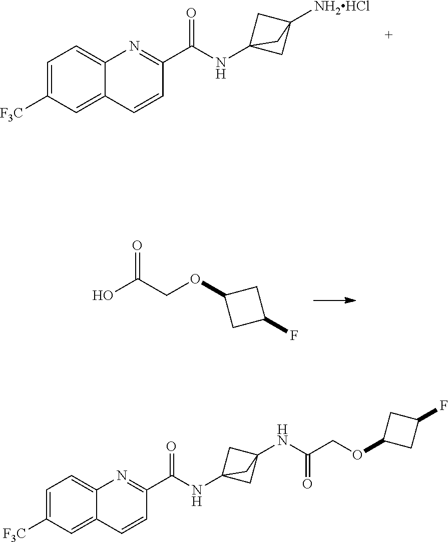

N-[1-[[2-(3-cis-fluorocyclobutoxy)acetyl]amino]-3-bicyclo[1.1.1]pentanyl]-6-(trifluoromethyl)quinoline-2-carboxamide: General Procedure A employing N-(1-amino-3-bicyclo[1.1.1]pentanyl)-6-(trifluoromethyl)quinoline-2-carboxamide HCl salt (70 mg, 0.20 mmol), 2-(3-cis-fluorocyclobutoxy)acetic acid (43 mg, 0.29 mmol), Et₃N (0.14 mL, 0.98 mmol), and T3P solution (0.35 mL, 0.59 mmol, 50% in EtOAc) in EtOAc (2.0 mL). The crude reaction mixture was purified employing reverse-phase HPLC to give the desired product. LCMS: m/z=452.7 [M+H]⁺. ¹H-NMR (400 MHz, CDCl₃): δ 8.66 (s, 1H), 8.44 (d, J=8.5 Hz, 1H), 8.40 (d, J=8.5 Hz, 1H), 8.26 (dd, J=8.9, 0.7 Hz, 1H), 8.23 (t, J=0.9 Hz, 1H), 7.96 (dd, J=9.0, 2.0 Hz, 1H), 6.95 (s, 1H), 4.75 (ddquintet, J=55.6, 6.6, 0.6 Hz, 1H), 3.86 (s, 2H), 3.70-3.62 (m, 1H), 2.89-2.79 (m, 2H), 2.65 (s, 6H), 2.36-2.22 (m, 2H).

### Example 55

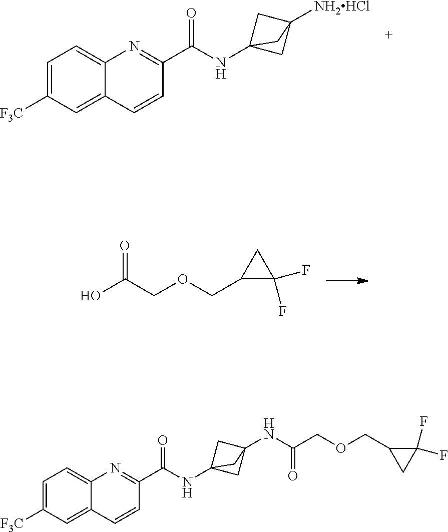

N-[1-[[2-[(2,2-difluorocyclopropyl)methoxy]acetyl]amino]-3-bicyclo[1.1.1]pentanyl]-6-(trifluoromethyl)quinoline-2-carboxamide: General Procedure A employing N-(1-amino-3-bicyclo[1.1.1]pentanyl)-6-(trifluoromethyl)quinoline-2-carboxamide HCl salt (70 mg, 0.20 mmol), 2-[(2,2-difluorocyclopropyl)methoxy]acetic acid (49 mg, 0.29 mmol), Et₃N (0.14 mL, 0.98 mmol), and T3P solution (0.35 mL, 0.59 mmol, 50% in EtOAc) in EtOAc (2.0 mL). The crude reaction mixture was purified employing reverse-phase HPLC to give the desired product. LCMS: m/z=470.7 [M+H]⁺. ¹H-NMR (400 MHz, CDCl₃): δ 8.66 (s, 1H), 8.44 (d, J=8.5 Hz, 1H), 8.40 (d, J=8.5 Hz, 1H), 8.26 (dd, J=8.9, 0.7 Hz, 1H), 8.23 (t, J=0.9 Hz, 1H), 7.96 (dd, J=9.0, 2.0 Hz, 1H), 6.99 (s, 1H), 3.97 (s, 2H), 3.76 (dddd, J=10.9, 6.3, 3.4, 1.1 Hz, 1H), 3.50 (ddd, J=10.8, 8.6, 2.0 Hz, 1H), 2.65 (s, 6H), 1.93 (ddddd, J=12.7, 11.5, 8.5, 7.4, 6.3 Hz, 1H), 1.57 (tdd, J=11.8, 7.7, 4.2 Hz, 1H), 1.19 (dtd, J=13.0, 7.7, 3.9 Hz, 1H).

### Example 56

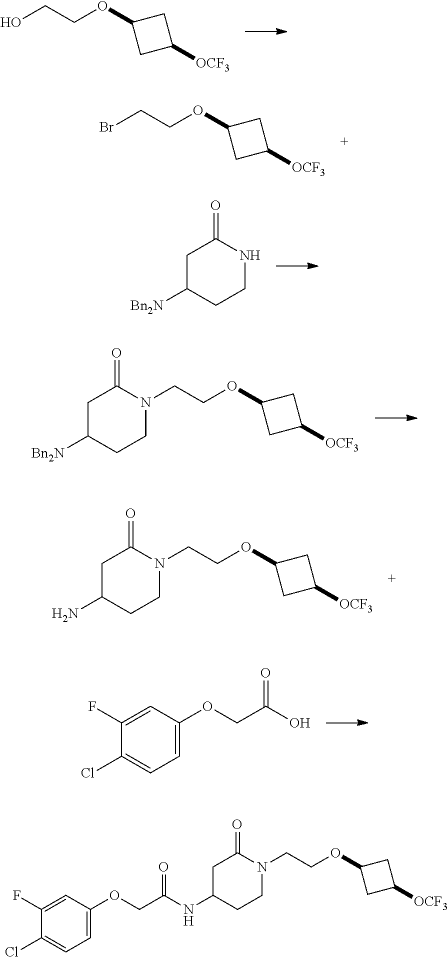

1-(2-bromoethoxy)-3-cis-(trifluoromethoxy)cyclobutane: To a solution of 2-(3-cis-(trifluoromethoxy)cyclobutoxy)ethanol (1.8 g, 8.99 mmol) in DCM (40 mL) were added PPh₃ (2.48 g, 9.44 mmol) and NBS (1.68 g, 9.44 mmol) at 0° C. The reaction mixture was stirred at 15° C. for 1 h. The reaction mixture cooled to 0° C. and was quenched by the addition of H₂O and extracted with DCM (3×40 mL). The combined organic layers were washed with brine (60 mL), dried over anhydrous Na₂SO₄, filtered, and concentrated under reduced pressure. The crude reaction mixture was purified by silica gel column chromatography to give the desired product. ¹H NMR (400 MHz, CDCl₃): δ 4.30 (m, 1H), 3.76-3.64 (m, 3H), 3.44 (t, 2H), 2.85-2.71 (m, 2H), 2.34-2.19 (m, 2H).

4-(dibenzylamino)-1-(2-(3-cis-(trifluoromethoxy)cyclobutoxy)ethyl)piperidin-2-one: Prepared using General Procedure C employing 4-(dibenzylamino)piperidin-2-one (503 mg, 1.71 mmol) and NaH (75 mg, 1.88 mmol, 60% oil immersion) and 1-(2-bromoethoxy)-3-cis-(trifluoromethoxy)cyclobutane (450 mg, 1.71 mmol) in DMF (10 mL). The crude residue was purified by silica gel column chromatography. LCMS: n/z=477.3 [M+H]⁺.

4-amino-1-(2-(3-cis-(trifluoromethoxy)cyclobutoxy)ethyl)piperidin-2-one: A mixture of 4-(dibenzylamino)-1-(2-(3-cis-(trifluoromethoxy)cyclobutoxy)ethyl)piperidin-2-one (0.30 g, 0.63 mmol), Pd(OH)₂ (44 mg, 0.63 mmol, 20 wt %) in MeOH (10 mL) was stirred under a hydrogen atmosphere at 35° C. for 15 h. The reaction mixture was then filtered and concentrated under reduced pressure. The crude residue was used directly. LCMS: m/z=297.1 [M+H]⁺. ¹H NMR (400 MHz, CDCl₃): δ 4.29 (m, 1H), 3.66-3.59 (m, 1H), 3.55-3.47 (m, 5H), 3.46-3.39 (m, 1H), 3.27 (br s, 1H), 2.83-2.71 (m, 2H), 2.69-2.58 (m, 1H), 2.24-2.10 (m, 3H), 2.05-1.92 (m, 1H), 1.70-1.60 (m, 1H).

2-(4-chloro-3-fluorophenoxy)-N-(2-oxo-1-(2-(3-cis-(trifluoromethoxy)cyclobutoxy)ethyl)piperidin-4-yl)acetamide: Prepared using General Procedure A employing 4-amino-1-(2-(3-cis-(trifluoromethoxy)cyclobutoxy)ethyl)piperidin-2-one (60 mg, 0.20 mmol), 2-(4-chloro-3-fluoro-phenoxy)acetic acid (50 mg, 0.24 mmol), Et₃N (102 mg, 1.01 mmol), and T3P solution (193 mg, 0.30 mmol, 0.18 mL, 50% in EtOAc) in EtOAc (5.0 mL). The crude reaction mixture was purified employing reverse-phase HPLC to give the desired product. LCMS: m/z=483.2 [M+H]⁺. ¹H-NMR (400 MHz, CDCl₃): δ 7.33 (t, 1H), 6.76 (m, 1H), 6.68 (m, 1H), 6.45 (m, 1H), 4.46 (s, 2H), 4.40-4.19 (m, 2H), 3.79-3.27 (m, 7H), 2.88-2.57 (m, 3H), 2.31 (m, 1H), 2.15 (m, 3H), 1.91-1.80 (m, 1H).

### Example 57

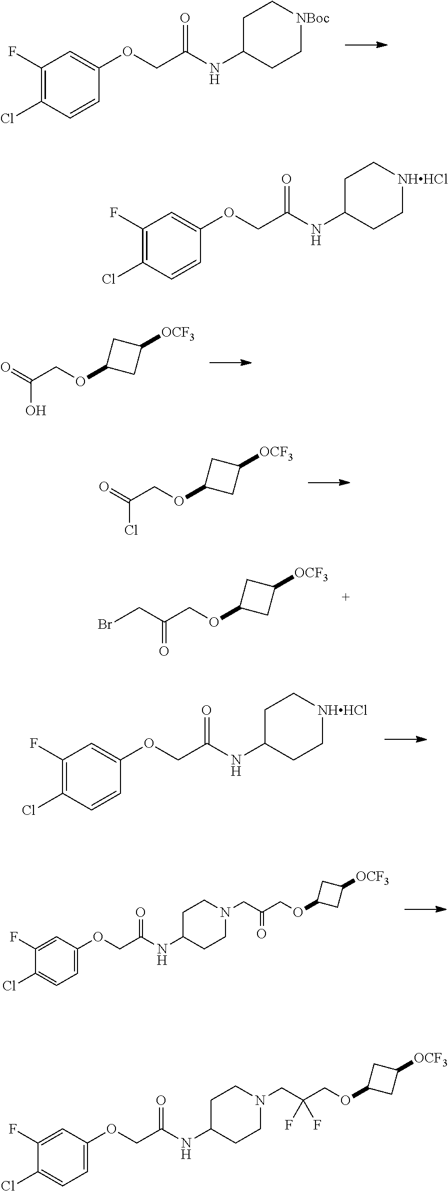

2-(4-chloro-3-fluorophenoxy)-N-(piperidin-4-yl)acetamide HCl salt: To a solution of tert-butyl 4-(2-(4-chloro-3-fluorophenoxy)acetamido)piperidine-1-carboxylate (1.0 g, 2.59 mmol) in EtOAc (10 mL) was added HCl (13 mL, 4 M in EtOAc). The reaction mixture was stirred at 20° C. for 1 h. The reaction mixture was then concentrated under reduced pressure and the crude residue was used directly.

2-(3-cis-(trifluoromethoxy)cyclobutoxy)acetyl chloride: To a solution of 2-(3-cis-(trifluoromethoxy)cyclobutoxy)acetic acid (0.5 g, 2.33 mmol) in DCM (10 mL) was added oxalyl dichloride (0.89 g, 7.00 mmol) and DMF (0.5 mL) at 0° C. The reaction mixture was stirred at 0° C. for 2 h. The reaction mixture was then concentrated under reduced pressure and the crude residue was used directly.

1-bromo-3-(3-cis-(trifluoromethoxy)cyclobutoxy)propan-2-one: To a mixture of 2-(3-cis-(trifluoromethoxy)cyclobutoxy)acetyl chloride (0.60 g, 2.58 mmol) in THF (6.0 mL) and MeCN (6.0 mL) was added TMSCHN₂ (1.29 mL, 2.58 mmol) at 0° C. The reaction mixture was stirred at 20° C. for 2 h. The reaction mixture was then cooled to 0° C. followed by the addition of HBr (1.57 g, 7.74 mmol, 40%). The reaction mixture further was stirred at 20° C. for 2 h. The reaction mixture was then cooled to 0° C., quenched by the addition of H₂O (50 mL), and extracted with EtOAc (3×30 mL). The combined organic layers were washed with brine (30 mL), dried over anhydrous Na₂SO₄, filtered, and concentrated under reduced pressure. The crude residue was purified by silica gel column chromatography to give the desired product. ¹H-NMR (400 MHz, CDCl₃): δ 4.36-4.29 (m, 1H), 4.28 (s, 1H), 4.20 (d, J=8.0 Hz, 2H), 4.03 (s, 1H), 3.73 (m, J=6.8, 10.2 Hz, 1H), 2.90-2.77 (m, 2H), 2.38-2.25 (m, 2H).

2-(4-chloro-3-fluorophenoxy)-N-(1-(2-oxo-3-(3-cis-(trifluoromethoxy)cyclobutoxy)propyl)piperidin-4-yl)acetamide: A mixture of 1-bromo-3-(3-cis-(trifluoromethoxy)cyclobutoxy)propan-2-one (122 mg, 0.42 mmol), 2-(4-chloro-3-fluorophenoxy)-N-(piperidin-4-yl)acetamide HCl salt (90 mg, 0.28 mmol) in MeCN (5.0 mL) was added Na₂CO₃ (119 mg, 1.11 mmol). The reaction mixture was stirred at 20° C. for 2 h. The reaction mixture was cooled to 0° C., quenched by addition of H₂O (5 mL), and extracted with EtOAc (3×5 mL). The combined organic layers were washed with brine (5 mL), dried over Na₂SO_{d}, filtered, and concentrated under reduced pressure. The crude residue was used directly.

LCMS: m/z=497.1 [M+H]⁺.

2-(4-chloro-3-fluorophenoxy)-N-(1-(2,2-difluoro-3-(3-cis-(trifluoromethoxy)cyclobutoxy)propyl)piperidin-4-yl)acetamide: To a mixture of 2-(4-chloro-3-fluorophenoxy)-N-(1-(2-oxo-3-(3-cis-(trifluoromethoxy)cyclobutoxy)propyl)piperidin-4-yl)acetamide (150 mg, 0.30 mmol) in anhydrous DCM (5.0 mL) at 0° C. was added DAST (243 mg, 1.51 mmol) at 0° C., and the reaction mixture was stirred at 20° C. for 12 h. The reaction mixture was cooled to 0° C., quenched by addition of H₂O (5 mL), and was extracted with DCM (3×5 mL). The combined organic layers were washed with brine (5 mL), dried over anhydrous Na₂SO₄, filtered, and concentrated under reduced pressure. The crude residue was purified by silica gel column chromatography and further purified employing reverse-phase HPLC to give the desired product. LCMS: m/z=519.1 [M+H]⁺. ¹H-NMR (400 MHz, CDCl₃): δ 7.34 (t, J=8.4 Hz, 1H), 6.76 (dd, J=2.8, 10.3 Hz, 1H), 6.68 (br d, J=9.2 Hz, 1H), 6.29 (br d, J=7.6 Hz, 1H), 4.45 (s, 2H), 4.30 (quin, J=7.2 Hz, 1H), 3.94-3.83 (m, 1H), 3.74 (quin, J=6.8 Hz, 1H), 3.62 (t, J=12.6 Hz, 2H), 2.91 (br d, J=12.4 Hz, 2H), 2.85-2.75 (m, 4H), 2.42 (br t, J=10.8 Hz, 2H), 2.32-2.23 (m, 2H), 1.92 (br d, J=12.8 Hz, 2H), 1.53-1.43 (m, 2H).

### Example 58

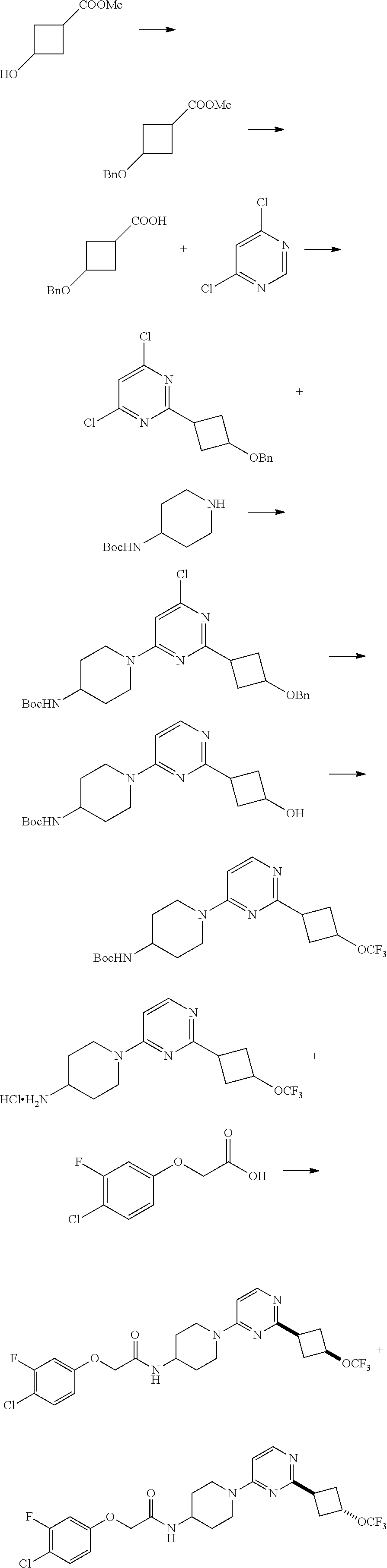

methyl 3-(benzyloxy)cyclobutanecarboxylate: Prepared using General Procedure C employing methyl 3-hydroxycyclobutanecarboxylate (20.0 g, 153.68 mmol), sodium hydride (7.38 g, 184.41 mmol, 60% oil immersion) and BnBr (27.6 g, 161.36 mmol) in anhydrous THF (100 mL). The crude residue was purified by silica gel column chromatography to give the desired product (cis:trans=3:1).

3-(benzyloxy)cyclobutanecarboxylic acid: To a mixture of methyl 3-(benzyloxy)cyclobutanecarboxylate (26.0 g, 118 mmol, cis:trans=3:1) in MeOH (300 mL) and H₂O (50 mL) was added LiOH·H₂O (19.8 g, 472 mmol) at 15° C. The reaction mixture was stirred for 16 h at 15° C. The reaction mixture was then concentrated under reduced pressure. The residue was diluted with water (200 mL) and extracted with MTBE (2×100 mL). The aqueous phase was adjusted to pH 3 using aqueous 2 M HCT and then extracted with EtOAc (2×100 mL). The combined organics were washed with brine (50 mL), dried over Na₂SO₄, filtered, and concentrated under reduced pressure to give the desired product (cis:trans=3:1) as a crude residue that was used directly.

2-(3-(benzyloxy)cyclobutyl)-4,6-dichloropyrimidine: To a solution of 4,6-dichloropyrimidine (2.0 g, 13.4 mmol) in MeCN (50 mL) were added 3-(benzyloxy)cyclobutanecarboxylic acid (8.31 g, 40.3 mmol, cis:trans=3:1) and AgNO₃ (9.12 g, 53.7 mmol). The reaction mixture was heated to 60° C. and then a solution of ammonium persulfate (15.3 g, 67.1 mmol) in H₂O (50 mL) was added dropwise. The reaction mixture was then stirred for 16 h at 60° C. The reaction mixture was concentrated under reduced pressure and the residue was partioned between EtOAc (50 mL) and H₂O (50 mL). The mixture was filtered, and the filter cake was washed with EtOAc (2×50 mL). The organics were separated, washed with water (50 mL), brine (50 mL), dried over anhydrous Na₂SO₄, filtered, and concentrated under reduced pressure. The crude residue was purified by silica gel column chromatography to give the desired product (cis:trans=3:1).

tert-butyl (1-(2-(3-(benzyloxy)cyclobutyl)-6-chloropyrimidin-4-yl)piperidin-4-yl)carbamate: To a mixture of 2-(3-benzyloxycyclobutyl)-4,6-dichloropyrimidine (700 mg, 2.26 mmol, cis:trans=3:1) and N,N-diisopropylethylamine (585 mg, 4.53 mmol) in MeCN (10 mL) at 0° C. was added tert-butyl piperidin-4-ylcarbamate (499 mg, 2.49 mmol). The reaction mixture was stirred for 16 h at 15° C. The reaction mixture was diluted with water (20 mL) and extracted with EtOAc (2×10 mL). The combined organics were washed with water (10 mL), aq. HCl (10 mL, 1 M), brine (10 mL), dried over Na₂SO₄, filtered, and concentrated under reduced pressure to give the desired product (cis:trans=3:1) as a crude residue that was used directly. LCMS: m/z=473.2 [M+H]⁺.

tert-butyl (1-(2-(3-hydroxycyclobutyl)pyrimidin-4-yl)piperidin-4-yl)carbamate: To a solution of tert-butyl (1-(2-(3-(benzyloxy)cyclobutyl)-6-chloropyrimidin-4-yl)piperidin-4-yl)carbamate (1 g, 2.11 mmol, cis:trans=3:1) in MeOH (20 mL) was added Pd (1.0 g, 10 wt % on carbon) in one portion. The reaction mixture was stirred for 2 h at 15° C. under a hydrogen atmosphere at 15 psi. The reaction mixture was filtered through a pad of celite and the filter cake was washed with MeOH (4×20 mL). The filtrate was concentrated under reduced pressure. The crude residue was purified by silica gel column chromatography to give the desired product (cis:trans=3:1). LCMS: m/z=349.2 [M+H]⁺.

tert-butyl (1-(2-(3-(trifluoromethoxy)cyclobutyl)pyrimidin-4-yl)piperidin-4-yl)carbamate: To a suspension of AgOTf (664 mg, 2.58 mmol) in anhydrous EtOAc (30 mL) at 15° C. protected from light were added SelectFluor (458 mg, 1.29 mmol), KF (200 mg, 3.44 mmol) and tert-butyl (1-(2-(3-hydroxycyclobutyl)pyrimidin-4-yl)piperidin-4-yl)carbamate (300 mg, 0.86 mmol, cis:trans=3:1). The reaction mixture was stirred for 5 min and then 2-fluoropyridine (251 mg, 2.58 mmol) and TMSCF₃ (367 mg, 2.58 mmol) were added. The reaction mixture was warmed to 25° C. and was stirred for 16 h. The reaction mixture was then filtered through a pad of celite and the filter cake was washed with EtOAc (2×20 mL). The filtrate was concentrated under reduced pressure. The crude residue was purified by silica gel column chromatography to give the desired product (cis:trans=3:1). LCMS: m/z=417.1 [M+H]⁺.

1-(2-(3-(trifluoromethoxy)cyclobutyl)pyrimidin-4-yl)piperidin-4-amine HCl salt: A mixture of ten-butyl (1-(2-(3-(trifluoromethoxy)cyclobutyl)pyrimidin-4-yl)piperidin-4-yl)carbamate (150 mg, 0.36 mmol, cis:trans=3:1) in HCl (10 mL, 4 N in EtOAc) was stirred for 1 h at 15° C. The reaction mixture was concentrated under reduced pressure to give the desired product (cis:trans=3:1) as a crude residue that was used directly. LCMS: m/z=317.1 [M+H]⁺.

2-(4-chloro-3-fluorophenoxy)-N-(1-(2-(3-cis-(trifluoromethoxy)cyclobutyl)pyrimidin-4-yl)piperidin-4-yl)acetamide and 2-(4-chloro-3-fluorophenoxy)-N-(1-(2-(3-trans-(trifluoromethoxy)cyclobutyl)pyrimidin-4-yl)piperidin-4-yl)acetamide: To a solution of 1-(2-(3-(trifluoromethoxy)cyclobutyl)pyrimidin-4-yl)piperidin-4-amine HCl salt (127 mg, 0.36 mmol, cis:trans=3:1) and 2-(4-chloro-3-fluorophenoxy)acetic acid (74 mg, 0.36 mmol) in DMF (5.0 mL) were added N,N-diisopropylethylamine (140 mg, 1.08 mmol) and HATU (205 mg, 0.54 mmol). The reaction mixture was stirred for 1 h at 15° C. The reaction mixture was diluted with water (20 mL) and extracted with EtOAc (2×10 mL). The combined organics were washed with aq. HCl (10 mL, 1M), brine (20 mL), dried over Na₂SO₄, filtered, and concentrated under reduced pressure. The crude residue was purified by preparative TLC and further purified employing reverse-phase HPLC to give:

2-(4-chloro-3-fluorophenoxy)-N-(1-(2-(3-cis-(trifluoromethoxy)cyclobutyl)pyrimidin-4-yl)piperidin-4-yl)acetamide: LCMS: m/z=503.2 [M+H]⁺. ¹H-NMR (400 MHz, CDCl₃): δ 8.15 (d, J=6.0 Hz, 1H), 7.32 (t, J=8.4 Hz, 1H), 6.74 (dd, J=2.8, 10.4 Hz, 1H), 6.68-6.64 (m, 1H), 6.42-6.35 (m, 2H), 4.70-4.62 (m, 1H), 4.58 (s, 3H), 4.46 (br s, 1H), 4.24-4.14 (m, 1H), 3.18-3.01 (m, 3H), 2.77-2.68 (m, 2H), 2.67-2.57 (m, 2H), 2.13-2.03 (m, 2H), 1.54-1.37 (m, 2H).

And 2-(4-chloro-3-fluorophenoxy)-N-(1-(2-(3-trans-(trifluoromethoxy)cyclobutyl)pyrimidin-4-yl)piperidin-4-yl)acetamide: LCMS: m/z=503.2 [M+H]⁺. ¹H-NMR (400 MHz, CDCl₃): δ 8.17 (d, J=6.0 Hz, 1H), 7.33 (t, J=8.4 Hz, 1H), 6.75 (dd, J=2.4, 10.0 Hz, 1H), 6.67 (dd, J=2.0, 8.8 Hz, 1H), 6.41-6.33 (m, 2H), 5.12-5.05 (m, 1H), 4.47 (s, 3H), 4.46-4.43 (m, 1H), 4.27-4.10 (m, 1H), 3.60-3.47 (m, 1H), 3.14-3.02 (m, 2H), 2.75-2.59 (m, 4H), 2.09-2.07 (m, 2H), 1.50-4.44 (m, 2H).

### Example 59

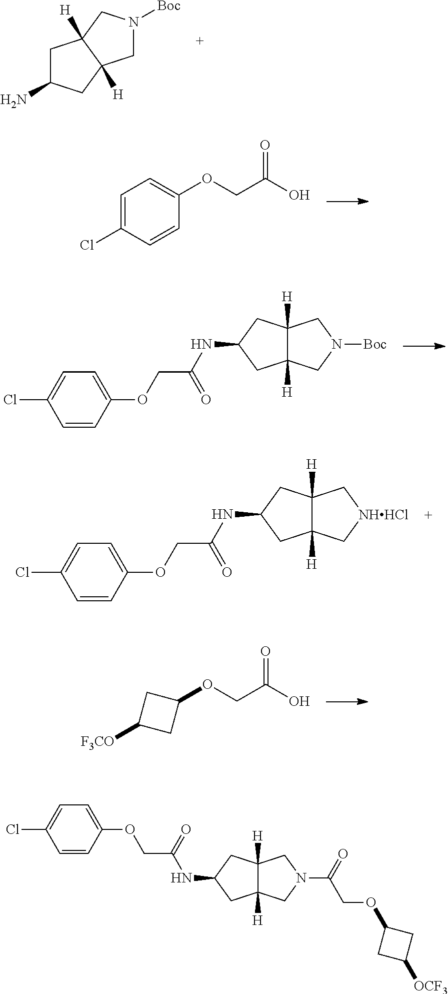

(3aR,5s,6aS)-tert-butyl 5-(2-(4-chlorophenoxy)acetamido)hexahydrocyclopenta[c]pyrrole-2(1H)-carboxylate: Prepared using General Procedure A employing (3aR,5s,6aS)-tert-butyl 5-aminohexahydrocyclopenta[c]pyrrole-2(1H)-carboxylate (100 mg, 0.38 mmol), 2-(4-chlorophenoxy)acetic acid (85 mg, 0.46 mmol), Et₃N (116 mg, 1.14 mmol), and T3P solution (969 mg, 1.52 mmol, 50% in EtOAc) in EtOAc (2.0 mL). The crude residue was purified by preparative TLC to give the desired product. LCMS: m/z=339.1 [M-55+H]⁺. ¹H-NMR (400 MHz, CDCl₃): δ 7.32-7.24 (m, 2H), 6.89-6.80 (m, 2H), 6.41 (br d, J=7.2 Hz, 1H), 4.55-4.46 (m, 1H), 4.43 (s, 2H), 3.56 (br s, 2H), 3.16 (br s, 2H), 2.77 (br s, 2H), 2.02-1.88 (m, 2H), 1.75 (br d, J=6.4 Hz, 2H), 1.46 (s, 9H).

2-(4-chlorophenoxy)-N-((3aR,5s,6aS)-octahydrocyclopenta[c]pyrrol-5-yl)acetamide HCl salt: A solution of (3aR,5s,6aS)-tert-butyl 5-(2-(4-chlorophenoxy)acetamido)hexahydrocyclopenta[c]pyrrole-2(1H)-carboxylate (150 mg, 0.38 mmol) in HCl (10 mL, 4 N in EtOAc) was stirred for 1 h at 20° C. The reaction mixture was then concentrated under reduced pressure and the crude residue was used directly. LCMS: n/z=295.1 [M+H]⁺.

2-(4-chlorophenoxy)-N-((3aR,5S,6aS)-2-(2-(3-cis-(trifluoromethoxy)cyclobutoxy)acetyl)octahydrocyclopenta[c]pyrrol-5-yl)acetamide: Prepared using General Procedure A employing 2-(4-chlorophenoxy)-N-((3aR,5s,6aS)-octahydrocyclopenta[c]pyrrol-5-yl)acetamide HCl salt (15 mg, 0.045 mmol), 2-(3-cis-(trifluoromethoxy)cyclo butoxy)acetic acid (12 mg, 0.054 mmol), T3P solution (115 mg, 0.18 mmol, 50% in EtOAc) and N,N-diisopropylethylamines (29 mg, 0.23 mmol) in EtOAc (1.0 mL). Purified by reverse-phase HPLC to give the desired product. LCMS: m/z=491.1 [M+H]⁺. ¹H-NMR (400 MHz, CDCl₃): δ 7.33-7.25 (m, 2H), 6.91-6.81 (m, 2H), 6.45 (br d, J=7.6 Hz, 1H), 4.52-4.42 (m, 3H), 4.32-4.25 (m, 1H), 4.05-3.95 (m, 2H), 3.86-3.63 (m, 3H), 3.38 (dd, J=4.8, 12.8 Hz, 1H), 3.28 (dd, J=4.8, 11.2 Hz, 1H), 2.97-2.73 (m, 4H), 2.28 (td, J=7.2, 12.8 Hz, 2H), 2.03-1.92 (m, 2H), 1.86-1.76 (m, 2H).

### Example 60

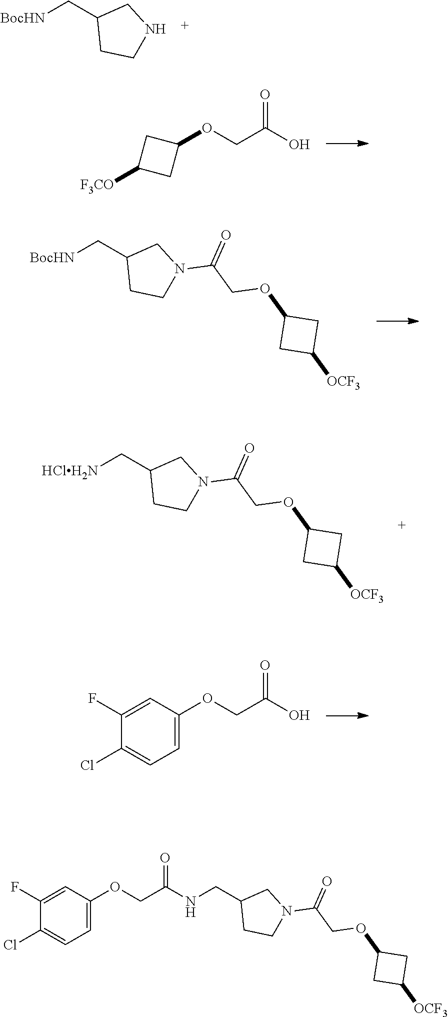

tert-butyl ((1-(2-(3-cis-(trifluoromethoxy)cyclobutoxy)acetyl)pyrrolidin-3-yl)methyl)carbamate: Prepared using General Procedure A employing tert-butyl N-(pyrrolidin-3-ylmethyl)carbamate (100 mg, 0.50 mmol), 2-(3-cis-(trifluoromethoxy)cyclobutoxy)acetic acid (118 mg, 0.55 mmol), T3P solution (953 mg, 1.50 mmol, 50% in EtOAc) and Et₃N (152 mg, 1.50 mmol) in EtOAc (5 mL). The crude residue was used directly. ¹H NMR (400 MHz, CDCl₃): δ 4.65 (br s, 1H), 4.29-4.25 (m, 1H), 4.04 (s, 2H), 3.79 (quin, J=7.0 Hz, 1H), 3.71-3.61 (m, 1H), 3.60-3.51 (m, 1H), 3.49-3.38 (m, 1H), 3.28-3.03 (m, 4H), 2.86-2.75 (m, 2H), 2.52-2.35 (m, 1H), 2.34-2.23 (m, 2H), 2.14-1.95 (m, 1H), 1.45 (s, 9H).

1-(3-(aminomethyl)pyrrolidin-1-yl)-2-(3-cis-(trifluoromethoxy)cyclobutoxy)ethenone HCl salt: A mixture of tert-butyl ((1-(2-(3-cis-(trifluoromethoxy)cyclobutoxy)acetyl)pyrrolidin-3-yl)methyl)carbamate (120 mg, 0.30 mmol) in HCl (10 mL, 4 N in EtOAc) was stirred at 20° C. for 3 h. The reaction mixture was concentrated under reduce pressure and the crude residue was used directly.

2-(4-chloro-3-fluorophenoxy)-N-((1-(2-(3-cis-(trifluoromethoxy)cyclobutoxy)acetyl)pyrrolidin-3-yl)methyl)acetamide: Prepared using General Procedure A employing 1-(3-(aminomethyl)pyrrolidin-1-yl)-2-(3-cis-(trifluoromethoxy)cyclobutoxy)ethanone HCl salt (130 mg, 0.39 mmol), T3P solution (678 mg, 1.07 mmol, 50% in EtOAc), Et₃N (108 mg, 1.07 mmol) and 2-(4-chloro-3-fluoro-phenoxy)acetic acid (73 mg, 0.36 mmol) in EtOAc (10 mL). The crude residue was purified by reverse-phase HPLC to give the desired product. LCMS: m/z=505.1 [M+Na]⁺. ¹H-NMR (400 MHz, CDCl₃): δ 7.35 (dt, J=1.9, 8.6 Hz, 1H), 6.76 (m, 1H), 6.69 (m, 1H), 6.64 (m, 1H), 4.48 (d, J=5.7 Hz, 2H), 4.28 (quin, J=7.2 Hz, 1H), 3.99 (d, J=6.6 Hz, 2H), 3.78 (quin, J=7.0 Hz, 1H), 3.72-3.64 (m, 1H), 3.62-3.42 (m, 3H), 3.26-3.15 (m, 1H), 2.86-2.76 (m, 2H), 2.60-2.42 (m, 1H), 2.34-2.22 (m, 2H), 2.15-1.98 (m, 1H), 1.76-1.70 (m, 1H), 1.62 (qd, J=8.3, 12.6 Hz, 1H).

### Example 61

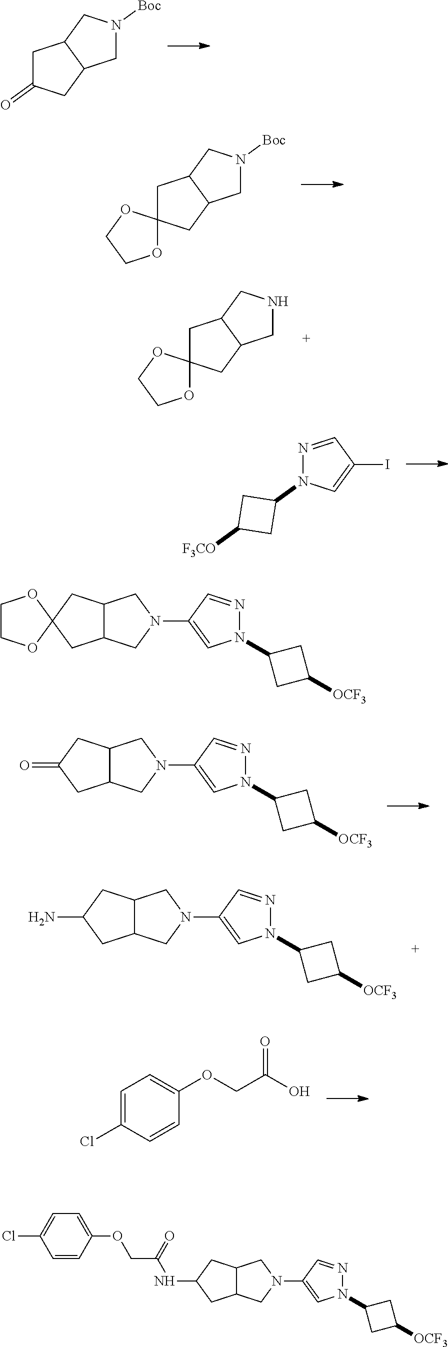

tert-butyl tetrahydro-1H-spiro[cyclopenta[c]pyrrole-5,2′-[1,3]dioxolane]-2(3H)-carboxylate: To a solution of tert-butyl 5-oxohexahydrocyclopenta[c]pyrrole-2(1H)-carboxylate (5.0 g, 22.2 mmol) and ethylene glycol (2.76 g, 44.4 mmol) in toluene (150 mL) was added p-TsOH·H₂O (422 mg, 2.22 mmol). The reaction mixture was stirred at 110° C. for 12 h. The reaction mixture was then cooled to 0° C., quenched by addition of H₂O (100 mL) at 0° C., and extracted with EtOAc (3×100 mL). The combined organic layers were washed with brine (50 mL), dried over anhydrous Na₂SO₄, filtered, and concentrated under reduced pressure. The crude residue was purified by silica gel column chromatography to give the desired product. ¹H-NMR (400 MHz, CDCl₃): δ 3.90 (s, 4H), 3.53 (br s, 2H), 3.25 (br d, J=15.0 Hz, 2H), 2.71 (br s, 2H), 2.06 (dd, J=8.5, 13.8 Hz, 2H), 1.75 (br dd, J=6.2, 13.9 Hz, 2H), 1.46 (s, 9H).

hexahydro-1H-spiro[cyclopenta[c]pyrrole-5,2′-[1,3]dioxolane]: A mixture of tert-butyl tetrahydro-1H-spiro[cyclopenta[c]pyrrole-5,2′-[1,3]dioxolane]-2(3H)-carboxylate (550 mg, 2.04 mmol) in 1,1,1,3,3,3-hexafluoropropan-2-ol (2.0 mL) were stirred at 150° C. for 3 h under microwave irradiation. The reaction mixture was concentrated under reduced pressure and the crude residue was used directly. LCMS: m/z=169.9 [M+H]⁺. ¹H-NMR (400 MHz, MeOD): δ 3.95-3.84 (m, 4H), 2.94-2.85 (m, 2H), 2.76-2.70 (m, 2H), 2.69-2.62 (m, 2H), 2.00 (br dd, J=9.0, 13.4 Hz, 2H), 1.60 (dd, J=4.6, 13.4 Hz, 2H).

2-(1-(3-cis-(trifluoromethoxy)cyclobutyl)-1H-pyrazol-4-yl)hexahydro-1H-spiro[cyclopenta[c]pyrrole-5,2′-[1,3]dioxolane]: A mixture of hexahydro-1H-spiro[cyclopenta[c]pyrrole-5,2′-[1,3]dioxolane] (1.4 g, 8.28 mmol), 4-iodo-1-(3-cis-(trifluoromethoxy)cyclobutyl)-1H-pyrazole (0.55 g, 1.66 mmol), CuI (126 mg, 0.66 mmol), L-proline (153 mg, 1.33 mmol) and K₂CO₃ (1.14 g, 8.28 mmol) in DMSO (10 mL) was degassed and purged with N₂ 3 times. The reaction mixture was then stirred at 100° C. for 12 h under N₂. The reaction mixture was cooled to 0° C., quenched by the addition of H₂O (50 mL), and extracted with EtOAc (3×20 mL). The combined organic layers were washed with brine (3×10 mL), dried over anhydrous Na₂SO₄, filtered, and concentrated under reduced pressure. The crude residue was purified by silica gel column chromatography to give the desired product. LCMS: m/z=374.1 [M+H]⁺. ¹H-NMR (400 MHz, CDCl₃): δ 7.17 (s, 1H), 6.93 (s, 1H), 4.53 (quin, J=7.2 Hz, 1H), 4.34 (quin, J=8.3 Hz, 1H), 3.92 (s, 4H), 3.09-3.02 (m, 2H), 3.01-2.90 (m, 4H), 2.81 (m, 4H), 2.13-2.01 (m, 2H), 1.76 (m, 2H).

2-(1-(3-cis-(trifluoromethoxy)cyclobutyl)-1H-pyrazol-4-yl)hexahydrocyclopenta[c]pyrrol-5(1H)-one: To a solution of 2-(1-(3-cis-(trifluoromethoxy)cyclobutyl)-1H-pyrazol-4-yl)hexahydro-1H-spiro[cyclopenta[c]pyrrole-5,2′-[1,3]dioxolane] (200 mg, 0.54 mmol) in EtOAc (3.0 mL) was added aq. HCl (2 M, 0.53 mL). The reaction mixture was stirred at 35° C. for 1 h. The reaction mixture was quenched by pouring onto ice-cold sat. aq. NaHCO₃ solution (10 mL) and then extracted with EtOAc (3×5 mL). The combined organic layers were washed with brine (3 mL), dried over anhydrous Na₂SO₄, filtered, and concentrated under reduced pressure. The crude residue was used directly. LCMS: n/z=330.1 [M+H]⁺. ¹H-NMR (400 MHz, CDCl₃): δ 7.14 (s, 1H), 6.90 (s, 1H), 4.53 (quin, J=7.3 Hz, 1H), 4.34 (quin, J=8.2 Hz, 1H), 3.31-3.23 (m, 2H), 3.10-2.91 (m, 6H), 2.88-2.75 (m, 2H), 2.59 (m, 2H), 2.26 (m, 2H).

2-(1-(3-cis-(trifluoromethoxy)cyclobutyl)-1H-pyrazol-4-yl)octahydrocyclopenta[c]pyrrol-5-amine: To a solution of 2-(1-(3-cis-(trifluoromethoxy)cyclobutyl)-1H-pyrazol-4-yl)hexahydrocyclopenta[c]pyrrol-5(1H)-one (140 mg, 0.42 mmol) in MeOH (3.0 mL) was added ammonium formate (335 mg, 5.31 mmol). The reaction mixture was stirred at 20° C. for 15 min and then NaBH₃CN (134 mg, 2.13 mmol) was added. The reaction mixture was further stirred at 20° C. for 2 h. The reaction mixture was then cooled to 0° C., quenched by addition of sat. aq. NH₄Cl (10 mL), and concentrated under reduced pressure. The residue was diluted with H₂O (10 mL) and extracted with EtOAc (3×10 mL). The combined organic layers were washed with brine (5 mL), dried over Na₂SO₄, filtered, and concentrated under reduced pressure. The residue was resuspeneded in H₂O (10 mL) and adjusted to pH=2-3 with 2 N aq. HCl at 0° C. The aqueous was then extracted with MTBE (3×10 mL). The water phase was then adjusted to pH=9-10 with 2 N NaOH at 0° C. and extracted with EtOAc (3×10 mL). The organics from the extraction of the basic aqueous layer were then combined, washed with brine (5 mL), dried over Na₂SO₄, filtered, and concentrated under reduced pressure to give a residue that was used directly. LCMS: n/z=331.2 [M+H]⁺.

2-(4-chlorophenoxy)-N-(2-(1-(3-cis-(trifluoromethoxy)cyclobutyl)-1H-pyrazol-4-yl)octahydrocyclopenta[c]pyrrol-5-yl)acetamide: Prepared using General Procedure A employing 2-(1-(3-cis-(trifluoromethoxy)cyclobutyl)-1H-pyrazol-4-yl)octahydrocyclopenta[c]pyrrol-5-amine (0.10 g, 0.30 mmol), 2-(4-chlorophenoxy)acetic acid (56 mg, 0.30 mmol), T3P solution (771 mg, 1.21 mmol, 50% in EtOAc), and Et₃N (123 mg, 1.21 mmol) in EtOAc (10 mL). The crude residue was purified by reverse-phase HPLC to give the desired product. LCMS: n/z=499.2 [M+H]⁺. ¹H-NMR (400 MHz, CDCl₃): δ 8.24 (br d, J=9.2 Hz, 1H), 7.34 (s, 1H), 7.19 (d, J=9.2 Hz, 2H), 6.96 (s, 1H), 6.42 (d, J=9.2 Hz, 2H), 4.69-4.55 (m, 2H), 4.43 (s, 2H), 4.42-4.33 (m, 1H), 3.24 (d, J=9.2 Hz, 2H), 3.09-2.97 (m, 2H), 2.96-2.77 (m, 6H), 2.38-2.27 (m, 2H), 1.69-1.63 (m, 2H).

### Example 62

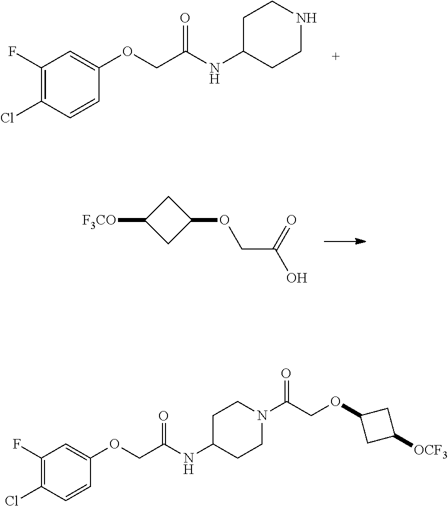

2-(4-chloro-3-fluoro-phenoxy)-N-[1-[2-[3-cis-(trifluoromethoxy)cyclobutoxy]acetyl]-4-piperidyl]acetamide: Prepared employing General Procedure A employing 2-(4-chloro-3-fluoro-phenoxy)-N-(4-piperidyl)acetamide (30 mg, 0.10 mmol), 2-[3-cis-(trifluoromethoxy)cyclobutoxy]acetic acid (27 mg, 0.13 mmol), N,N-diisopropylethylamine (41 mg, 0.31 mmol), and T3P solution (40 mg, 50% in EtOAc) in EtOAc (2 mL). Purified by reverse-phase HPLC to give the desired product. LCMS: m/z=483.3 [M+H]⁺. ¹H-NMR (400 MHz, CDCl₃): δ 7.35 (t, J=8.6 Hz, 1H), 6.77 (dd, J=10.3, 2.8 Hz, 1H), 6.69 (ddd, J=8.9, 2.9, 1.3 Hz, 1H), 6.39 (d, J=8.0 Hz, 1H), 4.55 (d, J=14.3 Hz, 1H), 4.47 (s, 2H), 4.30 (quintet, J=7.2 Hz, 1H), 4.17-4.03 (m, 3H), 3.92 (d, J=14.0 Hz, 1H), 3.80-3.73 (m, 1H), 3.20-3.13 (m, 1H), 2.86-2.75 (m, 3H), 2.32-2.22 (m, 2H), 2.11-1.99 (m, 2H), 1.48-1.37 (m, 2H).

### Example 63

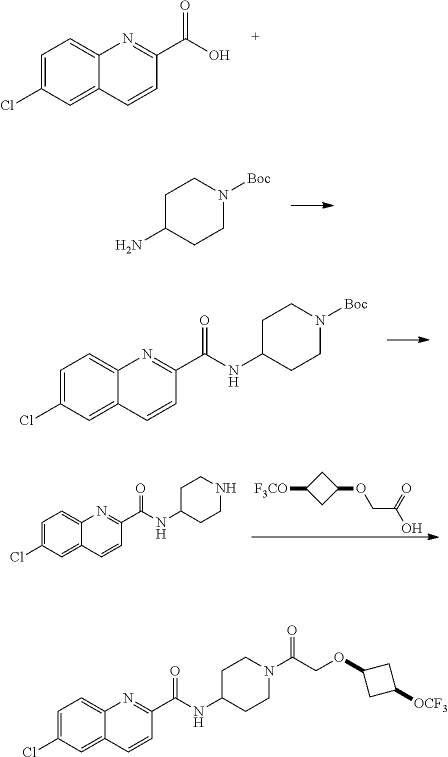

tert-butyl 4-[(6-chloroquinoline-2-carbonyl)amino]piperidine-1-carboxylate: Prepared employing General Procedure A employing 1-boc-4-aminopiperidine (50 mg, 0.25 mmol), 6-chloroquinoline-2-carboxylic acid (57 mg, 0.27 mmol), triethylamine (76 mg, 0.75 mmol), and T3P solution (95 mg, 50% in EtOAc) in EtOAc (2 mL). Purified employing silica gel column chromatography to provide the desired product.

6-chloro-N-(4-piperidyl)quinoline-2-carboxamide hydrochloride: tert-butyl 4-[(6-chloroquinoline-2-carbonyl)amino]piperidine-1-carboxylate (80 mg, 0.21 mmol) was dissolved in 4 M HCl in dioxane (1 mL) and was stirred for 2 h at 23° C. The resulting precipitate was collected and used directly.

6-chloro-N-[1-[2-[3-cis-(trifluoromethoxy)cyclobutoxy]acetyl]-4-piperidyl]quinoline-2-carboxamide: Prepared employing General Procedure A employing 6-chloro-N-(4-piperidyl)quinoline-2-carboxamide hydrochloride (66 mg, 0.20 mmol), 2-[3-cis-(trifluoromethoxy)cyclobutoxy]acetic acid (52 mg, 0.24 mmol), triethylamine (102 mg, 1.01 mmol), and T3P solution (77 mg, 50% in EtOAc) in EtOAc. Purified employing silica gel column chromatography to provide the desired product. LCMS: n/z=486.4 [M+H]j. ¹H-NMR (400 MHz, CDCl₃): δ 8.33 (d, J=8.5 Hz, 1H), 8.26-8.24 (m, 1H), 8.16 (d, J=8.2 Hz, 1H), 8.07 (d, J=9.0 Hz, 1H), 7.89 (d, J=2.3 Hz, 1H), 7.72 (dd, J=9.0, 2.3 Hz, 1H), 4.57 (d, J=13.2 Hz, 1H), 4.35-4.23 (m, 2H), 4.17-4.08 (m, 2H), 3.96 (d, J=14.2 Hz, 1H), 3.82-3.76 (m, 1H), 3.29-3.22 (m, 1H), 2.95-2.80 (m, 3H), 2.34-2.25 (m, 2H), 2.22-2.12 (m, 2H), 1.67-1.57 (m, 2H).

### Example 64

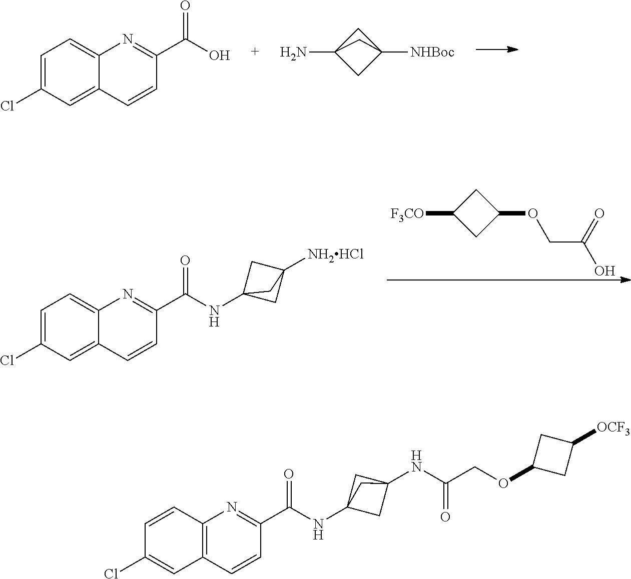

N-(1-amino-3-bicyclo[1.1.1]pentanyl)-6-chloro-quinoline-2-carboxamide hydrochloride: Prepared employing General Procedure A employing 2-methyl-2-propanyl (3-aminobicyclo[1.1.1]pent-1-yl)carbamate (110 mg, 0.55 mmol), 6-chloroquinoline-2-carboxylic acid (127 mg, 0.61 mmol), triethylamine (168 mg, 1.66 mmol), and T3P solution (212 mg, 50% in EtOAc) in EtOAc. The crude reaction mixture was dissolved in HCl solution in dioxane (4 M, 2 mL) and was stirred at 23° C. This precipitate was collected and used directly.

6-chloro-N-[1-[[2-[3-cis-(trifluoromethoxy)cyclobutoxy]acetyl]amino]-3-bicyclo[1.1.1]pentanyl]quinoline-2-carboxamide: Prepared employing General Procedure A employing N-(1-amino-3-bicyclo[1.1.1]pentanyl)-6-chloro-quinoline-2-carboxamide hydrochloride (43 mg, 0.13 mmol), 2-[3-cis-(trifluoromethoxy)cyclobutoxy]acetic acid (34 mg, 0.16 mmol), triethylamine (67 mg, 0.66 mmol), and T3P solution (50.6 mg, 50% in EtOAc) in EtOAc (2 mL). Purified by reverse-phase HPLC to give the desired product. LCMS: ma/z=484.1 [M+H]⁺. ¹H-NMR (400 MHz, CDCl₃): δ 8.62 (s, 1H), 8.31 (d, J=8.5 Hz, 1H), 8.25 (d, J=8.5 Hz, 1H), 8.07 (d, J=9.0 Hz, 1H), 7.89 (d, J=2.2 Hz, 1H), 7.73 (dd, J=9.0, 2.3 Hz, 1H), 6.92 (s, 1H), 4.35 (quintet, J=7.2 Hz, 1H), 3.85 (s, 2H), 3.74 (quintet, J=6.9 Hz, 1H), 2.84 (dtd, J=12.1, 5.8, 2.9 Hz, 2H), 2.64 (s, 6H), 2.34-2.27 (m, 2H).

### Example 65

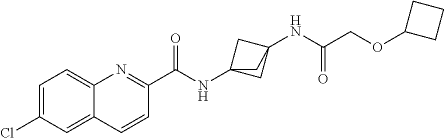 [SMILES: O=C(COC1CCC1)NC12CC(NC(=O)c3ccc4cc(Cl)ccc4n3)(C1)C2]

6-chloro-N-[1-[[2-(cyclobutoxy)acetyl]amino]-3-bicyclo[1.1.1]pentanyl]quinoline-2-carboxamide: Prepared using General Procedure A employing N-(1-amino-3-bicyclo[1.1.1]pentanyl)-6-chloro-quinoline-2-carboxamide hydrochloride (40 mg, 0.12 mmol), NEt₃ (62 mg, 0.62 mmol), T3P solution (47 mg, 0.15 mmol, 50% in EtOAc) and 2-(cyclobutoxy)acetic acid (19 mg, 0.15 mmol) in EtOAc (0.6 mL). Purified by reverse-phase HPLC to give the desired product. LCMS: m/z=400.1 [M+H]⁺. ¹H-NMR (400 MHz, CDCl₃): δ 8.48 (s, 1H), 8.18 (d, J=8.5 Hz, 1H), 8.13-8.10 (m, 1H), 7.96-7.94 (m, 1H), 7.76 (d, J=2.3 Hz, 1H), 7.60 (dd, J=9.0, 2.3 Hz, 1H), 6.85 (s, 1H), 3.91-3.84 (m, 1H), 3.70 (s, 2H), 2.50 (s, 6H), 2.13 (dddt, J=9.3, 8.0, 6.7, 2.6 Hz, 2H), 1.91-1.81 (m, 2H), 1.68-1.60 (m, 1H), 1.47-1.37 (m, 1H).

### Example 66

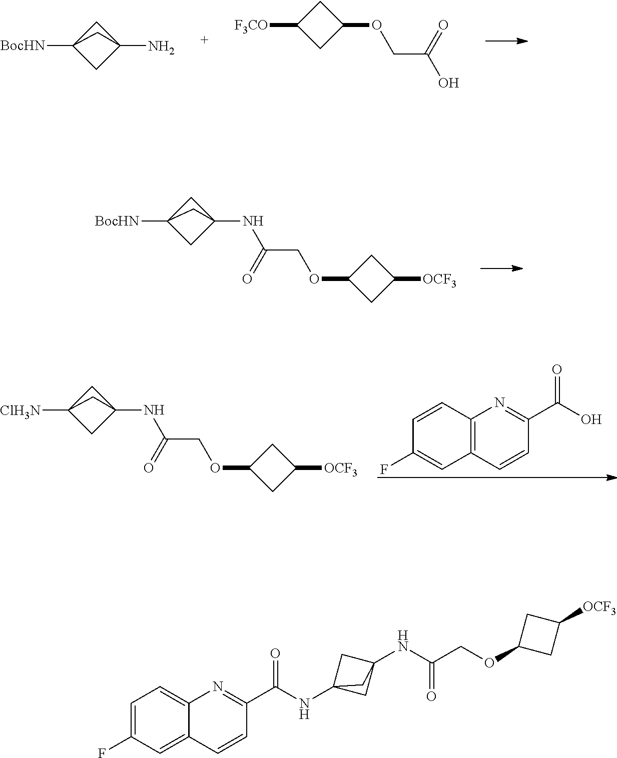

tert-butyl N-[1-[[2-[3-cis-(trifluoromethoxy)cyclobutoxy]acetyl]amino]-3-bicyclo[1.1.1]pentanyl]carbamate: Prepared employing General Procedure A employing 2-methyl-2-propanyl (3-aminobicyclo[1.1.1]pent-1-yl)carbamate (1.39 g, 7.0 mmol), 2-[3-cis-(trifluoromethoxy)cyclobutoxy]acetic acid (1.79 g, 8.4 mmol), triethylamine (3.54 g, 4.88 mmol), and T3P solution (6.68 g, 50% in EtOAc) in EtOAc (40 mL). The crude residue was used directly.

N-(1-amino-3-bicyclo[1.1.1]pentanyl)-2-[3-cis-(trifluoromethoxy)cyclobutoxy]acetamide hydrochloride: tert-butyl N-[1-[[2-[3-cis-(trifluoromethoxy)cyclobutoxy]acetyl]amino]-3-bicyclo[1.1.1]pentanyl]carbamate (2.76 g, 7.0 mmol) was dissolved in EtOAc (28 mL) and was treated with HCl solution (4 M in 1,4-dioxane, 5 mL). The resulting reaction mixture was stirred at 23° C. for 4 h and was then concentrated under reduced pressure and the residue was used directly.

6-fluoro-N-[1-[[2-[3-cis-(trifluoromethoxy)cyclobutoxy]acetyl]amino]-3-bicyclo[1.1.1]pentanyl]quinoline-2-carboxamide: Prepared using General Procedure A employing N-(1-amino-3-bicyclo[1.1.1]pentanyl)-2-[3-cis-(trifluoromethoxy)cyclobutoxy]acetamide (70 mg, 0.24 mmol), NEt₃ (120 mg, 1.19 mmol), T3P solution (91 mg, 0.29 mmol, 50% in EtOAc) and 6-fluoroquinoline-2-carboxylic acid (55 mg, 0.29 mmol) in EtOAc (1.1 mL) at 20° C. Purified by reverse-phase HPLC to give the desired product. LCMS: n/z=468.3 [M+H]J. ¹H-NMR (400 MHz, CDCl₃): δ 8.61 (s, 1H), 8.33-8.27 (m, 2H), 8.15 (dd, J=9.2, 5.3 Hz, 1H), 7.60-7.50 (m, 2H), 6.91 (t, J=0.2 Hz, 1H), 4.35 (quintet, J=7.2 Hz, 1H), 3.86 (s, 2H), 3.78-3.71 (m, 1H), 2.89-2.82 (m, 2H), 2.64 (s, 6H), 2.35-2.27 (m, 2H).

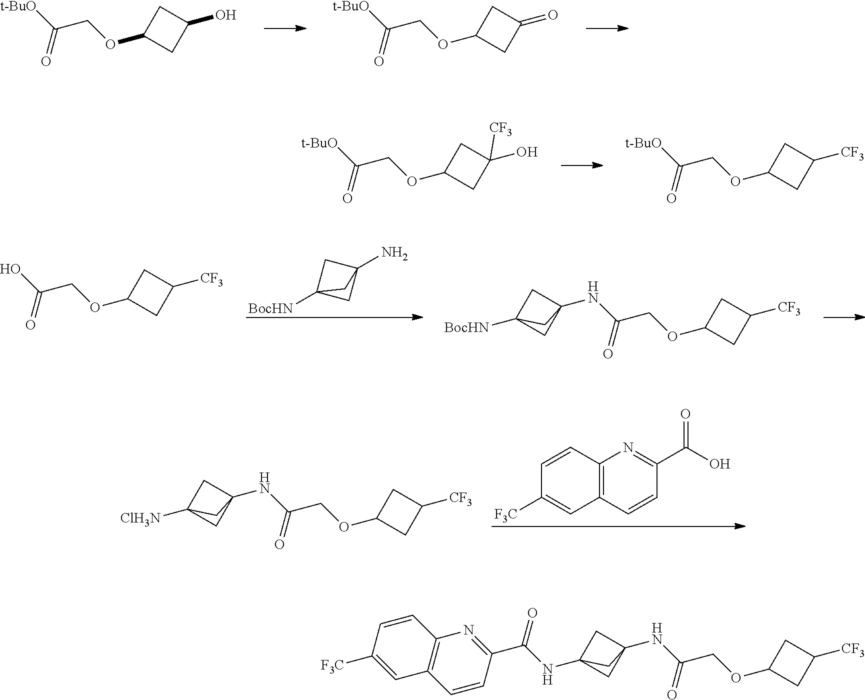

### Example 67

tert-butyl 2-(3-oxocyclobutoxy)acetate: To a solution of tert-butyl 2-(3-cis-hydroxycyclobutoxy)acetate (2.0 g, 9.89 mmol) in DCM (40 mL) at 0° C. was added DMP (6.29 g, 14.8 mmol) portion-wise. The mixture was warmed to 25° C. and was stirred for 4 h. The mixture was filtered, the filter cake was washed with DCM (2×20 mL), and the filtrate was concentrated under reduced pressure. The residue was purified by silica gel column chromatography to give the desired product. ¹H-NMR (400 MHz, CDCl₃): δ 4.46-4.42 (m, 11H), 3.99 (s, 2H), 3.28-3.19 (m, 4H), 1.48 (s, 9H).

tert-butyl 2-(3-hydroxy-3-(trifluoromethyl)cyclobutoxy)acetate: To a solution of tert-butyl 2-(3-oxocyclobutoxy)acetate (1.2 g, 5.99 mmol) in anhydrous THF (2.0 mL) at 0° C. was added TMSCF₃ (3.54 mL, 24.0 mmol) dropwise. TBAF (3.00 mL, 0.78 mmol, 1 M in THF) was then added dropwise, the mixture was warmed to 25° C. and stirred for 2 h. The reaction mixture was diluted with sat. NaHCO₃ solution (30 mL) and was extracted with EtOAc (2×20 mL). The combined organics were washed with brine (30 mL), dried over anhydrous Na₂SO₄, filtered, and concentrated under reduced pressure. The residue was purified by silica gel column chromatography to give the desired product. ¹H-NMR (400 MHz, CDCl₃): δ 3.91 (s, 2H), 3.89-3.83 (m, 1H), 3.29 (br s, 1H), 2.88-2.82 (m, 2H), 2.38-2.27 (m, 2H), 1.48 (s, 9H).

tert-butyl 2-(3-(trifluoromethyl)cyclobutoxy)acetate: To a mixture of tert-butyl 2-(3-hydroxy-3-(trifluoromethyl)cyclobutoxy)acetate (1.1 g, 4.07 mmol) in anhydrous THE (20 mL) at 0° C. was added NaH (244 mg, 6.11 mmol, 60% in mineral oil). The mixture was stirred for 30 min and 0-phenyl chloromethanethioate (1.05 g, 6.11 mmol) was added dropwise. The mixture was warmed to 25° C., stirred for 2 h, and then diluted with sat. NH₄Cl solution (20 mL) and water (20 mL). The aqueous phase was extracted with EtOAc (2×20 mL). The combined organics were washed with brine (20 mL), dried over anhydrous Na₂SO₄, filtered, and concentrated under reduced pressure. The residue was purified by silica gel column chromatography. This material was dissolved in anhydrous toluene (20 mL) and AIBN (662 mg, 4.03 mmol) was added followed by Bu₃SnH (3.20 g, 10.99 mmol). The reaction mixture was heated to 110° C. for 4 h and then diluted with aq. KF solution (40 mL, 2 M). The mixture was extracted with EtOAc (2×30 mL) and the combined organics were washed with brine (30 ML), dried over anhydrous Na₂SO₄, filtered, and concentrated under reduced pressure. The residue was purified by silica gel column chromatography to give the desired product (cis:trans=1:1). ¹H-NMR (400 MHz, CDCl₃): δ 4.23-4.18 (m, 0.51H), 4.05-3.93 (m, 0.51H), 3.89 (d, J=5.2 Hz, 211), 2.95-2.79 (m, 0.51H), 2.54-2.41 (m, 2.5H), 2.40-2.29 (m, 1H), 2.24-2.12 (m, 1H), 1.48 (s, 9H).

2-(3-(trifluoromethyl)cyclobutoxy)acetic acid: A solution of tert-butyl 2-(3-(trifluoromethyl)cyclobutoxy)acetate (200 mg, 0.79 mmol, cis:trans=1:1) in anhydrous DCM (5 mL) and TFA (1 mL) was stirred for 2 h at 25° C. The mixture was concentrated under reduced pressure and the crude residue was used directly.

tert-butyl (3-(2-(3-(trifluoromethyl)cyclobutoxy)acetamido)bicyclo[1.1.1]pentan-1-yl)carbamate: Prepared employing General Procedure A employing 2-(3-(trifluoromethyl)cyclobutoxy)acetic acid (150 mg, 0.76 mmol, cis:trans=1:1), tert-butyl (3-aminobicyclo[1.1.1]pentan-1-yl)carbamate (165 mg, 0.83 mmol), T3P solution (1.45 g, 2.27 mmol, 50% in EtOAc) and N,N-diisopropylethylamine (489 mg, 3.79 mmol) in EtOAc (3 mL). The residue was purified by silica gel column chromatography to give the desired product (cis:trans=1:1). LCMS: m/z=323.2 [M-55]⁺. ¹H-NMR (400 MHz, CDCl₃): δ 6.80 (br s, 1H), 4.97 (br s, 1H), 4.19-4.16 (m, 0.5H), 4.00-3.90 (m, 0.5H), 3.80 (d, J=3.6 Hz, 2H), 2.98-2.82 (m, 0.5H), 2.60-2.43 (m, 2.5H), 2.40-2.26 (m, 7H), 2.21-2.08 (m, 1H), 1.46 (s, 9H).

N-(3-aminobicyclo[1.1.1]pentan-1-yl)-2-(3-(trifluoromethyl)cyclobutoxy)acetamide HCl salt: A solution of tert-butyl (3-(2-(3-(trifluoromethyl)cyclobutoxy)acetamido)bicyclo[1.1.1]pentan-1-yl)carbamate (100 mg, 0.53 mmol, cis:trans=1:1) in HCl/EtOAc (10 mL, 4 M) was stirred for 1 h at 25° C. The mixture was concentrated under reduced pressure and the residue was used directly.

6-(trifluoromethyl)-N-(3-(2-(3-(trifluoromethyl)cyclobutoxy)acetamido)bicyclo[1.1.1]pentan-1-yl)quinoline-2-carboxamide: Prepared employing General Procedure A employing N-(3-aminobicyclo[1.1.1]pentan-1-yl)-²-(3-(trifluoromethyl)cyclobutoxy)acetamide HCl salt (80 mg, 0.25 mmol, cis:trans=1:1), 6-(trifluoromethyl)quinoline-2-carboxylic acid (61 mg, 0.25 mmol), T3P solution (484 mg, 0.76 mmol, 50% in EtOAc), and NEt₃ (129 mg, 1.27 mmol) in EtOAc (5 mL). Purified by reverse-phase HPLC to give the desired product (cis:trans=1:1). LCMS: m/z=502.3 [M+H]⁺. ¹H-NMR (400 MHz, CDCl₁3): δ 8.64 (s, 1H), 8.46-8.36 (m, 2H), 8.28-8.18 (m, 2H), 7.96 (dd, J=1.6, 8.8 Hz, 1H), 6.90 (s, 1H), 4.23-4.20 (m, 0.5H), 4.04-3.93 (m, 0.5H), 3.84 (d, J=3.6 Hz, 2H), 2.95-2.90 (m, 0.5H), 2.64 (s, 6H), 2.59-2.44 (m, 2.5H), 2.38-2.26 (m, 1H), 2.23-2.12 (m, 1H).

### Example 68

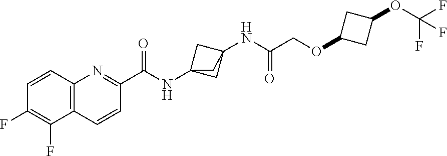

5,6-difluoro-N-(3-(2-(3-cis-(trifluoromethoxy)cyclobutoxy)acetamido)bicyclo[1.1.1]pentan-1-yl)quinoline-2-carboxamide: Prepared employing General Procedure A employing N-(3-aminobicyclo[1.1.1]pentan-1-yl)-2-(3-cis-(trifluoromethoxy)cyclobutoxy)acetamide (100 mg, 0.34 mmol), 5,6-difluoroquinoline-2-carboxylic acid (78 mg, 0.37 mmol), T3P solution (648 mg, 1.02 mmol, 0.61 mL, 50% in EtOAc) and NEt₃ (206 mg, 2.04 mmol) in EtOAc (5 mL). Purified by reverse-phase HPLC to give the desired product. LCMS: m/z=486.2 [M+H]⁺. ¹H-NMR (400 MHz, CDCl₃): δ 8.60 (d, J=8.8 Hz, 1H), 8.55 (s, 1H), 8.37 (d, J=8.7 Hz, 1H), 7.98-7.91 (m, 1H), 7.71-7.60 (m, 1H), 6.90 (s, 1H), 4.41-4.25 (m, 1H), 3.84 (s, 2H), 3.78-3.66 (m, 1H), 2.84 (dtd, J=3.4, 6.6, 9.9 Hz, 2H), 2.63 (s, 6H), 2.36-2.22 (m, 2H).

### Example 69

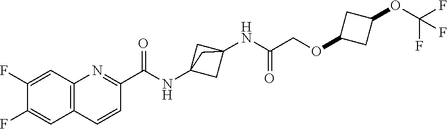

6,7-difluoro-N-(3-(2-(3-cis-(trifluoromethoxy)cyclobutoxy)acetamido)bicyclo[1.1.1]pentan-1-yl)quinoline-2-carboxamide: Prepared employing General Procedure A employing N-(3-aminobicyclo[1.1.1]pentan-1-yl)-2-(3-cis-(trifluoromethoxy)cyclobutoxy)acetamide, 6,7-difluoroquinoline-2-carboxylic acid (126 mg, 0.6 mmol), T3P solution (577 mg, 0.9 mmol, 50% in EtOAc) and NEt₃ (183 mg, 1.81 mmol) in EtOAc (10 mL). Purified by reverse-phase HPLC to give the desired product. LCMS: m/z=486.2 [M+H]⁺. ¹H-NMR (400 MHz, CDCl₃): δ 8.56 (s, 1H), 8.37-8.22 (m, 2H), 7.87 (dd, J=7.6, 10.8 Hz, 1H), 7.62 (dd, J=8.4, 10.0 Hz, 1H), 6.91 (s, 1H), 4.34 (quin, J=7.2 Hz, 1H), 3.84 (s, 2H), 3.73 (quin, J=6.9 Hz, 1H), 2.89-2.78 (m, 2H), 2.62 (s, 6H), 2.36-2.24 (m, 2H).

### Example 70

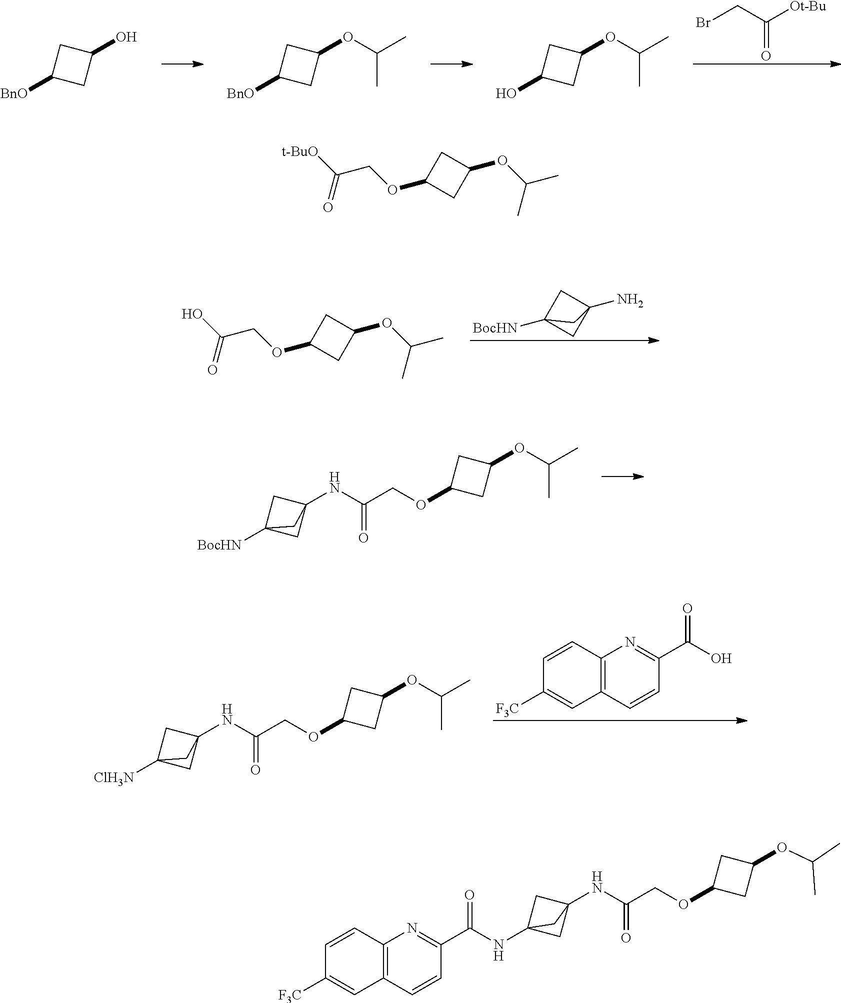

((3-cis-isopropoxycyclobutoxy)methyl)benzene: Prepared using General Procedure C employing 3-cis-(benzyloxy)cyclobutanol (5.0 g, 28.1 mmol), NaH (2.24 g, 56.1 mmol, 60% in mineral oil), and 2-iodopropane (28.6 g, 168.3 mmol) in DMF (100 mL) at 80° C. for 15 h. Purified by silica gel column chromatography to give the desired product. ¹H-NMR (400 MHz, CDCl₃): δ 7.23-7.38 (m, 5H), 4.36-4.46 (m, 2H), 3.63-3.73 (m, 2H), 3.54-3.62 (m, 1H), 2.58-2.68 (m, 2H), 1.91-2.03 (m, 2H), 1.14 (d, J=6.11 Hz, 6H).

3-cis-isopropoxycyclobutanol: A mixture of ((3-cis-isopropoxycyclobutoxy)methyl)benzene (1.5 g, 6.81 mmol), Pd/C (500 mg, 10 wt %) in MeOH (30 mL) was stirred at 50° C. under H₂ (50 psi) for 15 h. The mixture was filtered and was concentrated under reduced pressure to give a crude residue that was used directly. ¹H-NMR (400 MHz, CDCl₃): δ 3.84-3.96 (m, 1H), 3.52-3.65 (m, 2H), 2.64-2.77 (m, 2H), 1.82-1.94 (m, 2H), 1.13 (d, J=6.17 Hz, 6H).

tert-butyl 2-(3-cis-isopropoxycyclobutoxy)acetate: To a mixture of 3-cis-isopropoxycyclobutanol (300 mg, 2.30 mmol), tert-butyl 2-bromoacetate (674 mg, 3.46 mmol), tetrabutylammonium hydrogen sulfate (39 mg, 0.11 mmol) and H₂O (0.5 mL) in toluene (9 mL) was added dropwise NaOH (1.38 g, 34.57 mmol) in H₂O (2.5 mL), and the mixture was stirred at 25° C. for 2 h. The reaction mixture was diluted with ice water (30 mL), and then extracted was with MTBE (3×10 mL). The combined organic layers were washed with brine (20 mL), dried over anhydrous Na₂SO₄, filtered, and concentrated under reduced pressure. The residue was purified by silica gel column chromatography to give the desired product. ¹H-NMR (400 MHz, CDCl₃): δ 3.90 (m, 2H), 3.67-3.75 (m, 1H), 3.61-3.67 (m, 1H), 3.53-3.60 (m, 1H), 2.59-2.69 (m, 2H), 1.93-2.03 (m, 2H), 1.46-1.49 (s, 9H), 1.12 (d, J=6.11 Hz, 6H).

2-(3-cis-isopropoxycyclobutoxy)acetic acid: To a mixture of 2-(3-cis-isopropoxycyclobutoxy)acetate (400 mg, 1.64 mmol) in DCM (5 mL) was added TFA (1.54 g, 13.5 mmol), and the mixture was stirred at 40° C. for 2 h. The mixture was concentrated under reduced pressure and the residue was used directly. ¹H-NMR (400 MHz, CDCl₃): δ 4.06 (s, 2H), 3.74-3.82 (m, 1H), 3.69-3.74 (m, 1H), 3.60-3.68 (m, 1H), 2.63-2.78 (m, 2H), 1.97-2.09 (m, 2H), 1.17 (d, J=6.11 Hz, 6H).

tert-butyl (3-(2-(3-cis-isopropoxycyclobutoxy)acetamido)bicyclo[1.1.1]pentan-1-yl)carbamate: Prepared using General Procedure A employing tert-butyl N-(1-amino-3-bicyclo[1.1.1]pentanyl)carbamate (100 mg, 0.50 mmol), 2-(3-cis-isopropoxycyclobutoxy)acetic acid (113 mg, 0.60 mmol), NEt₃ (204 mg, 2.02 mmol), T3P solution (641 mg, 1.01 mmol, 50% in EtOAc) in EtOAc (5 mL). The residue was used directly. ¹H-NMR (400 MHz, CDCl₃): δ 6.87 (s, 1H), 3.78 (s, 2H), 3.62-3.71 (m, 2H), 3.53-3.62 (m, 1H), 2.62-2.72 (m, 2H), 2.35 (s, 6H), 1.89-1.98 (m, 2H), 1.45 (s, 9H), 1.14 (d, J=5.95 Hz, 6H).

N-(3-aminobicyclo[1.1.1]pentan-1-yl)-2-(3-cis-isopropoxycyclobutoxy)acetamide: A mixture of tert-butyl (3-(2-(3-cis-isopropoxycyclobutoxy)acetamido)bicyclo[1.1.1]pentan-1-yl)carbamate (160 mg, 0.43 mmol) in HCl (4 M in EtOAc, 10 mL) was stirred at 25° C. for 2 h. The mixture was concentrated under reduced pressure and the residue was used directly. ¹H-NMR (400 MHz, MeOD): δ 3.81 (s, 2H), 3.56-3.75 (m, 3H), 2.58-2.71 (m, 2H), 2.40 (s, 6H), 1.83-1.95 (m, 2H), 1.12 (d, J=6.24 Hz, 6H).

N-(3-(2-(3-cis-isopropoxycyclobutoxy)acetamido)bicyclo[1.1.1]pentan-1-yl)-6-(trifluoromethyl)quinoline-2-carboxamide: Prepared using General Procedure A employing N-(3-aminobicyclo[1.1.1]pentan-1-yl)-2-(3-cis-isopropoxycyclobutoxy)acetamide, HCl salt (120 mg, 0.39 mmol), 6-(trifluoromethyl)quinoline-2-carboxylic acid (104 mg, 0.43 mmol), NEt₃ (159 mg, 1.57 mmol), T3P solution (501 mg, 0.78 mmol, 0.47 mL, 50% in EtOAc) in EtOAc (10 mL). Purified by reverse-phase HPLC to give the desired product. LCMS: m/z=492.4 [M+H]⁺. ¹H-NMR (400 MHz, CDCl₃): δ 8.64 (s, 1H), 8.35-8.47 (m, 2H), 8.25 (d, J=8.80 Hz, 1H), 8.21 (s, 1H), 7.95 (dd, J=8.86, 1.90 Hz, 1H), 6.98 (s, 1H), 3.82 (s, 2H), 3.68 (m, 2H), 3.54-3.63 (m, 1H), 2.66-2.75 (m, 2H), 2.63 (s, 6H), 1.91-2.04 (m, 2H), 1.15 (d, J=6.11 Hz, 6H).

### Example 71

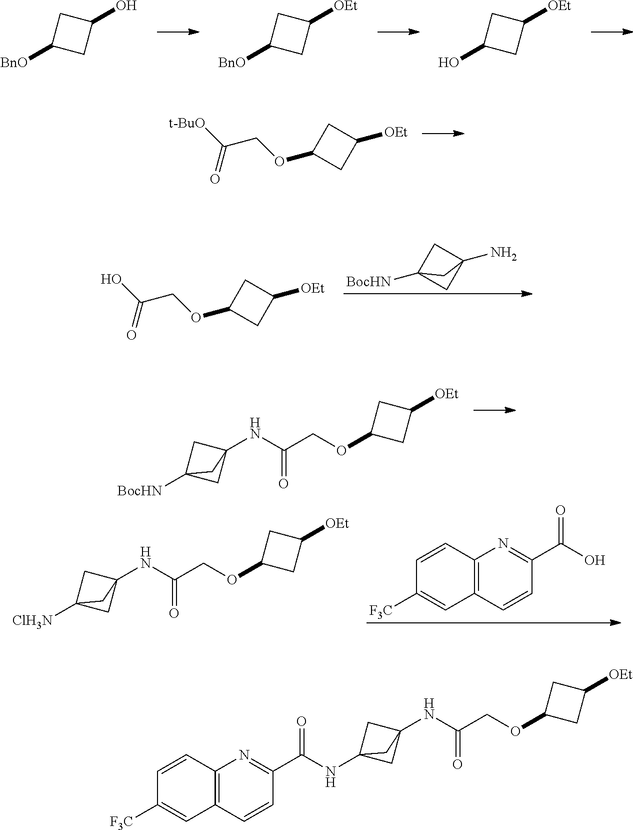

((3-cis-ethoxycyclobutoxy)methyl)benzene: Prepared employing General Procedure C employing 3-cis-(benzyloxy)cyclobutanol (5.0 g, 28.1 mmol), NaH (1.23 g, 30.9 mmol, 60% dispersion in mineral oil) and iodoethane (4.8 g, 30.9 mmol, 2.47 mL) in THF (100 mL) for 15 h. Purified by silica gel column chromatography to give the desired product. ¹H NMR (400 MHz, CDCl₃): δ 7.27 (s, 5H), 4.45-4.39 (m, 2H), 3.72-3.63 (m, 1H), 3.61-3.52 (m, 1H), 3.39 (q, J=7.1 Hz, 2H), 2.68-2.56 (m, 2H), 2.02-1.90 (m, 2H), 1.19 (t, J=7.0 Hz, 3H).

3-cis-ethoxycyclobutanol: To a solution of ((3-cis-ethoxycyclobutoxy)methyl)benzene (1.7 g, 8.24 mmol) in MeOH (20 mL) was added Pd/C (600 mg, 0.56 mmol, 10 wt %). The reaction mixture was degassed under vacuum, purged with H₂ three times, and stirred under H₂ atmosphere (50 psi) at 50° C. for 15 h. The reaction mixture was filtered and concentrated under reduced pressure to give a residue that was used directly. ¹H NMR (400 MHz, CDCl₃): δ 3.88 (quin, J=7.2 Hz, 1H), 3.59-3.46 (m, 1H), 3.37 (q, J=7.0 Hz, 2H), 2.78-2.64 (m, 3H), 1.92-1.80 (m, 2H), 1.17 (t, J=7.0 Hz, 3H).

tert-butyl 2-(3-cis-ethoxycyclobutoxy)acetate: To a mixture of 3-cis-ethoxycyclobutanol (500 mg, 4.30 mmol) and tert-butyl 2-bromoacetate (1.26 g, 6.46 mmol, 0.95 mL) and tetrabutylammonium hydrogen sulfate (73.1 mg, 0.215 mmol) in toluene (10 mL) and H₂O (1 mL) was added NaOH (2.58 g, 64.57 mmol) in H₂O (5 mL). The reaction mixture was stirred at 20° C. for 4 h. The mixture was poured into H₂O (20 mL), the aqueous phase was extracted with EtOAc (3×30 mL), washed with brine (2×30 mL), dried over anhydrous Na₂SO₄, and was filtered and concentrated under reduced pressure. The residue was purified by silica gel column chromatography to give the desired product. ¹H NMR (400 MHz, CDCl₃): δ 3.87 (s, 2H), 3.74-3.67 (m, 1H), 3.55 (quin, J=7.1 Hz, 1H), 3.37 (q, J=7.1 Hz, 2H), 2.68-2.57 (m, 2H), 2.01-1.91 (m, 2H), 1.51-1.43 (m, 9H), 1.20-1.13 (m, 3H).

2-(3-cis-ethoxycyclobutoxy)acetic acid: To a mixture of tert-butyl 2-(3-cis-ethoxycyclobutoxy)acetate (980 mg, 4.26 mmol) in DCM (10 mL) was added TFA (3.08 g, 27.01 mmol, 2 mL), and the mixture was stirred at 40° C. for 2 h. The mixture was concentrated under reduced pressure to give a residue that was used directly. ¹H NMR (400 MHz, CDCl₃): δ 4.09 (s, 2H), 3.83-3.67 (m, 2H), 3.58-3.47 (m, 2H), 2.79-2.65 (m, 2H), 2.11-2.01 (m, 2H), 1.23 (t, J=7.1 Hz, 3H).

tert-butyl (3-(2-(3-cis-ethoxycyclobutoxy)acetamido)bicyclo[1.1.1]pentan-1-yl)carbamate: Prepared employing General Procedure A employing tert-butyl (3-aminobicyclo[1.1.1]pentan-1-yl)carbamate (100 mg, 0.5 mmol), 2-(3-cis-ethoxycyclobutoxy)acetic acid, T3P solution (962 mg, 1.51 mmol, 0.90 mL, 50% in EtOAc), and NEt₃ (306 mg, 3.03 mmol, 0.421 mL) in EtOAc (5 mL) for 4 h. The reaction mixture was purified by silica gel column chromatography to give the desired product. ¹H NMR (400 MHz, CDCl₃): δ 6.87 (s, 1H), 4.97 (br s, 1H), 3.79 (s, 2H), 3.71-3.57 (m, 2H), 3.41 (q, J=7.1 Hz, 2H), 2.71-2.64 (m, 2H), 2.35 (s, 6H), 1.98-1.88 (m, 2H), 1.45 (s, 9H), 1.20 (t, J=6.9 Hz, 3H).

N-(3-aminobicyclo[1.1.1]pentan-1-yl)-2-(3-cis-ethoxycyclobutoxy)acetamide HCl salt: A mixture of tert-butyl (3-(2-(3-cis-ethoxycyclobutoxy)acetamido)bicyclo[1.1.1]pentan-1-yl)carbamate (162 mg, 0.457 mmol) in HCl (10 mL, 4 M in EtOAc) was stirred at 20° C. for 1 h. The mixture was concentrated under reduced pressure and the crude residue was used directly.

N-(3-(2-(3-cis-ethoxycyclobutoxy)acetamido)bicyclo[1.1.1]pentan-1-yl)-6-(trifluoromethyl)quinoline-2-carboxamide: Prepared employing General Procedure A employing N-(3-aminobicyclo[1.1.1]pentan-1-yl)-2-(3-cis-ethoxycyclobutoxy)acetamide HCl salt (110 mg, 0.378 mmol), 6-(trifluoromethyl)quinoline-2-carboxylic acid (100 mg, 0.416 mmol), T3P solution (722 mg, 1.13 mmol, 0.67 mL, 50% in EtOAc) and NEt₃ (229 mg, 2.27 mmol, 0.3 mL) in EtOAc (10 mL) for 4 h. Purified by reverse-phase HPLC to give the desired product. LCMS: m/z=478.1 [M+H]⁺. ¹H-NMR (400 MHz, CDCl₃): δ 8.63 (s, 1H), 8.41 (q, J=8.6 Hz, 2H), 8.28-8.19 (m, 2H), 7.95 (dd, J=2.0, 8.9 Hz, 1H), 6.97 (s, 1H), 3.83 (s, 2H), 3.73-3.60 (m, 2H), 3.42 (q, J=7.1 Hz, 2H), 2.74-2.67 (m, 2H), 2.63 (s, 6H), 2.04-1.91 (m, 2H), 1.22 (t, J=7.0 Hz, 3H).

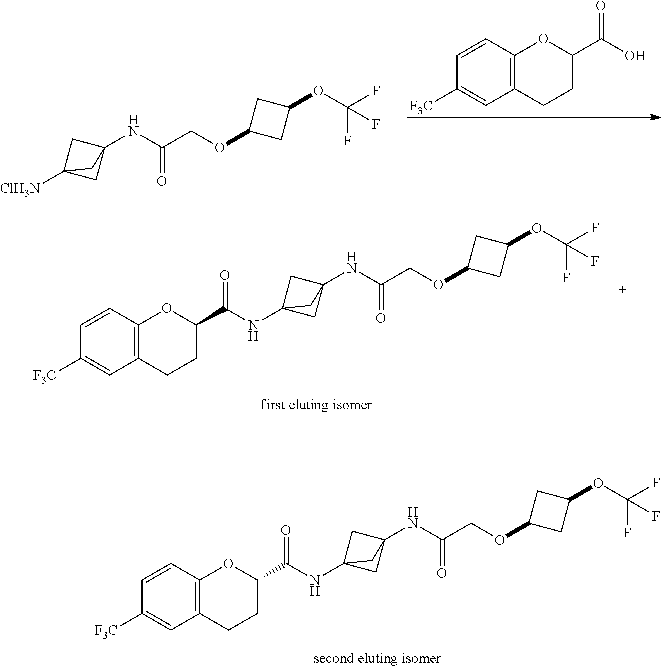

### Example 72

N-(3-(2-(3-cis-(trifluoromethoxy)cyclobutoxy)acetamido)bicyclo[1.1.1]pentan-1-yl)-6-(trifluoromethyl)chroman-2-carboxamide: Prepared using General Procedure A employing N-(3-aminobicyclo[1.1.1]pentan-1-yl)-2-(3-cis-(trifluoromethoxy)cyclobutoxy)acetamide HCl salt (100 mg, 0.30 mmol), 6-(trifluoromethyl)chromane-2-carboxylic acid (81 mg, 0.33 mmol), NEt₃ (183 mg, 1.81 mmol, 0.25 mL) and T3P solution (577 mg, 0.91 mmol, 0.54 mL, 50% in EtOAc) in EtOAc (10 ML). Purified by reverse-phase HPLC (column: HUAPU C8 Extreme BDS 150 mm×30 mm, 5 μM; mobile phase: A: 10 mM NH₄HCO₃ in water, B: MeCN; B % in A: 45%-75% over 10 min) and further purified by SFC (column: DAICEL CHIRALCEL OD-H (250 mm×30 mm, 5 μM; mobile phase: A: 0.1% NH₃ in H₂O, B: IPA; B % in A: 35%-35% over 6 min; first eluting isomer retention time: 2.87 min, second eluting isomer retention time: 3.11 min) to give:

First eluting isomer: LCMS: m/z=523.0 [M+H]⁺. ¹H-NMR (400 MHz, CDCl₃): δ LCMS, m/z=523.0 [M+H]⁺. ¹H NMR (400 MHz, CDCl₃): δ 7.40 (d, J=8.3 Hz, 1H), 7.36 (s, 1H), 7.01-6.96 (m, 2H), 6.85 (s, 1H), 4.50 (dd, J=2.6, 10.1 Hz, 1H), 4.33 (quin, J=7.1 Hz, 1H), 3.82 (s, 2H), 3.72 (quin, J=6.9 Hz, 1H), 2.98-2.88 (m, 1H), 2.87-2.79 (m, 3H), 2.51 (s, 6H), 2.49-2.44 (m, 1H), 2.32-2.24 (m, 2H), 2.05-1.96 (m, 1H).

Second eluting isomer: LCMS, m/z=523.0 [M+H]⁺. ¹H NMR (400 MHz, CDCl₃): δ 7.39 (d, J=8.3 Hz, 1H), 7.36 (s, 1H), 7.01-6.96 (m, 2H), 6.86 (s, 1H), 4.49 (dd, J=2.6, 10.1 Hz, 1H), 4.33 (quin, J=7.1 Hz, 1H), 3.82 (s, 2H), 3.71 (quin, J=6.9 Hz, 1H), 2.91 (dd, J=5.9, 10.7 Hz, 1H), 2.86-2.79 (m, 3H), 2.51 (s, 6H), 2.48-2.43 (m, 1H), 2.32-2.24 (m, 2H), 2.06-1.95 (m, 1H).

### Example 73

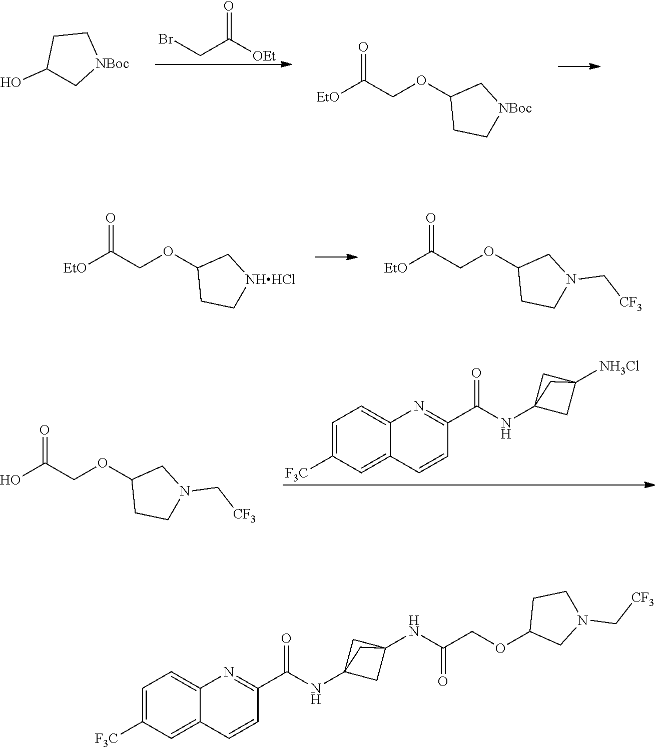

tert-butyl 3-(2-ethoxy-2-oxoethoxy)pyrrolidine-1-carboxylate: Prepared employing General Procedure C employing tert-butyl 3-hydroxypyrrolidine-1-carboxylate (5.0 g, 26.7 mmol), NaH (1.12 g, 28.0 mmol, 60% in mineral oil), and ethyl 2-bromoacetate (4.91 g, 29.4 mmol) in THE (50 mL). Purified by silica gel column chromatography to give the desired product. ¹H NMR (400 MHz, CDCl₃): δ 4.22 (q, J=7.2 Hz, 2H), 4.18-4.12 (m, 1H), 4.10-4.04 (m, 2H), 3.54-3.38 (m, 4H), 2.15-2.01 (m, 1H), 2.00-1.89 (m, 1H), 1.46 (s, 9H), 1.29 (t, J=7.2 Hz, 3H).

ethyl 2-(pyrrolidin-3-yloxy)acetate HCl salt: To a solution of tert-butyl 3-(2-ethoxy-2-oxoethoxy)pyrrolidine-1-carboxylate (2.0 g, 7.32 mmol) in EtOAc (5 mL) was added HCl/EtOAc (4 M, 20 mL), and the mixture was stirred at 20° C. for 2 h. The mixture was concentrated under reduced pressure to give a residue that was used directly.

ethyl 2-((1-(2,2,2-trifluoroethyl)pyrrolidin-3-yl)oxy)acetate: To a mixture of ethyl 2-(pyrrolidin-3-yloxy)acetate HCl salt (1.78 g, 8.49 mmol) and 2,2,2-trifluoroethyl trifluoromethanesulfonate (2.56 g, 11.04 mmol) in DMF (20 mL) was added N,N-diisopropylethylamine (5.49 g, 42.45 mmol, 7.39 mL), and the mixture was stirred at 20° C. for 15 h. The mixture was poured onto water (100 mL), the layers were separated and the aqueous phase was extracted with EtOAc (3×15 mL). The combined organic phases were washed with brine (2×10 mL), dried over anhydrous Na₂SO₄, filtered, and concentrated under reduced pressure. The residue was purified by silica gel column chromatography to give the desired product. ¹H NMR (400 MHz, CDCl₃): δ 4.22 (q, J=7.2 Hz, 2H), 4.19-4.12 (m, 1H), 4.05 (d, J=2.0 Hz, 2H), 3.17-3.05 (m, 3H), 2.90-2.75 (m, 3H), 2.16-2.03 (m, 1H), 1.92 (m, J=3.2, 4.8, 10.6 Hz, 1H), 1.29 (t, J=7.2 Hz, 3H).

2-((1-(2,2,2-trifluoroethyl)pyrrolidin-3-yl)oxy)acetic acid: A solution of ethyl 2-((1-(2,2,2-trifluoroethyl)pyrrolidin-3-yl)oxy)acetate (100 mg, 0.39 mmol) in HCl (6 M, 5 mL) was heated to 70° C. for 5 h. The mixture was concentrated under reduced pressure to give a residue that was used directly.

N-(3-(2-((1-(2,2,2-trifluoroethyl)pyrrolidin-3-yl)oxy)acetamido)bicyclo[1.1.1]pentan-1-yl)-6-(trifluoromethyl)quinoline-2-carboxamide: Prepared employing General Procedure A employing 2-((1-(2,2,2-trifluoroethyl)pyrrolidin-3-yl)oxy)acetic acid (104 mg, 0.46 mmol), N-(3-aminobicyclo[1.1.1]pentan-1-yl)-6-(trifluoromethyl)quinoline-2-carboxamide HCl salt (110 mg, 0.31 mmol), NEt₃ (186 mg, 1.84 mmol) and T3P solution (586 mg, 0.92 mmol, 0.55 mL, 50% in EtOAc) in EtOAc (5 mL). Purified by reverse-phase HPLC to give the desired product. LCMS: m/z=531.0 [M+H]⁺. ¹H-NMR (400 MHz, CDCl₃): δ 8.64 (s, 1H), 8.46-8.37 (m, 2H), 8.31-8.17 (m, 2H), 7.95 (dd, J=2.0, 8.8 Hz, 1H), 7.02 (s, 1H), 4.20-4.09 (m, 1H), 4.01-3.81 (m, 2H), 3.17-3.08 (m, 2H), 3.06-2.99 (m, 1H), 2.95-2.90 (m, 1H), 2.90-2.84 (m, 1H), 2.72-2.65 (m, 1H), 2.63 (s, 6H), 2.22-2.11 (m, 1H), 1.99-1.88 (m, 1H).

### Examples 74-75

The following compounds were made via similar procedures as those described throughout.

| Ex. | Compound | [M + H]⁺ |
|---|---|---|
| 74 | 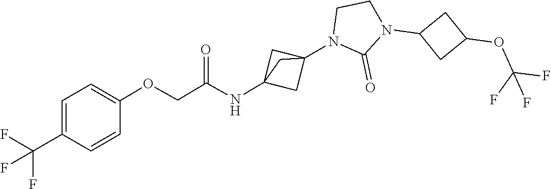 [SMILES: O=C(COc1ccc(C(F)(F)F)cc1)NC12CC(N3CCN(C4CC(OC(F)(F)F)C4)C3=O)(C1)C2] | 508.2 |
| 75 | 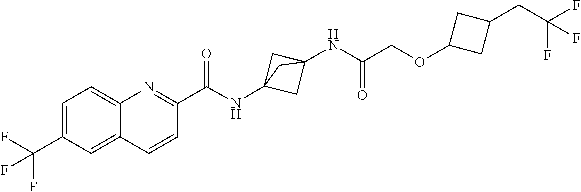 [SMILES: O=C(COC1CC(CC(F)(F)F)C1)NC12CC(NC(=O)c3ccc4cc(C(F)(F)F)ccc4n3)(C1)C2] | 515.9 |

### Biological Example 1

### Biochemical Assay of the Compounds

Cellular stress leads to activation of the integrated stress response pathway through one of four eukaryotic initiation factor 2a kinases and halts global translation, while allowing for the translation of select transcripts like ATF4 (activating transcription factor 4) that are important for the response to cellular stress. During normal conditions, small open reading frames (ORFs) in the 5′ UTR of ATF4 occupy the ribosome and prevent translation of the coding sequence of ATF4. During stress conditions however, the ribosome scans past these upstream ORFs and preferentially begins translation at the coding sequence of ATF4. In this way, the translation, and thus protein level of ATF4 is a readout of ISR pathway activation. Thus, a fusion of the uORFs and the beginning of the coding sequence of ATF to a common cellular reporter like nano-luciferase allows for a sensitive and high-throughput readout of ISR pathway activity.

Compounds as provided herein were tested in the following assay. The ATF4 Nano Luciferase reporter was constructed by fusing the human full length 5′ untranslated region (5′-UTR) and a small portion of the coding sequence of the ATF4 gene upstream of the Nano Luciferase (NLuc) coding sequence lacking it's start codon. Specifically, nucleotides +1 through +364 (relative to the transcriptional start site) of ATF4 transcript variant 2 (NCBI NM_182810.2) flanked 5′ by EcoRI and 3′ by BamHI restriction enzyme sites were synthesized and cloned into the EcoRI/BamHI cloning sites of pLVX-EF1a-IRES-Puro lentivirus vector (Clontech). Lentiviral particles were produced with Lenti-X single shots (VSV-G, Clontech) according to the manufacturer's instructions and used to transduce a human H4 neuroglioma cell line (ATCC HTB-148). H4 cells were selected with 1.25 μg/mL Puromycin, and clonal cell lines generated by limiting dilution. We utilized this cell line to generate an integrated stress response (ISR) assay to evaluate the activity of ISR pathway inhibitors via luminescence readout. The H4 ATF4-NLuc (clone 17) cell line is plated at a density of 15,000 or 2.50 cells in 96-well or 384-well respectively in DMEM +10% fetal bovine serum. 24-hours later test compounds diluted in dimethyl sulfoxide (DMSO) are added for 30 minutes at 37 degree Celsius, followed by ISR pathway activation with 50 um sodium arsenite aqueous solution for 6 additional hours. Nano Glo luciferase reagent (N1150, Promega) is added according to manufacturer instructions and the luminescence signal (corresponding to the level of ATF4 translation and thus ISR pathway activation) is read with a standard plate reader with luminescence detection capabilities.

In the table below, activity of the tested compounds is provided in Table 3 as follows: +++=IC₅₀<1 μM; ++=IC₅₀ 1-10 μM; +=IC₅₀>10 μM.

TABLE 3

| Example No. | MH+ | IC₅₀ |
|---|---|---|
| 1 | 379.2 | +++ |
| 2 | 415.13 | +++ |
| 3 (first | 447.15 | +++ |
| eluting isomer) | | |
| 3 (second | 447.15 | +++ |
| eluting isomer) | | |
| 4 | 419.25 | +++ |
| 5 | 407.23 | +++ |
| 6 | 404.21 | +++ |
| 7 | 422.19 | +++ |
| 8 | 418.2 | ++ |
| 9 | 415.17 | +++ |
| 10 | 397.19 | +++ |
| 11 | 436.19 | +++ |
| 12 (first | 465.14 | +++ |
| eluting isomer) | | |
| 12 (second | 465.26 | +++ |
| eluting isomer) | | |
| 13 | 397.15 | ++ |
| 14 | 464.3 | +++ |
| 15 | 439.3 | +++ |
| 16 | 507.24 | +++ |
| 17 | 479.21 | +++ |
| 18 | 478.29 | +++ |
| 19 | 411.18 | +++ |
| 20 | 436.3 | +++ |
| 21 | 495.22 | +++ |
| 22 | 495.3 | +++ |
| 23 | 495.3 | +++ |
| 24 | 455.3 | ++ |
| 25 | 465.4 | ++ |
| 26 | 493.3 | +++ |
| 27 | 470.6 | +++ |
| 28 | 492.5 | +++ |
| 29 | 474.3 | +++ |
| 30 | 502.8 | +++ |
| 31 | 404.3 | +++ |
| 32 | 514.3 | +++ |
| 33 | 504.3 | +++ |
| 34 | 463.3 | +++ |
| 35 | 427.4 | +++ |
| 36 | 393.4 | +++ |
| 37 | 481.4 | +++ |
| 38 | 491.4 | +++ |
| 39 | 491.4 | +++ |
| 40 | 425.4 | +++ |
| 41 | 425.4 | +++ |
| 42 | 456.3 | +++ |
| (isomer 1) | | |
| 42 | 456.3 | +++ |
| (isomer 2) | | |
| 43 | 490.4 | +++ |
| 44 | 475.3 | +++ |
| 46 | 491.4 | +++ |
| 47 | 505.2 | +++ |
| 48 | 499.3 | +++ |
| 49 | 499.3 | +++ |
| 50 | 533.3 | +++ |
| 51 | 518.4 | +++ |
| 52 | 490.3 | +++ |
| 53 | 452.6 | +++ |
| 54 | 452.7 | +++ |
| 55 | 470.7 | +++ |
| 56 | 483.2 | +++ |
| 57 | 519.1 | +++ |
| 58 | 503.2 | +++ |
| (cis isomer) | | |
| 58 | 503.2 | +++ |
| (trans isomer) | | |
| 59 | 491.1 | +++ |
| 60 | 505.1 | +++ |
| 61 | 499.2 | + |
| 62 | 483.3 | +++ |
| 63 | 486.4 | +++ |
| 64 | 484.1 | +++ |
| 65 | 400.1 | +++ |
| 66 | 468.3 | +++ |
| 67 | 502.3 | +++ |
| 68 | 486.2 | +++ |
| 69 | 486.2 | +++ |
| 70 | 492.4 | +++ |
| 71 | 478.1 | +++ |
| 72 (first | 523.0 | +++ |
| eluting isomer) | | |
| 72 (second | 523.0 | +++ |
| eluting isomer) | | |
| 73 | 531.0 | +++ |
| 74 | 508.2 | +++ |
| 75 | 515.9 | +++ |

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control. It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A compound of Formula A: 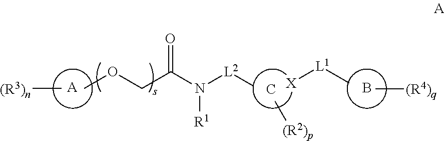
or a pharmaceutically acceptable salt, isotopically enriched analog, stereoisomer, mixture of stereoisomers or prodrug thereof, wherein:
A and B are independently C₃₋₁₀ cycloalkyl, heterocyclyl, aryl, or heteroaryl, provided that at least one of A or B is C₃₋₁₀ cycloalkyl;
X is C or N;
C is cycloalkyl when X is C or heterocyclyl when X is N;
L¹ is —NR⁹C(O)CH₂O—, —C(O)NR⁹CH₂O—, a heteroalkylene optionally substituted with one to six R⁵, or L¹ is a heterocyclyl or heteroaryl, each of which is optionally substituted with one to six R¹³;
provided that when C is a bicyclo[1.1.1]pentane or bicyclo[2.1.1]hexane, then L¹ is —NR⁹C(O)CH₂O— or —C(O)NR⁹CH₂O— and L² is a bond;
L² is a bond or a C₁₋₂ alkylene optionally substituted with one to four R⁵;
n is 0, 1, 2, 3, 4, 5, or 6;
p is 0, 1, 2, 3, 4, 5, 6, 7, or 8;
q is 0, 1, 2, 3, 4, 5, or 6;
s is 0 or 1;
R¹ is hydrogen, C₁₋₁₂ alkyl, C₂₋₁₂ alkenyl, C₂₋₁₂ alkynyl, C₃₋₁₀ cycloalkyl or heterocyclyl, each of which, other than hydrogen, is optionally substituted with one to six R¹¹;
each R² is independently halo, cyano, —NR⁶R⁷, hydroxyl, oxo, —C(O)OR⁶, —OC(O)NR⁶R⁷, —C(O)NR⁶R⁷, —NR⁶C(O)R⁷, C₁₋₁₂ alkoxy, C₁₋₁₂ haloalkoxy, C₁₋₁₂ alkyl, C₁₋₁₂ haloalkyl, heteroaryl, heterocyclyl, and cycloalkyl, wherein each of heterocyclyl, heteroaryl, and cycloalkyl are independently optionally substituted with one to six cyano, halo, C₁₋₁₂ alkyl, or C₁₋₁₂ haloalkyl, or two R² on non-adjacent ring atoms together form a bond, C₁₋₁₃ alkylene optionally substituted with one to six R⁵, or C₁₋₂ heteroalkylene optionally substituted with one to four R⁵, provided that when C is cyclobutyl then R² is not oxo;
each R⁵ is independently halo, C₁₋₆ alkyl or C₁₋₆ haloalkyl;
R³ and R⁴ are independently R¹¹;
each R¹¹ is independently halo, cyano, nitro, oxo, —OR⁶, —SR⁶, —SF₅, —NR⁶R⁷, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R⁶, —C(O)OR⁶, —OC(O)OR⁶, —OC(O)R⁶, —C(O)NR⁶R⁷, —OC(O)NR⁶R⁷, —NR⁶C(O)NR⁷R⁸, —S(O)₁₋₂R⁶, —S(O)₁₋₂NR⁶, —NR⁶S(O)₁₋₂R⁷, —NR⁶S(O)₁₋₂NR⁷R⁸, —NR⁶C(O)R⁷ or —NR⁶C(O)OR⁷, wherein each C₁₋₁₂ alkyl, C₂₋₁₂alkenyl, C₂₋₁₂ alkynyl, C₃₋₁₀ cycloalkyl, heterocyclyl, aryl and heteroaryl of R¹¹ is independently optionally substituted with one to six R¹²;
each of R⁶, R⁷, and R⁸ is independently hydrogen, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R²⁰, —C(O)OR²⁰, —C(O)NR²⁰R²¹, —S(O)₁₋₂R²⁰ or —S(O)₁₋₂NR²⁰, wherein each C₁₋₁₂alkyl, C₂₋₁₂ alkenyl, C₂₋₁₂ alkynyl, C₃₋₁₀ cycloalkyl, heterocyclyl, aryl and heteroaryl of R⁶, R⁷, and R⁸ is independently optionally substituted with one to six R¹²; or
two of R⁶, R⁷, and R⁸ are taken together with the atoms to which they are attached to form heterocyclyl independently optionally substituted by one to six halo, or C₁₋₁₂ alkyl independently optionally substituted by one to six oxo, halo, hydroxyl or amino;
R⁹ is independently hydrogen or C₁₋₁₂ alkyl optionally substituted with one to six halo;
each R¹² is independently halo, cyano, nitro, oxo, —OR³⁰, —SR³⁰, —SF₅, —NR³⁰R³¹, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R³⁰, —C(O)OR³⁰, —OC(O)OR³⁰, —OC(O)R³⁰, —C(O)NR³⁰R³¹, —OC(O)NR³⁰R³¹, —NR³⁰C(O)NR³⁰R³¹, —S(O)₁₋₂R³⁰, —S(O)₁₋₂NR³⁰, —NR³⁰S(O)₁₋₂R³¹, —NR³⁰S(O)₁₋₂NR³⁰R³¹, —NR³⁰C(O)R³¹ or —NR³⁰C(═O)OR³¹, wherein each C₁₋₁₂ alkyl, C₂₋₁₂ alkenyl, C₂₋₁₂ alkynyl, C₃₋₁₀ cycloalkyl, heterocyclyl, aryl and heteroaryl of R¹² is independently optionally substituted with one to six halo or C₁₋₁₂ alkyl independently optionally substituted by one to six oxo, halo, hydroxyl or amino;
each R¹³ is independently halo, cyano, nitro, oxo, —OR³⁰, —SR³⁰, —SF₅, —NR³⁰R³¹, C₁₋₁₂ alkyl, C₂₋₁₂ alkenyl, C₂₋₁₂ alkynyl, C₃₋₁₀ cycloalkyl, heterocyclyl, aryl, heteroaryl, —C(O)R³⁰, —C(O)OR³⁰, —OC(O)OR³⁰, —OC(O)R³⁰, —C(O)NR³⁰R³¹, —OC(O)NR³⁰R³¹, —NR³⁰C(O)NR³⁰R³¹, —S(O)₁₋₂R³⁰, —S(O)₁₋₂NR³⁰, —NR³⁰S(O)₁₋₂R³¹, —NR³⁰S(O)₁₋₂NR³⁰R³¹, —NR³⁰C(O)R³¹ or —NR³⁰C(═O)OR³¹, wherein each C₁₋₁₂ alkyl, C₂₋₁₂ alkenyl, C₂₋₁₂ alkynyl, C₃₋₁₀ cycloalkyl, heterocyclyl, aryl and heteroaryl of R¹³ is independently optionally substituted with one to six halo or C₁₋₁₂ alkyl independently optionally substituted by one to six oxo, halo, hydroxyl or amino;
each R²⁰ and R²¹ is independently hydrogen or C₁₋₁₂ alkyl independently optionally substituted with one to six oxo, halo, hydroxyl or amino; or
R²⁰ and R²¹ are taken together with the atoms to which they are attached to form heterocyclyl independently optionally substituted by one to six halo or C₁₋₁₂ alkyl independently optionally substituted by one to six oxo, halo, hydroxyl or amino; and
each R³⁰ and R³¹ is independently hydrogen or C₁₋₁₂ alkyl independently optionally substituted with one to six oxo, halo, hydroxyl or amino;
or R³⁰ and R³¹ are taken together with the atoms to which they are attached to form heterocyclyl independently optionally substituted by one or to six halo or C₁₋₁₂ alkyl independently optionally substituted by one to six oxo, halo, hydroxyl or amino; provided that:
the compound is not -(4-chloro-2-methylphenoxy)-N-[1-(5-cyclopropyl-1H-pyrazol-3-yl)-4-piperidinyl]-acetamide or N-[1-(5-cyclopropyl-1H-pyrazol-3-yl)-4-piperidinyl]-2-phenoxy-acetamide;
and when s is 0, two of R² on non-adjacent ring atoms together form a bond, C₁₋₃ alkylene optionally substituted with one to six R⁵, or C₁₋₂ heteroalkylene optionally substituted with one to four R⁵.

2. The compound of claim 1, wherein the compound is represented by Formula I: 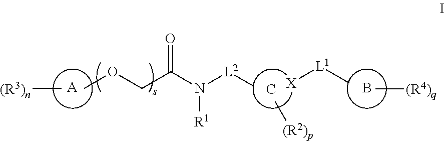
or a pharmaceutically acceptable salt, isotopically enriched analog, stereoisomer, mixture of stereoisomers or prodrug thereof, wherein
L¹ is a heteroalkylene optionally substituted with one to six R⁵ or L¹ is a heterocyclyl or heteroaryl, each of which is optionally substituted with one to six R¹³.

3. The compound of claim 1, wherein when X is C and L¹ is 5-membered heterocyclyl attached to X at a nitrogen atom, then R² is not oxo.

4. The compound of claim 1 or 2, wherein the compound is represented by Formula IA: 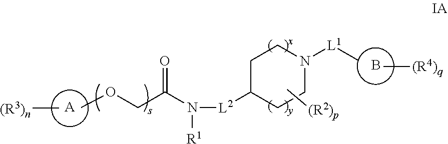
or a pharmaceutically acceptable salt, isotopically enriched analog, stereoisomer, mixture of stereoisomers or prodrug thereof, wherein x and y are independently 0, 1, or 2.

5. The compound of claim 4, wherein the compound is represented by Formula IA-1, Formula IA-2, Formula IA-3, Formula IA-4, Formula IA-5, Formula IA-6 or Formula IA-7: 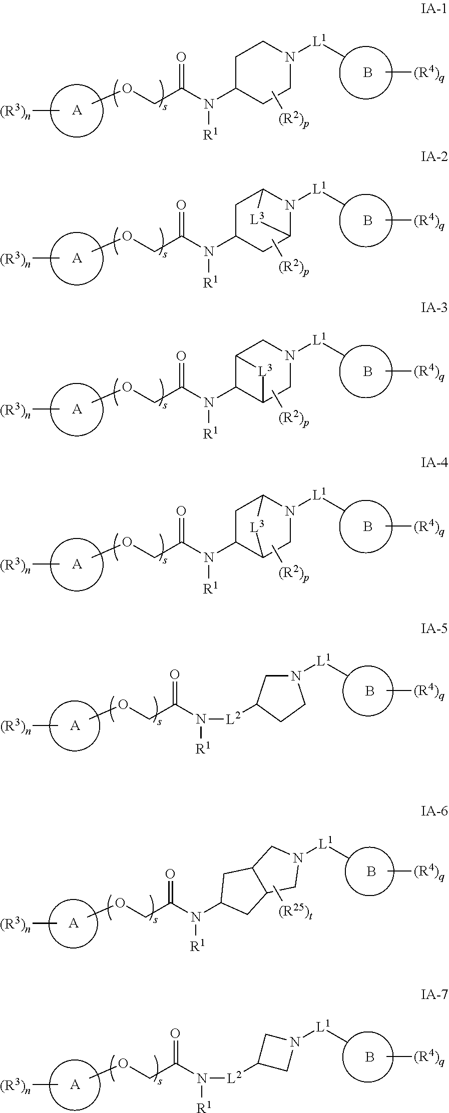
or a pharmaceutically acceptable salt, isotopically enriched analog, stereoisomer, mixture of stereoisomers or prodrug thereof, wherein
L³ is a bond or C₁₋₃ alkylene optionally substituted with one to six R⁵, or C₁₋₂ heteroalkylene optionally substituted with one to four R⁵;
R²⁵ is independently halo, C₁₋₆ alkyl or C₁₋₆ haloalkyl; and
t is 0, 1, 2, 3, 4, 5, 6, 7, or 8.

6. The compound of claim 5, wherein L³ is —CH₂—, —CH₂CH₂—, —CH₂CH₂CH₂—, or —CH₂OCH₂—, each of which is optionally substituted with one to six R⁵.

7. The compound of claim 1, wherein the compound is represented by Formula II: 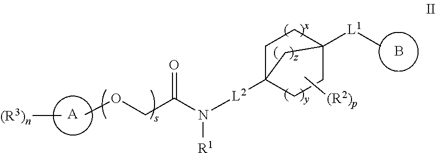
or a pharmaceutically acceptable salt, isotopically enriched analog, stereoisomer, mixture of stereoisomers or prodrug thereof, wherein x, y, and z are independently 0, 1, or 2.

8. The compound of claim 7, wherein z is 0.

9. The compound of claim 1 or 7, wherein the compound is represented by Formula IIA, Formula IIB, Formula IIC, Formula IID, or Formula IIE: 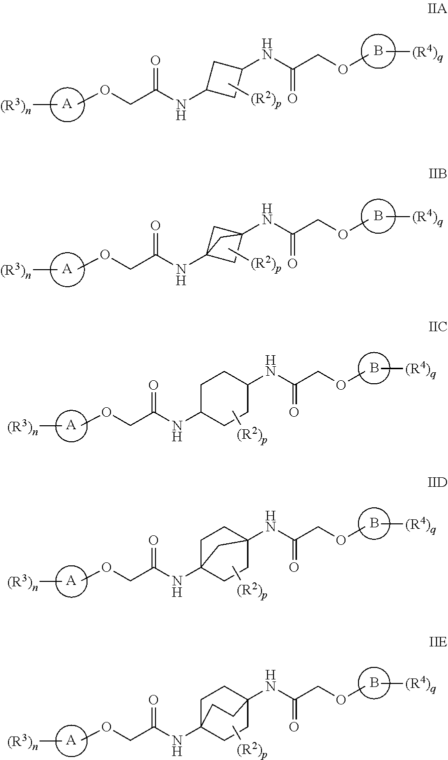
or a pharmaceutically acceptable salt, isotopically enriched analog, stereoisomer, mixture of stereoisomers or prodrug thereof.

10. The compound of claim 1, wherein the compound is represented by Formula IIIA or Formula IIIB: 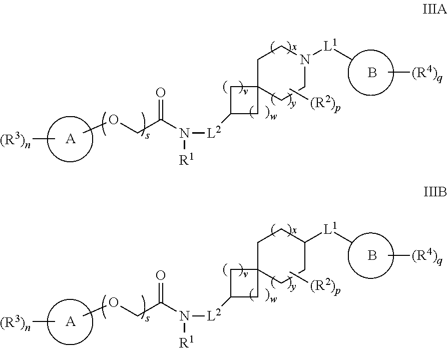
or a pharmaceutically acceptable salt, isotopically enriched analog, stereoisomer, mixture of stereoisomers or prodrug thereof, wherein x and y are independently 0, 1, or 2, and v and w are independently 1 or 2.

11. The compound of claim 10, wherein v, w, x, and y are each 1.

12. The compound of claim 10, wherein v and w are each 1, and x and y are each 0.

13. The compound of any one of claims 1-8 or 10-12, wherein L¹ is an optionally substituted heteroaryl ring.

14. The compound of claim 13, wherein L¹ is a five membered C₂₋₄ heteroaryl ring optionally substituted with one to six R¹³.

15. The compound of claim 13, wherein L¹ is a five membered C₂₋₄ heteroaryl ring having 1 to 3 nitrogen ring atoms and optionally substituted with one to six R¹³.

16. The compound of claim 13, wherein L¹ is a pyrazolyl, triazolyl, oxazolyl, imidazolyl, oxadiazolyl, or isoxazolyl, each optionally substituted with one to six R¹³.

17. The compound of any one of claims 1-8 or 10-12, wherein L¹ is a heterocyclyl ring optionally substituted with one to six R¹³.

18. The compound of claim 17, wherein L¹ is a five membered C₂₋₁ heterocyclyl optionally substituted with one to six R¹³.

19. The compound of claim 17, wherein L¹ is a five membered C₂₋₄ heterocyclyl ring having 1 to 3 nitrogen ring atoms and optionally substituted with one to six R¹³.

20. The compound of claim 17, wherein L¹ is a imidazolidinonyl, dihydroisoxazolyl or oxazolidinyl, each optionally substituted with one to six R¹³.

21. The compound of any one of claims 1-8 or 10-20, wherein L¹ is substituted with one to five R¹³ where each R¹³ is independently selected from halo, cyano, oxo, C₁₋₆ alkyl, C₁₋₆ alkoxy, C₁₋₆ haloalkyl, or C₁₋₆ haloalkoxy.

22. The compound of any one of claims 1-8 or 10-12, wherein L¹ is —C(O)CH₂O—, —OCH₂CH₂O—, —CH₂CH₂O—, or —CH₂CF₂CH₂O—.

23. The compound of any one of claims 1-8 or 10-22, wherein L² is a bond.

24. The compound of any one of claims 1-8 or 10-22, wherein L² is CH₂.

25. The compound of any one of claims 1-8 or 10-22, wherein R¹ is H.

26. The compound of any one of the preceding claims, wherein p is 0.

27. The compound of any one of the preceding claims, wherein p or t is 1 or 2.

28. The compound of any one of the preceding claims, wherein R² or R²⁵ is halo.

29. The compound of any one of the preceding claims, wherein R² or R²⁵ is C₁₋₆ alkoxy.

30. The compound of any one of the preceding claims, wherein R² or R²⁵ is methoxy.

31. The compound of any one of the preceding claims, wherein q is 0.

32. The compound of any one of the preceding claims, wherein q is 1.

33. The compound of any one of the preceding claims, wherein q is 2.

34. The compound of any one of the preceding claims, wherein n is 1.

35. The compound of any one of the preceding claims, wherein n is 2.

36. The compound of any one of the preceding claims, wherein R³ and R⁴ are independently hydroxyl, halo(C₁₋₆ alkoxy), halo, heteroaryl, heterocyclyl, cycloalkyl, cycloalkoxy, phenyl, C₁₋₆ alkoxycarbonyl, cyano, halo(C₁₋₆ alkyl), halo(C₁₋₆ alkoxy)cycloalkoxy, halo(C₁₋₆ alkoxy)alkyl, halo(heterocyclyl) or halophenoxy.

37. The compound of any one of the preceding claims, wherein R³ or R⁴ is halo(C₁₋₆ alkoxy).

38. The compound of any one of the preceding claims, wherein R³ or R⁴ is trifluoromethoxy.

39. The compound any one of the preceding claims, wherein A is C₃₋₁₀ cycloalkyl, aryl, or heteroaryl, each of which is optionally substituted with (R³)ₙ.

40. The compound of any one of claims 1-28, wherein A is cyclobutyl, triazolyl, phenyl, benzothiazolyl, quinolinyl, or chromanyl, each of which is optionally substituted with (R³)ₙ.

41. The compound of any one of claims 1-38, wherein A is phenyl optionally substituted with (R³)ₙ.

42. The compound of any one of claims 1-38, wherein A is phenyl optionally substituted with one to six R³ independently selected from halo, cyano, C₁₋₁₂ alkyl optionally substituted with one to six halo, or C₁₋₁₂ alkoxy optionally substituted with one to six halo.

43. The compound of any one of claims 1-38, wherein A is phenyl substituted with chloro, fluoro or a combination thereof.

44. The compound of any one of claims 1-38, wherein A is 4-chlorophenyl, 4-fluorophenyl, 4-chloro-3-fluorophenyl, 4-chloro-2-fluorophenyl, 2,4-difluorophenyl, 3,4-difluorophenyl, 4-methylphenyl, 2-((trifluoromethoxy)methyl)cyclopropyl or 3-(trifluoromethoxy)cyclobutyl.

45. The compound of any one of the preceding claim, wherein B is cyclopropyl, cyclobutyl, cyclopentyl, phenyl, azetidinyl, pyrrolidinyl, or tetrahydrofuranyl, each optionally substituted with (R⁴)_{q}.

46. The compound of any one of claims 1-44, wherein B is cyclobutyl optionally substituted with (R⁴)_{q}.

47. The compound of any one of claims 1-44, wherein B is phenyl optionally substituted with (R⁴)_{q}.

48. The compound of any one of claims 1-44, wherein the —B(R⁴)_{q} moiety is 1-fluorocyclopropyl, 2-methylcyclopropyl, 2,2-difluorocyclopropyl, 3-(difluoromethoxy)cyclobutyl, 3-(trifluoromethoxy)cyclobutyl, 3-(trifluoromethyl)cyclobutyl, 3-cyanocyclobutyl, 4-chloro-3-fluoro-phenyl, 4-chlorophenyl, cyanocyclobutyl, cyclobutyl, cyclopentyl, cyclopropyl, hydroxycyclobutyl, N-tert-butoxy(carbonyl)azetidin-3-yl, N-tert-butoxy(carbonyl)pyrrolidin-3-yl, tetrahydrofuranyl, 3-(1,1-difluoroethyl)cyclobutyl, 3-(1,1,1-trifluoroethyl)azetidinyl, 3-(triazol-2-yl)cyclobutyl, 3-(trifluoromethylthio)cyclobutyl, 3-(cyclopropyl)cyclobutyl, 1-(2,2,2-trifluoro-1-methyl-ethyl)azetidin-3-yl, 1-(2,2,2-trifluoroethyl)azetidin-3-yl, 1-(2,2,2-trifluoroethyl)pyrazol-3-yl, 1-(2,2,2-trifluoroethyl)pyrazol-4-yl, 1-(2,2-difluoroethyl)azetidin-3-yl, 1-tert-butoxycarbonyl-2-methylazetidin-3-yl, 2-(4-chloro-3-fluoro-phenyl, 2-(difluoromethyl)cyclopropyl, 2-(trifluoromethoxymethyl)cyclopropyl, 2-methyl-1-(2,2,2-trifluoroethyl)azetidin-3-yl, 3-(trifluoromethoxymethyl)cyclobutyl, 3-(trifluoromethyl)azetidin-1-yl, 3-fluoro-1-(2,2,2-trifluoroethyl)azetidin-3-yl, 4-(2,2,2-trifluoroethyl)morpholin-2-yl, 4-tert-butoxycarbonyl-morpholin-2-yl, 5-(trifluoromethoxymethyl)tetrahydrofuran-2-yl, 2-((trifluoromethoxy)methyl)cyclopropyl, 5-fluoro-3-pyridyl, 1-(2,2,2-trifluoroethyl)pyrrolidin-3-yl, 3-ethoxycyclobutanyl, 3-(2,2,2-trifluoroethyl)cyclobutyl or 3-isopropoxycyclobutanyl.

49. The compound of any one of claims 1-44, wherein the —B(R⁴)_{q} moiety is 1-fluorocyclopropyl, 2-methylcyclopropyl, 2,2-difluorocyclopropyl, 3-(difluoromethoxy)cyclobutyl, 3-(trifluoromethoxy)cyclopropyl, 3-(trifluoromethyl)cyclobutyl, 3-cyanocyclobutyl, 4-chloro-3-fluoro-phenyl, 4-chlorophenyl, phenyl, 3-cyanocyclobutyl, cyclobutyl, cyclopentyl, cyclopropyl, cyanocyclopropyl, hydroxycyclobutyl, N-tert-butoxy(carbonyl)azetidin-3-yl, N-(2,2,2-trifluoroethyl)azetidin-3-yl, N-tert-butoxy(carbonyl)pyrrolidin-3-yl, tetrahydrofuranyl, 3-(difluoromethoxy)cyclobutyl, 3-(trifluoromethoxy)cyclobutyl, 3-(1,1-difluoroethyl)cyclobutyl, 3-(1,1,1-trifluoroethyl)azetidinyl, 3-(triazol-2-yl)cyclobutyl, 3-(trifluoromethylthio)cyclobutyl, 3-(2,2,2-trifluoroethyl)cyclobutyl, or 3-(cyclopropyl)cyclobutyl.

50. The compound of any one of claims 1-44, wherein the —B(R⁴)_{q} moiety is cyclopropyl, 3,3-difluorocyclobutyl, 3-(trifluoromethyl)cyclobutyl, spiro[3.3]heptan-2-yl, 3,3-dimethylcyclobutyl, 3-cyanocyclobutyl, 3-cyano-1-methylcyclobutyl, 3-fluorocyclobutyl, 3-cyano-3-methylcyclobutyl, 3-(triazol-2-yl)cyclobutyl, 3-(difluoromethoxy)cyclobutyl, 3-methoxycyclobutyl, 3-methylcyclobutyl, 3-(difluoromethyl)cyclobutyl,

51. The compound of any one of claims 1-44, wherein the —B(R⁴)_{q} moiety is 4-chloro-3-fluoro-phenyl, N-(2,2,2-trifluoroethyl)azetidin-3-yl, 3-(difluoromethoxy)cyclobutyl, or 3-(trifluoromethoxy)cyclobutyl.

52. The compound of any one of claims 1-8, 10-12, 23-30 or 34-44, wherein the -L¹-B—(R⁴)_{q} moiety is (4-chloro-3-fluoro-phenoxy)methyl]-1,3,4-oxadiazol-2-yl, 1-(3-cyanocyclobutyl)triazol-4-yl, 1-(3-hydroxycyclobutyl)triazol-4-yl, 1-(4-chlorophenyl)triazol-4-yl, 1-benzyltriazol-4-yl, 1-cyclobutyltriazol-4-yl, 1H-1,2,3-triazol-4-yl, 2-(3-cyanocyclobutyl)triazol-4-yl, 2-(trifluoromethoxy)ethyl]-1,3,4-oxadiazol-2-yl, 2-cyclobutyltriazol-4-yl, 3-[(trifluoromethoxy)cyclobutoxy]-imidazol-1-yl, 3-cyanocyclobutyl)triazol-4-yl, 3-cyclobutylisoxazol-5-yl, 4-(cyclobutylmethyl)imidazol-1-yl, 4-[3-(trifluoromethoxy)cyclobutyl]imidazol-1-yl, 4-cyclobutylimidazol-1-yl, 4-cyclobutyloxazol-2-yl, 5-((4-chloro-3-fluorophenoxy)methyl)-4H-1,2,4-triazol-3-yl, 5-(1-fluorocyclopropyl)-1,3,4-oxadiazol-2-yl, 5-(2-cyclopropylethyl)-1,3,4-oxadiazol-2-yl, 5-(2,2-difluorocyclopropyl)-1,3,4-oxadiazol-2-yl, 5-(2,2,2-trifluoroethyl)-1,3,4-oxadiazol-2-yl, 5-(3-cyanocyclobutyl)-1,3,4-oxadiazol-2-yl, 5-(3,3-difluoro-1-methyl-propyl)-1,3,4-oxadiazol-2-yl, 5-(4-chloro-3-fluoro-phenyl)-1,3,4-oxadiazol-2-yl, 5-(cyclobutoxymethyl)-1,3,4-oxadiazol-2-yl, 5-(cyclobutylmethyl)-1,3,4-oxadiazol-2-yl, 5-(cyclopropylmethyl)-1,3,4-oxadiazol-2-yl, 5-(trifluoromethoxymethyl)-1,3,4-oxadiazol-2-yl, 5-[(4-chloro-3-fluoro-phenoxy)methyl]-1,3,4-oxadiazol-2-yl, 5-[[3-(trifluoromethoxy)cyclobutoxy]methyl]-1,3,4-oxadiazol-2-yl, 5-[2-methylcyclopropyl]-1,3,4-oxadiazol-2-yl, 5-[3-(trifluoromethoxy)cyclobutyl]-1,3,4-oxadiazol-2-yl, 5-[3-(trifluoromethoxy)propyl]-1,3,4-oxadiazol-2-yl, 5-[3-(trifluoromethyl)cyclobutyl]-1,3,4-oxadiazol-2-yl, 5-[N-(1,1,1-trifluoroethyl)azetidin-3-yl]-1,3,4-oxadiazol-2-yl, 5-[N-(1,1,1-trifluoroethyl)pyrrolidin-3-yl]-1,3,4-oxadiazol-2-yl, 5-[N-tert-butoxy(carbonyl)azetidin-3-yl]-1,3,4-oxadiazol-2-yl, 5-[N-tert-butoxy(carbonyl)pyrrolidin-3-yl]-1,3,4-oxadiazol-2-yl, 5-cyclobutyl-1,3,4-oxadiazol-2-yl, 5-cyclobutyl-4,5-dihydroisoxazol-3-yl, 5-cyclobutylisoxazol-3-yl, 5-cyclobutyloxazol-2-yl, 5-cyclopentyl-4,5-dihydroisoxazol-3-yl, oxazolidin-2-one-5-yl, (3-(trifluoromethoxy)cyclobutoxy)eth-2-yl, 1-(3-(trifluoromethoxy)cyclobutyl)-1H-pyrazol-4-yl, 2,2-difluoro-3-(3-(trifluoromethoxy)cyclobutoxy)propyl, 2-(3-(trifluoromethoxy)cyclobutyl)pyrimidin-4-yl, 3-(2,2,2-trifluoroethyl)cyclobutoxy)methyl, 2-oxo-3-[3-(trifluoromethoxy)cyclobutyl]imidazolidin-1-yl, or 2-(3-(trifluoromethoxy)cyclobutoxy)acetyl.

53. A compound or a pharmaceutically acceptable salt, isotopically enriched analog, stereoisomer, mixture of stereoisomers or prodrug thereof selected from:

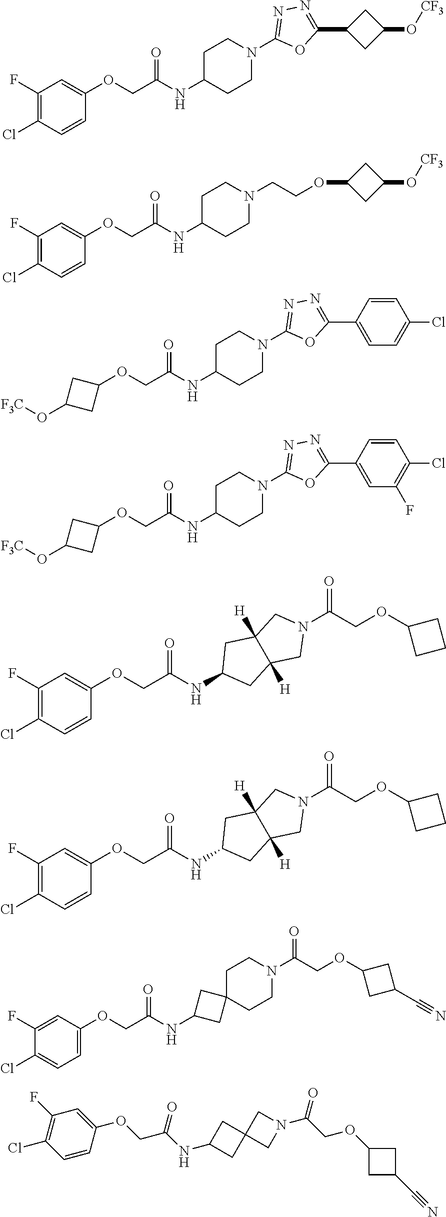

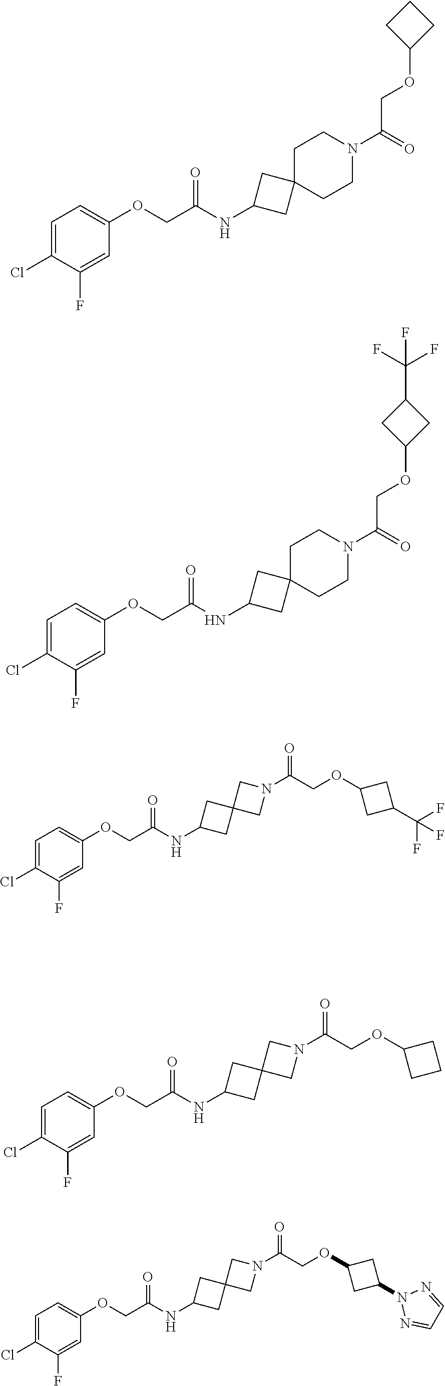 [SMILES: O=C(COc1ccc(Cl)c(F)c1)NC1CC2(CCN(C(=O)COC3CCC3)CC2)C1 ; O=C(COc1ccc(Cl)c(F)c1)NC1CC2(CCN(C(=O)COC3CC(C(F)(F)F)C3)CC2)C1 ; O=C(COc1ccc(Cl)c(F)c1)NC1CC2(C1)CN(C(=O)COC1CC(C(F)(F)F)C1)C2 ; O=C(COc1ccc(Cl)c(F)c1)NC1CC2(C1)CN(C(=O)COC1CCC1)C2 ; O=C(COc1ccc(Cl)c(F)c1)NC1CC2(C1)CN(C(=O)CO[C@H]1C[C@@H](n3nccn3)C1)C2]

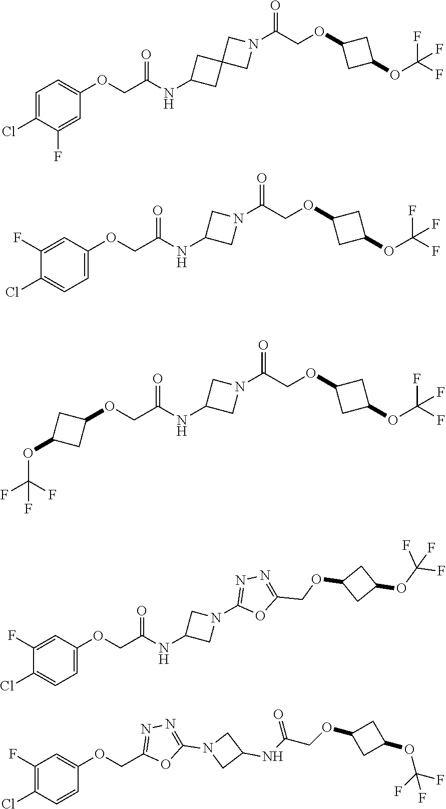

54. A compound or a pharmaceutically acceptable salt, isotopically enriched analog, stereoisomer, mixture of stereoisomers or prodrug thereof selected from:

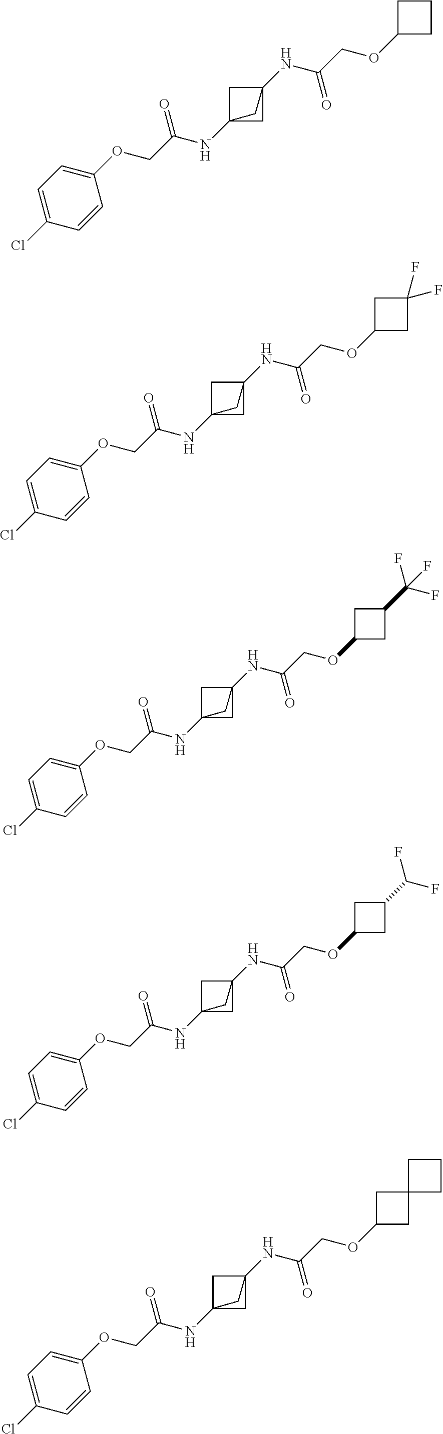 [SMILES: O=C(COc1ccc(Cl)cc1)NC12CC(NC(=O)COC3CCC3)(C1)C2 ; O=C(COc1ccc(Cl)cc1)NC12CC(NC(=O)COC3CC(F)(F)C3)(C1)C2 ; O=C(COc1ccc(Cl)cc1)NC12CC(NC(=O)CO[C@H]3C[C@@H](C(F)(F)F)C3)(C1)C2 ; O=C(COc1ccc(Cl)cc1)NC12CC(NC(=O)CO[C@H]3C[C@H](C(F)F)C3)(C1)C2 ; O=C(COc1ccc(Cl)cc1)NC12CC(NC(=O)COC3CC4(CCC4)C3)(C1)C2]

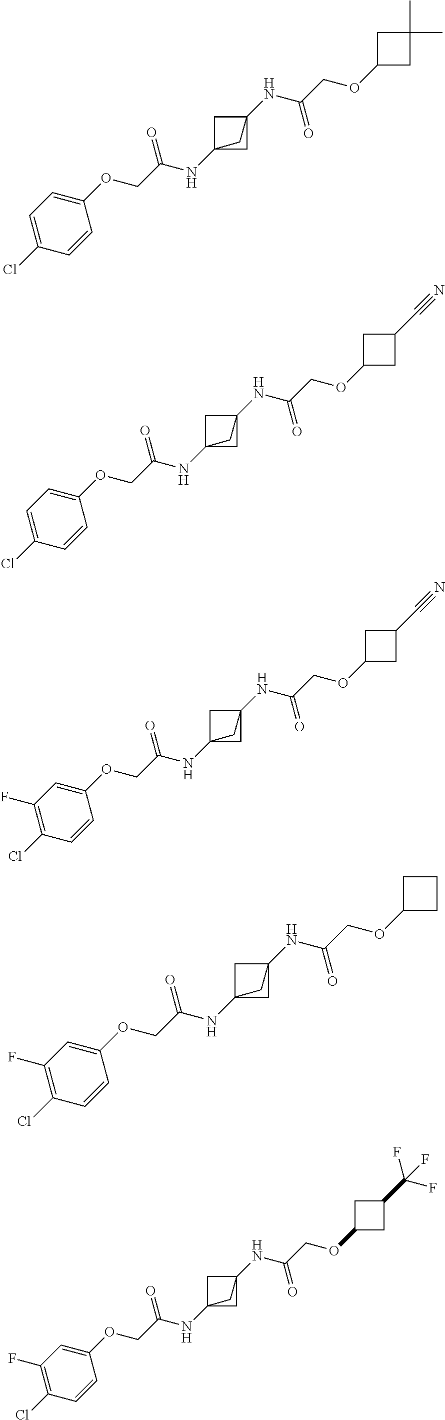 [SMILES: CC1(C)CC(OCC(=O)NC23CC(NC(=O)COc4ccc(Cl)cc4)(C2)C3)C1 ; N#CC1CC(OCC(=O)NC23CC(NC(=O)COc4ccc(Cl)cc4)(C2)C3)C1 ; N#CC1CC(OCC(=O)NC23CC(NC(=O)COc4ccc(Cl)c(F)c4)(C2)C3)C1 ; O=C(COc1ccc(Cl)c(F)c1)NC12CC(NC(=O)COC3CCC3)(C1)C2 ; O=C(COc1ccc(Cl)c(F)c1)NC12CC(NC(=O)CO[C@H]3C[C@@H](C(F)(F)F)C3)(C1)C2]

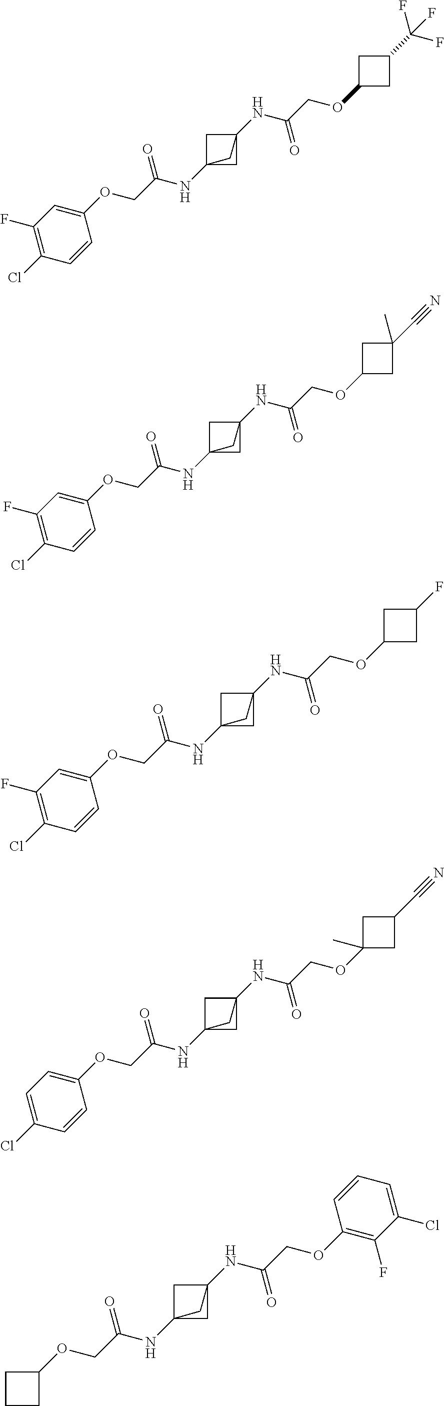 [SMILES: O=C(COc1ccc(Cl)c(F)c1)NC12CC(NC(=O)CO[C@H]3C[C@H](C(F)(F)F)C3)(C1)C2 ; CC1(C#N)CC(OCC(=O)NC23CC(NC(=O)COc4ccc(Cl)c(F)c4)(C2)C3)C1 ; O=C(COc1ccc(Cl)c(F)c1)NC12CC(NC(=O)COC3CC(F)C3)(C1)C2 ; CC1(OCC(=O)NC23CC(NC(=O)COc4ccc(Cl)cc4)(C2)C3)CC(C#N)C1 ; O=C(COc1cccc(Cl)c1F)NC12CC(NC(=O)COC3CCC3)(C1)C2]

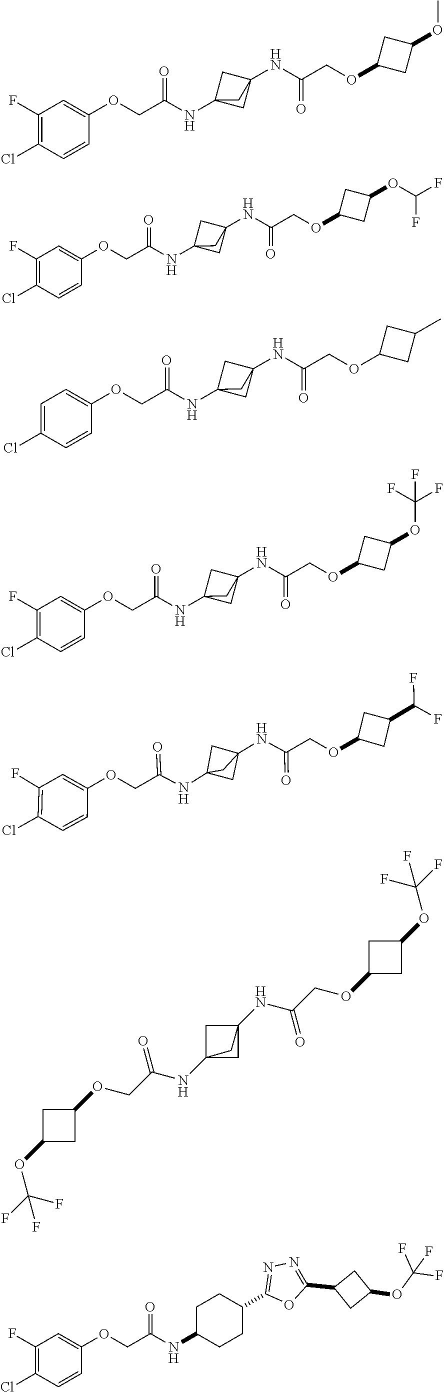

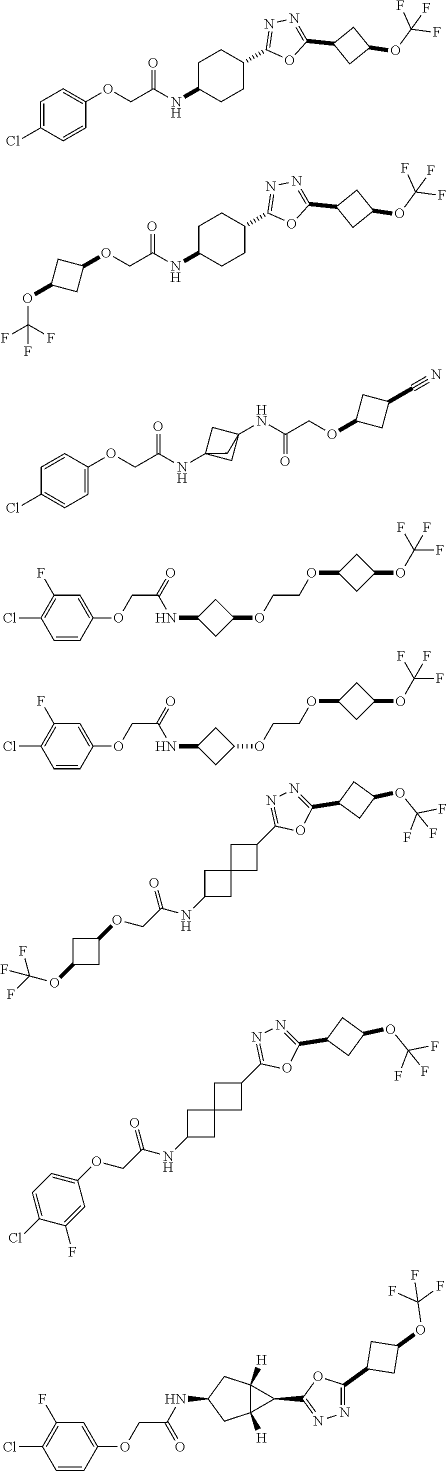

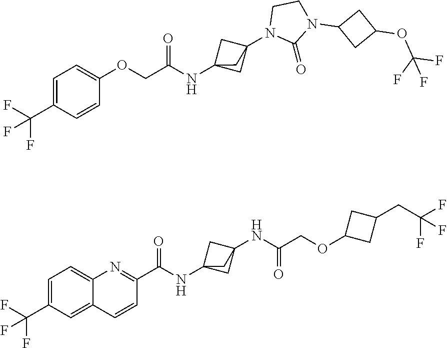 [SMILES: O=C(COc1ccc(C(F)(F)F)cc1)NC12CC(N3CCN(C4CC(OC(F)(F)F)C4)C3=O)(C1)C2 ; O=C(COC1CC(CC(F)(F)F)C1)NC12CC(NC(=O)c3ccc4cc(C(F)(F)F)ccc4n3)(C1)C2]

55. A pharmaceutical composition comprising a compound of any one of the preceding claims, or a pharmaceutically acceptable salt, stereoisomer, mixture of stereoisomers or prodrug thereof, and a pharmaceutically acceptable carrier.

56. A method for treating a disease or condition mediated, at least in part, by eukaryotic initiation factor 2B, the method comprising administering an effective amount of the pharmaceutical composition of claim 55 to a subject in need thereof.

57. The method of claim 56, wherein the disease or condition is a neurodegenerative disease.

58. The method of claim 56, wherein the disease is Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, insulin resistance, Tabes dorsalis or vanishing white matter (VWM) disease.

59. The method of claim 57, wherein the neurodegenerative disease is Alzheimer's disease, ALS, or vanishing white matter disease.

60. The method of claim 56, wherein the disease or condition is cancer.

61. A method for enhancing cognitive memory, the method comprising administering an effective amount of the pharmaceutical composition of claim 55 to a subject in need thereof.

62. Use of a compound of any one of claims 1-54, or a pharmaceutically acceptable salt, stereoisomer, mixture of stereoisomers, or prodrug thereof, for treating a disease or condition mediated, at least in part, by eukaryotic initiation factor 2B.

63. The use of claim 62, wherein the disease or condition is a neurodegenerative disease.

64. The use of claim 62, wherein the disease is Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, narcolepsy, Neuroborreliosis, Parkinson's disease, Pelizacus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, vanishing white matter (VWM) disease, insulin resistance or Tabes dorsalis.

65. A compound of any one of claims 1-54, or a pharmaceutically acceptable salt, stereoisomer, mixture of stereoisomers, or prodrug thereof, for use in therapy.

66. A compound of any one of claims 1-54, or a pharmaceutically acceptable salt, stereoisomer, mixture of stereoisomers, or prodrug thereof, 54 use in treating a neurodegenerative disease.

67. A compound of any one of claims 1-54, or a pharmaceutically acceptable salt, stereoisomer, mixture of stereoisomers, or prodrug thereof, for use in treating cancer.

68. A compound of any one of claims 1-54, or a pharmaceutically acceptable salt, stereoisomer, mixture of stereoisomers, or prodrug thereof, for use in enhancing cognitive memory.

69. The use of a compound of claims 1-54, or a pharmaceutically acceptable salt, stereoisomer, mixture of stereoisomers, or prodrug thereof, for the manufacture of a medicament for treating a neurodegenerative disease, treating cancer or enhancing cognitive memory.
